(19) United States
(12) Patent Application Publication
(10) Pub. No.: US 2026/0297511 A1
(43) Pub. Date: Oct. 1, 2026

(54) METHODS AND COMPOSITIONS FOR PRENATAL TESTING

(71) Applicant: EarlyCell, Inc., Cleveland, OH (US)

(72) Inventors: Paul David JACKSON, Cleveland, OH (US); Kazusa SATO, Cleveland, OH (US)

(21) Appl. No.: 19/401,916

(22) Filed: Nov. 26, 2025

Related U.S. Application Data
(63) Continuation of application No. PCT/US2024/031784, filed on May 30, 2024.
(60) Provisional application No. 63/505,228, filed on May 31, 2023.

(51) Int. Cl. C12N 5/073; C12N 5/00

(52) U.S. Cl. CPC C12N 5/0605; C12N 5/0018; C12N 5/0081

(57) ABSTRACT

The present disclosure provides methods and systems directed to isolation of circulating fetal cells (CFCs) from maternal biological samples of pregnancy-related states. A method for identifying genetic disorders of the fetus (or fetuses) of a subject may involve assaying a biological sample derived from the subject to isolate CFCs, and characterizing the biomarkers, such as the sequence of genetic material or abundance of gene expression material, from the CFCs to determine the presence or absence of a pregnancy-related states, such as a genetic order or ongoing health risk to the mother or fetus.

### CROSS-REFERENCE TO RELATED APPLICATIONS

This application is a continuation of International Application No. PCT/US2024/031784, filed on May 30, 2024, which claims the benefit of U.S. Provisional Application No. 63/505,228, filed on May 31, 2023, each of which is incorporated by reference herein in its entirety for all purposes.

### BACKGROUND

Every year, about 7.9 million infants (e.g., 6% of worldwide births) are born with serious birth defect. Of those 7.9 million infants, about 3.2 million of these infants are disabled for life. Current prenatal testing methods for these birth defects are limited to imperfect (e.g., damaged) testing material (e.g., cell-free fetal DNA) and are unable to be performed until 10 weeks of pregnancy.

### SUMMARY

In some embodiments, the present disclosure provides fetal-specific biomarkers and maternal-specific biomarkers. In some embodiments, the fetal-specific biomarkers and the maternal-specific biomarkers can be used individually or in combinations to distinguish circulating fetal cells from maternal cells in maternal blood samples. In some embodiments, the fetal-specific biomarkers and the maternal-specific biomarkers can purify the fetal cells from the maternal blood sample. In some embodiments, the purified fetal cells are used for prenatal genetic and gene expression analysis. In some embodiments, the present disclosure provides methods of purifying the circulating fetal cells from a maternal blood sample using the fetal-specific biomarkers and the maternal-specific biomarkers disclosed herein. In some embodiments, the circulating fetal cells can be purified from a maternal blood sample acquired during the first trimester of gestation. In some embodiments, the present disclosure provides methods for performing genetic analyses on the purified fetal cells. In some embodiments, the genetic analysis comprises fetal gene expression analysis. In some embodiments, the genetic analysis of the purified fetal cells can identify fetal genetic risk factors or gene expression markers of fetal health defects or pregnancy health defects.

In some embodiments, the present disclosure provides a composition comprising: an ex vivo population of cells from a maternal blood sample, wherein the ex vivo population of cells comprises a subpopulation of maternal cells and a subpopulation of fetal cells, wherein the subpopulation of fetal cells is at least about 75% of cells in the ex vivo population of cells.

In some embodiments, the subpopulation of fetal cells is at least about 80% of the cells in the ex vivo population of cells. In some embodiments, the subpopulation of fetal cells is at least about 90% of the cells in the ex vivo population of cells.

In some embodiments, the subpopulation of maternal cells is no greater than about 30% of the cells in the ex vivo population of cells. In some embodiments, the subpopulation of maternal cells is no greater than about 20% of the cells in the ex vivo population of cells. In some embodiments, the subpopulation of maternal cells is no greater than about 10% of the cells in the ex vivo population of cells.

In some embodiments, the subpopulation of fetal cells comprises trophoblasts.

In some embodiments, the composition further comprises a second subpopulation of fetal cells, wherein the second subpopulation of fetal cells is different in cell type from the subpopulation of fetal cells. In some embodiments, the second subpopulation of fetal cells comprises erythroblasts. In some embodiments, the second subpopulation of fetal cells comprises megakaryocytes. In some embodiments, the second subpopulation of fetal cells comprises vascular endothelial cells. In some embodiments, the second subpopulation of fetal cells comprises fetal stromal cells. In some embodiments, the subpopulation of fetal cells comprises early gestation fetal cells, and the second subpopulation of fetal cells comprises later gestation fetal cells.

In some embodiments, the subpopulation of maternal cells and the subpopulation of fetal cells in the ex vivo population of cells is quantified by flow cytometry.

In some embodiments, the present disclosure provides a method comprising: sequencing a nucleic acid sequence, wherein the nucleic acid sequence is from an ex vivo population of cells from a maternal blood sample, wherein the ex vivo population of cells comprises a subpopulation of maternal cells and a subpopulation of fetal cells, wherein the subpopulation of fetal cells is at least about 70% of cells in the ex vivo population of cells.

In some embodiments, the subpopulation of fetal cells is at least about 80% of the cells in the ex vivo population of cells. In some embodiments, the subpopulation of fetal cells is at least about 90% of the cells in the ex vivo population of cells.

In some embodiments, the subpopulation of maternal cells is no greater than about 30% of the cells in the ex vivo population of cells. In some embodiments, the subpopulation of maternal cells is no greater than about 20% of the cells in the ex vivo population of cells. In some embodiments, the subpopulation of maternal cells is no greater than about 10% of the cells in the ex vivo population of cells.

In some embodiments, the method further comprises, prior to the sequencing the nucleic acid sequence, extracting the subpopulation of maternal cells and the subpopulation of fetal cells from the maternal blood sample to provide an extract, wherein the extract comprises the subpopulation of maternal cells and the subpopulation of fetal cells from the maternal blood sample. In some embodiments, the method further comprises enriching the subpopulation of fetal cells in the extract to provide a sample enriched in fetal cells and depleted in maternal cells. In some embodiments, the method further comprises sorting the subpopulation of fetal cells in the extract from the subpopulation of maternal cells in the extract to provide a sample enriched in fetal cells and depleted in maternal cells.

In some embodiments, prior to the sequencing the nucleic acid sequence, the subpopulation of maternal cells and the subpopulation of fetal cells are extracted from the maternal blood sample to provide an extract, wherein the extract comprises the subpopulation of maternal cells and the subpopulation of fetal cells from the maternal blood sample. In some embodiments, the subpopulation of fetal cells in the extract is enriched from the subpopulation of maternal cells in the extract to provide a sample enriched in fetal cells and depleted in maternal cells. In some embodiments, the subpopulation of fetal cells in the extract is sorted from the subpopulation of maternal cells in the extract to provide a sample enriched in fetal cells and depleted in maternal cells.

In some embodiments, the subpopulation of fetal cells comprises trophoblasts.

In some embodiments, the ex vivo population of cells further comprises a second subpopulation of fetal cells, wherein the second subpopulation of fetal cells is different in cell type from the subpopulation of fetal cells. In some embodiments, the second subpopulation of fetal cells comprises erythroblasts. In some embodiments, the second subpopulation of fetal cells comprises megakaryocytes. In some embodiments, the second subpopulation of fetal cells comprises vascular endothelial cells. In some embodiments, the second subpopulation of fetal cells comprises stromal cells. In some embodiments, the subpopulation of fetal cells comprises early gestation fetal cells and the second subpopulation of fetal cells comprises later gestation fetal cells.

In some embodiments, the method further comprises, prior to the sequencing the nucleic acid sequence, isolating the subpopulation of fetal cells from the maternal blood sample by contacting the maternal blood sample with a first isolation agent and coupling the first isolation agent to a first biomarker in the subpopulation of fetal cells. In some embodiments, the method further comprises incubating the maternal blood sample after the contacting. In some embodiments, the first isolation agent is a nucleic acid probe. In some embodiments, the first isolation agent is a ligand. In some embodiments, the first isolation agent couples to an intracellular biomarker. In some embodiments, the first isolation agent couples to a cell surface biomarker. In some embodiments, the first isolation agent couples to a soluble cell surface biomarker. In some embodiments, the first isolation agent couples to a nucleic acid biomarker.

In some embodiments, prior to the sequencing the nucleic acid sequence, the subpopulation of fetal cells is isolated from the maternal blood sample, wherein the maternal blood sample is contacted with a first isolation agent and the first isolation agent couples to a first biomarker in the subpopulation of fetal cells. In some embodiments, after the contacting, the maternal blood sample is incubated. In some embodiments, the first isolation agent is a nucleic acid probe. In some embodiments, the first isolation agent is a ligand. In some embodiments, the first isolation agent couples to a cell surface biomarker. In some embodiments, the first isolation agent couples to a soluble cell surface biomarker. In some embodiments, the first isolation agent couples to an intracellular biomarker. In some embodiments, the first isolation agent couples to a nucleic acid biomarker.

In some embodiments, prior to the sequencing the nucleic acid sequence, the subpopulation of maternal cells is isolated from the maternal blood sample, wherein the maternal blood sample is contacted with a second isolation agent and the second isolation agent couples to a second biomarker in the subpopulation of maternal cells. In some embodiments, after the contacting, the maternal blood sample is incubated. In some embodiments, the second isolation agent is a nucleic acid probe. In some embodiments, the second isolation agent is a ligand. In some embodiments, the second isolation agent couples to a cell surface biomarker. In some embodiments, the second isolation agent couples to a soluble cell surface biomarker. In some embodiments, the second isolation agent couples to an intracellular biomarker. In some embodiments, the second isolation agent couples to a nucleic acid biomarker.

In some embodiments, the method further comprises, prior to the sequencing the nucleic acid sequence, isolating the subpopulation of maternal cells from the maternal blood sample by contacting the maternal blood sample with a second isolation agent and coupling the second isolation agent to a second biomarker in the subpopulation of maternal cells. In some embodiments, the method further comprises incubating the maternal blood sample after the contacting. In some embodiments, the second isolation agent is a nucleic acid probe. In some embodiments, the second isolation agent is a ligand. In some embodiments, the second isolation agent couples to a cell surface biomarker. In some embodiments, the second isolation agent couples to a soluble cell surface biomarker. In some embodiments, the second isolation agent couples to an intracellular biomarker. In some embodiments, the second isolation agent couples to a nucleic acid biomarker.

In some embodiments, the sequencing is performed on a next generation sequencing platform.

In some embodiments, the nucleic acid sequence is from the subpopulation of fetal cells. In some embodiments, the nucleic acid sequence is a DNA sequence. In some embodiments, the nucleic acid sequence is a RNA sequence.

In some embodiments, the subpopulation of maternal cells and the subpopulation of fetal cells in the ex vivo population of cells is quantified by flow cytometry.

In some embodiments, the present disclosure provides a method comprising: performing an assay on an ex vivo population of cells from a maternal blood sample, wherein the ex vivo population of cells comprises a subpopulation of maternal cells and a subpopulation of fetal cells, wherein the subpopulation of fetal cells is at least about 70% of cells in the ex vivo population of cells.

In some embodiments, the assay is a cell morphology assay. In some embodiments, the assay is a cell counting assay. In some embodiments, the assay is a cell viability assay. In some embodiments, the assay is a cell proliferation assay. In some embodiments, the assay is a cytotoxicity assay.

In some embodiments, the assay indicates a likelihood that a fetus associated with the subpopulation of fetal cells is susceptible to a genetic condition. In some embodiments, the genetic condition is down syndrome. In some embodiments, the genetic condition is cystic fibrosis. In some embodiments, the genetic condition is muscular dystrophy. In some embodiments, the genetic condition is sickle cell disease. In some embodiments, the genetic condition is Tay-Sachs disease.

In some embodiments, the subpopulation of maternal cells and the subpopulation of fetal cells in the ex vivo population of cells is quantified by flow cytometry. In some embodiments, the subpopulation of maternal cells and the subpopulation of fetal cells in the ex vivo population of cells is quantified by microscopy.

In some embodiments, the present disclosure provides a method comprising: (a) sequencing a first nucleic acid sequence obtained from a first type of fetal cell from an ex vivo population of fetal cells, (b) sequencing a second nucleic acid sequence obtained from a second type of fetal cell from the ex vivo population of fetal cells, (c) sequencing a third nucleic acid sequence obtained from a third type of fetal cell from the ex vivo population of fetal cells, (d) sequencing a fourth nucleic acid sequence obtained from a fourth type of fetal cell from the ex vivo population of fetal cells, (e) sequencing a fifth nucleic acid sequence obtained from a fifth type of fetal cell from the ex vivo population of fetal cells, and (f) analyzing at least one of the first nucleic acid sequence, the second nucleic acid sequence, the third nucleic acid sequence, the fourth nucleic acid sequence, or the fifth nucleic acid sequence to determine a likelihood that a fetus associated with an ex vivo population of fetal cells is susceptible to a genetic condition.

In some embodiments, at least one of the first nucleic acid sequence, the second nucleic acid sequence, the third nucleic acid sequence, the fourth nucleic acid sequence, or the fifth nucleic acid sequence comprises a DNA sequence. In some embodiments, at least one of the first nucleic acid sequence, the second nucleic acid sequence, the third nucleic acid sequence, the fourth nucleic acid sequence, or the fifth nucleic acid sequence comprises a RNA sequence.

In some embodiments, the ex vivo population of fetal cells is in a solution comprising a Artiodactyla serum.

In some embodiments, the method further comprises, prior to (a)-(f), extracting an ex vivo population of maternal cells and the ex vivo population of fetal cells from a maternal blood sample to provide an extract, wherein the extract comprises the ex vivo population of maternal cells and the ex vivo population of fetal cells. In some embodiments, the maternal blood sample is from a pregnant subject. In some embodiments, the maternal blood sample is blood plasma. In some embodiments, the maternal blood sample is blood serum. In some embodiments, the maternal blood sample is buffy coat. In some embodiments, the maternal blood sample comprises peripheral blood mononuclear white blood cells. In some embodiments, the maternal blood sample comprises nucleated white blood cells. In some embodiments, the maternal blood sample is whole blood. In some embodiments, the method further comprises enriching the ex vivo population of fetal cells in the extract from the ex vivo population of maternal cells in the extract to provide a sample enriched in fetal cells and depleted in maternal cells. In some embodiments, the method further comprises sorting the ex vivo population of fetal cells in the extract from the ex vivo population of maternal cells in the extract to provide a sample enriched in fetal cells and depleted in maternal cells.

In some embodiments, prior to (a)-(f), the ex vivo population of fetal cells and an ex vivo population of maternal cells are extracted from a maternal blood sample to provide an extract, wherein the extract comprises the ex vivo population of fetal cells and an ex vivo population of maternal cells. In some embodiments, the maternal blood sample is from a pregnant subject. In some embodiments, the maternal blood sample is blood plasma. In some embodiments, the maternal blood sample is blood serum. In some embodiments, the maternal blood sample is buffy coat. In some embodiments, the maternal blood sample comprises peripheral blood mononuclear white blood cells. In some embodiments, the maternal blood sample comprises nucleated white blood cells. In some embodiments, the maternal blood sample is whole blood. In some embodiments, the ex vivo population of fetal cells in the extract is enriched from the ex vivo population of maternal cells in the extract to provide a sample enriched in fetal cells and depleted in maternal cells. In some embodiments, the ex vivo population of fetal cells in the extract is sorted from the ex vivo population of maternal cells in the extract to provide a sample enriched in fetal cells and depleted in maternal cells.

In some embodiments, the method further comprises, prior to (a)-(f), incubating a blood sample comprising the ex vivo population of fetal cells and an ex vivo population of maternal cells with a first isolation agent and coupling the first isolation agent to a first biomarker in the ex vivo population of fetal cells. In some embodiments, the method further comprises sorting the ex vivo population of fetal cells based on a presence of the first isolation agent that couples to the first biomarker in the ex vivo population of fetal cells. In some embodiments, the method further comprises sorting the ex vivo population of fetal cells based on an absence of a second isolation agent that couples to a second biomarker in the ex vivo population maternal cells. In some embodiments, the first isolation agent is a nucleic acid probe. In some embodiments, the first isolation agent is a ligand. In some embodiments, the first isolation agent couples to a cell surface biomarker. In some embodiments, the first isolation agent couples to a soluble cell surface biomarker. In some embodiments, the first isolation agent couples to an intracellular biomarker. In some embodiments, the first isolation agent couples to a nucleic acid biomarker.

In some embodiments, prior to (a)-(f), a blood sample comprising the ex vivo population of fetal cells and an ex vivo population of maternal cells is incubated with a first isolation agent that couples to a first biomarker in the ex vivo population of fetal cells. In some embodiments, the ex vivo population of fetal cells is sorted based on a presence of the first isolation agent that couples to the first biomarker in the ex vivo population of fetal cells. In some embodiments, the ex vivo population of fetal cells is sorted based on an absence of a second isolation agent that couples to a second biomarker in the ex vivo population maternal cells. In some embodiments, the first isolation agent is a nucleic acid probe. In some embodiments, the first isolation agent is a ligand. In some embodiments, the first isolation agent couples to a cell surface biomarker. In some embodiments, the first isolation agent couples to a soluble cell surface biomarker. In some embodiments, the first isolation agent couples to an intracellular biomarker. In some embodiments, the first isolation agent couples to a nucleic acid biomarker.

In some embodiments, the method further comprises, prior to (a)-(f), incubating a blood sample comprising the ex vivo population of fetal cells and an ex vivo population of maternal cells with a second isolation agent, and coupling the second isolation agent to a second biomarker in the ex vivo population of maternal cells. In some embodiments, the method further comprises sorting the ex vivo population of maternal cells based on a presence of the second isolation agent that couples to the second biomarker in the ex vivo population of maternal cells. In some embodiments, the method further comprises sorting the ex vivo population of maternal cells based on an absence of a first isolation agent that couples to a first biomarker in the ex vivo population of fetal cells. In some embodiments, the second isolation agent couples to an intracellular biomarker. In some embodiments, the second isolation agent is a nucleic acid probe. In some embodiments, the second isolation agent is a ligand. In some embodiments, the second isolation agent couples to a cell surface biomarker. In some embodiments, the second isolation agent couples to a soluble cell surface biomarker. In some embodiments, the second isolation agent couples to a nucleic acid biomarker.

In some embodiments, prior to (a)-(f), a blood sample comprising the ex vivo population of fetal cells and an ex vivo population of maternal cells is incubated with a second isolation agent that couples to a second biomarker in the ex vivo population of maternal cells. In some embodiments, the ex vivo population of maternal cells is sorted based on a presence of the second isolation agent that couples to the second biomarker in the ex vivo population of maternal cells. In some embodiments, the ex vivo population of maternal cells is sorted based on an absence of a first isolation agent that couples to a first biomarker in the ex vivo population of fetal cells. In some embodiments, the second isolation agent is a nucleic acid probe. In some embodiments, the second isolation agent is a ligand. In some embodiments, the second isolation agent couples to a cell surface biomarker. In some embodiments, the second isolation agent couples to a soluble cell surface biomarker. In some embodiments, the second isolation agent couples to an intracellular biomarker. In some embodiments, the second isolation agent couples to a nucleic acid biomarker.

In some embodiments, the ex vivo population of fetal cells comprises early gestation trophoblasts. In some embodiments, the ex vivo population of fetal cells comprises later gestation trophoblasts. In some embodiments, the ex vivo population of fetal cells comprises erythroblasts. In some embodiments, the ex vivo population of fetal cells comprises megakaryocytes. In some embodiments, the ex vivo population of fetal cells comprises stromal cells. In some embodiments, the ex vivo population of fetal cells comprises endothelial cells. In some embodiments, the ex vivo population of fetal cells comprises lymphocytes. In some embodiments, the ex vivo population of fetal cells comprises nucleated red blood cells. In some embodiments, the ex vivo population of fetal cells comprises hematopoietic progenitor cells. In some embodiments, the ex vivo population of fetal cells comprises mesenchymal progenitor cells.

In some embodiments, the present disclosure provides a method comprising: performing a single round of enrichment of a population of fetal cells from a population of maternal cells in a cell sample, wherein the single round of enrichment enriches the population of fetal cells from the cell sample to a level that provides a quantity of fetal nucleic acid that is sufficient for characterization as fetal nucleic acid rather than maternal nucleic acid.

In some embodiments, the single round of enrichment provides an enriched sample, wherein the level that provides the quantity of fetal nucleic acid that is sufficient for characterization as fetal nucleic acid rather than maternal nucleic acid is at least about 50% of fetal cells of the enriched sample. In some embodiments, the method further comprises performing one and no greater than one round of the enrichment.

In some embodiments, the cell sample is a blood sample. In some embodiments, the blood sample is blood plasma. In some embodiments, the blood sample is blood serum. In some embodiments, the blood sample is buffy coat. In some embodiments, the maternal blood sample comprises peripheral blood mononuclear white blood cells. In some embodiments, the maternal blood sample comprises nucleated white blood cells. In some embodiments, the blood sample is whole blood.

In some embodiments, the cell sample is from a human subject.

In some embodiments, the method further comprises, prior to the performing, extracting the population of maternal cells and the population of fetal cells from the cell sample to provide an extract, wherein the extract comprises the population of maternal cells and the population of fetal cells. In some embodiments, the method further comprises sorting the population of fetal cells in the extract from the population of maternal cells in the extract to provide a sample enriched in fetal cells and depleted in maternal cells.

In some embodiments, prior to the performing, the population of fetal cells and the population of maternal cells are extracted from the cell sample to provide an extract, wherein the extract comprises the population of maternal cells and the population of fetal cells. In some embodiments, the population of fetal cells in the extract is sorted from the population of maternal cells in the extract to provide a sample enriched in fetal cells and depleted in maternal cells.

In some embodiments, prior to the performing, the cell sample is incubated with a first isolation agent specific for a first biomarker in the population of fetal cells. In some embodiments, the first isolation agent is a nucleic acid probe. In some embodiments, the first isolation agent is a ligand. In some embodiments, the first biomarker comprises a cell surface marker. In some embodiments, the first biomarker comprises a soluble cell surface marker. In some embodiments, the first biomarker comprises an intracellular biomarker. In some embodiments, the first biomarker comprises a nucleic acid marker.

In some embodiments, the method further comprises, prior to the performing, incubating the cell sample with a first isolation agent specific for a first biomarker in the population of fetal cells. In some embodiments, the first isolation agent is a nucleic acid probe. In some embodiments, the first isolation agent is a ligand. In some embodiments, the first biomarker comprises a cell surface marker. In some embodiments, the first biomarker comprises a soluble cell surface marker. In some embodiments, the first biomarker comprises an intracellular biomarker. In some embodiments, the first biomarker comprises a nucleic acid marker.

In some embodiments, prior to the performing, the cell sample is incubated with a second isolation agent specific for a second biomarker in the population of maternal cells. In some embodiments, the second isolation agent is a nucleic acid probe. In some embodiments, the second isolation agent is a ligand. In some embodiments, the second biomarker comprises a cell surface marker. In some embodiments, the second biomarker comprises a soluble cell surface marker. In some embodiments, the second biomarker comprises an intracellular biomarker. In some embodiments, the second biomarker comprises a nucleic acid marker.

In some embodiments, the method further comprises, prior to the performing, incubating the cell sample with a second isolation agent specific for a second biomarker in the population of maternal cells. In some embodiments, the second isolation agent is a nucleic acid probe. In some embodiments, the second isolation agent is a ligand. In some embodiments, the second biomarker comprises a cell surface marker. In some embodiments, the second biomarker comprises a soluble cell surface marker. In some embodiments, the second biomarker comprises an intracellular biomarker. In some embodiments, the second biomarker comprises a nucleic acid marker.

In some embodiments, the method further comprises sequencing a nucleic acid sequence from the population of fetal cells.

In some embodiments, the present disclosure provides a method comprising: a) extracting an ex vivo subpopulation of maternal cells and an ex vivo subpopulation of fetal cells from a maternal blood sample, b) after the extracting, enriching the ex vivo subpopulation of fetal cells from the ex vivo subpopulation of maternal cells, c) after the enriching, sorting the ex vivo subpopulation of fetal cells from the ex vivo subpopulation from maternal cells to provide a sample enriched in fetal cells and depleted in maternal cells, and d) after the sorting, sequencing a nucleic acid from the ex vivo subpopulation of fetal cells.

In some embodiments, the present disclosure provides a method comprising: a) contacting a maternal blood sample comprising an ex vivo subpopulation of fetal cells and an ex vivo subpopulation of maternal cells with an isolation agent that couples to a biomarker in the ex vivo subpopulation of fetal cells, b) after the contacting, incubating the isolation agent with the maternal blood sample, c) after the incubating, isolating the ex vivo subpopulation of fetal cells in the maternal blood sample from the ex vivo subpopulation of maternal cells in the maternal blood sample, and d) after the isolating, sequencing a nucleic acid from the ex vivo subpopulation of fetal cells.

In some embodiments, the present disclosure provides a method comprising: a) contacting a maternal blood sample comprising an ex vivo subpopulation of fetal cells and an ex vivo subpopulation of maternal cells with an isolation agent that couples to a biomarker in the ex vivo subpopulation of fetal cells, b) after the contacting, incubating the isolation agent with the maternal blood sample, c) after the incubating, isolating the ex vivo subpopulation of fetal cells in the maternal blood sample from the ex vivo subpopulation of maternal cells in the maternal blood sample, d) after the isolating, sorting the ex vivo subpopulation of fetal cells from the ex vivo subpopulation of maternal cells, and e) after the sorting, sequencing a nucleic acid from the ex vivo subpopulation of fetal cells.

In an aspect, the present disclosure provides a method comprising: incubating a population of cells from a sample of a subject with a first isolation agent, a second isolation agent, a third isolation agent, a fourth isolation agent, a fifth isolation agent, and a sixth isolation agent, wherein the population of cells comprises a subpopulation of fetal cells and a subpopulation of maternal cells, wherein the first isolation agent is specific for a first biomarker, the second isolation agent is specific for a second biomarker, the third isolation agent is specific for a third biomarker, the fourth isolation agent is specific for a fourth biomarker, and the fifth isolation agent is specific for a fifth biomarker in the subpopulation of fetal cells, and wherein the sixth isolation agent is specific for a sixth biomarker in the subpopulation of maternal cells. In some embodiments, the subject is a human subject.

In some embodiments, the sample is a blood sample. In some embodiments, the blood sample is blood plasma. In some embodiments, the blood sample is blood serum. In some embodiments, the blood sample is buffy coat. In some embodiments, the maternal blood sample comprises peripheral blood mononuclear white blood cells. In some embodiments, the maternal blood sample comprises nucleated white blood cells. In some embodiments, the blood sample is whole blood.

In some embodiments, one of the first isolation agent, the second isolation agent, the third isolation agent, the fourth isolation agent, the fifth isolation agent, and the sixth isolation agent is a nucleic acid probe.

In some embodiments, one of the first isolation agent, the second isolation agent, the third isolation agent, the fourth isolation agent, the fifth isolation agent, and the sixth isolation agent is a ligand.

In some embodiments, one of the first isolation agent, the second isolation agent, the third isolation agent, the fourth isolation agent, the fifth isolation agent, and the sixth isolation agent is specific for a cell surface marker.

In some embodiments, one of the first isolation agent, the second isolation agent, the third isolation agent, the fourth isolation agent, the fifth isolation agent, and the sixth isolation agent is specific for a soluble cell surface marker.

In some embodiments, one of the first isolation agent, the second isolation agent, the third isolation agent, the fourth isolation agent, the fifth isolation agent, and the sixth isolation agent is specific for an intracellular biomarker.

In some embodiments, one of the first isolation agent, the second isolation agent, the third isolation agent, the fourth isolation agent, the fifth isolation agent, and the sixth isolation agent is specific for a nucleic acid marker.

In some embodiments, the method further comprises, after the incubating, enriching the subpopulation of fetal cells from the subpopulation of maternal cells.

In some embodiments, after the incubating, the subpopulation of fetal cells is enriched from the subpopulation of maternal cells.

In some embodiments, the present disclosure provides a method comprising: incubating a population of cells a first isolation agent, a second isolation agent, a third isolation agent, a fourth isolation agent, a fifth isolation agent, and a sixth isolation agent, wherein the population of cells comprises a subpopulation of fetal cells and a subpopulation of maternal cells from a sample of a subject, and wherein the first isolation agent is specific for a first biomarker in the subpopulation of fetal cells, the second isolation agent is specific for a second biomarker in the subpopulation of fetal cells, the third isolation agent is specific for a third biomarker in the subpopulation of fetal cells, the fourth isolation agent is specific for a fourth biomarker in the subpopulation of maternal cells, the fifth isolation agent is specific for a fifth biomarker in the subpopulation of maternal cells, and the sixth isolation agent is specific for a sixth biomarker in the subpopulation of maternal cells.

In some embodiments, the subject is a human subject.

In some embodiments, the sample is a blood sample. In some embodiments, the blood sample is blood plasma. In some embodiments, the blood sample is blood serum. In some embodiments, the blood sample is buffy coat. In some embodiments, the maternal blood sample comprises peripheral blood mononuclear white blood cells. In some embodiments, the maternal blood sample comprises nucleated white blood cells. In some embodiments, the blood sample is whole blood.

In some embodiments, one of the first isolation agent, the second isolation agent, the third isolation agent, the fourth isolation agent, the fifth isolation agent, and the sixth isolation agent is a nucleic acid probe.

In some embodiments, one of the first isolation agent, the second isolation agent, the third isolation agent, the fourth isolation agent, the fifth isolation agent, and the sixth isolation agent is a ligand.

In some embodiments, one of the first isolation agent, the second isolation agent, the third isolation agent, the fourth isolation agent, the fifth isolation agent, and the sixth isolation agent is specific for a cell surface marker.

In some embodiments, one of the first isolation agent, the second isolation agent, the third isolation agent, the fourth isolation agent, the fifth isolation agent, and the sixth isolation agent is specific for a soluble cell surface marker.

In some embodiments, one of the first isolation agent, the second isolation agent, the third isolation agent, the fourth isolation agent, the fifth isolation agent, and the sixth isolation agent is specific for an intracellular biomarker.

In some embodiments, one of the first isolation agent, the second isolation agent, the third isolation agent, the fourth isolation agent, the fifth isolation agent, and the sixth isolation agent is specific for a nucleic acid marker.

In some embodiments, the method further comprises, after the incubating, enriching the subpopulation of fetal cells from the subpopulation of maternal cells.

In some embodiments, after the incubating, the subpopulation of fetal cells is enriched from the subpopulation of maternal cells.

In some embodiments, the present disclosure provides a method comprising: incubating a population of cells with a first isolation agent, a second isolation agent, a third isolation agent, a fourth isolation agent, a fifth isolation agent, a sixth isolation agent, and a seventh isolation agent, wherein the population of cells comprises a subpopulation of fetal cells and a subpopulation of maternal cells from a sample of a subject, wherein the first isolation agent is specific for a first biomarker, the second isolation agent is specific for a second biomarker, the third isolation agent is specific for a third biomarker, the fourth isolation agent is specific for a fourth biomarker, the fifth isolation agent is specific for a fifth biomarker, and the sixth isolation agent is specific for a sixth biomarker in the subpopulation of maternal cells, and wherein the seventh isolation agent is specific for a seventh biomarker in the subpopulation of fetal cells.

In some embodiments, the subject is a human subject.

In some embodiments, the sample is a blood sample. In some embodiments, the blood sample is blood plasma. In some embodiments, the blood sample is blood serum. In some embodiments, the blood sample is buffy coat. In some embodiments, the maternal blood sample comprises peripheral blood mononuclear white blood cells. In some embodiments, the maternal blood sample comprises nucleated white blood cells. In some embodiments, the blood sample is whole blood.

In some embodiments, one of the first isolation agent, the second isolation agent, the third isolation agent, the fourth isolation agent, the fifth isolation agent, the sixth isolation agent, and the seventh isolation agent comprises a nucleic acid probe.

In some embodiments, one of the first isolation agent, the second isolation agent, the third isolation agent, the fourth isolation agent, the fifth isolation agent, the sixth isolation agent, and the seventh isolation agent comprises a ligand.

In some embodiments, one of the first isolation agent, the second isolation agent, the third isolation agent, the fourth isolation agent, the fifth isolation agent, the sixth isolation agent, and the seventh isolation agent is specific for a cell surface marker.

In some embodiments, one of the first isolation agent, the second isolation agent, the third isolation agent, the fourth isolation agent, the fifth isolation agent, the sixth isolation agent, and the seventh isolation agent is specific for a soluble cell surface marker.

In some embodiments, one of the first isolation agent, the second isolation agent, the third isolation agent, the fourth isolation agent, the fifth isolation, the sixth isolation agent, and the seventh isolation agent is specific for an intracellular biomarker.

In some embodiments, one of the first isolation agent, the second isolation agent, the third isolation agent, the fourth isolation agent, the fifth isolation agent, the sixth isolation agent, and the seventh isolation agent is specific for a nucleic acid marker.

In some embodiments, the method further comprises, after the incubating, enriching the subpopulation of fetal cells from the subpopulation of maternal cells.

In some embodiments, after the incubating, the subpopulation of fetal cells is enriched from the subpopulation of maternal cells.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a schematic for mouse breeding and experimental protocol for the identification of fetal cell-specific biomarkers and maternal cell-specific biomarkers.

FIG. 2 illustrates flow plots depicting the isolation of fetal cells from a maternal blood sample. The 201 plot depicts the total number of cells in the maternal blood sample. The 202 plot depicts the total number of single cells (e.g., not clustered cells) of the total number of cells in the maternal blood sample. The 203 plot depicts the number of viable cells (e.g., Calcein violet-positive cells) of the single cells in the maternal blood sample. The 204 plot depicts the number of nucleated cells (e.g., DyeCycle-positive cells) in the viable cells in the maternal blood sample. The 205 plot depicts the number of live cells (e.g., Sytox-orange-negative cells) in the nucleated cells in the maternal blood sample. The 206 table shows the number of cells in each of 201, 202, 203, 204, 205, and 207 plots. The 207 plot shows the number of circulating fetal cells (e.g., eGFP-positive cells) I the live cells in the maternal blood sample.

FIG. 3A depicts a single-cell gene expression analysis of maternal blood enriched for eGFP-expressing fetal cells. The clusters of cells enclosed by the black circles are the cells with the most robust expression of fetal-specific biomarkers.

FIG. 3B depicts single-cell gene expression profiles. The cells enclosed in the black circles are determined to be fetal trophoblasts, fetal stromal cells, and additional fetal cell types by single-cell gene expression analysis. The clusters of cells not enclosed in the black circles are determined to be different maternal cell types (e.g., megakaryocytes, B cells, T cells, NK cells, Basophils, Dendritic cells, macrophages, neutrophils, and erythroblasts).

FIG. 4A depicts the identification of fetal cells using a combination of fetal cell-specific biomarkers (left panel) against a background of maternal blood cell types. The right panel depicts the identification of different fetal cell types using a combination of fetal cell-specific biomarkers and maternal cell-specific biomarkers against a background of maternal blood cell types. FIG. 4B depicts the detection of early gestation stromal cells using a combination of fetal cell-specific biomarkers and maternal cell-specific biomarkers (left panel). The right panel depicts the identification of early gestation trophoblasts and late gestation trophoblasts using fetal cell-specific biomarkers.

FIG. 5 depicts a high-resolution clustering model of the isolated CFCs. The clustering model shows that placenta-related CFCs and hematopoietic CFCs are distinguished from each other based on different expression ratios of the fetal cell-specific cell biomarker Malat1.

FIG. 6A-6H depict heatmaps showing the patterns of cell type-specific expression of fetal cell-specific biomarkers and maternal cell-specific biomarkers. The x-axis of the heatmap depicts the different cell types (e.g., B cells, erythroblasts, macrophages, megakaryocytes, neutrophils, stromal cells, T cells, trophoblasts) and γ-axis shows the name of the fetal cell-specific biomarker or the maternal cell-specific biomarker.

FIG. 7A depicts the purity and yield of circulating fetal cells (CFCs) isolated from a maternal blood sample using sequential positive selections for fetal cells that are double-positive for two fetal biomarkers.

FIG. 7B depicts the purity and yield of CFCs isolated from a maternal blood sample using sequential positive selections for fetal cells that are double-positive for two fetal biomarkers.

FIG. 7C depicts the purity and yield of CFCs isolated from a maternal blood sample using sequential positive selections from fetal cells using two different combinations of fetal biomarkers.

FIG. 7D depicts the purity and yield of CFCs isolated from a maternal blood sample using sequential positive selections from fetal cells using two different combinations of fetal biomarkers.

FIG. 7E depicts the purity and yield of CFCs isolated from a maternal blood sample using a combination of negative selection with a combination of maternal biomarkers and sequential positive selections using two different combinations of fetal biomarkers.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In some embodiments, the present disclosure provides methods, compositions, and kits for isolating fetal cells from a maternal sample. In some embodiments, fetal cells and maternal cells are isolated from a maternal sample. In some embodiments, the fetal cells are enriched (e.g., sorted) from the maternal cells. In some embodiments, the fetal cells are enriched using isolation agents. In some embodiments, the fetal cells are incubated (e.g., contacted) with the isolation agents. In some embodiments, the isolation agents couple to a fetal cell-specific biomarker. In some embodiments, fetal nucleic acid molecules are extracted from the fetal cells. In some embodiments, the fetal nucleic acid molecules are sequenced. In some embodiments, the sequenced fetal nucleic acid molecules are used for prenatal testing. In some embodiments, the prenatal testing determines the presence or absence of genetic disorders or conditions. In some embodiments, the fetal cells are used to perform an assay.

### Compositions

### Cellular Compositions

In some embodiments, the present disclosure provides an ex vivo population of cells from a subject. In some embodiments, the ex vivo population of cells from the subject is from a pregnant subject. In some embodiments, the pregnant subject has two types of cells. In some embodiments, the two types of cells are fetal cells and maternal cells. In some embodiments, the fetal cells are cells that are derived from the fetus and/or the placenta. In some embodiments, the fetal cells have genetic information from the mother and genetic information from the father. In some embodiments, the maternal cells are cells that are derived from maternal organs and tissues. In some embodiments, the maternal cells have genetic information from the mother. In some embodiments, the maternal cells do not have genetic information from the father. In some embodiments, a sample is taken from the pregnant subject. In some cases, the sample has fetal cells and maternal cells. In some cases, the ex vivo population of cells is isolated from the sample taken from the pregnant subject.

In some embodiments, the ex vivo population of cells is isolated from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the subject is pregnant. In some embodiments, the sample is a biological sample. In some embodiments, the biological sample is a blood sample. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof.

In some embodiments, the ex vivo population of cells is isolated through density gradient centrifugation. In some embodiments, the ex vivo population of cells is isolated through density sedimentation. In some embodiments, the ex vivo population of cells is isolated through immunomagnetic cell separation. In some embodiments, the ex vivo population of cells is isolated through fluorescence-activated cell sorting (FACS). In some embodiments, the ex vivo population of cells is isolated through magnetic-activated cell sorting (MACS). In some embodiments, the ex vivo population of cells is isolated through microscopic microdissection. In some embodiments, the ex vivo population of cells is isolated through microscopic imaging. In some embodiments, the ex vivo population of cells is isolated from plasma and/or platelets from the blood sample.

In some embodiments, the ex vivo population of cells has a subpopulation of maternal cells. Non-limiting examples of maternal cell include red blood cells, T cells, B cells, dendritic cells, macrophages, monocytes, erythrocytes, neutrophils, and lymphocytes. In some embodiments, the ex vivo population of cells has a subpopulation of fetal cells. In some embodiments, the subpopulation of fetal cells is circulating fetal cells (CFCs). CFCs can be fetal cells that circulate in the maternal bloodstream. In some embodiments, the subpopulation of fetal cells contains one or more subpopulations of fetal cells, for example, a first subpopulation and a second subpopulation. In some embodiments, the second subpopulation of fetal cells is different in cell type from the first subpopulation of fetal cells. In some embodiments, the first subpopulation of fetal cells comprises early gestation fetal cells and the second subpopulation of fetal cells comprises later gestation fetal cells. Non-limiting examples of types of fetal cells include early gestation trophoblasts (e.g., early gestation trophoblasts), later gestation trophoblasts (e.g., late gestation trophoblasts), erythroblasts, megakaryocytes, stromal cells, endothelial cells, lymphocytes, nucleated red blood cells, hematopoietic progenitor cells, vascular endothelial cells, and mesenchymal progenitor cells.

In some embodiments, the first subpopulation of fetal cells comprises early gestation trophoblasts and the second subpopulation of fetal cells comprises later gestation trophoblasts. In some embodiments, the first subpopulation of fetal cells comprises early gestation trophoblasts and the second subpopulation of fetal cells comprises erythroblasts. In some embodiments, the first subpopulation of fetal cells comprises later gestation trophoblasts and the second subpopulation of fetal cells comprises erythroblasts. In some embodiments, the first subpopulation of fetal cells comprises early gestation trophoblasts and the second subpopulation of fetal cells comprises megakaryocytes. In some embodiments, the first subpopulation of fetal cells comprises later gestation trophoblasts and the second subpopulation of fetal cells comprises megakaryocytes. In some embodiments, the first subpopulation of fetal cells comprises early gestation trophoblasts and the second subpopulation of fetal cells comprises stromal cells. In some embodiments, the first subpopulation of fetal cells comprises later gestation trophoblasts and the second subpopulation of fetal cells comprises stromal cells. In some embodiments, the first subpopulation of fetal cells comprises early gestation trophoblasts and the second subpopulation of fetal cells comprises endothelial cells. In some embodiments, the first subpopulation of fetal cells comprises later gestation trophoblasts and the second subpopulation of fetal cells comprises endothelial cells. In some embodiments, the first subpopulation of fetal cells comprises early gestation trophoblasts and the second subpopulation of fetal cells comprises lymphocytes. In some embodiments, the first subpopulation of fetal cells comprises later gestation trophoblasts and the second subpopulation of fetal cells comprises lymphocytes. In some embodiments, the first subpopulation of fetal cells comprises early gestation trophoblasts and the second subpopulation of fetal cells comprises nucleated red blood cells. In some embodiments, the first subpopulation of fetal cells comprises later gestation trophoblasts and the second subpopulation of fetal cells comprises nucleated red blood cells. In some embodiments, the first subpopulation of fetal cells comprises early gestation trophoblasts and the second subpopulation of fetal cells comprises hematopoietic progenitor cells. In some embodiments, the first subpopulation of fetal cells comprises later gestation trophoblasts and the second subpopulation of fetal cells comprises hematopoietic progenitor cells. In some embodiments, the first subpopulation of fetal cells comprises early gestation trophoblasts and the second subpopulation of fetal cells comprises mesenchymal progenitor cells. In some embodiments, the first subpopulation of fetal cells comprises later gestation trophoblasts and the second subpopulation of fetal cells comprises mesenchymal progenitor cells. In some embodiments, the first subpopulation of fetal cells comprises early gestation trophoblasts and the second subpopulation of fetal cells comprises vascular endothelial cells. In some embodiments, the first subpopulation of fetal cells comprises later gestation trophoblasts and the second subpopulation of fetal cells comprises vascular endothelial cells.

In some embodiments, the first subpopulation of fetal cells comprises erythroblasts and the second subpopulation of fetal cells comprises megakaryocytes. In some embodiments, the first subpopulation of fetal cells comprises erythroblasts and the second subpopulation of fetal cells comprises stromal cells. In some embodiments, the first subpopulation of fetal cells comprises erythroblasts and the second subpopulation of fetal cells comprises endothelial cells. In some embodiments, the first subpopulation of fetal cells comprises erythroblasts and the second subpopulation of fetal cells comprises lymphocytes. In some embodiments, the first subpopulation of fetal cells comprises erythroblasts and the second subpopulation of fetal cells comprises nucleated red blood cells. In some embodiments, the first subpopulation of fetal cells comprises erythroblasts and the second subpopulation of fetal cells comprises hematopoietic progenitor cells. In some embodiments, the first subpopulation of fetal cells comprises erythroblasts and the second subpopulation of fetal cells comprises mesenchymal progenitor cells. In some embodiments, the first subpopulation of fetal cells comprises erythroblasts and the second subpopulation of fetal cells comprises vascular endothelial cells.

In some embodiments, the first subpopulation of fetal cells comprises megakaryocytes and the second population of fetal cells comprises stromal cells. In some embodiments, the first subpopulation of fetal cells comprises megakaryocytes and the second population of fetal cells comprises endothelial cells. In some embodiments, the first subpopulation of fetal cells comprises megakaryocytes and the second population of fetal cells comprises lymphocytes. In some embodiments, the first subpopulation of fetal cells comprises megakaryocytes and the second subpopulation of fetal cells comprises nucleated red blood cells. In some embodiments, the first subpopulation of fetal cells comprises megakaryocytes and the second subpopulation of fetal cells comprises hematopoietic progenitor cells. In some embodiments, the first subpopulation of fetal cells comprises megakaryocytes and the second subpopulation of fetal cells comprises mesenchymal progenitor cells. In some embodiments, the first subpopulation of fetal cells comprises megakaryocytes and the second subpopulation of fetal cells comprises vascular endothelial cells.

In some embodiments, the first subpopulation of fetal cells comprises stromal cells and the second subpopulation of fetal cells comprises endothelial cells. In some embodiments, the first subpopulation of fetal cells comprises stromal cells and the second subpopulation of fetal cells comprises lymphocytes. In some embodiments, the first subpopulation of fetal cells comprises stromal cells and the second subpopulation of fetal cells comprises nucleated red blood cells. In some embodiments, the first subpopulation of fetal cells comprises stromal cells and the second subpopulation of fetal cells comprises hematopoietic progenitor cells. In some embodiments, the first subpopulation of fetal cells comprises stromal cells and the second subpopulation of fetal cells comprises mesenchymal progenitor cells. In some embodiments, the first subpopulation of fetal cells comprises stromal cells and the second subpopulation of fetal cells comprises vascular endothelial cells.

In some embodiments, the first subpopulation of fetal cells comprises endothelial cells and the second subpopulation of fetal cells comprises lymphocytes. In some embodiments, the first subpopulation of fetal cells comprises endothelial cells and the second subpopulation of fetal cells comprises nucleated red blood cells. In some embodiments, the first subpopulation of fetal cells comprises endothelial cells and the second subpopulation of fetal cells comprises hematopoietic progenitor cells. In some embodiments, the first subpopulation of fetal cells comprises endothelial cells and the second subpopulation of fetal cells comprises mesenchymal progenitor cells. In some embodiments, the first subpopulation of fetal cells comprises endothelial cells and the second subpopulation of fetal cells comprises vascular endothelial cells.

In some embodiments, the first subpopulation of fetal cells comprises lymphocytes and the second subpopulation of fetal cells comprises nucleated red blood cells. In some embodiments, the first subpopulation of fetal cells comprises lymphocytes and the second subpopulation of fetal cells comprises hematopoietic progenitor cells. In some embodiments, the first subpopulation of fetal cells comprises lymphocytes and the second subpopulation of fetal cells comprises mesenchymal progenitor cells. In some embodiments, the first subpopulation of fetal cells comprises lymphocytes and the second subpopulation of fetal cells comprises vascular endothelial cells.

In some embodiments, the first subpopulation of fetal cells comprises nucleated red blood cells and the second subpopulation of fetal cells comprises hematopoietic progenitor cells. In some embodiments, the first subpopulation of fetal cells comprises nucleated red blood cells and the second subpopulation of fetal cells comprises mesenchymal progenitor cells. In some embodiments, the first subpopulation of fetal cells comprises nucleated red blood cells and the second subpopulation of fetal cells comprises vascular endothelial cells.

In some embodiments, the first subpopulation of fetal cells comprises hematopoietic progenitor cells and the second subpopulation of fetal cells comprises mesenchymal progenitor cells. In some embodiments, the first subpopulation of fetal cells comprises mesenchymal progenitor cells and the second subpopulation of fetal cells comprises hematopoietic progenitor cells. In some embodiments, the first subpopulation of fetal cells comprises mesenchymal progenitor cells and the second subpopulation of fetal cells comprises vascular endothelial cells. In some embodiments, the first subpopulation of fetal cells comprises hematopoietic progenitor cells and the second subpopulation of fetal cells comprises vascular endothelial cells. In some embodiments, the ex vivo population of cells has subpopulation of fetal cells and a subpopulation of maternal cells.

In some embodiments, the subpopulation of fetal cells is from about 50% to about 100% of cells in the ex vivo population of cells. In some embodiments, the subpopulation of fetal cells is from about 50% to about 60%, about 60% to about 70%, about 70% to about 80%, about 80% to about 90%, or about 90% to about 100% of cells in the ex vivo population of cells. In some embodiments, the subpopulation of fetal cells is at least about 50%, at least about 55%, at least about 60%, at least about 65%, at least about 70%, at least about 75%, at least about 80%, at least about 85%, at least about 90%, at least about 95%, at least about 100%, or more of cells in the ex vivo population of cells. In some embodiments, the subpopulation of fetal cells is at most about 100%, at most about 95%, at most about 90%, at most about 85%, at most about 80%, at most about 75%, at most about 70%, at most about 65%, at most about 60%, at most about 55%, at most about 50%, or less of cells in the ex vivo population of cells. In some embodiments, the subpopulation of fetal cells is at least about 50% of cells in the ex vivo population of cells. In some embodiments, the subpopulation of fetal cells is at least about 60% of cells in the ex vivo population of cells. In some embodiments, the subpopulation of fetal cells is at least about 70% of cells in the ex vivo population of cells. In some embodiments, the subpopulation of fetal cells is at least about 80% of cells in the ex vivo population of cells. In some embodiments, the subpopulation of fetal cells is at least about 90% of cells in the ex vivo population of cells.

In some embodiments, the subpopulation of maternal cells is no greater than about 0%, about 5%, about 10%, about 15%, about 20%, about 25%, about 30%, about 35%, about 40%, about 45%, or about 50% of cells in the ex vivo population of cells. In some embodiments, the subpopulation of maternal cells is no greater than about 50% of cells in the ex vivo population of cells. In some embodiments, the subpopulation of maternal cells is no greater than about 40% of cells in the ex vivo population of cells. In some embodiments, the subpopulation of maternal cells is no greater than about 30% of cells in the ex vivo population of cells. In some embodiments, the subpopulation of maternal cells is no greater than about 20% of cells in the ex vivo population of cells. In some embodiments, the subpopulation of maternal cells is no greater than about 10% of cells in the ex vivo population of cells. In some embodiments, the subpopulation of maternal cells is no greater than about 5% of cells in the ex vivo population of cells. In some embodiments, the subpopulation of maternal cells is no greater than about 0% of cells in the ex vivo population of cells.

In some embodiments, the ex vivo population of cells has a purity between about 50% to about 100% of fetal cells. In some embodiments, the ex vivo population of cells has a purity from about 50% to about 60%, about 60% to about 70%, about 70% to about 80%, about 80% to about 90%, or about 90% to about 100% of fetal cells. In some embodiments, the ex vivo population of cells has a purity of at least about 50%, at least about 55%, at least about 60%, at least about 65%, at least about 70%, at least about 75%, at least about 80%, at least about 85%, at least about 90%, at least about 95%, at least about 100%, or more of fetal cells. In some embodiments, the ex vivo population of cells has a purity of at most about 100%, at most about 95%, at most about 90%, at most about 85%, at most about 80%, at most about 75%, at most about 70%, at most about 65%, at most about 60%, at most about 55%, at most about 50%, or less of fetal cells. In some embodiments, the ex vivo population of cells has a purity from about 50% to about 60%, about 60% to about 70%, about 70% to about 80%, about 80% to about 90%, or about 90% to about 100% of fetal cells.

In some embodiments, the subpopulation of fetal cells has one or more fetal cell biomarkers. In some embodiments, the one or more biomarkers for the subpopulation of fetal cells are different from one or more maternal cell biomarkers. Non-limiting examples of fetal cell biomarkers include H19, 1600025M17Rik, Sct, Ctsr, Rhox9, Prl2c5, Rhox6, Fthl17a, Ctsj, Prl3b1, Nrk, Prl2b1, Ctsq, Hand1, Rhox5, Prl2c2, Krt8, Prl2c3, Prl7a1, Trap1a, Prl3d1, Igf2, Krt18, Nup62c1, Prl7d1, Rhox12, Prl2a1, Krt19, Plac1, Prl3d2, Serpinb9g, Slc38a4, Prl4a1, Serpinb9d, Serpinb9e, Prl8a9, Cited1, Sparc, Gm9112, Ldoc1, Serpinb9f, 3830417A13Rik, Serpinb9c, Tinagl1, Tfpi, Dusp9, Cts6, Hspb1, Nxf7, Sult1e1, Fnd3c2, Rarres2, Procr, Fermt2, Ghrh, Prl2c1, Plet1, Sbsn, Cdh5, Psg19, Igf2bp1, Tpbpa, Cda, Erv3, Pramef12, Shroom1, Peg10, 1700011M02Rik, Psg29, Cyr61, Crct1, Prl8a1, Psg22, Chrdl2, Xlr5a, Prl3d3, Ceacam9, Inhbb, Krt7, Serpinb9b, Cdkn1c, Rbp1, Mrgprg, Gm7120, Aoc1, Pla2g5, Rhox1, Tfap2c, A2m, Tpbpb, Lmcd1, Cryaa, Prl5a1, Zfp3613, Gm9, Prl6a1, Rgs17, Bex1, Cdh3, Prl7b1, Hsd17b2, Cyp17a1, Gm9513, Tex19.1, Parva, Papl, Map1b, Gpr50, 1600015l10Rik, Ceacam13, Prom2, Slco5a1, Cts7, Nos2, Nppc, Arhgap42, Ppp2r2c, Peg3, Mfap5, Tnfrsf9, Prl8a2, Ceacam15, Rimklb, Aard, Prl7a2, Slc30a2, 4933402E13Rik, Mmp1a, Epn3, Irx3, Fam83b, Tead4, Lama1, Htra1, Pfpl, Cts8, Adgrf5, Prl8a6, Drd4, Bmp1, Cystm1, Gml2, Gml3889, Krtdap, Pappa2, Sema3e, Homer2, Krt14, Pla2g2f, Klhl13, 2310030G06Rik, Car4, Gm7257, Prss8, Dsc2, Ptprn2, Des, Hsd3b6, Pxdn, 270033N17Rik, Eps812, 1700001C19Rik, Ceacam11, 1700089L19Rik, Trpm5, 1600012P17Rik, Got111, Dmrtc1b, Mt4, Spint1, Mt2, Fscn1, Gm14547, Serpine1, Col4a1, Ndufa412, Dst, Pvrl2, Doxl2, Cyp11a1, Klk6, Atf7ip2, Gm805, Pou2f3, Gpr173, Afap112, Nkain1, Fabp6, Triml2, Cryab, Pdgfra, Psca, Scnn1b, Rab15, Fmr1nb, Adamts7, Igfbpl1, A4galt, Hrct1, Aifl1, Gm16136, Sema3f, Fam189a1, Psg27, Gm22, Gm26829, Igf2os, Rpl29, Eif2s3y, Il25, Actn3, Sez6l, Unc5d, Frem2, Gm20714, Tff1, Hrc, Hspg2, Pdia5, Ccdc155, 2310016G11Rik, Fam90a1b, Vgf, Cts3, Klk4, Ttc9, Ces1a, Zfp385b, 4631405K08Rik, Nptx2, Adra2b, Mett124, Gm10251, Gm4758, Prl7c1, Col4a2, Cmtm5, Il24, Ephb3, Cartpt, Foxf1, Gm11755, Spdye4a, Mytl1, 4930562A09Rik, 9930038B18Rik, Msx1, Elf3, Adgrf4, Dio3, Rgs5, Star, Bcat1, Podxl, Lepr, Col5a3, Slco2a1, Myom3, Gm9979, Gpnmb, Fam159a, Trim29, Aqp8, Syt9, Kng2, Wnt7b, St8sia5, Tmem125, Dysf, Uaca, Fetub, Bcar1, Pcdh12, Gm14393, Fabp3, Stra6, Osmr, Lamc2, Muc1, RP23-378L12.3, Bok, Fdx1, Pkp2, Acbd7, Cyb5r3, Sdc1, Sfrp5, Lama5, Slc39a2, Zfp42, Sox3, Vvstm4, Gpx3, Dmkn, Gjb3, Lgi2, Stard10, Slc6a2, Prune2, H19, Sct, Rhox9, Krt18, Prl2c5, Rhox6, Krt8, Prl2c3, Ctsj, Krt19, Prl3b1, Prl3d1, Prl7d1, Prl7a1, Tfpi, Trap1a, Fthl17a, Ctsr, Sparc, Igf2, Prl2c2, Hspb1, Nrk, Ctsq, Rhox5, Prl2b1, Rarres2, Hand1, Peg10, Prl2a1, Procr, Sbsn, Prl4a1, Rhox12, Cdkn1c, Ghrh, Cdh5, Cited1, Gm9112, Krt7, Plac1, 1600025M17Rik, Serpinb9d, Cts6, Prl3d2, Serpinb9e, Dusp9, Map1b, Plet1, Nup62c1, Serpinb9g, Fermt2, Slc38a4, Ldoc1, Serpinb9f, 3830417A13Rik, Prl8a1, Hsd17b2, Prl8a9, Psg29, Nxf7, Psg22, Fnd3c2, Serpinb9c, Bex1, 1700011M02Rik, Tpbpa, Prl3d3, Prl2c1, Parva, Tinagl1, Prl5a1, Adgrf5, Igf2bp1, Rbp1, Mt2, Serpinh1, Tfap2c, Serpinb9b, Klhl13, Prl8a2, Sult1e1, Cyp11a1, Gjb3, Gm7120, Htra1, Cyp17a1, Cystm1, Pramef12, Tnfrsf9, Hsd3b6, Crip2, Ceacam9, Lgals1, Ctsl, Cryab, Homer2, Slc30a2, Cdh3, Zfp3613, Nppc, Ceacam15, Syngr1, Shroom1, Fscn1, Hspg2, Fmr1nb, Mmp1a, Sdc1, Pfpl, Krt14, Tex19.1, Psg19, Cb5r3, Sema3e, Peg3, Pdia5, Chrdl2, Dst, Dbi, Meis2, Sema3f, Rgs17, Col4a2, Fdx1, Dmrtc1b, Slco2a1, Tpml, Stard10, Mdk, Irx3, Maged1, Star, Csrp1, Nbl1, Rnd2, Prdx4, Des, Alp1, Xlr5a, Afao112, Aprt, Arhgap42, Vgf, Erv3, Tbpbp, Cst8, P4 hb, Tbrg1, Cyr61, Muc1, Tmem37, Podxl, Fabp3, Fkbp9, Fstl3, Car4, Epas1, Tpm2, Rhox1, Chchd10, Stra6, Egf17, Rab15, Copz2, Nos2, Lgals3, Sin3b, Slc6a2, Epcam, Pkp2, Pla2g5, Pvrl2, Rhox10, Prl7b1, Ada, Lmcd1, Erdr1, Selm, 1700086L19Rik, Dmrtc1a, Bok, Ldhb, Epn3, Mrgprg, Basp1, Gm9, Aoc1, Serpinb6b, Uaca, 2310030G06Rik, Ckb, Cstb, Ssr4, Fox04, Ngef, Plpp1, A2m, Dstn, Cox6a1, Gale, Pde10a, Krtdap, Rab6b, Esam, Crct1, Col5a2, A4galt, Dnase113, Sfn, Bmp1, Calr, Papl, Kank1, Prl6a1, Cmtm5, Ildr2, Rhoc, Ccl27a, Efs, Hrct1, Dsc2, Bcar1, Cd63, Inhbb, Ppp2r2c, Mpdz, Mt1, Fetub, Nid1, Bicc1, Psg23, Hdlbp, Rasd1, Arhgap22, Maged2, Aard, Prl8a6, Cald1, Prom2, Pdia6, Nostrin, Prss8, 1600012P17Rik, Dsp, Adm, Fam90a1b, Exoc314, Rbfox2, Mgst3, Gm648, Rgs5, Bgn, Galk1, Adra2b, AT036118, Nenf, Aifl1, Nfib, Acvr2b, Htatsf1, Rpn1, Psg27, Phldb2, Myof, Fbx119, H1f0, Cobl, Emp2, Myrf, Adamts7, Fam89a, Gm9513, Tmem98, Mfsd2a, Mgat4b, Cav1, Il33, C77080, Lars2, Gm21887, 9930038B18Rik, Prom1, Ptov1, Ltbp4, Mmp23, Prl7a2, Cts3, Fabp6, Prkcdbp, Lmna, Rpn2, Caskin1, Lama1, Fam83b, Cryaa, Lamc2, 4933402E13Rik, Fn1, Dlc1, Lrrn4, Tff1, Elf3, Serpine1, Bcat1, Perp, Lama5, Gpx3, Car2, Pawr, Phactr1, Oaf, Lamb2, Prnp, Scin, Parm1, Smim1, Jup, Gpx8, Atf5, Ift43, Gkap1, H1fx, Cenpt, Rhou, Rrm2, Cul7, Asns, Tubb3, 2200002D01Rik, Abhd6, Hdac6, Rgs16, Cgnl1, Atp9a, Src, 4930486L24Rik, Elf3, Ly6c1, transferrin receptor (TfR), ZNF614, ZNF539, ZNF283, ZFYVE9, WRN, UBL4A, TPM3, TP73, TMEFF2, THC2274391, THC2265980, t (BRACHYURY), SYT9, SLAMF1, RPS27, RPL39, RPL26, RPL23A, RPL23, RP11-78J21.1, RGPD2, QPRT, PPIA, PGK1, PF4, NTRK1, NBR2, MYL6, MAPK3, MAD2L1, LOC389286, KY, KMO, KCNK4, ITGA7, HMGN2, HMGA1, HLA-C, HIST1H2AJ, GSK3A, GCC2, FYN, F1135740, ENST00000356196, EEF1A1, EDN1, DKFZP434F142, D4ST1, CRYL1, CPS1, CD7, CCNA2, CB123670, BC111482, 6C089454, ALS2CL, AK000420, AHNAK, ADAM11, ACVR2B, ABHD2, CD105, HLA-G, galectin 13 (LGAGLS13/PP13), galectin 14 (LGALS14), placental growth factor (PGF), pregnancy-associated plasma protein A (PAPPA), alpha fetal protein (AFP), endoglin (ENG), fns-related tyrosine kinase 1 (FLT-1), keratin-7, PLAP, MCAM, laeverin, H315, FT1.41.1, NDOG-1, NDOG-5, BC1, AB-154, AB-340 (PAR-1), Glut-12, factor XIII, hPLH, HLA-C, JunD, Fra2, NDPK-A, CAR, HASH2, αHCG, IGF-11, PAI-1, p57 (KIP2), PP5, PLAC1, PLAC8 and PLAC9, CHL1, H119, HLA-C, Fra2, NDPK-A, the CAR protein, HASH2, human chorion gonadotropin alpha (hCGα), TFPI-2, annexin IV, cytokeratin-7, cytokeratin-8, cytokeratin-19, H19, IFG2, ESX1L, MASH2, ASH2, Stra13, FosB, Cyclin D1, GCM1, caspase-8, factor XIII, tapasin, Connexin 31, Connexin 43, HAND1, Syncytin, MMP9, APAF-1, caspase-3, caspase-9, FAS, Fas ligand, FLIP, AP-2γ, 313-HSD VI, CDX2, ERR2, PLX3, PTHrP, ASCL2, ID2, MET, TEF5, UPA, 11β-HSD2, c-ETs1, HMGI(Y), estrogen receptor, GemI, TTPA, 1503-7E, J42-4d, J2r(3), J2r(12), J2r(13), 305-4G, K1-1a, K2r/1f(50), K2r/1f(59), K(1)157-2A, K3r(HIGH)76, 597-10C, NT7-T3, N9r.Mf, 334-2C, O19r-T3, 01-1a, 332-9E, P60-1a, P1-1a, P3r (9), 305-9E, R5′-T3, R6r/1-6H, 369-8G, U2f-T3, 305-6G, L15-1a, L21-la, 252, 120r, clone-1, D19-2g, CD146, CD141, vimentin, VCAM, ICAM, VEGFR-1, VEGFR-2, VEGFR-3, ITGA5, ITGB5, CDH11, CDH3, CK1, CK2, CK3, CK4, CK5, CK6, CK7, CK8, CK9, CK10, CK13, CK14, CK15, CK16, CK17, CK18, CK19, CD9, CD39, CD200, EPHB4, EPCR, PAR-1, hPL, CSH2, KISS1, GDF15, CRH, TFPI2, B-hCG, LOC90625, FN1, COL1A2, PSG9, HBE, AFP, GC, APOC3, SERPINC1, APOB, AHSG, HPX, CPB2, ITIH1, APOH, AMBP, Thy-1, CD90, thromobomodulin, Flt-1, EPCR, CDH11, CDH3, CD59, CD71, CD36, Glycophorin A, hexokinase 2, placental alkaline phosphatase, hyalouronic acid, BCL-2, VE-cadherin, PECAM-1, α1-integrin, placental lactogen, PSG-1, a6 integrin, E-cadherin, MMP14, KCNQ4, CLDN6, F3, EPCAM, TROP2, CD147, fetal hemoglobin, embryonic hemoglobin, ITGA6, FSHR, CD45, LHCGR, CD270, Trop2, FOLR1, CD114, CSF3R, SLC40A1, CD156a, ERVW1, GPC3, TREML2, ALPP (866), CD163, CD144, ALPP (GM022), FOLR1, CD115, TSPAN1, SLC22A1, SCL22A11, EFNA1, SLC62A2, SLC6A2, NET, SLC40A1, CD71, CD166, TGF-beta2, human placental lactogen, c-erbB2, PAPPA2, PRG2, cytokeratin 7, beta-HCG, alpha-HCG, GCM1, syncytin, c-erbB2, leptin, INSL4, TGF-beta1, CSH1, KISS1, PEG10, PAGE4, p63, miR1246, miR1323, miR-512-3p, has-miR-516b-5p, miR-517-5p, has-miR525, miR-526b, ath-miR159a, RNU44, RNU48, U6, CDH9, HLA-G, MIH61, ALPP (8B6), CD49e, JAM2, transferrin receptor, glycophorin a (GPA), EGFR, thrombospondin receptor (CD36), CD34, HbF, HAE 9, FB3-2, H3-3, erythropoietin receptor, HBE, AFP, APOC3, SERPINC1, AMBP, CPB2, ITIH1, APOH, HPX, AHSG, APOB, J42-4-d, 2,3-biophosphoglycerate (BPG), carbonic anhydrase (CA), thymidine kinase (TK), MMP14, lectin, CD235a, carbohydrates, selectin, GPA, ANTIGEN-I, EpCAM, E-cadhrin, Muc-1, Hpl, CHS2, KISS1, GDF15, CRH, TFP12, CGB, LOC90625, FN-1, COL1A2, PSG9, PSG1, EGFR, CD105, cytokeratin, TLS1, CD147, CD141, PTPRN, BMPR2, TNFRSF19, STIM1, SELPLG, KLP-1, HECA452, GPR160, KLRD1, TNFSF18, LRP11, ABCA1, GPR176, SCARF, ESYT1, SCARB1, LTB4R, TLT2, CD141, CD90, Vimentin (Vim), VCAM, ICAM-1, VEGFR-1, Flt-1, VEGFR-1, VEGFR-2, VEGFR-3, PAI-1, EPCR, CD146, ITGA5, ITGB5, CDH11, CDH3, CD59, CSH-1, CSH-2, PSG2, PSG3, PSG11, HBG2, XAGE3, CSHL1, HBG1, ERVH48, phCG, any human orthologue of any of the foregoing, and any combination thereof.

In some embodiments, the subpopulation of fetal cells can have any combination of fetal biomarkers disclosed in the present disclosure.

In some embodiments, the different fetal cell types of the subpopulation of fetal cells can have expression of different fetal biomarkers.

In some embodiments, the fetal biomarkers of the present disclosure have human orthologues. Non-limiting examples of human orthologues of the fetal biomarkers include: AOC1, FAM236B, FAM236D, FAM236A, FAM236C, C11orf52, MAGEA10, A2M, A4GALT, AARD, ACVR2B, ADA, ADAMTS7, ADGRF5, ADM, ADRA2B, AFAP1L2, AIF1L, ALPL, APRT, ARHGAP22, ARHGAP42, BASP1, BCAR1, BEX1, BEX2, BGN, BICC1, BMP1, BOK, BSG, KIAA1522, CALD1, CALR, CA4, CASKIN1, CAV1, CCL27, CD63, CDA, CDH3, CDH5, CDKN1C, CEACAM4, PSG3, PSG8, PSG1, PSG6, PSG7, PSG11, PSG2, PSG5, PSG4, PSG9, CHCHD10, CHRDL2, CITED1, CKB, CMTM5, COBL, COL4A1, COL4A2, COL5A2, COPZ2, COX6A1, CRIP2, CRYAA, CRYAB, CSRP1, CSTB, CYB5R3, CYP11A1, CYP17A1, CCN1, CYSTM1, DB1, DCN, DES, DLC1, DMKN, DNASE1L3, DSC2, DSP, DST, DSTN, DUSP9, EFS, EGFL7, EMP2, EPAS1, EPCAM, EPN3, EPS8L2, ESAM, EXOC3L4, FABP3, FABP6, FAM83B, FAM89A, FAM90A1, FAM90A26, FAM90A7, FAM90A22, FAM90A23, FAM90A14, FAM90A18, FAM90A16, FAM90A8, FAM90A17, FAM90A19, FAM90A9, FAM90A10, FBXL19, FDX1, FERMT2, FETUB, FKBP9, FMR1NB, FN1, FOXO4, FSCN1, FSTL3, GALE, GALK1, GHRH, GJB3, CT45A1, CT45A3, CT45A5, CT45A6, CT45A2, CT45A7, CT45A8, CT45A9, CT45A10, SAGE1, TMEM267, GPR50, H1-0, HAND1, HDLBP, HMGN1, HOMER2, HRCT1, HSD17B2, HSPB1, HSPG2, HTATSF1, HTRA1, IGF2, IGF2BP1, IL33, ILDR2, INHBB, IRX3, KANKI, KLHL13, KRT18, KRT19, KRT7, KRT8, KRTDAP, LAMA1, LAMC2, LARS2, LDHB, LSOC1, LEPR, LGALS1, LGALS3, LMCD1, LMNA, LRRN4, LTBP4, MAGED1, MAGED2, MAP1B, MDK, MEIS2, MFSD2A, MGAT4B, MGST3, MMP1, MMP23B, MPDZ, MRGPRG, MT1G, MT2A, MUC1, MYOF, MYRF, NBL1, NENF, NFIB, NGEF, NID1, NOS2, NOSTRIN, NPC2, NPPC, NRK, NUP62CL, NXF5, NXF2, NXF2B, P4HB, ACP7, PARVA, PED10A, PDIA5, PDIA6, PEG10, PEG3, MPEG1, PHLDB2, PKP2, PLA2G5, PLAC1, PLET1, PLPP1, PODXL, PPIB, PPP2R2C, PRAMEF12, PRAMEF1, PRAMEF11, PRAMEF2, PRAMEF4, PRAMEF10, PRAMEF7, PRAMEF6, PRAMEF27, PRAMEF25, PRAMEF26, PRAMEF9, PRAMEF13, PRAMEF18, PRAMEF5, PRAMEF8, PRAMEF33, PRAMEF 15, PRAMEF14, PRAMEF19, PRAMEF17, PRAMEF20, PRDX4, PRKCD, PROCR, PROM1, PROM2, PRSS8, CEACAM4, PTOV1, NECTIN2, RAB15, RAB6B, RARRES2, RASD1, RBFOX2, RBP1, RGS17, RGS5, RHOC, RND2, RPN1, RPN2, S100A6, SBSN, SCT, SDC1, SEC61G, SEMA3E, SEMA3F, SERBINB6, SERBINB9, SERPINH1, SFN, SHROOM1, SIN3B, SLC30A2, SLC38A4, SLC6A2, SLCO2A1, SPARC, SSR4, STAR, STARD10, STRA6, SULT1E1, SYNGR1, TBRG1, TEX19, TFAP2C, TFF1, TFPI, TINAGL1, TMEM37, TMEM98, TNFRSF9, TPM1, TPM2, UACA, UQCRB, ATP5MK, VGF, FAM9A, FAM9B, FAM6C, C19orf33, ABHD6, ASNS, ATF5, ATP9A, BCAT1, CA2, CENPT, CGNL1, CTSL, CUL7, ELF3, FBXL19, GKAP1, GPX3, GPX8, H1FX, HDAC6, IFT43, JUP, LAMA5, LAMB2, OAF, PARM1, PAWR, PERP, PFPL, PHACTR1, PRNP, RGS16, RHOU, RRM2, SCIN, SERPINB6, SERPINB9, SERPINE1, SMIM1, SRC, TUBB3, and any combination thereof.

In some embodiments, the biomarkers for the subpopulation of fetal cells are Sct, H19, Krt8, Krt18, Tfpi, or Fthl17a. In some embodiments, the biomarker for the subpopulation of fetal cells is Sct. In some embodiments, the biomarker for the subpopulation of fetal cells is H19. In some embodiments, the biomarker for the subpopulation of fetal cells is Tfpi. In some embodiments, the biomarker for the subpopulation of fetal cells is Fthl17a.

In some embodiments, the subpopulation of fetal cells are stromal cells. Non-limiting examples of fetal biomarkers on fetal stromal cells include 4930486L24Rik, Cryab, Ctsl, Gpx3, Hsd3b6, Ly6c1, Mt1, Prl8a2, Sbsn, Htra1, Mfap5, Rrm2, Tfpi, Des, Dmkn, Mt2, Prnp, Cdh5, Col4a1, Cts7, H19, Hspb1, Lamb2, Lepr, Hspg2, Tfpi, Procr, Slc38a4, Mfap5, Des, Plet1, Cdh5, H19, Krt19, Plac1, Adgrf5, and Sct.

In some embodiments, the subpopulation of fetal cells are trophoblasts. Non-limiting examples of fetal biomarkers on fetal trophoblast cells include Rho9, Sct, H19, Prl2c5, Rhox6, Krt18, Krt8, Peg10, Tfpi, Ctsj, Hspb1, Cdkn1c, Prl2c3, Trap1a, Igf2, Fthl17a, Krt7, Krt19, Prl3b1, Prl7d1, Nrk, Rhox5, Sparc, Prl2c2, Procr, Prl7a1, Prl3d1, Ctsl, Ctsr, Crip2, Prl2b1, Sbsn, Cdh5, Hand1, Hsd17b2, Dusp9, Prl2a1, Rhox12, Ctsq, Serpinb9e, Fscn1, Plet1, Tnfrsf9, Cyp11a1, Nup62c1, 1600025M17Rik, Cited1, Prl4a1, Sdc1, Serpinb9b, Tinagl1, Bex1, Plac2, Car2, Gm7120, Cystm1, Ghrh, Gm9112, Nxf7, Rarres2, Serpinb9c, Slc38a4, Ceacam9, Fermt2, Serpinb9d, Syngr1, 3830417A13Rik, Mfap5, Serpinb9g, Klhl13, Map1b, Psg22, Rbp1, Ldoc1, Prl8a2, Serpinb9f, Adgrf5, Fnd3c2, Parva, Prl2c1, Cyp17a1, Lepr, Prl3d2, Prl8a9, Psg29, Cts6, Gpx3, Mt1, Prl3d3, Prl8a1, Col4a1, Fmr1nb, Homer2, Perp, Cts7, Prl5a1, Serpine1, Slc30a2, Tfap2c, Hsd3b6, Igf2bp1, Oaf, Pramef12, Shroom1, Tpbpa, 1700011N02Rik, Sult1e1, 4930486L24Rik, Bcat1, Elf2, Ly6C1, Mt2, Nppc, Pfpl, Rrm2, Hspg2, Mmp1a, Phactr1, Prl8a2, Vgf, Cdh3, Cryab, Prnp, Zfp3613, 1600015l10Rik, Prl2c5, Rhox9, Krt18, Sct, Serpinb9d, Krt14, Hand1, Serpinb9g, Slc38a4, Cyp17a1, Nppc, Hsd3b6, Prl7a1, Prl3d1, Rbp1, Bcat1, Prl3b1, Ctsj, Fthl17a, Hsd17b2, Nppc, Cdkn1c, Syngr1, Ctsj, Cdh5, Tfpi, Sdc1, Hspg2, Lepr, Plac1, Cryab, Procr, Plet1, Adgrf5, Hspg2, Hspb1, Lepr, Tnfrsf9, Sdc1, Nup62c1, Ctsr, Zfp3613, Map1b, Igf2, Trap1a, Nrk, and Chrdl2.

In some embodiments, the subpopulation of fetal cells are early gestation trophoblasts (e.g., early gestation trophoblasts). Non-limiting examples fetal biomarkers on early gestation trophoblasts include Prl3d1, H19, Krt18, Prl7a1, Sct, Krt8, Prl2c3, Rhox9, Rhox6, Prl2c5, Cdkn1c, Krt19, Prl2c2, Hspb1, Krt7, Igf2, Prl4a1, Sbsn, Prl2a1, Cyp11a1, Hand1, Peg10, Procr, Trap1a, Ctsl, Tfpi, Cdh5, Psg22, Sdc1, Serpinb9b, Plac1, Rhox5, Dusp9, Gm7120, Nrk, Serpinb9e, Plet1, Sparc, Cystm1, Fscn1, Fthl17a, Prl3d2, Prl3d3, Prl8a1, Crip2, Ctsj, Parva, Psg29, Serpinb9c, Serpinb9d, Tnfrsf9, Hsd17b2, Nup62c1, Tinagl1, 1600025M17Rik, Serpinb9g, 3830417A13Rik, Car2, Nxf7, Prl7d1, Prl8a9, Rhox12, Serpinb9f, Cyp17a1, Homer2, Ldoc1, Prl2c1, Prl5a1, Adgrf5, Hsd3b6, Igf2bp1, Rbp1, Col4a1, Fermt2, Map1b, Prl2b1, Prl3b1, Tfap2c, Serpine1, Bcat1, Cryab, Fmr1nb, Nppc, Oaf, Sult1e1, Syngr1, Vgf, Hspg2, Mt2, Perp, Rrm2, Slc30a2, 1600015l10Rik, Ceacam9, Chrdl2, Cited1, Ctsr, Gm9112, Mt1, Pramef12, Slc38a4, Dst, Map1b, Gm7120, Map1b, Rere, Prl3d1, Prl5a1, Krt7, Prl8a2, Prl7a1, Rbp1, Serpine1, Procr, Serpinb9b, Sdc1, Prnp, Fscn1, Psg22, Fthl17a, Krt14, Cdh3, Fnd3c2, 1700011M02Rik, Prl3d2, Gm9112, Ldoc1, 3830417A13Rik, Serpinb9f, Igf2bp1, Sult1e1, Pramef12, Zfp3613, Rhox5, Rhox12, Plac1, Serpinb9g, Prl2c1, Adgrf5, Slc38a4, Rrm2, Cryab, Htra1, Des, Mfap5, Dmkn, Ces1a, Plac1, Tfpi, Hspg2, Plet1, Cdh5, Lepr, Procr, Sdc1, Chrdl2, Prl5a1, Krt14, Cdh3, Fnd3c2, Prl3d2, Ldoc1, Pramef12, Psg29, Prl3d3, Psg22, Prl8a1, Parva, Slc30a2, Ghrh, Mmp1a, Hsd3b6, Prl5a1, Prl3d2, Trap1a, Nrk, Plet1, Fthl17a, Plac1, Tfpi, Hspg2, Cdh5, Lepr, Procr, Cryab, Sdc1, Hsd3b6, Des, Mfap5, Htra1, Dmkn, and Tpbpa.

In some embodiments, the subpopulation of fetal cells comprises later gestation trophoblasts (e.g., late gestation trophoblasts). Non-limiting examples of fetal biomarkers on later gestation trophoblasts include Ctsq, Ctsr, Prl3b1, Prl7d1, Prl3d1, Cyp11a1, Cdh5, Hand1, Cdh3, 1600025M17Rik, Rhox5, Fermt2, Serpinb9d, Serpinb9f, 3830417A13Rik, Fnd3c2, Pramef12, Serpinb9g, Nup62c1, Prl2c1, Fnd3c2, 170011M02Rik, 3830417A13Rik, Nup62c1, Adgrf5, Plac1, Tpbpa, Pel3d3, Tfap2c, Serpinb9e, Psg29, Parva, Serpinb9c, Prl8a9, Psg22, Slc30a2, Hsd3b6, Prl8a1, Nxf7, Prl2a1, Htra1, Prl4a1, Dusp9, Tinagl1, Plet1, Pel7a1, Prl7d1, Ctsj, Sbsn, Prpf4b, Erdr1, Gm7120, Prl3d2, Igf2bp1, Ldoc1, Sult1e1, Krt14, Feng3c2, Fthl17a, Rhox12, Adgrf5, Fermt2, Prpf4b, Ctsl, Prl2b1, Zfp3613, Fnd3c2, Rhox6, Rhox9, Sct, Ctsj, Prl2c5, H19, Prl2b1, Sparc, Trap1a, Peg10, Rhox6, Tfpi, Bex1, Krt18, Cited1, Cts6, Ghrh, Hsd17b2, Hspb1, Rarres2, Crip2, Krt8, Lepr, Mfap5, Mt1, Nrk, Prl3d2, Procr, Fthl17a, Prl2c3, Slc38a4, Fermt2, Gm9112, Krt19, Cdkn1c, Krt7, Map1b, Rhox12, Igf2, Sbsn, and Syngr1.

In some embodiments, the fetal cell biomarker is an extracellular biomarker. In some embodiments, the fetal cell biomarker is an extracellular protein. In some embodiments, the extracellular protein is a cell surface protein. In some embodiments, the cell surface protein is a cell-surface receptor. In some embodiments, the extracellular protein is a soluble cell surface protein. In some embodiments, the extracellular protein is a transmembrane protein. In some embodiments, the extracellular protein is a transmembrane channel protein.

In some embodiments, the fetal cell biomarker is an intracellular biomarker. In some embodiments, the fetal cell biomarker is an intracellular protein. In some embodiments, the intracellular protein is an enzyme. In some embodiments, the intracellular protein is a phosphoprotein. In some embodiments, the intracellular protein is a growth factor. In some embodiments, the intracellular protein is a structural protein. In some embodiments, the fetal cell biomarker is a nucleic acid molecule. In some embodiments, the nucleic acid molecule is DNA. In some embodiments, the nucleic acid molecule is RNA. In some embodiments, the RNA is mRNA. In some embodiments, the RNA is rRNA. In some embodiments, the RNA is lncRNA. In some embodiments, the RNA is miRNA. In some embodiments, the RNA is siRNA.

In some embodiments, the subpopulation of maternal cells has one or more maternal cell biomarkers. In some embodiments, the maternal cell biomarker is a cell-surface protein. In some embodiments, the maternal cell biomarkers is a cell-surface receptor. In some embodiments, the maternal cell biomarkers is a nucleic acid biomarker. In some embodiments, the nucleic acid biomarker is a RNA biomarker. In some embodiments, the nucleic acid biomarker is a DNA biomarker. In some embodiments, the biomarkers for the subpopulation of maternal cells are different from one or more fetal cell biomarkers. Non-limiting examples of maternal cell biomarkers include Coro1a, Rac2, Malat1, Laptm5, H2-D1, B2m, Cytip, Alox5ap, Cd52, Junb, Cyba, Fyb, Btg1, Srgn, Actb, Klf2, Mcl1, H3f3a, Hmha1, Tmsb4x, Cd53, Ptprc, Fxyd5, Myl12b, Tyrobp, Gmfg, Fcer1g, Ier2, H2-K1, Itm2b, Jund, Rasgrp2, Nfkbia, Itgb2, Pfn1, Ifngr1, Ltb, Pim1, Ssh2, Rbm39, Sell, Trbc2, Msn, Ddx5, Stk17b, Lyz2, Cd37, Csf3r, Host, Ifitm2, Pnrc1, Rap1b, Meat1, Fth1, Cd3d, Arhgdib, Pglyrp1, H3f3b, Ms4a6b, Akap13, S100a9, Lyn, Sorl1, Mbnl1, Cd44, Ier5, Lsp1, Cdc42, Aes, Ms4a6c, Msrb1, Gdpd3, Btg2, Sat1, Arhgap30, Lrg1, Itga4, Oaz1, Rhoa, Eif1, Zfp3612, Selplg, Cd3g, Zfp36, Arhgap15, Syk, Arpc2, Sf3b1, Ighm, Psmb9, Gnai2, Actr3, Lst1, Il1b, Satb1, Amp32a, Foxp1, Lcp1, Wfdc17, Taldo1, Slfn2, Rgs10, Ccnd3, Ncor1, Cd3e, Vim, Cebpb, Tpm3, Adgre5, S100a8, Ccl6, Ptpn18, Napsa, Rps9, C5ar1, Hnmnpf, Iqgap1, Ly6e, Klf13, Ifitm3, Psmb8, H2-Q4, Gsr, Hmgb2, Fos, Sh3bgrl3, Dusp1, Spi1, Cd84, Tspo, Lef1, Cyp4f18, Atp11b, Ccr1, Gpx1, Cd47, Samhd1, AB124611, Trac, AI467606, Shisa5, My16, Tspan13, Cfl1, Gm5150, Itgal, Calm2, Plek, Ucp2, Tsc22d3, Ikzf1, Grk6, Gimap1, Ybx1, Gm8797, Clic1, Rara, Limd2, Ifi2712a, Ubb, Gngt2, Cd14, Pla2g7, Ets1, Tgfb1, Actg1, Il17ra, Celf2, Srsf5, Gimap6, Tmem50a, Ctss, Ube2d3, Tln1, Apbb1ip, Gabarap, Arpc5, Kmt2e, Klf3, Cap1, Itgam, Emp3, Clk1, Ogt, Crlf3, Samsn1, Stk38, Tax1bp1, Lat, Clec4e, Cdkn2d, Rel, Srrm2, Smc6, Morf411, Rsrp1, Ptpn1, Rgs2, Fmnl1, Gpsm3, Klf6, Fcgr3, Mef2c, Birc2, Kdm6b, Tra2b, Ptprcap, Son, Rassf5, Rpl22, Gimap3, Hnrnpa2b1, Lbr, Lck, Calm1, Cdkn1b, Ccr7, Ypel3, Ptpn6, Cd74, Ppp2r5a, Cd9, Rcsd1, Svi1, Hmgb1, Rhog, Hacd4, 9930111J21Rik2, Stat1, Ncf4, Fam105a, Rsbn1l, Hp, Ankrd11, Emb, Dusp2, Pfdn5, Txnip, Psme1, Slfn1, Ostf1, Jak1, Sirpa, Rap1a, Fam49b, Gda, Picalm, Ywhaz, Pde4b, Dazap2, Tsc22d4, Pkm, Slc25a5, Kdm7a, Lcp2, Fus, H2-Q7, Mxd1, Glipr1, Bnip31, Luc712, Tnfaip812, Pdcd4, Map1lc3b, Plbd1, Skap1, Unc93b1, Ncf2, Rnf149, Bin2, Mycbp2, Cnp, S100a10, Ubc, Cd300ld, Fam32a, Ptp4a2, Adrb2, Prdx5, Crip1, Sna32, Csk, Tnrc6b, Arhgef1, Pou2f2, Hspa8, Pilra, Bcl11b, Zfp292, Cnn2, Aldh2, Ywhah, Zbtb7a, Pycard, Cotl1, Retnlg, Sp100, Irf2, Fgr, Igsf6, Ablim1, Fam65b, Fermt3, Tmbim6, Tpd52, Scp2, Ube2b, Notch2, Gnb2, Il7r, Capzb, Jun, Ptma, 2810474O19Rik, Prr13, Mrpl33, Ppp1r15a, Lilrb4a, Zyx, St8sia4, Arpc3, H2afz, S100a11, Pag1, Irf1, Tm6sf1, Rsf1, Frina, Rasa3, Smap2, Dok3, Skap2, Add3, Stx11, Mkrn1, Hck, Ccnl1, Ppp1ca, R3hdm4, Cd48, Chd2, Rgs19, Cth4, Atp2b1, Peli1, Fam107b, Ptbp3, Adipor1, Serp1, Pak2, Hspa5, Tpr, Zc3hav1, Sdcbp, Dock2, Rnf130, Ftl1, B4galnt1, Cd300a, Hcls1, Anxa1, Cst3, Ankrd44, Elf1, Prpf38b, Atp6vlg1, Snx20, Il6ra, Whsc1l1, Tmem71, Stk4, Sla, Mbp, Dnaja1, Vgll4, Capza2, Ldha, Pyhin1, Rapgef6, Hdc, Fli1, Rbm25, H2afy, Prdx6, Rac1, Stap1, Supt4a, Hnrnpk, Ubl5, Cdk2ap2, Ppp1r2, Psenen, Bcl2, Tprg1, Arpc1b, Cd69, Phf20l1, Ppt1, Rhoh, Coro1a, Cytip, Malat1, Rac2, Cd52, Laptm5, B2m, H2-D1, Cyba, Ptprc, Btg1, Gmfg, Junb, Srgn, Fxyd5, Itgb2, Ltb, Tyrobp, Mcl1, Cd53, Klf2, Fcer1g, H3f3a, Hmha1, Itga4, Alox5ap, Ptpn18, Myl12b, Cd3d, Spi1, Ier2, Ccl6, Ms4a6b, Msn, Selplg, Pglyrp1, S100a9, Lyn, Slfn2, H2-K1, Ier5, Cyp4f18, Csf3r, Hp, Lst1, Jund, Fyb, Rap1b, Pim1, Il1b, Lyz2, Stk17b, Cd3e, Gpsm3, Lef1, Sell, Hcst, Gm5150, Ms4a6c, Fmnl1, Adgre5, Arhgap30, Lrg1, 9930111J21Rik2, Unc93b1, Rbm39, Pnrc1, Ptpn6, Trac, Akap13, Actr3, Ifitm2, Crlf3, Msrb1, Samsn1, Ets1, Lck, Pla2g7, Zfp3612, Lat, Ncor1, Sorl1, Ccnd3, Aes, Rasgrp2, Samhd1, Psmb8, S100a8, Wfdc17, Ctss, Neat1, Arhgap15, Ncf4, Lcp1, AB124611, Ssh2, Btg2, March1, Snx20, Fam105a, Zfp36, C5ar1, Tspan13, Hck, Apbb1ip, Mbnl1, Cd37, Limd2, Skap1, Iqgap1, Cybb, Lcp2, Ly6e, Slfn1, Bcl11b, Syk, Sf3b1, Ccr1, Ddx5, Stk38, Sat1, Anp32a, Dock2, Cd74, Satb1, Ighm, Foxp1, Tspo, Klf13, Ptpn1, AI467606, Nfam1, Cap1, Hnmnpf, Shisa5, Fgr, Rhog, Ifngr1, Psmb9, Rhoh, Lilrb4a, Plek, Rasa3, Sla, Atp11b, Fam65b, Gimap1, Hmgb2, Ypel3, Tnfaip812, Pirb, Rgs2, Tmem50a, Fcgr3, Cd84, Kmt2e, Mpeg1, Retnlg, Ikzf1, Il6ra, Prdx5, Ostf1, Cd14, Itgam, Cd300ld, Rnf149, Cd79b, Dusp1, Tsc22d4, Ifi2712a, Il17ra, Sirpb1c, Klf3, Celf2, Clic1, Cebpb, Grk6, Pira2, Grap, Ppp2r5a, Fam49b, Cd47, Tgfb1, Wfdc21, Hcar2, Ncf2, Cd300a, Ms4a4b, Itgal, Trbc2, Dusp2, Tra2b, Pilra, Cyth4, Hcls1, Pyhin1, Son, Tgfbi, Tspan32, Vav1, Cd3g, Irf1, Dok3, Cxcr2, Ifitm3, Rara, 2810474O19Rik, Cd33, Adrb2, Snap23, Fos, B4galnt1, Lilr4b, Tpm3, Prkcq, Cd44, Rcsd1, Sp100, S1pr1, Cdk2ap2, Cd247, Cotl1, Lrrfip1, Slc11a1, Srsf5, Cnp, Klf6, Clec4e, Inpp5d, Glipr1, Lpcat2, Fermt3, Clec4a3, Fus, Itgb7, Tbc1d10c, Napsa, Pld4, Add3, Jak1, Aldh2, Cdkn1b, Gsr, Myo1f, Ccr7, Mxd1, St8sia4, Gimap3, Arpc5, Ly6d, Rassf5, Ifitm1, Arhgef1, Gdpd3, Ifitm6, Smap2, Fam107b, Cd224, Mcemp1, Bin2, Hdc, Fam32a, Pou2f2, Kdm6b, Cdkn2d, Vim F630028O10Rik, H2-Q4, Pag1, Rnf130, Smpdl3a, Tpd52, Tmcc1, Grina, Fgl2, Csk, Trbc1, Cd27, DIErtd622e, Rasal3, Gabarap, Cd28, Ipcef1, Rgs19, Bcl2, 4930523C07Rik, Slc25a5, Rapgef6, Ppt1, Gngt2, Irf2, Psme1, Clec4d, Slc15a3, Pilrb2, Andrk11, Peli1, Tsc22d3, Bank1, Iglc2, Il7r, Stfa211, Stat1, Igsf6, Tax1bp1, Nedd9, Tm6sf1, Ly86, Tprg1, 2310001H17Rik, H2-DMa, H2-DMb2, Sirpb1b, Srrm2, Stk4, H2-T23, Abcg1, Klhl6, Ablim1, Evi2a, Bnip31, Arhgap25, Lrrc25, Ogfrl1, Emp3, Clk1, Mkm1, Ggnt2, Hacd4, Picalm, Adipor1, Man2b1, Cd48, Lyl1, Sp140, Sephs2, Lbr, Cd69, Phf20l1, Scp2, Ms4a1, Cdc42se1, Rgs10, Sirpa, Stap1, Ddx6, Ppp1r2, Abi3, Acap1, Csf2rb, Tnfrsf18, Alox12, Ebf1, Luc712, Flna, Ikzf3, Sept1, Arid4a, Pi16, Gimap6, Dazap2, Ppplr16b, Lyst, Adrbk1, Nlrp3, Ptprcap, Pycard, Themis2, Lbh, Mycbp2, Rel, Apobec1, Cd2, Chd2, AF251705, Trem1, Dennd1c, Ram189b, Ncf1, Tnfaip2, Pde4b, Ccnl1, Fcgr4, Skap2, Elf1, Il2rg, Slc7a11, Trim30b, Smchd1, H2-Q7, Rasgrp1, Ptpn22, Mrgpra2b, Ptpn22, Mrgpra2b, Cmip, Dgka, 1600010M07Rik, Igkc, Slc2a6, Atg3, Arl4c, Il16, Btk, Il21r, Fli1, Gda, Mbd2, Fam134b, Myo1g, Pygl, Ptpre, Kdm7a, Ctse, Plbd1, Map3k3, Akna, Mmp8, Ywhaz, H2afy, Svi1, Mfsd6, Emilin2, Hnrnpa0, Psenen, Pdcd4, Cd5, Chill, Pnpla2, Macf1, Gm8369, Nfatc1, Plcg2, Rhof, Gm9733, Map1lc3b, Capzb, Ube2b, Ccpg1, Il1f9, Xist, Gimap5, Rsbn1l, Cbl, Cd300lb, Siglece, Slco3a1, Supt4a, Mbp, Ms4a4c, Cd79a, Nrros, Gimap7, Birc2, Ccdc12, Mapk14, Tcf7, Whsc1l1, 1830077J02Rik, Rbm25, Xrn2, Jakmip1, Dock10, Vsir, Siglecg, Ramp1, Cd6, Phip, Ifi203, Tpr, Kpna4, Ptafr, Sh3bp5, Itk, Tnrc6c, Notch2, Myd88, Nipbl, Prkcd, Slc2a3, Bcl10, Il10ra, Dhrs7, Clec4a1, Slpr4, Cd300lf, Ogt, Lrmp, Ttc7, Gm43603, Treml1, Bcl2alb, Marcks, Mier1, Vasp, Clec4a2, Gpcpd1, Traf3ip3, Prpf38b, Runx3, R3hdm4, Lfng, 1110008F13Rik, Slamf6, Pdcd10, Stx11, Srsf11, Gp5, Hnrnpl, BC028528, Gm26740, Pid1, Nfe2, Mzb1, Lilra6, Snca, Parp14, Vgll4, Socs3, Sh2d1a, Dapl1, Gpr183, AW011738, Hbb-bs, Lmnb1, Strbp, H2-Aa, Il18rap, Hipk1, Stag2, Orai1, Smc6, Stk10, Fam111, Treml4, Ctage5, Ebi3, Ap1s2, Neurl3, Trib2, Zc3hav1, Baz1a, Hectd1, Sec11c, Tnrc6b, Amica1, Prkd2, Rnase6, Ev1, Vav3, Rab27a, Trim30a, Adam8, Phf3, Gpr132, Sirt7, Tcp11l2, Zcchc7, Itpr2, Bptf, Ace, Icam2, Slfn4, Fcmr, Prkar1a, Asap1, Clec2i, Prr13, Mef2c, Kansl1, Slc40a1, Vps37b, Sdcbp, Cd8b1, Phf11b, Zeb2, Sfpq, Ccl3, Anxa1, Pik3cd, Serp1, Arrb2, Ccdc88b, Ctsc, Atf7ip, Treml2, Nfkb1, Vamp8, Slc16a3, Kif2a, Lmo2, Capza2, Tbc1d1, Gimap8, Rassf3, Ifngr2, Ltb4r1, Trem3, Pf4, Clec2d, Pten, Cd226, H2-Ab1, Sgk1, Zcchc11, Lcn2, Rgs14, Ptp4a2, Tmem156, Mctp1, H2-Q6, N4bp1, Arhgap9, Birc3, Txk, Nptn, Psme2, Arl6ip5, Pstpip1, Tuba4a, Sertad1, Diaph1, Tnfrsf13b, Batf, AW112010, Il10rb, Arl5c, Cxcl2, Ccdc88c, Cdk9, Sh3bp1, Gimap4, Rnf144a, Bcl11a, Rassf2, Gramd1a, Nadk, Tnfrsf13c, Tra2a, Ggnbp2, Isg15, Def6, Stat4, Prrc2c, Gm15987, Card19, Clec12a, Gsap, Mfng, BC021614, Cmtm6, Cpr65, Tctex1d2, Cntrl, Tacc1, Zfp292, Sh3kbp1, Ifi47, Gpr18, Syf2, Twistnb, Rinl, Gp9, Aim2, Ctcf, Cd300e, Dck, B430306N03Rik, Otulin, Pip4k2a, Max, Tnfsf14, Krit1, Tmpo, Tnf, Ubl3, Csnk1a1, Fpr2, Cecam1, Bmx, Vrk1, Prkcb, Pfkfb4, G3bp2, Abtb1, Epsti1, Rin3, Itpkb, Gimap9, Adgre4, Ms4a6d, Ly9, Tap2, Prkar2b, Sf3b6, Fam78a, Adhb8, 1700097N02Rik, Zdhhc18, Atp1a3, Ppp1r10, Ifi30, Mcmbp, Twf2, C130050O18Rik, Kras, Arhgap4, Tmem71, Alcam, Notch1, Morc3, Sun2, Haus8, Rsrc2, Cdc42ep3, Ikbkb, Chd7, Arl11, Clec4n, Cxcr4, Stk24, Nfkbiz, Serinc3, Timm10b, Ctsw, Rchy1, Tap1, Maf1, Rbm5, Sema4a, Mat2b, Cth1, Nfkbid, Cd4, Ly6c2, Mef2a, March2, Grap2, Av19, Fcer2a, Cd24a, Hba-a1, Bcl2a1a, Arf6, Rnf13, Blnk, Il27ra, Kcnn4, Hn1, Fpr1, Sash3, Atp8b4, Sfxn5, Gpr141, Rsu1, Prr51, Scnn1a, Tmem243, Fcrl1, Pik3r1, Nlrp12, Milr1, THBD, F3, FLT3, MCAM, CD141, CD135, CD142/F3, CD146, CD33, CD68, CD144, CDH5, CD62E, CD45, CD90, SELE, GAP/GLA5, ITGA5, KCNK3/TASK-1, CNTFR, JAM-1 (f11r), SDC4/Syndecan 4, ACVR2B, ACVR2A, CD44v6+CD44v4/5, STXIA, TEK/Tie2, CD171, L1CAM, NCAM1/CD56, ALPP, ALPPL2, MMP23A/MMP23B, CD14, CD4, CD8, CD3, CD19, CD32, CD16, HLA-A, HLA-B, CD15 FSHR, CD45, LHCGR, CD270, Trop2, FOLR1, CD114, CSF3R, SLC40A1, CD156a, ERVW1, GPC3, TREML2, ALPP (866), CD163, CD144, ALPP (GM022), FOLR1, CD115, TSPAN1, SLC22A1, EFNA1, SLC62A2, SLC40A1, CD71, CD166, CDH12, HLA-A, HLA-B, PRSS8, CD45, CD230, ALPP (8B6), CD49e, JAM2, TREML2, MMP14, lectin, CD47, CD45, CD35, CD12, CD14, CD32, CD235a, carbohydrates, selectin, GPA, ANTIGEN-I, EpCAM, E-cadherin, Muc-1, Hpl, CHS2, KISS1, GDF15, CRH, TFP12, CGB, LOC90625, FN-1, COL1A2, PSG9, PSG1, CD105, TLS1, CD147, CD15, TNFRSF19, STIM1, CD19, CD14, any human orthologue of any of the foregoing, and any combination thereof.

In some embodiments, the maternal biomarkers of the present disclosure have human orthologues. Non-limiting examples of human orthologues of the maternal biomarkers include: CYBA, CORO1A, CYTIP, LAPTM5, CD53, RAC2, CD52, PTPRC, FCER1G, ARHGAP45, GPM6A, MAF, C3, CFH, EFEMP1, BICC1, COL1A2, COLIA1, B2M, BTG1, GMFG, JUNB, SRGN, ITGB2, LTB, TYROBP, MCL1, KLF2, NFKBIA, H3F3A, ITGA4, ALOX5AP, PTPN18, CD3D, SPI1, IER2, CCL15-CCL14, CCL14, CCL15, CCL23, MS4A6A, MS4A6E, MSN, SELPLG, ACTB, PGLYRP1, TMSB4Y, LYN, SLFN12, SLFN12L, IER5, CYP4F3, CSF3R, HP, HPR, LST1, ITM2B, JUND, FYB1, LSP1, RAP1B, PFN1, PIM1, IL1B, STK17B, CD3E, GPSM3, LEF1, SELL, HCST, AL049634.2, SIRPB1, SIRPG, SIRPA, MS4A6A, MS4A6E, FMNL1, ADGRE5, ARHGAP30, LRG1, UNC93B1, RBM39, PNRC1, PTPN6, AKAP13, ACTR3, IFITM2, IFITM1, IFITM3, ARHGDIB, CRLF3, MSRB1, SAMSN1, ETS1, LCK, PLA2G7, ZFP36L2, LAT, NCOR1, SORL1, CCND3, AES, RASGRP2, SAMHD1, PSMB8, S100A8, CTSS, ARHGAP15, NCF4, LCP1, C19orf38, SSH2, BTG2, MARCH1, GNAI2, SNX20, FAM105A, ZFP36, C5AR1, TSPAN13, HCK, APBB1IP, MBNL1, CD37, LIMD2, SKAP1, IQGAP1, CYBB, LCP2, EMB, LY6E, SLFN12, SLFN12L, BCL11B, SYK, SF3B1, CCR1, DDX5, STK38, SAT1, ANP32D, ANP32A, TALDO1, CDC42, DOCK2, CD74, SATB1, IGHM, TSPO, KLF13, H3F3B, PTPN1, C16orf54, NFAM1, CAP1, HNRNPF, SHISA5, FGR, RHOG, IFNGR1, PSMB9, RHOH, PLEK, RASA3, SLA, ATP11B, RIPOR2, SH3BGRL3, GIMAP1, HMGB2, RHOA, YPEL3, TNFAIP8L2, LILRB3, LILRA6, LILRB5, LILRA4, ARPC2, RGS2, TMEM50A, FCGR2A, FCGR2C, FCGR2B, CD84, KMT2E, MPEG1, RETNLB, TXNIP, IKZF1, IL6R, PRDX5, OSTF1, CD14, ITGAM, CD300LF, RNF149, CD79B, DUSP1, TSC22D4, IL17RA, AL049634.2, KLF3, CELF2, CLIC1, CEBPB, GPX1, GRK6, LILRA5, GRAP, GRAPL, PPP2R5A, FAM49B, CD47, TGFB1, HCAR2, HCAR3, NCF2, CD300A, CD300LD, ITGAL, TRBC1, TRBC2, OAZ1, DUSP2, TRA2B, CFL1, CYTH4, ANKRD44, HCLS1, SON, TGFBI, TSPAN32, VAV1, CD3G, IRF1, MRPL33, DOK3, CXCR2, IFITM1, IFITM3, IFITM2, RARA, KIAA1551, CD33, SIGLEC6, ADRB2, SNAP23, FOS, B4GALNT1, PRKCQ, CD44, RCSD1, UCP2, SP140, SIPR1, TLN1, CDK2AP2, CD247, COTL1, LRRFIP1, SLC11A1, SRSF5, CNP, CNN2, KLF6, CLEC4E, INPP5D, GLIPR1, LPCAT2, FERMT3, CLEC4C, FUS, ITGB7, TBC1D10C, NAPSA, PLD4, ADD3, JAK1, AC002996.1, ALDH2, CDKN1B, GSR, MYO1F, CCR7, MXD1, ST8SIA4, GIMAP1-GIMAP5, GIMAP1, GIMAP5, ARPC5, LY6D, RASSF5, IFITM1, IFITM2, IFITM3, ARHGEF1, GDPD3, SMAP2, FAM107B, MCEMP1, BIN2, HDC, FAM32A, POU2F2, KDM6B, CDKN2D, VIM, PAG1, RNF130, SMPDL3A, AC036214.3, TPD52, TMCC1, GRINA, FGL2, CSK, TRBC1, TRBC2, CD27, MACIR, RASAL3, GABARAP, CD28, IPCEF1, RGS19, BCL2, KIAA0040, SLC25A5, RAPGEF6, AC008695.1, PPT1, GNGT2, IRF2, PSME1, CLEC4D, SLC15A3, ANKRD11, PELI1, TSC22D3, RSRP1, YBX1, BANK1, IGLL1, IL7R, CTSA, STAT1, IGSF6, TAX1BP1, NEDD9, TM6SF1, LY86, TRPG1L, HLA-DMA, HLA-DMB, SRRM2, STK4, ABCG1, KLHL6, ABLIM1, RAP1A, EVI2A, BNIP3L, ARHGAP35, LRRC25, OGFRL1, EMP3, CLK1, MKRN1, GCNT2, HACD4, PICALM, UBE2D3, ADIPOR1, MAN2B1, CD48, LYL1, PAK2, SEPHS2, LBR, CD69, PHF20L1, SCP2, MS4A1, CDC42SE1, RGS10, STAP1, DDX6, PPPIR2, PPPIR2B, ABI3, ACAP1, PKM, CSF2RB, TNFRSF18, ALOX12, EBF1, LUC7L2, FLNA, IKZF3, PRDX6, SEPTIN1, ARID4A, PI16, GIMAP6, DAZAP2, PPP1R16B, LYST, GRK2, NLRP3, HNRNPA2B1, PTPRCAP, PYCARD, THEMIS2, LBH, MYCBP2, REL, APOBEC1, CD2, CHD2, TREM1, DENND1C, ENTREP3, NCF1, TNFAIP2, PDE4B, ATP6V1G1, CCNL1, FCGR3A, FCGR3B, SKAP2, ELF1, IL2RG, SLC7A11, SMCHD1, CD9, RASGRP1, PBXIP1, PTPN22, MRGPRX3, MRGPRX4, MRGPRX1, MRGPRX2, CMIP, DGKA, IGKC, SLC2A6, ATG3, ARL4C, IL16, BTK, IL21R, PPP1CA, FLI1, GDA, MBD2, MYO1G, PYGL, SCAND1, PTPRE, KDM7A, CTSE, PLBD1, MAP3K3, AKNA, MMP8, YWHAZ, MACROH2A1, SVIL, H2AJ, MFSD6, ZYX, EMILIN2, HNRNPA0, PSENEN, PDCD4, CD5, CHI3L1, PNPLA2, MACF1, NFATC1, PLCG2, RHOF, CALM2, ROCK1, MAPILC3B2, MAPILC3B, CAPZB, UBE2B, CCPG1, IL36G, EIF1, GIMAP1-GIMAP5, GIMAP1, GIMAP5, MORF4L1, RSBNIL, CBL, CD300LB, SIGLEC9, SIGLEC7, SIGLEC8, SIGLEC12, SLCO3A1, SUPT4H1, MBP, PPP1R15A, CD79A, NRROS, PPP1R18, PTBP3, GIMAP7, BIRC2, CCDC12, MAPK14, TCF7, C1orf162, RBM25, XRN2, JAKMIP1, DOCK10, VSIR, SIGLEC11, SIGLEC10, RAMP1, CD6, PHIP, TPR, KPNA4, PTAFR, SH3BP5, ITK, TNRC6C, NOTCH2, MYD88, NIPBL, PRKCD, BCL10, IL10RA, DHRS7, CLEC4A, SIPR4, CD300LF, OGT, IRAG2, TTC7A, TREML1, FAU, BCL2A1, MARCKS, MIER1, VASP, CLEC4C, GPCPD1, TRAF3IP3, RUNX3, R3HDM4, LFNG, SLAMF6, PDCD10, STX11, SRSF11, GP5, HNRNPL, C1orf54, PID1, NFE2, MZB1, LILRA5, SCNA, PFDN5, PARP14, VGLL4, ENO1, ARL6IP1, SOCS3, SH2D1A, DAPL1, GPR183, LMNB1, STRBP, IL18RAP, HIPK1, STAG2, ORAI1, SMC6, STK10, FAM111A, GNB2, TREML4, EBI3, AP1S2, ARPC1B, NEURL3, TRIB2, ZC3HAV1, BAZ1A, HECTD1, SEC11C, TNRC6B, PRKD2, RNASE6, EVL, VAV3, RAB27A, TRIM5, ADAM8, PHF3, SIRT7, TCP11L2, ZCCHC7, ATP2B1, ITPR2, BPTF, ACE, ICAM2, SLFN12, SLFN12L, FCMR, PRKAR1A, ASAP1, CLEC2D, MEF2C, KANSL1, SLC40A1, VPS37B, SDCBP, CD8B, CD8B2, PHF11, ZEB2, SFPQ, CCL18, CCL3, CCL3L1, ANXA1, YWHAH, HMGB1, PIK3CD, SERP1, ARRB2, CCDC88B, CTSC, ATF7IP, TREML2, NFKB1, VAMP8, SLC16A3, KIF2A, LMO2, CAPZA2, TBCID1, GIMAP8, RASSF3, IFNGR2, TONSL, CLEC2D, PTEN, CD226, HLA-DQB1, HLA-DQB2, SGK1, LCN2, RGS14, PTP4A2, TMEM156, MCTP1, ARF1, N4BP1, ARHGAP9, BIRC3, TXK, NPTN, PSME2, ARL6IP5, PSTPIP1, TUBA4A, SERTAD1, DIAPH1, TNFRSF13B, BATF, IL10RB, ARL5C, CCDC88C, CDK9, SH3BP1, GIMAP4, RNF144A, BCL11A, UBE2D2, RASSF2, ARPC3, C19orf53, GRAMD1A, NADK, TNFRSF13C, TRA2A, GGNBP2, ISG15, DEF6, STAT4, ATP6V0E1, PRRC2C, CARD19, CLEC12A, GSAP, MENG, CMTM6, GPR65, DYNLT2B, CNTRL, S100A11, TACC1, TMEM59, ZNF292, SH3KBP1, GPR18, SYF2, POLR1F, RINL, GP9, AIM2, CTCF, CD300E, DCK, OTULIN, PIP4K2A, MAX, TNFSF14, CALM1, KRIT1, MYH9, TMPO, TNF, UBL3, CSNK1A1, CEACAM7, CEACEM5, CEACAM6, CEACAM3, CEACAM1, CEACAM8, BMX, YY1, VRK1, PRKCB, PFKFB4, G3BP2, ABTB1, EPSTI1, RIN3, ITPKB, MYL6, MS4A6A, MS4A6E, LY9, TAP2, PRKAR2B, SF3B6, FAM78A, ABHD8, ZDHHC18, ATP1A3, PPP1R10, MCMBP, TWF2, KRAS, ARHGAP4, TMEM71, ALCAM, NOTCH1, MORC3, SUN2, ALDOA, HAUS8, RSRC2, CDC42EP3, IKBKB, CHD7, ARL11, UBB, CLEC6A, CXCR4, STK24, NFKBIZ, SERINC3, TIMM10B, CTSW, RCHY1, TAP1, MAF1, RBM5, SEMA4A, MAT2B, CYTH1, NFKBID, CD4, GPI, MEF2A, MARCHF2, JUN, GRAP2, AVL9, FCER2, HBA1, HBA2, BCL2A1, ARF6, RNF13, BLNK, IL27RA, KCNN4, RAC1, FPR1, SASH3, FIS1, ATP8B4, SFXN5, GPR141, RSU1, PRR5L, SCNN1A, TMEM243, FCRLA, PIK3R1, NLRP12, MILR1, and any combination thereof.

In some embodiments, the biomarkers for the subpopulation of maternal cells is Coro1a, Cd52, Cyba, Rac2, Laptm5, or Ptprc. In some embodiments, the biomarkers for the subpopulation of maternal cells is Coro1a, Cd52, Cyba, Rac2, Laptm5, or Ptprc. In some embodiments, the biomarker for the subpopulation of maternal cells is Coro1a. In some embodiments, the biomarker for the subpopulation of maternal cells is Cd52. In some embodiments, the biomarker for the subpopulation of maternal cells is Cyba. In some embodiments, the biomarker for the subpopulation of maternal cells is Rac2. In some embodiments, the biomarker for the subpopulation of maternal cells is Laptm5. In some embodiments, the biomarker for the subpopulation of maternal cells is Ptprc.

In some embodiments, the one or more maternal cell biomarkers are located in the subpopulation of fetal cells. In some embodiments, the one or more maternal cell biomarkers are located on the subpopulation of fetal cells.

In some embodiments, the one or more maternal cell biomarker is an extracellular biomarker. In some embodiments, the one or more maternal cell biomarker is an extracellular protein. In some embodiments, the extracellular protein is a cell surface protein. In some embodiments, the cell surface protein is a cell-surface receptor. In some embodiments, the extracellular protein is a soluble cell surface protein. In some embodiments, the extracellular protein is a transmembrane protein. In some embodiments, the extracellular protein is a transmembrane channel protein.

In some embodiments, the one or more maternal cell biomarkers is an intracellular biomarker. In some embodiments, the one or more maternal cell biomarkers is an intracellular protein. In some embodiments, the intracellular protein is an enzyme. In some embodiments, the intracellular protein is a phosphoprotein. In some embodiments, the intracellular protein is a growth factor. In some embodiments, the intracellular protein is a structural protein. In some embodiments, the one or more maternal cell biomarkers is a nucleic acid molecule. In some embodiments, the nucleic acid molecule is DNA. In some embodiments, the nucleic acid molecule is RNA. In some embodiments, the RNA is mRNA. In some embodiments, the RNA is rRNA. In some embodiments, the RNA is lncRNA. In some embodiments, the RNA is miRNA. In some embodiments, the RNA is siRNA.

In some embodiments, the ex vivo population of cells is quantified. In some embodiments, the subpopulation of fetal cells in the ex vivo population of cells is quantified. In some embodiments, the subpopulation of maternal cells in the ex vivo population of cells is quantified. In some embodiments, the subpopulation of fetal cells and the subpopulation of maternal cells in the ex vivo population of cells is quantified. In some embodiments, the ex vivo population of cells is quantified by flow cytometry. In some embodiments, the ex vivo population of cells is quantified by fluorescence-activated cell sorting (FACS) flow cytometry. In some embodiments, the ex vivo population of cells is isolated through magnetic-activated cell sorting (MACS). In some embodiments, the ex vivo population of cells is quantified by microscopic microdissection. In some embodiments, the ex vivo population of cells is quantified by microscopic imaging. In some embodiments, the ex vivo population of cells is quantified by acoustic focusing flow cytometry. In some embodiments, the ex vivo population of cells is quantified by a hemocytometer. In some embodiments, the ex vivo population of cells is quantified by an automated cell counter. In some embodiments, the ex vivo population of cells is quantified by spectrophotometry.

### Reagent Compositions

In some embodiments, the present disclosure provides reagents for use in the methods disclosed herein. In some embodiments, the reagents are isolation agents. In some embodiments, the isolation agents couple to one or more biomarkers. In some embodiments, the isolation agents hybridize to the one or more biomarkers. In some embodiments, the isolation agents bind to the one or more biomarkers. In some embodiments, the isolation agents covalently bind to the one or more biomarkers. In some embodiments, the isolation agents bind to the one or more biomarkers via a polar covalent bond. In some embodiments, the isolation agents bind to the one or more biomarkers via a non-polar covalent bond. In some embodiments, the isolation agents non-covalently bind to the one or more biomarkers. In some embodiments, the isolation agents bind to the one or more biomarkers via hydrogen bonding. In some embodiments, the isolation agents bind to the one or more biomarkers via ionic bonding. In some embodiments, the isolation agents bind to the one or more biomarkers via halogen bonding. In some embodiments, the isolation agents bind to the one or more biomarkers via Van der Waals forces. In some embodiments, the isolation agents bind to the one or more biomarkers via electrostatic interactions. In some embodiments, the isolation agents bind to the one or more biomarkers via London dispersion forces. In some embodiments, the isolation agents bind to the one or more biomarkers via hydrophobic effect. In some embodiments, the isolation agents couple to one or more fetal biomarkers. In some embodiments, the isolation agents couple to one or more maternal biomarkers. In some embodiments, the isolation agents couple to one or more fetal biomarkers and one or more maternal biomarkers. In some embodiments, the reagent is a biological agent. In some embodiments, a biological agent is an isolation agent. In some embodiments, the biological agent is an affinity reagent.

In some embodiments, the biological agent recognizes a fetal biomarker. In some embodiments, the fetal biomarker is located in and/or on a fetal cell. Non-limiting examples of fetal cell biomarkers include H19, 1600025M17Rik, Sct, Ctsr, Rhox9, Prl2c5, Rhox6, Fthl17a, Ctsj, Prl3b1, Nrk, Prl2b1, Ctsq, Hand1, Rhox5, Prl2c2, Krt8, Prl2c3, Prl7a1, Trap1a, Prl3d1, Igf2, Krt18, Nup62c1, Prl7d1, Rhox12, Prl2a1, Krt19, Plac1, Prl3d2, Serpinb9g, Slc38a4, Prl4a1, Serpinb9d, Serpinb9e, Prl8a9, Cited1, Sparc, Gm9112, Ldoc1, Serpinb9f, 3830417A13Rik, Serpinb9c, Tinagl1, Tfpi, Dusp9, Cts6, Hspb1, Nxf7, Sult1e1, Fnd3c2, Rarres2, Procr, Fermt2, Ghrh, Prl2c1, Plet1, Sbsn, Cdh5, Psg19, Igf2bp1, Tpbpa, Cda, Erv3, Pramef12, Shroom1, Peg10, 1700011M02Rik, Psg29, Cyr61, Crct1, Prl8a1, Psg22, Chrdl2, Xlr5a, Prl3d3, Ceacam9, Inhbb, Krt7, Serpinb9b, Cdkn1c, Rbp1, Mrgprg, Gm7120, Aoc1, Pla2g5, Rhox1, Tfap2c, A2m, Tpbpb, Lmcd1, Cryaa, Prl5a1, Zfp3613, Gm9, Prl6a1, Rgs17, Bex1, Cdh3, Prl7b1, Hsd17b2, Cyp17a1, Gm9513, Tex19.1, Parva, Papl, Map1b, Gpr50, 1600015l10Rik, Ceacam13, Prom2, Slco5a1, Cts7, Nos2, Nppc, Arhgap42, Ppp2r2c, Peg3, Mfap5, Tnfrsf9, Prl8a2, Ceacam15, Rimklb, Aard, Prl7a2, Slc30a2, 4933402E13Rik, Mmp1a, Epn3, Irx3, Fam83b, Tead4, Lama1, Htra1, Pfpl, Cts8, Adgrf5, Prl8a6, Drd4, Bmp1, Cystm1, Gml2, Gm13889, Krtdap, Pappa2, Sema3e, Homer2, Krt14, Pla2g2f, Klhl13, 2310030G06Rik, Car4, Gm7257, Prss8, Dsc2, Ptprn2, Des, Hsd3b6, Pxdn, 270033N17Rik, Eps812, 1700001C19Rik, Ceacam11, 1700089L19Rik, Trpm5, 1600012P17Rik, Got111, Dmrtc1b, Mt4, Spint1, Mt2, Fscn1, Gm14547, Serpine1, Col4a1, Ndufa412, Dst, Pvrl2, Doxl2, Cyp11a1, Klk6, Atf7ip2, Gm805, Pou2f3, Gpr173, Afap112, Nkain1, Fabp6, Triml2, Cryab, Pdgfra, Psca, Scnn1b, Rab15, Fmr1nb, Adamts7, Igfbpl1, A4galt, Hrct1, Aifl1, Gm16136, Sema3f, Fam189a1, Psg27, Gm22, Gm26829, Igf2os, Rpl29, Eif2s3y, Il25, Actn3, Sez61, Unc5d, Frem2, Gm20714, Tff1, Hrc, Hspg2, Pdia5, Ccdc155, 2310016G11Rik, Fam90a1b, Vgf, Cts3, Klk4, Ttc9, Ces1a, Zfp385b, 4631405K08Rik, Nptx2, Adra2b, Mett124, Gm10251, Gm4758, Prl7c1, Col4a2, Cmtm5, Il24, Ephb3, Cartpt, Foxf1, Gm11755, Spdye4a, Mytl1, 4930562A09Rik, 9930038B18Rik, Msx1, Elf3, Adgrf4, Dio3, Rgs5, Star, Bcat1, Podxl, Lepr, Col5a3, Slco2a1, Myom3, Gm9979, Gpnmb, Fam159a, Trim29, Aqp8, Syt9, Kng2, Wnt7b, St8sia5, Tmem125, Dysf, Uaca, Fetub, Bcar1, Pcdh12, Gm14393, Fabp3, Stra6, Osmr, Lamc2, Muc1, RP23-378L12.3, Bok, Fdx1, Pkp2, Acbd7, Cyb5r3, Sdc1, Sfrp5, Lama5, Slc39a2, Zfp42, Sox3, Vvstm4, Gpx3, Dmkn, Gjb3, Lgi2, Stard10, Slc6a2, Prune2, H19, Sct, Rhox9, Krt18, Prl2c5, Rhox6, Krt8, Prl2c3, Ctsj, Krt19, Prl3b1, Prl3d1, Prl7d1, Prl7a1, Tfpi, Trap1a, Fthl17a, Ctsr, Sparc, Igf2, Prl2c2, Hspb1, Nrk, Ctsq, Rhox5, Prl2b1, Rarres2, Hand1, Peg10, Prl2a1, Procr, Sbsn, Prl4a1, Rhox12, Cdkn1c, Ghrh, Cdh5, Cited1, Gm9112, Krt7, Plac1, 1600025M17Rik, Serpinb9d, Cts6, Prl3d2, Serpinb9e, Dusp9, Map1b, Plet1, Nup62c1, Serpinb9g, Fermt2, Slc38a4, Ldoc1, Serpinb9f, 3830417A13Rik, Prl8a1, Hsd17b2, Prl8a9, Psg29, Nxf7, Psg22, Fnd3c2, Serpinb9c, Bex1, 1700011M02Rik, Tpbpa, Prl3d3, Prl2c1, Parva, Tinagl1, Col4a1, Prl5a1, Adgrf5, Igf2bp1, Rbp1, Mt2, Serpinh1, Tfap2c, Serpinb9b, Klhl13, Prl8a2, Lepr, Sult1e1, Cyp11a1, Gjb3, Gm7120, Htra1, Cts7, Cyp17a1, Cystm1, Pramef12, Tnfrsf9, Hsd3b6, Crip2, Ceacam9, Lgals1, Ctsl, Cryab, Homer2, Slc30a2, Cdh3, Zfp3613, Dmkn, Nppc, Ceacam15, Syngr1, Shroom1, Fscn1, Hspg2, Fmr1nb, Mmp1a, Sdc1, Pfpl, Krt14, Tex19.1, Psg19, Cb5r3, Sema3e, Peg3, Pdia5, Chrdl2, Dst, Dbi, Meis2, Sema3f, Rgs17, Col4a2, Fdx1, Dmrtc1b, Slco2a1, Tpml, Stard10, Mdk, Irx3, Maged1, Star, Csrp1, Nbl1, Rnd2, Prdx4, Des, Alp1, Xlr5a, Afao112, Aprt, Arhgap42, Vgf, Erv3, Tbpbp, Cst8, P4 hb, Tbrg1, Cyr61, Muc1, Tmem37, Podxl, Fabp3, Fkbp9, Fst13, Car4, Epas1, Tpm2, Rhox1, Chchd10, Stra6, Egf17, Rab15, Copz2, Nos2, Lgals3, Sin3b, Slc6a2, Epcam, Pkp2, Pla2g5, Pvrl2, Rhox10, Prl7b1, Ada, Lmcd1, Erdr1, Selm, 1700086L19Rik, Dmrtc1a, Bok, Ldhb, Epn3, Mrgprg, Basp1, Gm9, Aoc1, Serpinb6b, Uaca, 2310030G06Rik, Ckb, Cstb, Ssr4, Fox04, Ngef, Plpp1, A2m, Dstn, Cox6a1, Gale, Pde10a, Krtdap, Rab6b, Esam, Crct1, Col5a2, A4galt, Dnasell3, Sfn, Bmp1, Calr, Papl, Kank1, Prl6a1, Cmtm5, Ildr2, Rhoc, Ccl27a, Efs, Hrct1, Dsc2, Bcar1, Cd63, Inhbb, Ppp2r2c, Mpdz, Mt1, Fetub, Nid1, Bicc1, Psg23, Hdlbp, Rasd1, Arhgap22, Maged2, Aard, Prl8a6, Cald1, Prom2, Pdia6, Nostrin, Prss8, 1600012P17Rik, Dsp, Adm, Fam90a1b, Exoc314, Rbfox2, Mgst3, Gm648, Rgs5, Bgn, Galk1, Adra2b, AT036118, Nenf, Aifl1, Nfib, Acvr2b, Htatsf1, Rpn1, Psg27, Phldb2, Myof, Fbx119, H1f0, Cobl, Emp2, Myrf, Adamts7, Fam89a, Gm9513, Tmem98, Mfsd2a, Mgat4b, Cav1, Il33, C77080, Lars2, Gm21887, 9930038B18Rik, Prom1, Ptov1, Ltbp4, Mmp23, Prl7a2, Cts3, Fabp6, Prkcdbp, Lmna, Rpn2, Caskin1, Lama1, Fam83b, Cryaa, Lamc2, 4933402E13Rik, Fn1, Dlc1, Lrrn4, Tff1, Elf3, Serpine1, Bcat1, Perp, Lama5, Gpx3, Car2, Pawr, Phactr1, Oaf, Lamb2, Prnp, Scin, Parm1, Smim1, Jup, Gpx8, Atf5, Ift43, Gkap1, H1fx, Cenpt, Rhou, Rrm2, Cul7, Asns, Tubb3, 2200002D01Rik, Abhd6, Hdac6, Rgs16, Cgnl1, Atp9a, Src, 4930486L24Rik, Elf3, Ly6c1, transferrin receptor (TfR), ZNF614, ZNF539, ZNF283, ZFYVE9, WRN, UBL4A, TPM3, TP73, TMEFF2, THC2274391, THC2265980, t (BRACHYURY), SYT9, SLAMF1, RPS27, RPL39, RPL26, RPL23A, RPL23, RP11-78J21.1, RGPD2, QPRT, PPIA, PGK1, PF4, NTRK1, NBR2, MYL6, MAPK3, MAD2L1, LOC389286, KY, KMO, KCNK4, ITGA7, HMGN2, HMGA1, HLA-C, HIST1H2AJ, GSK3A, GCC2, FYN, F1135740, ENST00000356196, EEF1A1, EDN1, DKFZP434F142, D4ST1, CRYL1, CPS1, CD7, CCNA2, CB123670, BC111482, 6C089454, ALS2CL, AK000420, AHNAK, ADAM11, ACVR2B, ABHD2, CD105, HLA-G, galectin 13 (LGAGLS13/PP13), galectin 14 (LGALS14), placental growth factor (PGF), pregnancy-associated plasma protein A (PAPPA), alpha fetal protein (AFP), endoglin (ENG), fns-related tyrosine kinase 1 (FLT-1), keratin-7, PLAP, MCAM, laeverin, H315, FT1.41.1, NDOG-1, NDOG-5, BC1, AB-154, AB-340 (PAR-1), Glut-12, factor XIII, hPLH, HLA-C, JunD, Fra2, NDPK-A, CAR, HASH2, αHCG, IGF-11, PAI-1, p57 (KIP2), PP5, PLAC1, PLAC8 and PLAC9, CHL1, H119, HLA-C, Fra2, NDPK-A, the CAR protein, HASH2, human chorion gonadotropin alpha (hCGα), TFPI-2, annexin IV, cytokeratin-7, cytokeratin-8, cytokeratin-19, H19, IFG2, ESX1L, MASH2, ASH2, Stra13, FosB, Cyclin D1, GCM1, caspase-8, factor XIII, tapasin, Connexin 31, Connexin 43, HAND1, Syncytin, MMP9, APAF-1, caspase-3, caspase-9, FAS, Fas ligand, FLIP, AP-2γ, 313-HSD VI, CDX2, ERR2, PLX3, PTHrP, ASCL2, ID2, MET, TEF5, UPA, 11B-HSD2, c-ETs1, HMGI(Y), estrogen receptor, GemI, TTPA, 1503-7E, J42-4d, J2r(3), J2r(12), J2r(13), 305-4G, K1-1a, K2r/1f(50), K2r/1f(59), K(1)157-2A, K3r(HIGH)76, 597-10C, NT7-T3, N9r.Mf, 334-2C, O19r-T3, O1-1a, 332-9E, P60-1a, P1-1a, P3r(9), 305-9E, R5′-T3, R6r/1-6H, 369-8G, U2f-T3, 305-6G, L15-1a, L21-1a, 252, 120r, clone-1, D19-2g, CD146, CD141, vimentin, VCAM, ICAM, VEGFR-1, VEGFR-2, VEGFR-3, ITGA5, ITGB5, CDH11, CDH3, CK1, CK2, CK3, CK4, CK5, CK6, CK7, CK8, CK9, CK10, CK13, CK14, CK15, CK16, CK17, CK18, CK19, CD9, CD39, CD200, EPHB4, EPCR, PAR-1, hPL, CSH2, KISS1, GDF15, CRH, TFPI2, β-hCG, LOC90625, FN1, COL1A2, PSG9, HBE, AFP, GC, APOC3, SERPINC1, APOB, AHSG, HPX, CPB2, ITIH1, APOH, AMBP, Thy-1, CD90, thromobomodulin, Flt-1, EPCR, CDH11, CDH3, CD59, CD71, CD36, Glycophorin A, hexokinase 2, placental alkaline phosphatase, hyalouronic acid, BCL-2, VE-cadherin, PECAM-1, α1-integrin, placental lactogen, PSG-1, α6 integrin, E-cadherin, MMP14, KCNQ4, CLDN6, F3, EPCAM, TROP2, CD147, fetal hemoglobin, embryonic hemoglobin, ITGA6, FSHR, CD45, LHCGR, CD270, Trop2, FOLR1, CD114, CSF3R, SLC40A1, CD156a, ERVW1, GPC3, TREML2, ALPP (866), CD163, CD144, ALPP (GM022), FOLR1, CD115, TSPAN1, SLC22A1, EFNA1, SLC62A2, SLC40A1, CD71, CD166, TGF-beta2, human placental lactogen, c-erbB2, PAPPA2, PRG2, cytokeratin 7, beta-HCG, alpha-HCG, GCM1, syncytin, c-erbB2, leptin, INSL4, TGF-beta1, CSH1, KISS1, PEG10, PAGE4, p63, miR1246, miR1323, miR-512-3p, has-miR-516b-5p, miR-517-5p, has-miR525, miR-526b, ath-miR159a, RNU44, RNU48, U6, CDH9, HLA-G, MIH61, ALPP (8B6), CD49e, JAM2, transferrin receptor, glycophorin a (GPA), EGFR, thrombospondin receptor (CD36), CD34, HbF, HAE 9, FB3-2, H3-3, erythropoietin receptor, HBE, AFP, APOC3, SERPINC1, AMBP, CPB2, ITIH1, APOH, HPX, AHSG, APOB, J42-4-d, 2,3-biophosphoglycerate (BPG), carbonic anhydrase (CA), thymidine kinase (TK), MMP14, lectin, CD235a, carbohydrates, selectin, GPA, ANTIGEN-I, EpCAM, E-cadhrin, Muc-1, Hpl, CHS2, KISS1, GDF15, CRH, TFP12, CGB, LOC90625, FN-1, COL1A2, PSG9, PSG1, EGFR, CD105, cytokeratin, TLS1, CD147, CD147, PTPRN, BMPR2, TNFRSF19, STIM1, SELPLG, KLP-1, HECA452, GPR160, KLRD1, TNFSF18, LRP11, ABCA1, GPR176, SCARF, ESYT1, SCARB1, LTB4R, TLT2, CD141, CD90, Vimentin (Vim), VCAM, ICAM-1, VEGFR-1, Flt-1, VEGFR-1, VEGFR-2, VEGFR-3, PAI-1, EPCR, CD146, ITGA5, ITGB5, CDH11, CDH3, CD59, CSH-1, CSH-2, PSG2, PSG3, PSG11, HBG2, XAGE3, CSHL1, HBG1, ERVH48, phCG, any human orthologue of any of the foregoing, and any combination thereof.

In some embodiments, the fetal biomarkers of the present disclosure have human orthologues. Non-limiting examples of human orthologues of the fetal biomarkers include: AOC1, FAM236B, FAM236D, FAM236A, FAM236C, C11orf52, MAGEA10, A2M, A4GALT, AARD, ACVR2B, ADA, ADAMTS7, ADGRF5, ADM, ADRA2B, AFAP1L2, AIF1L, ALPL, APRT, ARHGAP22, ARHGAP42, BASP1, BCAR1, BEX1, BEX2, BGN, BICC1, BMP1, BOK, BSG, KIAA1522, CALD1, CALR, CA4, CASKIN1, CAV1, CCL27, CD63, CDA, CDH3, CDH5, CDKN1C, CEACAM4, PSG3, PSG8, PSG1, PSG6, PSG7, PSG11, PSG2, PSG5, PSG4, PSG9, CHCHD10, CHRDL2, CITED1, CKB, CMTM5, COBL, COL4A1, COL4A2, COL5A2, COPZ2, COX6A1, CRIP2, CRYAA, CRYAB, CSRP1, CSTB, CYB5R3, CYP11A1, CYP17A1, CCN1, CYSTM1, DB1, DCN, DES, DLC1, DMKN, DNASE1L3, DSC2, DSP, DST, DSTN, DUSP9, EFS, EGFL7, EMP2, EPAS1, EPCAM, EPN3, EPS8L2, ESAM, EXOC3L4, FABP3, FABP6, FAM83B, FAM89A, FAM90A1, FAM90A26, FAM90A7, FAM90A22, FAM90A23, FAM90A14, FAM90A18, FAM90A16, FAM90A8, FAM90A17, FAM90A19, FAM90A9, FAM90A10, FBXL19, FDX1, FERMT2, FETUB, FKBP9, FMR1NB, FN1, FOXO4, FSCN1, FSTL3, GALE, GALK1, GHRH, GJB3, CT45A1, CT45A3, CT45A5, CT45A6, CT45A2, CT45A7, CT45A8, CT45A9, CT45A10, SAGE1, TMEM267, GPR50, H1-0, HAND1, HDLBP, HMGN1, HOMER2, HRCT1, HSD17B2, HSPB1, HSPG2, HTATSF1, HTRA1, IGF2, IGF2BP1, IL33, ILDR2, INHBB, IRX3, KANK1, KLHL13, KRT18, KRT19, KRT7, KRT8, KRTDAP, LAMA1, LAMC2, LARS2, LDHB, LSOC1, LEPR, LGALS1, LGALS3, LMCD1, LMNA, LRRN4, LTBP4, MAGED1, MAGED2, MAP1B, MDK, MEIS2, MFSD2A, MGAT4B, MGST3, MMP1, MMP23B, MPDZ, MRGPRG, MT1G, MT2A, MUC1, MYOF, MYRF, NBL1, NENF, NFIB, NGEF, NID1, NOS2, NOSTRIN, NPC2, NPPC, NRK, NUP62CL, NXF5, NXF2, NXF2B, P4HB, ACP7, PARVA, PED10A, PDIA5, PDIA6, PEG10, PEG3, MPEG1, PHLDB2, PKP2, PLA2G5, PLAC1, PLET1, PLPP1, PODXL, PPIB, PPP2R2C, PRAMEF12, PRAMEF1, PRAMEF11, PRAMEF2, PRAMEF4, PRAMEF10, PRAMEF7, PRAMEF6, PRAMEF27, PRAMEF25, PRAMEF26, PRAMEF9, PRAMEF13, PRAMEF18, PRAMEF5, PRAMEF8, PRAMEF33, PRAMEF 15, PRAMEF14, PRAMEF19, PRAMEF17, PRAMEF20, PRDX4, PRKCD, PROCR, PROM1, PROM2, PRSS8, CEACAM4, PTOV1, NECTIN2, RAB15, RAB6B, RARRES2, RASD1, RBFOX2, RBP1, RGS17, RGS5, RHOC, RND2, RPN1, RPN2, S100A6, SBSN, SCT, SDC1, SEC61G, SEMA3E, SEMA3F, SERBINB6, SERBINB9, SERPINH1, SFN, SHROOM1, SIN3B, SLC30A2, SLC38A4, SLC6A2, SLCO2A1, SPARC, SSR4, STAR, STARD10, STRA6, SULT1E1, SYNGR1, TBRG1, TEX19, TFAP2C, TFF1, TFPI, TINAGL1, TMEM37, TMEM98, TNFRSF9, TPM1, TPM2, UACA, UQCRB, ATP5MK, VGF, FAM9A, FAM9B, FAM6C, C19orf33, ABHD6, ASNS, ATF5, ATP9A, BCAT1, CA2, CENPT, CGNL1, CTSL, CUL7, ELF3, FBXL19, GKAP1, GPX3, GPX8, H1FX, HDAC6, IFT43, JUP, LAMA5, LAMB2, OAF, PARM1, PAWR, PERP, PFPL, PHACTR1, PRNP, RGS16, RHOU, RRM2, SCIN, SERPINB6, SERPINB9, SERPINE1, SMIM1, SRC, TUBB3, and any combination thereof.

In some embodiments, the biological agent recognizes a Sct biomarker or a human orthologue of a Sct biomarker. In some embodiments, the biological agent recognizes a H19 biomarker or a human orthologue of a H19 biomarker. In some embodiments, the biological agent recognizes a Tfpi biomarker or a human orthologue of a Tfpi biomarker. In some embodiments, the biological agent recognizes a Fthl17a biomarker or a human orthologue of a Fthl17a biomarker.

In some embodiments, the biological agent recognizes a maternal biomarker. In some embodiments, the maternal biomarker is located in a maternal cell. In some embodiments, the maternal biomarker is located on a maternal cell. Non-limiting examples of maternal cell biomarkers include Coro1a, Rac2, Malat1, Laptm5, H2-D1, B2m, Cytip, Alox5ap, Cd52, Junb, Cyba, Fyb, Btg1, Srgn, Actb, Klf2, Mcl1, H3f3a, Hmha1, Tmsb4x, Cd53, Ptprc, Fxyd5, Myl12b, Tyrobp, Gmfg, Fcer1g, Ier2, H2-K1, Itm2b, Jund, Rasgrp2, Nfkbia, Itgb2, Pfn1, Ifngr1, Ltb, Pim1, Ssh2, Rbm39, Sell, Trbc2, Msn, Ddx5, Stk17b, Lyz2, Cd37, Csf3r, Hcst, Ifitm2, Pnrc1, Rap1b, Meat1, Fth1, Cd3d, Arhgdib, Pglyrp1, H3f3b, Ms4a6b, Akap13, S100a9, Lyn, Sorl1, Mbnl1, Cd44, Ier5, Lsp1, Cdc42, Aes, Ms4a6c, Msrb1, Gdpd3, Btg2, Sat1, Arhgap30, Lrg1, Itga4, Oaz1, Rhoa, Eif1, Zfp3612, Selplg, Cd3g, Zfp36, Arhgap 15, Syk, Arpc2, Sf3b1, Ighm, Psmb9, Gnai2, Actr3, Lst1, Il1b, Satb1, Amp32a, Foxp1, Lcp1, Wfdc17, Taldo1, Slfn2, Rgs10, Ccnd3, Ncor1, Cd3e, Vim, Cebpb, Tpm3, Adgre5, S100a8, Ccl6, Ptpn18, Napsa, Rps9, C5ar1, Hnmnpf, Iqgap1, Ly6e, Klf13, Ifitm3, Psmb8, H2-Q4, Gsr, Hmgb2, Fos, Sh3bgrl3, Dusp1, Spi1, Cd84, Tspo, Lef1, Cyp4f18, Atp11b, Ccr1, Gpx1, Cd47, Samhd1, AB124611, Trac, AI467606, Shisa5, My16, Tspan13, Cfl1, Gm5150, Itgal, Calm2, Plek, Ucp2, Tsc22d3, Ikzf1, Grk6, Gimap1, Ybx1, Gm8797, Clic1, Rara, Limd2, Ifi2712a, Ubb, Gngt2, Cd14, Pla2g7, Ets1, Tgfb1, Actg1, Il17ra, Celf2, Srsf5, Gimap6, Tmem50a, Ctss, Ube2d3, Tln1, Apbb1ip, Gabarap, Arpc5, Kmt2e, Klf3, Cap1, Itgam, Emp3, Clk1, Ogt, Crlf3, Samsn1, Stk38, Tax1bp1, Lat, Clec4e, Cdkn2d, Rel, Srrm2, Smc6, Morf411, Rsrp1, Ptpn1, Rgs2, Fmnl1, Gpsm3, Klf6, Fcgr3, Mef2c, Birc2, Kdm6b, Tra2b, Ptprcap, Son, Rassf5, Rpl22, Gimap3, Hnrnpa2b1, Lbr, Lck, Calm1, Cdkn1b, Ccr7, Ypel3, Ptpn6, Cd74, Ppp2r5a, Cd9, Rcsd1, Svi1, Hmgb1, Rhog, Hacd4, 9930111J21Rik2, Stat1, Ncf4, Fam105a, Rsbn1l, Hp, Ankrd11, Emb, Dusp2, Pfdn5, Txnip, Psme1, Slfn1, Ostf1, Jak1, Sirpa, Rap1a, Fam49b, Gda, Picalm, Ywhaz, Pde4b, Dazap2, Tsc22d4, Pkm, Slc25a5, Kdm7a, Lcp2, Fus, H2-Q7, Mxd1, Glipr1, Bnip31, Luc712, Tnfaip812, Pdcd4, Map1lc3b, Plbd1, Skap1, Unc93b1, Ncf2, Rnf149, Bin2, Mycbp2, Cnp, S100a10, Ubc, Cd300ld, Fam32a, Ptp4a2, Adrb2, Prdx5, Crip1, Sna32, Csk, Tnrc6b, Arhgef1, Pou2f2, Hspa8, Pilra, Bcl11b, Zfp292, Cnn2, Aldh2, Ywhah, Zbtb7a, Pycard, Cotl1, Retnlg, Sp100, Irf2, Fgr, Igsf6, Ablim1, Fam65b, Fermt3, Tmbim6, Tpd52, Scp2, Ube2b, Notch2, Gnb2, Il7r, Capzb, Jun, Ptma, 2810474O19Rik, Prr13, Mrpl33, Ppp1r15a, Lilrb4a, Zyx, St8sia4, Arpc3, H2afz, S100a11, Pag1, Irf1, Tm6sf1, Rsf1, Frina, Rasa3, Smap2, Dok3, Skap2, Add3, Stx11, Mkrn1, Hck, Ccnl1, Ppp1ca, R3hdm4, Cd48, Chd2, Rgs19, Cth4, Atp2b1, Peli1, Fam107b, Ptbp3, Adipor1, Serp1, Pak2, Hspa5, Tpr, Zc3hav1, Sdcbp, Dock2, Rnf130, Ftl1, B4galnt1, Cd300a, Hcls1, Anxa1, Cst3, Ankrd44, Elf1, Prpf38b, Atp6vlg1, Snx20, Il6ra, Whsc1l1, Tmem71, Stk4, Sla, Mbp, Dnaja1, Vgll4, Capza2, Ldha, Pyhin1, Rapgef6, Hdc, Fli1, Rbm25, H2afy, Prdx6, Rac1, Stap1, Supt4a, Hnrnpk, Ubl5, Cdk2ap2, Ppp1r2, Psenen, Bcl2, Tprg1, Arpc1b, Cd69, Phf20l1, Ppt1, Rhoh, Coro1a, Cytip, Malat1, Rac2, Cd52, Laptm5, B2m, H2-D1, Cyba, Ptprc, Btg1, Gmfg, Junb, Srgn, Fxyd5, Itgb2, Ltb, Tyrobp, Mcl1, Cd53, Klf2, Fcer1g, H3f3a, Hmha1, Itga4, Alox5ap, Ptpn18, Myl12b, Cd3d, Spi1, Ier2, Ccl6, Ms4a6b, Msn, Selplg, Pglyrp1, S100a9, Lyn, Slfn2, H2-K1, Ier5, Cyp4f18, Csf3r, Hp, Lst1, Jund, Fyb, Rap1b, Pim1, Il1b, Lyz2, Stk17b, Cd3e, Gpsm3, Lef1, Sell, Hcst, Gm5150, Ms4a6c, Fmnl1, Adgre5, Arhgap30, Lrg1, 9930111J21Rik2, Unc93b1, Rbm39, Pnrc1, Ptpn6, Trac, Akap13, Actr3, Ifitm2, Crlf3, Msrb1, Samsn1, Ets1, Lck, Pla2g7, Zfp3612, Lat, Ncor1, Sorl1, Ccnd3, Aes, Rasgrp2, Samhd1, Psmb8, S100a8, Wfdc17, Ctss, Neat1, Arhgap15, Ncf4, Lcp1, AB124611, Ssh2, Btg2, March1, Snx20, Fam105a, Zfp36, C5ar1, Tspan13, Hck, Apbb1ip, Mbnl1, Cd37, Limd2, Skap1, Iqgap1, Cybb, Lcp2, Ly6e, Slfn1, Bcl11b, Syk, Sf3b1, Ccr1, Ddx5, Stk38, Sat1, Anp32a, Dock2, Cd74, Satb1, Ighm, Foxp1, Tspo, Klf13, Ptpn1, AI467606, Nfam1, Cap1, Hnmnpf, Shisa5, Fgr, Rhog, Ifngr1, Psmb9, Rhoh, Lilrb4a, Plek, Rasa3, Sla, Atp11b, Fam65b, Gimap1, Hmgb2, Ypel3, Tnfaip812, Pirb, Rgs2, Tmem50a, Fcgr3, Cd84, Kmt2e, Mpeg1, Retnlg, Ikzf1, Il6ra, Prdx5, Ostf1, Cd14, Itgam, Cd300ld, Rnf149, Cd79b, Dusp1, Tsc22d4, Ifi2712a, Il17ra, Sirpb1c, Klf3, Celf2, Clic1, Cebpb, Grk6, Pira2, Grap, Ppp2r5a, Fam49b, Cd47, Tgfb1, Wfdc21, Hcar2, Ncf2, Cd300a, Ms4a4b, Itgal, Trbc2, Dusp2, Tra2b, Pilra, Cyth4, Hcls1, Pyhin1, Son, Tgfbi, Tspan32, Vav1, Cd3g, Irf1, Dok3, Cxcr2, Ifitm3, Rara, 2810474O19Rik, Cd33, Adrb2, Snap23, Fos, B4galnt1, Lilr4b, Tpm3, Prkcq, Cd44, Rcsd1, Sp100, S1pr1, Cdk2ap2, Cd247, Cotl1, Lrrfip1, Slc11a1, Srsf5, Cnp, Klf6, Clec4e, Inpp5d, Glipr1, Lpcat2, Fermt3, Clec4a3, Fus, Itgb7, Tbc1d10c, Napsa, Pld4, Add3, Jak1, Aldh2, Cdkn1b, Gsr, Myo1f, Ccr7, Mxd1, St8sia4, Gimap3, Arpc5, Ly6d, Rassf5, Ifitm1, Arhgef1, Gdpd3, Ifitm6, Smap2, Fam107b, Cd224, Mcemp1, Bin2, Hdc, Fam32a, Pou2f2, Kdm6b, Cdkn2d, Vim F630028O10Rik, H2-Q4, Pag1, Rnf130, Smpdl3a, Tpd52, Tmcc1, Grina, Fgl2, Csk, Trbc1, Cd27, D1Ertd622e, Rasal3, Gabarap, Cd28, Ipcef1, Rgs19, Bcl2, 4930523C07Rik, Slc25a5, Rapgef6, Ppt1, Gngt2, Irf2, Psme1, Clec4d, Slc15a3, Pilrb2, Andrk11, Peli1, Tsc22d3, Bank1, Iglc2, Il7r, Stfa211, Stat1, Igsf6, Tax1bp1, Nedd9, Tm6sf1, Ly86, Tprg1, 2310001H17Rik, H2-DMa, H2-DMb2, Sirpb1b, Srrm2, Stk4, H2-T23, Abcg1, Klhl6, Ablim1, Evi2a, Bnip31, Arhgap25, Lrrc25, Ogfrl1, Emp3, Clk1, Mkrn1, Ggnt2, Hacd4, Picalm, Adipor1, Man2b1, Cd48, Lyl1, Sp140, Sephs2, Lbr, Cd69, Phf20l1, Scp2, Ms4a1, Cdc42se1, Rgs10, Sirpa, Stap1, Ddx6, Ppp1r2, Abi3, Acap1, Csf2rb, Tnfrsf18, Alox12, Ebf1, Luc712, Flna, Ikzf3, Sept1, Arid4a, Pi16, Gimap6, Dazap2, Ppplr16b, Lyst, Adrbk1, Nlrp3, Ptprcap, Pycard, Themis2, Lbh, Mycbp2, Rel, Apobec1, Cd2, Chd2, AF251705, Trem1, Dennd1c, Ram189b, Ncf1, Tnfaip2, Pde4b, Ccnl1, Fcgr4, Skap2, Elf1, Il2rg, Slc7a11, Trim30b, Smchd1, H2-Q7, Rasgrp1, Ptpn22, Mrgpra2b, Ptpn22, Mrgpra2b, Cmip, Dgka, 1600010M07Rik, Igkc, Slc2a6, Atg3, Arl4c, Il16, Btk, Il21r, Fli1, Gda, Mbd2, Fam134b, Myo1g, Pygl, Ptpre, Kdm7a, Ctse, Plbd1, Map3k3, Akna, Mmp8, Ywhaz, H2afy, Svi1, Mfsd6, Emilin2, Hnrnpa0, Psenen, Pdcd4, Cd5, Chill, Pnpla2, Macf1, Gm8369, Nfatc1, Plcg2, Rhof, Gm9733, Map1lc3b, Capzb, Ube2b, Ccpg1, Il1f9, Xist, Gimap5, Rsbn1l, Cbl, Cd300lb, Siglece, Slco3a1, Supt4a, Mbp, Ms4a4c, Cd79a, Nrros, Gimap7, Birc2, Ccdc12, Mapk14, Tcf7, Whsc1l1, 1830077J02Rik, Rbm25, Xrn2, Jakmip1, Dock10, Vsir, Siglecg, Ramp1, Cd6, Phip, Ifi203, Tpr, Kpna4, Ptafr, Sh3bp5, Itk, Tnrc6c, Notch2, Myd88, Nipbl, Prkcd, Slc2a3, Bcl10, Il10ra, Dhrs7, Clec4a1, S1pr4, Cd300lf, Ogt, Lrmp, Ttc7, Gm43603, Treml1, Bcl2alb, Marcks, Mier1, Vasp, Clec4a2, Gpcpd1, Traf3ip3, Prpf38b, Runx3, R3hdm4, Lfng, 1110008F13Rik, Slamf6, Pdcd10, Stx11, Srsf11, Gp5, Hnrnpl, BC028528, Gm26740, Pid1, Nfe2, Mzb1, Lilra6, Snca, Parp14, Vgll4, Socs3, Sh2d1a, Dapl1, Gpr183, AW011738, Hbb-bs, Lmnb1, Strbp, H2-Aa, Il18rap, Hipk1, Stag2, Orai1, Smc6, Stk10, Fam111, Treml4, Ctage5, Ebi3, Ap1s2, Neurl3, Trib2, Zc3hav1, Baz1a, Hectd1, Sec11c, Tnrc6b, Amica1, Prkd2, Rnase6, Ev1, Vav3, Rab27a, Trim30a, Adam8, Phf3, Gpr132, Sirt7, Tcp11l2, Zcchc7, Itpr2, Bptf, Ace, Icam2, Slfn4, Fcmr, Prkar1a, Asap1, Clec2i, Prr13, Mef2c, Kansl1, Slc40a1, Vps37b, Sdcbp, Cd8b1, Phf11b, Zeb2, Sfpq, Ccl3, Anxa1, Pik3cd, Serp1, Arrb2, Ccdc88b, Ctsc, Atf7ip, Treml2, Nfkb1, Vamp8, Slc16a3, Kif2a, Lmo2, Capza2, Tbc1d1, Gimap8, Rassf3, Ifngr2, Ltb4r1, Trem3, Pf4, Clec2d, Pten, Cd226, H2-Ab1, Sgk1, Zcchc11, Lcn2, Rgs14, Ptp4a2, Tmem156, Mctp1, H2-Q6, N4bp1, Arhgap9, Birc3, Txk, Nptn, Psme2, Arl6ip5, Pstpip1, Tuba4a, Sertad1, Diaph1, Tnfrsf13b, Batf, AW112010, Il10rb, Arl5c, Cxcl2, Ccdc88c, Cdk9, Sh3bp1, Gimap4, Rnf144a, Bcl11a, Rassf2, Gramd1a, Nadk, Tnfrsf13c, Tra2a, Ggnbp2, Isg15, Def6, Stat4, Prrc2c, Gm15987, Card19, Clec12a, Gsap, Mfng, BC021614, Cmtm6, Cpr65, Tctex1d2, Cntrl, Tacc1, Zfp292, Sh3kbp1, Ifi47, Gpr18, Syf2, Twistnb, Rinl, Gp9, Aim2, Ctcf, Cd300e, Dck, B430306N03Rik, Otulin, Pip4k2a, Max, Tnfsf14, Krit1, Tmpo, Tnf, Ubl3, Csnk1a1, Fpr2, Cecam1, Bmx, Vrk1, Prkcb, Pfkfb4, G3bp2, Abtb1, Epsti1, Rin3, Itpkb, Gimap9, Adgre4, Ms4a6d, Ly9, Tap2, Prkar2b, Sf3b6, Fam78a, Adhb8, 1700097N02Rik, Zdhhc18, Atp1a3, Ppp1r10, Ifi30, Mcmbp, Twf2, C130050O18Rik, Kras, Arhgap4, Tmem71, Alcam, Notch1, Morc3, Sun2, Haus8, Rsrc2, Cdc42ep3, Ikbkb, Chd7, Arl11, Clec4n, Cxcr4, Stk24, Nfkbiz, Serinc3, Timm10b, Ctsw, Rchy1, Tap1, Maf1, Rbm5, Sema4a, Mat2b, Cth1, Nfkbid, Cd4, Ly6c2, Mef2a, March2, Grap2, Av19, Fcer2a, Cd24a, Hba-a1, Bcl2a1a, Arf6, Rnf13, Blnk, Il27ra, Kcnn4, Hn1, Fpr1, Sash3, Atp8b4, Sfxn5, Gpr141, Rsu1, Prr51, Scnn1a, Tmem243, Fcrl1, Pik3r1, Nlrp12, Milr1, THBD, F3, FLT3, MCAM, CD141, CD135, CD142/F3, CD146, CD33, CD68, CD144, CDH5, CD62E, CD45, CD90, SELE, GAP/GLA5, ITGA5, KCNK3/TASK-1, CNTFR, JAM-1 (f11r), SDC4/Syndecan 4, ACVR2B, ACVR2A, CD44v6+CD44v4/5, STXIA, TEK/Tie2, CD171, L1CAM, NCAM1/CD56, ALPP, ALPPL2, MMP23A/MMP23B, CD14, CD4, CD8, CD3, CD19, CD32, CD16, HLA-A, HLA-B, CD15, FSHR, CD45, LHCGR, CD270, Trop2, FOLR1, CD114, CSF3R, SLC40A1, SLC22A1, SLC22A1 CD156a, ERVW1, GPC3, TREML2, ALPP (866), CD163, CD144, ALPP (GM022), FOLR1, CD115, TSPAN1, SLC22A1, EFNA1, SLC62A2, SLC40A1, CD71, CD166, CDH12, HLA-A, HLA-B, PRSS8, CD45, CD230, ALPP (8B6), CD49e, JAM2, TREML2, MMP14, lectin, CD47, CD45, CD35, CD12, CD14, CD32, CD235a, carbohydrates, selectin, GPA, ANTIGEN-I, EpCAM, E-cadherin, Muc-1, Hpl, CHS2, KISS1, GDF15, CRH, TFP12, CGB, LOC90625, FN-1, COL1A2, PSG9, PSG1, CD105, TLS1, CD147, CD15, TNFRSF19, STIM1, CD19, CD14, any human orthologue of any of the foregoing, and any combination thereof.

In some embodiments, the maternal biomarkers of the present disclosure have human orthologues. Non-limiting examples of human orthologues of the maternal biomarkers include: CYBA, CORO1A, CYTIP, LAPTM5, CD53, RAC2, CD52, PTPRC, FCER1G, ARHGAP45, GPM6A, MAF, C3, CFH, EFEMP1, BICC1, COL1A2, COL1A1, B2M, BTG1, GMFG, JUNB, SRGN, ITGB2, LTB, TYROBP, MCL1, KLF2, NFKBIA, H3F3A, ITGA4, ALOX5AP, PTPN18, CD3D, SPI1, IER2, CCL15-CCL14, CCL14, CCL15, CCL23, MS4A6A, MS4A6E, MSN, SELPLG, ACTB, PGLYRP1, TMSB4Y, LYN, SLFN12, SLFN12L, IER5, CYP4F3, CSF3R, HP, HPR, LST1, ITM2B, JUND, FYB1, LSP1, RAP1B, PFN1, PIM1, IL1B, STK17B, CD3E, GPSM3, LEF1, SELL, HCST, AL049634.2, SIRPB1, SIRPG, SIRPA, MS4A6A, MS4A6E, FMNL1, ADGRE5, ARHGAP30, LRG1, UNC93B1, RBM39, PNRC1, PTPN6, AKAP13, ACTR3, IFITM2, IFITM1, IFITM3, ARHGDIB, CRLF3, MSRB1, SAMSN1, ETS1, LCK, PLA2G7, ZFP36L2, LAT, NCOR1, SORL1, CCND3, AES, RASGRP2, SAMHD1, PSMB8, S100A8, CTSS, ARHGAP15, NCF4, LCP1, C19orf38, SSH2, BTG2, MARCH1, GNAI2, SNX20, FAM105A, ZFP36, C5AR1, TSPAN13, HCK, APBB1IP, MBNL1, CD37, LIMD2, SKAP1, IQGAP1, CYBB, LCP2, EMB, LY6E, SLFN12, SLFN12L, BCL11B, SYK, SF3B1, CCR1, DDX5, STK38, SAT1, ANP32D, ANP32A, TALDO1, CDC42, DOCK2, CD74, SATB1, IGHM, TSPO, KLF13, H3F3B, PTPN1, C16orf54, NFAM1, CAP1, HNRNPF, SHISA5, FGR, RHOG, IFNGR1, PSMB9, RHOH, PLEK, RASA3, SLA, ATP11B, RIPOR2, SH3BGRL3, GIMAP1, HMGB2, RHOA, YPEL3, TNFAIP8L2, LILRB3, LILRA6, LILRB5, LILRA4, ARPC2, RGS2, TMEM50A, FCGR2A, FCGR2C, FCGR2B, CD84, KMT2E, MPEG1, RETNLB, TXNIP, IKZF1, IL6R, PRDX5, OSTF1, CD14, ITGAM, CD300LF, RNF149, CD79B, DUSP1, TSC22D4, IL17RA, AL049634.2, KLF3, CELF2, CLIC1, CEBPB, GPX1, GRK6, LILRA5, GRAP, GRAPL, PPP2R5A, FAM49B, CD47, TGFB1, HCAR2, HCAR3, NCF2, CD300A, CD300LD, ITGAL, TRBC1, TRBC2, OAZ1, DUSP2, TRA2B, CFL1, CYTH4, ANKRD44, HCLS1, SON, TGFBI, TSPAN32, VAV1, CD3G, IRF1, MRPL33, DOK3, CXCR2, IFITM1, IFITM3, IFITM2, RARA, KIAA1551, CD33, SIGLEC6, ADRB2, SNAP23, FOS, B4GALNT1, PRKCQ, CD44, RCSD1, UCP2, SP140, SIPR1, TLN1, CDK2AP2, CD247, COTL1, LRRFIP1, SLC11A1, SRSF5, CNP, CNN2, KLF6, CLEC4E, INPP5D, GLIPR1, LPCAT2, FERMT3, CLEC4C, FUS, ITGB7, TBC1D10C, NAPSA, PLD4, ADD3, JAKI, AC002996.1, ALDH2, CDKN1B, GSR, MYO1F, CCR7, MXD1, ST8SIA4, GIMAP1-GIMAP5, GIMAP1, GIMAP5, ARPC5, LY6D, RASSF5, IFITM1, IFITM2, IFITM3, ARHGEF1, GDPD3, SMAP2, FAM107B, MCEMP1, BIN2, HDC, FAM32A, POU2F2, KDM6B, CDKN2D, VIM, PAG1, RNF130, SMPDL3A, AC036214.3, TPD52, TMCC1, GRINA, FGL2, CSK, TRBC1, TRBC2, CD27, MACIR, RASAL3, GABARAP, CD28, IPCEF1, RGS19, BCL2, KIAA0040, SLC25A5, RAPGEF6, AC008695.1, PPT1, GNGT2, IRF2, PSME1, CLEC4D, SLC15A3, ANKRD11, PELI1, TSC22D3, RSRP1, YBX1, BANK1, IGLL1, IL7R, CTSA, STAT1, IGSF6, TAX1BP1, NEDD9, TM6SF1, LY86, TRPG1L, HLA-DMA, HLA-DMB, SRRM2, STK4, ABCG1, KLHL6, ABLIM1, RAP1A, EVI2A, BNIP3L, ARHGAP35, LRRC25, OGFRL1, EMP3, CLK1, MKRN1, GCNT2, HACD4, PICALM, UBE2D3, ADIPOR1, MAN2B1, CD48, LYL1, PAK2, SEPHS2, LBR, CD69, PHF20L1, SCP2, MS4A1, CDC42SE1, RGS10, STAP1, DDX6, PPPIR2, PPPIR2B, ABI3, ACAP1, PKM, CSF2RB, TNFRSF18, ALOX12, EBF1, LUC7L2, FLNA, IKZF3, PRDX6, SEPTIN1, ARID4A, PI16, GIMAP6, DAZAP2, PPP1R16B, LYST, GRK2, NLRP3, HNRNPA2B1, PTPRCAP, PYCARD, THEMIS2, LBH, MYCBP2, REL, APOBEC1, CD2, CHD2, TREM1, DENND1C, ENTREP3, NCF1, TNFAIP2, PDE4B, ATP6VIG1, CCNL1, FCGR3A, FCGR3B, SKAP2, ELF1, IL2RG, SLC7A11, SMCHD1, CD9, RASGRP1, PBXIP1, PTPN22, MRGPRX3, MRGPRX4, MRGPRX1, MRGPRX2, CMIP, DGKA, IGKC, SLC2A6, ATG3, ARL4C, IL16, BTK, IL21R, PPP1CA, FLI1, GDA, MBD2, MYO1G, PYGL, SCAND1, PTPRE, KDM7A, CTSE, PLBD1, MAP3K3, AKNA, MMP8, YWHAZ, MACROH2A1, SVIL, H2AJ, MFSD6, ZYX, EMILIN2, HNRNPA0, PSENEN, PDCD4, CD5, CHI3L1, PNPLA2, MACF1, NFATC1, PLCG2, RHOF, CALM2, ROCK1, MAPILC3B2, MAP1LC3B, CAPZB, UBE2B, CCPG1, IL36G, EIF1, GIMAP1-GIMAP5, GIMAP1, GIMAP5, MORF4L1, RSBN1L, CBL, CD300LB, SIGLEC9, SIGLEC7, SIGLEC8, SIGLEC12, SLCO3A1, SUPT4H1, MBP, PPP1R15A, CD79A, NRROS, PPP1R18, PTBP3, GIMAP7, BIRC2, CCDC12, MAPK14, TCF7, C1orf162, RBM25, XRN2, JAKMIP1, DOCK10, VSIR, SIGLEC11, SIGLEC10, RAMP1, CD6, PHIP, TPR, KPNA4, PTAFR, SH3BP5, ITK, TNRC6C, NOTCH2, MYD88, NIPBL, PRKCD, BCL10, IL10RA, DHRS7, CLEC4A, SIPR4, CD300LF, OGT, IRAG2, TTC7A, TREML1, FAU, BCL2A1, MARCKS, MIER1, VASP, CLEC4C, GPCPD1, TRAF3IP3, RUNX3, R3HDM4, LFNG, SLAMF6, PDCD10, STX11, SRSF11, GP5, HNRNPL, C1orf54, PID1, NFE2, MZB1, LILRA5, SCNA, PFDN5, PARP14, VGLL4, ENO1, ARL6IP1, SOCS3, SH2D1A, DAPL1, GPR183, LMNB1, STRBP, IL18RAP, HIPK1, STAG2, ORAI1, SMC6, STK10, FAM111A, GNB2, TREML4, EBI3, AP1S2, ARPC1B, NEURL3, TRIB2, ZC3HAV1, BAZ1A, HECTD1, SEC11C, TNRC6B, PRKD2, RNASE6, EVL, VAV3, RAB27A, TRIM5, ADAM8, PHF3, SIRT7, TCP11L2, ZCCHC7, ATP2B1, ITPR2, BPTF, ACE, ICAM2, SLFN12, SLFN12L, FCMR, PRKAR1A, ASAP1, CLEC2D, MEF2C, KANSL1, SLC40A1, VPS37B, SDCBP, CD8B, CD8B2, PHF11, ZEB2, SFPQ, CCL18, CCL3, CCL3L1, ANXA1, YWHAH, HMGB1, PIK3CD, SERP1, ARRB2, CCDC88B, CTSC, ATF7IP, TREML2, NFKB1, VAMP8, SLC16A3, KIF2A, LMO2, CAPZA2, TBCID1, GIMAP8, RASSF3, IFNGR2, TONSL, CLEC2D, PTEN, CD226, HLA-DQB1, HLA-DQB2, SGK1, LCN2, RGS14, PTP4A2, TMEM156, MCTP1, ARF1, N4BP1, ARHGAP9, BIRC3, TXK, NPTN, PSME2, ARL6IP5, PSTPIP1, TUBA4A, SERTAD1, DIAPH1, TNFRSF13B, BATF, IL10RB, ARL5C, CCDC88C, CDK9, SH3BP1, GIMAP4, RNF144A, BCL11A, UBE2D2, RASSF2, ARPC3, C19orf53, GRAMD1A, NADK, TNFRSF13C, TRA2A, GGNBP2, ISG15, DEF6, STAT4, ATP6V0E1, PRRC2C, CARD19, CLEC12A, GSAP, MENG, CMTM6, GPR65, DYNLT2B, CNTRL, S100A11, TACC1, TMEM59, ZNF292, SH3KBP1, GPR18, SYF2, POLR1F, RINL, GP9, AIM2, CTCF, CD300E, DCK, OTULIN, PIP4K2A, MAX, TNFSF14, CALM1, KRIT1, MYH9, TMPO, TNF, UBL3, CSNK1A1, CEACAM7, CEACEM5, CEACAM6, CEACAM3, CEACAM1, CEACAM8, BMX, YY1, VRK1, PRKCB, PFKFB4, G3BP2, ABTB1, EPSTI1, RIN3, ITPKB, MYL6, MS4A6A, MS4A6E, LY9, TAP2, PRKAR2B, SF3B6, FAM78A, ABHD8, ZDHHC18, ATP1A3, PPP1R10, MCMBP, TWF2, KRAS, ARHGAP4, TMEM71, ALCAM, NOTCH1, MORC3, SUN2, ALDOA, HAUS8, RSRC2, CDC42EP3, IKBKB, CHD7, ARL11, UBB, CLEC6A, CXCR4, STK24, NFKBIZ, SERINC3, TIMM10B, CTSW, RCHY1, TAP1, MAF1, RBM5, SEMA4A, MAT2B, CYTH1, NFKBID, CD4, GPI, MEF2A, MARCHF2, JUN, GRAP2, AVL9, FCER2, HBA1, HBA2, BCL2A1, ARF6, RNF13, BLNK, IL27RA, KCNN4, RAC1, FPR1, SASH3, FIS1, ATP8B4, SFXN5, GPR141, RSU1, PRR5L, SCNN1A, TMEM243, FCRLA, PIK3R1, NLRP12, MILR1, and any combination thereof.

In some embodiments, the biological agent recognizes a Coro1a biomarker or a human orthologue of a Coro1a biomarker. In some embodiments, the biological agent recognizes a Cd52 biomarker or a human orthologue of a Cd52 biomarker. In some embodiments, the biological agent recognizes a Cyba biomarker or a human orthologue of a Cyba biomarker. In some embodiments, the biological agent recognizes a Rac2 biomarker or a human orthologue of a Rac2 biomarker. In some embodiments, the biological agent recognizes a Laptm5 biomarker or a human orthologue of a Laptm5 biomarker. In some embodiments, the biological agent recognizes a Ptprc biomarker or a human orthologue of a Ptprc biomarker.

In some embodiments, one or more maternal biomarkers are bispecific. In some embodiments, the bispecific one or more maternal biomarkers are located in maternal cells and fetal cells. In some embodiments, the bispecific one or more maternal biomarkers are located on maternal cells and fetal cells.

In some embodiments, the biological agent is a protein. In some embodiments, the biological agent is a ligand. In some embodiments, the biological agent is an antibody. In some embodiments, the antibody has two heavy chains and two light chains. In some embodiments, the heavy chain has a constant region and a variable region. In some embodiments, the light chain has a constant region and a variable region. In some embodiments, the antibody has one heavy chain and one light chain. In some embodiments, the biological agent is an antigen binding fragment of an antibody (Fab). In some embodiments, the Fab has a light chain variable region and a heavy chain variable region. In some embodiments, the Fab has a light chain variable region. In some embodiments, the Fab has a light chain variable region and a light chain constant region. In some embodiments, the Fab has a heavy chain variable region. In some embodiments, the Fab has a heavy chain variable region and the heavy chain constant region. In some embodiments, the biological agent is a single chain variable fragment (scFv). In some embodiments, the scFv has a heavy chain variable region and a light chain constant region. In some embodiments, the scFv has a light chain variable region and a heavy chain constant region. In some embodiments, the biological agent is a nanobody. In some embodiments, the nanobody has a light chain variable region. In some embodiments, the nanobody has a heavy chain variable region.

In some embodiments, the biological agent is configured to couple to a biomarker. In some embodiments, the biological agent is configured to couple directly to a biomarker. In some embodiments, the biological agent couples with a biomarker via a covalent bond. In some embodiments, the covalent bond is a peptide bond. In some embodiments, the biological agent couples to a biomarker via a non-covalent interaction. In some embodiments, the non-covalent interaction is an ionic interaction. In some embodiments, the non-covalent interaction is a hydrogen bond. In some embodiments, the non-covalent interaction is van der Waals interactions. In some embodiments, the non-covalent interaction is a hydrophobic interaction.

In some embodiments, the biological agent is configured to couple indirectly to a biomarker. In some embodiments, the biological agent couples to the biomarker via a linker. In some embodiments, the linker is a cleavable linker. In some embodiments, the cleavable linker is a chemical labile linker. Non-limiting examples of chemical labile linkers include 6-(maleimidocaproyl) hydrazone, (4-(4-acetylphenoxy) butanoic acid) hydrazone, N-acetyl-y-calicheamicin dimethyl hydrazide, and any combination thereof. In some embodiments, the cleavable linker is a reducible linker. In some embodiments, the reducible linker is a disulfide linker or a glutathione-sensitive disulfide linker. In some embodiments, the cleavable linker is an enzyme-cleavable linker. Non-limiting examples of enzyme-cleavable linkers include peptide-based linkers, valine-citrulline, phenylalanine-lysine, valine-alanine, para-aminobenzyl carbamate, PEG linkers, β-glucuronide linker, β-galactoside linkers, and any combination thereof. In some embodiments, the cleavable linker is a phosphatase-cleavable linker. In some embodiments, the phosphatase-cleavable linker is a phosphate diester. In some embodiments, the linker is a non-cleavable linker. Non-limiting examples of non-cleavable linkers include thioethers, maleimidocapropy, N-succinimidyl-4-(maleimidomethyl)cyclohexane-1-carboxylate linker, and any combination thereof.

In some embodiments, the biological agent is a nucleic acid probe. In some embodiments, the nucleic acid probe is RNA. In some embodiments, the nucleic acid probe is DNA. The nucleic acid probes can be nucleic acid molecules having sequence complementarity with nucleic acid sequences (e.g., RNA or DNA) of the one or more genomic loci (e.g., fetal-associated genomic loci). In some embodiments, the nucleic acid probe can couple to the nucleic acid sequence with sequence complementarity to the nucleic acid probe. In some embodiments, the nucleic acid probe can hybridize to the nucleic acid sequence with sequence complementarity to the nucleic acid probe. These nucleic acid molecules can be primers or enrichment sequences. The assaying of the biological sample using probes that are selective for the one or more genomic loci (e.g., fetal-associated genomic loci) can comprise use of array hybridization (e.g., microarray-based), polymerase chain reaction (PCR), or nucleic acid sequencing (e.g., RNA sequencing or DNA sequencing). In some embodiments, DNA or RNA can be assayed by one or more of: isothermal DNA/RNA amplification methods (e.g., loop-mediated isothermal amplification (LAMP), helicase dependent amplification IA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS). In some embodiments, the nucleic acid probe can be an aptamer.

In some embodiments, the biological agent is labeled. In some embodiments, the biological agent is labeled with a protein or peptide. In some embodiments, the peptide is a fluorescent protein or peptide. Non-limiting examples of fluorescent protein or peptide include: CFP (cyan fluorescent protein), YFP (yellow fluorescent protein), mVenus, cyan ECFP (enhanced cyan fluorescent protein), yellow EYFP (enhanced yellow fluorescent protein), green EGFP (enhanced green fluorescent protein), GFP, BPF (blue fluorescent protein), blue EBFP (enhanced blue fluorescent protein), red mCherry, SYTO®9, propidium iodide, mCitrine, YPet, aquamarine, mTurquoise2, mCeruean3, LUMP (lumazine binding protein), mTFP1 (monomeric teal fluorescent protein), NowGFP, Clover, mClover3, mNeonGreen, mRuby2 mRuby3, mPlum, eqFP650, mCardinal, IFP1.4^{m}, iRFP™, mAmetrine, LSS-mOrange, tdTomato, mKate2, ShadowG, REACh1 (Resonance Energy-Accepting Chromoprotein 1), REACh2, sREACh, rsTagRFP, PA-GFP (photo activatable green fluorescent protein), Phanta, T-Sapphire, mTagBFP, sfGFP (superfolder GFP), CyOFP1, mOrange2, mKOκ, TagRFP, DsRed, muGFP, AF660 (Alex Fluor 660), hydroxycoumarin, aminocoumarin, methoxycoumarin, cascade blue, lucifer yellow, NBD, R-Phycoerythrin, PE-Cy5, PE-Cy7, APC-Cy7, fluorescein, FluorX, BODIPY-FL, tetramethylrhodamine, tetramethylrhodamine isothiocyanate, X-Rhodamine, Lissamine, Rhodamine B, PerCP, Texas Red, Allophycocyanin, TruRed, Alexa Fluor 350, Alexa Fluor 430, Alexa Fluor 488, Alexa Fluor 532, Alexa Fluor 546, Alexa Fluor 555, Alexa Fluor 568, Alexa Fluor 594, Alexa Flour 633, Alexa Fluor 647, Alexa Fluor 660, Alexa Flour 680, Alexa Fluor 700, Alexa Fluor 750, SpectrumOrange, SpectrumGreen1, SpectrumGreen2, SpectrumAqua, SpectrumBlue, SpectrumGold, SpectrumRed, SpectrumFRed, Cy2, Cy3, Cy3.5, Cy5, Cy5.5, Cy7, or Ni-NTA-Atto, and fragments thereof.

In some embodiments, the biological agent is coupled with a fluorescent protein or peptide. In some embodiments, the biological agent is coupled with a fluorescent protein or peptide via a covalent bond. In some embodiments, the covalent bond is a peptide bond. In some embodiments, the biological agent is coupled to a fluorescent protein or peptide via a non-covalent interaction. In some embodiments, the non-covalent interaction is an ionic bond. In some embodiments, the non-covalent interaction is a hydrogen bond. In some embodiments, the non-covalent interaction is van der Waals interactions. In some embodiments, the non-covalent interaction is a hydrophobic interaction.

In some embodiments, the biological agent is indirectly coupled with a fluorescent protein or peptide. In some embodiments, the biological agent is indirectly coupled with a fluorescent protein or peptide via a linker. In some embodiments, the linker is a short amino acid sequence. In some embodiments, the short amino acid sequence is from one amino acid residue to about 10 amino acid residues. In some embodiments, the short amino acid sequence is made up of glycine residues. In some embodiments, the short amino acid sequence is made up of serine residues. In some embodiments, the short amino acid sequence is made up of glycine and serine residues. In some embodiments, the linker is polyethylene glycol. In some embodiments, the linker is streptavidin. In some embodiments, the linker is an alkylene group. The linker can be flexible, rigid, and/or cleavable.

The linker can comprise a polymeric spacer, a polyether spacer, and/or an alkylene spacer (e.g., methylene, ethylene, propylene, or butylene). In some embodiments, the linker can be a bond (e.g., a covalent bond). The spacer can comprise polyethylene glycol (PEG) groups, alkylene groups, and/or amino acid residues.

In some embodiments, the linker can have from about 1 PEG group to about 10 PEG groups. In some embodiments, the linker can have at least about 1 PEG group, at least about 2 PEG groups, at least about 3 PEG groups, at least about 4 PEG groups, at least about 5 PEG groups, at least about 6 PEG groups, at least about 7 PEG groups, at least about 8 PEG groups, at least about 9 PEG groups, at least about 10 PEG groups, or more. In some embodiments, the linker can have at most about 10 PEG groups, at most about 9 PEG groups, at most about 8 PEG groups, at most about 7 PEG groups, at most about 6 PEG groups, at most about 5 PEG groups, at most about 4 PEG groups, at most about 3 PEG groups, at most about 2 PEG groups, or at most about 1 PEG groups. In some embodiments, the linker can have about 1 PEG group, about 2 PEG groups, about 3 PEG groups, about 4 PEG groups, about 5 PEG groups, about 6 PEG groups, about 7 PEG groups, about 8 PEG groups, about 9 PEG groups, or about 10 PEG groups.

In some embodiments, the linker can have from about 1 to about 10 alkylene groups. In some embodiments, the linker can have at least about 1 alkylene group, at least about 2 alkylene groups, at least about 3 alkylene groups, at least about 4 alkylene groups, at least about 5 alkylene groups, at least about 6 alkylene groups, at least about 7 alkylene groups, at least about 8 alkylene groups, at least about 9 alkylene groups, at least about 10 alkylene groups, or more. In some embodiments, the linker can have at most about 10 alkylene groups, at most about 9 alkylene groups, at most about 8 alkylene groups, at most about 7 alkylene groups, at most about 6 alkylene groups, at most about 5 alkylene groups, at most about 4 alkylene groups, at most about 3 alkylene groups, at most about 2 alkylene groups, at most about 1 alkylene group, or less. In some embodiments, the linker can have about 1 alkylene group, about 2 alkylene groups, about 3 alkylene groups, about 4 alkylene groups, about 5 alkylene groups, about 6 alkylene groups, about 7 alkylene groups, about 8 alkylene groups, about 9 alkylene groups, or about 10 alkylene groups.

In some embodiments, the linker can have from about 1 amino acid residue to about 10 amino acid residues. In some embodiments, the linker can have at least about one amino acid residue, at least about two amino acid residues, at least about three amino acid residues, at least about four amino acid residues, at least about five amino acid residues, at least about six amino acid residues, at least about seven amino acid residues, at least about eight amino acid residues, at least about nine amino acid residues, at least about ten amino acid residues, or more. In some embodiments, the linker can have at most about ten amino acid residues, at most about nine amino acid residues, at most about eight amino acid residues, at most about seven amino acid residues, at most about six amino acid residues, at most about five amino acid residues, at most about four amino acid residues, at most about three amino acid residues, at most about two amino acid residues, or at most about one amino acid residue. In some embodiments, the linker can have about one amino acid residue, about two amino acid residues, about three amino acid residues, about four amino acid residues, about five amino acid residues, about six amino acid residues, about seven amino acid residues, about eight amino acid residues, about nine amino acid residues, or about ten amino acid residues.

In some embodiments, the biological agent is attached to a surface. In some embodiments, the surface is a plate. In some embodiments, the surface is a slide. In some embodiments, the surface is a bead. In some embodiments, the bead is plastic. In some embodiments, the bead is glass. In some embodiments, the bead is metal. In some embodiments, the bead is magnetic.

In some embodiments, one or more biological agents are attached to the surface. In some embodiments, up to about 10 biological agents are attached to the surface. In some embodiments, at least about one biological agents, at least about two biological agents, at least about three biological agents, at least about four biological agents, at least about five biological agents, at least about six biological agents, at least about seven biological agents, at least about eight biological agents, at least about nine biological agents, or at least about ten biological agents are attached to the surface.

In some embodiments, at most about ten biological agents, at most about nine biological agents, at most about eight biological agents, at most about seven biological agents, at most about six biological agents, at most about five biological agents, at most about four biological agents, at most about three biological agents, at most about two biological agents, or at most about one biological agent is attached to the surface.

In some embodiments, about one biological agent, about two biological agents, about three biological agents, about four biological agents, about five biological agents, about six biological agents, about seven biological agents, about eight biological agents, about nine biological agents, or about ten biological agents are attached to the surface.

In some embodiments, the one or more biological agents (e.g., isolation agents or agents) that specifically couple (e.g., are specific for) to one or more biomarkers couple to the one or more biomarkers. In some embodiments, the one or more biological agents that are specific for the one or more biomarkers do not couple to other biomarkers that are different from the one or more biomarkers.

In some embodiments, the one or more biological agents (e.g., isolation agents) are biological agents that specifically couple to one or more fetal biomarkers. In some embodiments, the one or more biological agents (e.g., isolation agents) that specifically couple (e.g., are specific for) to one or more fetal biomarkers couple to the one or more fetal biomarkers. In some embodiments, the one or more biological agents that are specific for the one or more fetal biomarkers do not couple to other fetal biomarkers that are different from the one or more fetal biomarkers. In some embodiments, the one or more biological agents that are specific for the one or more fetal biomarkers may not couple to one or more maternal biomarkers.

In some embodiments, the one or more biological agents are biological agents that specifically couple to one or more maternal biomarkers. In some embodiments, the one or more biological agents (e.g., isolation agents) that specifically couple (e.g., are specific for) to one or more maternal biomarkers couple to the one or more maternal biomarkers. In some embodiments, the one or more biological agents that are specific for the one or more maternal biomarkers may not couple to other maternal biomarkers that are different from the one or more maternal biomarkers. In some embodiments, the one or more biological agents that are specific for the one or more maternal biomarkers may not couple to one or more fetal biomarkers.

### Methods

In some embodiments, the present disclosure provides a method of analyzing a nucleic acid sequence. In some embodiments, the nucleic acid sequence is DNA. In some embodiments, the DNA is genomic DNA. In some embodiments, the nucleic acid sequence is RNA. In some embodiments, the RNA is mRNA. In some embodiments, the RNA is rRNA. In some embodiments, the RNA is lncRNA. In some embodiments, the nucleic acid sequence is isolated from a biological sample. In some embodiments, the biological sample is a blood sample. In some embodiments, the blood sample is blood plasma. In some embodiments, the blood sample is blood serum. In some embodiments, the blood sample is a buffy coat. In some embodiments, the blood sample is peripheral blood mononuclear cells. In some embodiments, the blood sample is nucleated white blood cells. In some embodiments, the blood sample is whole blood. In some embodiments, the blood sample is whole blood depleted of red blood cell. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some cases, the biological fluid sample is blood, saliva, interstitial fluid, umbilical cord blood, extracellular fluid, or any combination thereof. In some embodiments, the sample is from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the sample is from a pregnant subject. In some embodiments, the sample is a maternal biological sample. In some embodiments, the biological blood sample is from a pregnant subject.

In some embodiments, the nucleic acid sequence is from an ex vivo population of cells. In some embodiments, the ex vivo population of cells has a subpopulation of fetal cells. In some embodiments, the ex vivo population of cells has a subpopulation of maternal cells. In some embodiments, the ex vivo population of cells has a subpopulation of fetal cells and a subpopulation of maternal cells.

In some embodiments, the method further comprising depleting red blood cells from the ex vivo population of cells. In some embodiments, red blood cells are depleted from the ex vivo population of cells to remove non-nucleated cells from the sample. In some embodiments, red blood cells are depleted from the ex vivo population of cells to decrease background interference in subsequent analyses and/or assays performed on the ex vivo population of cells. In some embodiments, red blood cells are depleted from the ex vivo population of cells to increase the accuracy in subsequent analyses and/or assays performed on the ex vivo population of cells. In some embodiments, red blood cells (RBCs) are depleted from the ex vivo population of cells via ammonium chloride lysis, density gradient centrifugation, hypotonic lysis, immunomagnetic cell separation, sedimentation, or any combination thereof.

In some embodiments, the method further comprises providing an extract prior to sequencing. In some embodiments, the extract is made by extracting the ex vivo population of cells from the maternal biological sample. In some embodiments, the ex vivo population of cells is extracted from the maternal biological sample. In some embodiments, the subpopulation of fetal cells is extracted from the maternal biological sample. In some embodiments, the subpopulation of maternal cells is extracted from the maternal biological sample. In some embodiments, the subpopulation of maternal cells and the subpopulation of fetal cells are extracted from the maternal biological sample.

In some embodiments, the subpopulation of fetal cells has from about one fetal cell type to about ten fetal cell types. Non-limiting examples of fetal cell types include early gestation trophoblasts, late gestation trophoblasts, erythroblasts, megakaryocytes, stromal cells, endothelial cells, lymphocytes, nucleated red blood cells, hematopoietic progenitor cells, vascular endothelial cells, and mesenchymal progenitor cells. In some embodiments, the subpopulation of fetal cells has at least about one fetal cell type, at least about two fetal cell types, at least about three fetal cell types, at least about four fetal cell types, at least about five fetal cell types, at least about six fetal cell types, at least seven fetal cell types, at least about eight fetal cell types, at least about nine fetal cell types, or more. In some embodiments, the subpopulation of fetal cells has at most about ten fetal cell types, at most about nine fetal cell types, at most about eight fetal cell types, at most about seven fetal cell types, at most about six fetal cell types, at most about five fetal cell types, at most about four fetal cell types, at most about three fetal cell types, at most about two fetal cell types, at most about one fetal cell type, or less. In some embodiments, the subpopulation of fetal cells has about one fetal cell type, about two fetal cell types, about three fetal cell types, about four fetal cell types, about five fetal cell types, about six fetal cell types, about seven fetal cell types, about eight fetal cell types, about nine fetal cell types, or about ten fetal cell types.

In some embodiments, the subpopulation of maternal cells has from about one maternal cell type to about 10 maternal cell types. In some embodiments, the subpopulation of maternal cells has at least about one maternal cell types, at least about two maternal cell types, at least about three maternal cell types, at least about four maternal cell types, at least about five maternal cell types, at least about six maternal cell types, at least about seven maternal cell types, at least about eight maternal cell types, at least about nine maternal cell types, at least about ten maternal cell types, or more. In some embodiments, the subpopulation of maternal cells has at most about ten maternal cell types, at most about nine maternal cell types, at most about eight maternal cell types, at most about seven maternal cell types, at most about six maternal cell types, at most about five maternal cell types, at most about four maternal cell types, at most about three maternal cell types, at most about two maternal cell types, at most about one maternal cell type, or less. In some embodiments, the subpopulation of maternal cells has about one maternal cell types, about two maternal cell types, about three maternal cell types, about four maternal cell types, about five maternal cell types, about six maternal cell types, about seven maternal cell types, about eight maternal cell types, about nine maternal cell types, or about ten maternal cell types.

In some embodiments, the ex vivo population of cells is extracted from a mother. In some embodiments, the ex vivo population of cells is extracted from a mother by taking a biological sample. In some embodiments, the ex vivo population of cells is extracted from a mother by taking a biological fluid sample. In some embodiments, the ex vivo population of cells is extracted from a mother by taking a blood sample. In some embodiments, the ex vivo population of cells is extracted from a mother by taking a tissue sample and/or biopsy. In some embodiments, the ex vivo population of cells has an ex vivo population of fetal cells. In some embodiments, the ex vivo population of cells has an ex vivo population of maternal cells. In some embodiments, the ex vivo population of cells has an ex vivo population of the fetal cells and an ex vivo population of maternal cells.

In some embodiments, the ex vivo population of cells is extracted from the biological sample. In some cases, the ex vivo population of cells is extracted from the biological sample by removing non-nucleated cells (e.g., red blood cells), extracellular proteins, extracellular nucleic acid molecules, extracellular metabolites, or any combination thereof from the biological sample.

In some embodiments, subsequent to extracting the ex vivo population of cells from the biological sample, the ex vivo population of cells can undergo one or more washing operations. In some embodiments, the one or more washing operations can eliminate extracellular molecules (e.g., lipids, sugars, minerals, vitamins, nucleic acids, metabolites) remaining on the cells. In some embodiments, the one or more washing operations can remove excess reagents and/or labels. In some embodiments, the method can comprise from about one to about two wash operations, from about two to about three wash operations, from about three to about four wash operations, from about four wash operations to about five wash operations, from about five wash operations to about six wash operations, from about six wash operations to about seven wash operations, from about seven wash operations to about eight wash operations, from about eight wash operations to about nine wash operations, or from about nine wash operations to about ten wash operations. In some embodiments, the method can comprise at least about one wash operation, at most about two wash operations, at least about three wash operations, at least about four wash operations, at least about five wash operations, at least about six wash operations, at least about seven wash operations, at least about eight wash operations, at least about nine wash operations, at least about ten wash operations or more. In some cases, the method can comprise at most about ten wash operations, at most about nine wash operations, at most about eight wash operations, at most about seven wash operations, at most about six wash operations, at most about five wash operations, at most about four wash operations, at most about three wash operations, at most about two wash operations, at most about one wash operation, or less. In some cases, the method can comprise about one wash operation, about two wash operations, about three wash operations, about four wash operations, about five wash operations, about six wash operations, about seven wash operations, about eight wash operations, about nine wash operations, or about ten wash operations. In some embodiments, the washing operation uses a washing buffer. Non-limiting examples of washing buffers include water, methanol, ethanol, phosphate buffered saline (PBS), saline, Tris-buffered solution, Tween buffer, glycine buffer, and any combination thereof.

In some embodiments, the ex vivo population of cells extracted from the maternal biological sample is used to provide an extract. In some embodiments, the ex vivo population of cells is extracted from the maternal biological sample using density gradient centrifugation. In some embodiments, the ex vivo population of cells is extracted from the maternal biological sample using magnetic isolation. In some embodiments, the ex vivo population of cells is extracted from the maternal biological sample using shear-induced diffusion. In some embodiments, the extract is made up of the subpopulation of fetal cells. In some embodiments, the extract is made up of the subpopulation of maternal cells. In some embodiments, the extract is made up of the subpopulation of fetal cells and the subpopulation of maternal cells.

In some embodiments, the method further comprises enriching the subpopulation of fetal cells in the extract to provide an enriched sample. In some embodiments, the extract is enriched to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, the enriched sample is depleted in maternal cells and subsequently enriched for fetal cells. In some embodiments, the enriched sample is enriched for fetal cells and subsequently depleted for maternal cells. In some embodiments, the extract is enriched for fetal cells by immunomagnetic cell separation. In some embodiments, the immunomagnetic cell separation is positive immunomagnetic cell selection. In some embodiments, the extract is enriched for fetal cells by fluorescence-activated cell sorting (FACS). In some embodiments, the extract is enriched for fetal cells by magnetic-activated cell sorting (MACS). In some embodiments, the extract is enriched for fetal cells using one or more fetal cell biomarkers. In some embodiments, the extract is enriched for fetal cells by negative immunomagnetic cell separation. In some embodiments, the negative immunomagnetic cell separation enriches for fetal cells using one or more maternal cell biomarkers. In some embodiments, the extract is enriched for fetal cells by microscopic microdissection. In some embodiments, the extract is enriched for fetal cells by microscopic imaging.

In some embodiments, the extract is enriched for fetal cells using one or more isolation agents. In some embodiments, the extract is enriched for fetal cells by incubating one or more isolation agents with the ex vivo population of cells in the extract. In some embodiments, the one or more isolation agents incubated with the ex vivo population of cells in the extract are specific for one or more fetal biomarkers. In some embodiments, the one or more isolation agents incubated with the ex vivo population of cells in the extract are specific for one or more maternal biomarkers. In some embodiments, the one or more isolation agents incubated with the ex vivo population of cells in the extract are specific for one or more fetal biomarkers and specific for one or more maternal biomarkers.

In some embodiments, subsequent to incubating (e.g., contacting) the ex vivo population of cells with the one or more isolation agents, the ex vivo population of fetal cells can undergo one or more washing operations. In some embodiments, the one or more washing operations can eliminate any unbound isolation agents. In some embodiments, the one or more washing operations can remove excess reagents and/or labels. In some embodiments, the method can comprise from about one to about two wash operations, from about two to about three wash operations, from about three to about four wash operations, from about four wash operations to about five wash operations, from about five wash operations to about six wash operations, from about six wash operations to about seven wash operations, from about seven wash operations to about eight wash operations, from about eight wash operations to about nine wash operations, or from about nine wash operations to about ten wash operations. In some embodiments, the method can comprise at least about one wash operation, at most about two wash operations, at least about three wash operations, at least about four wash operations, at least about five wash operations, at least about six wash operations, at least about seven wash operations, at least about eight wash operations, at least about nine wash operations, at least about ten wash operations or more. In some embodiments, the method can comprise at most about ten wash operations, at most about nine wash operations, at most about eight wash operations, at most about seven wash operations, at most about six wash operations, at most about five wash operations, at most about four wash operations, at most about three wash operations, at most about two wash operations, at most about one wash operation, or fewer. In some embodiments, the method can comprise about one wash operation, about two wash operations, about three wash operations, about four wash operations, about five wash operations, about six wash operations, about seven wash operations, about eight wash operations, about nine wash operations, or about ten wash operations.

In some embodiments, the extract is sorted to isolate the ex vivo population of fetal cells. In some embodiments, the extract is sorted to separate the ex vivo population of fetal cells from the ex vivo population of maternal cells from the extract. In some embodiments, the extract is sorted for fetal cells by immunomagnetic cell separation. In some embodiments, the immunomagnetic cell separation is positive immunomagnetic cell selection. In some embodiments, the extract is sorted for fetal cells by fluorescence-activated cell sorting (FACS). In some embodiments, the extract is sorted for fetal cells by magnetic-activated cell sorting (MACS). In some embodiments, the extract is sorted for fetal cells using a presence of one or more fetal cell biomarkers. In some embodiments, the extract is sorted for fetal cells by negative immunomagnetic cell separation. In some embodiments, the negative immunomagnetic cell separation sorts fetal cells using an absence of one or more maternal cell biomarkers. In some embodiments, the extract is sorted for fetal cells by microscopic microdissection. In some embodiments, the extract is sorted for fetal cells by microscopic imaging.

In some embodiments, the extract is sorted to isolate the ex vivo population of maternal cells. In some embodiments, the extract is sorted to separate the ex vivo population of fetal cells from the ex vivo population of maternal cells from the extract. In some embodiments, the extract is sorted for maternal cells by immunomagnetic cell separation. In some embodiments, the immunomagnetic cell separation is positive immunomagnetic cell selection. In some embodiments, the extract is sorted for maternal cells by fluorescence-activated cell sorting (FACS). In some embodiments, the extract is sorted for maternal cells by magnetic-activated cell sorting (MACS). In some embodiments, the extract is sorted for maternal cells using a presence of one or more fetal cell biomarkers. In some embodiments, the extract is sorted for maternal cells by negative immunomagnetic cell separation. In some embodiments, the negative immunomagnetic cell separation sorts maternal cells using an absence of one or more fetal cell biomarkers. In some embodiments, the extract is sorted for maternal cells by microscopic microdissection. In some embodiments, the extract is sorted for maternal cells by microscopic imaging.

In some embodiments, the ex vivo population of fetal cells is isolated from the extract. In some embodiments, the ex vivo population of fetal cells is separated from the extract. In some embodiments, the fetal cells are isolated from the extract by immunomagnetic cell separation. In some embodiments, the immunomagnetic cell separation is positive immunomagnetic cell selection. In some embodiments, the fetal cells are isolated from the extract by fluorescence-activated cell sorting (FACS). In some embodiments, the fetal cells are isolated from the extract by magnetic-activated cell sorting (MACS). In some embodiments, the fetal cells are isolated from the extract using a presence of one or more fetal cell biomarkers. In some embodiments, the fetal cells are isolated from the extract by negative immunomagnetic cell separation. In some embodiments, the negative immunomagnetic cell separation isolates fetal cells using an absence of one or more maternal cell biomarkers. In some embodiments, the fetal cells are isolated from the extract by microscopic microdissection. In some embodiments, the fetal cells are isolated from the extract by microscopic imaging.

In some embodiments, the ex vivo population of maternal cells is isolated from the extract. In some embodiments, the ex vivo population of maternal cells is separated from the extract. In some embodiments, the maternal cells are isolated from the extract by immunomagnetic cell separation. In some embodiments, the immunomagnetic cell separation is positive immunomagnetic cell selection. In some embodiments, the maternal cells are isolated from the extract by fluorescence-activated cell sorting (FACS). In some embodiments, the maternal cells are isolated from the extract by magnetic-activated cell sorting (MACS). In some embodiments, the maternal cells are isolated from the extract using a presence of one or more maternal cell biomarkers. In some embodiments, the maternal cells are isolated from the extract by negative immunomagnetic cell separation. In some embodiments, the negative immunomagnetic cell separation isolates maternal cells using an absence of one or more fetal cell biomarkers. In some embodiments, the maternal cells are isolated from the extract by microscopic microdissection. In some embodiments, the maternal cells are isolated from the extract by microscopic imaging.

In some embodiments, the extract is enriched for fetal cells using from about one isolation agent to about five isolation agents, or from about five isolation agents to about ten isolation agents. In some embodiments, the extract is enriched for fetal cells using at least about one isolation agent, at least about two isolation agents, at least about three isolation agents, at least about four isolation agents, at least about five isolation agents, at least about six isolation agents, at least about seven isolation agents, at least about eight isolation agents, at least about nine isolation agents, at least about ten isolation agents, or more. In some embodiments, the extract is enriched for fetal cells using at most about ten isolation agents, at most about nine isolation agents, at most about eight isolation agents, at most about seven isolation agents, at most about six isolation agents, at most about five isolation agents, at most about four isolation agents, at most about three isolation agents, at most about two isolation agents, at most about one isolation agent, or less. In some embodiments, the extract is enriched for fetal cells using about one isolation agent, about two isolation agents, about three isolation agents, about four isolation agents, about five isolation agents, about six isolation agents, about seven isolation agents, about eight isolation agents, about nine isolation agents, or about ten isolation agents.

In some embodiments, the one or more isolation agents has a first isolation agent. In some embodiments, the first isolation agent couples to a first biomarker in the subpopulation of fetal cells. In some embodiments, the extract is enriched for fetal cells by density gradient centrifugation. In some embodiments, the extract is enriched for fetal cells by immunodensity cell isolation. In some embodiments, the extract is enriched for fetal cells by microfluidic cell sorting.

In some embodiments, the method further comprises enriching the subpopulation of maternal cells in the extract to provide an enriched sample. In some embodiments, the extract is enriched to provide an enriched sample. In some embodiments, the enriched sample is enriched in maternal cells. In some embodiments, the enriched sample is depleted in fetal cells. In some embodiments, the enriched sample is enriched in maternal cells and depleted in fetal cells. In some embodiments, the enriched sample is depleted in fetal cells and subsequently enriched for maternal cells. In some embodiments, the enriched sample is enriched for maternal cells and subsequently depleted in fetal cells. In some embodiments, extract is enriched for maternal cells by immunomagnetic cell separation. In some embodiments, the immunomagnetic cell separation is positive immunomagnetic cell selection. In some embodiments, the extract is enriched for maternal cells by fluorescence-activated cell sorting (FACS). In some embodiments, the extract is enriched for maternal cells by magnetic-activated cell sorting (MACS). In some embodiments, the extract is enriched for maternal cells by microscopic microdissection. In some embodiments, the extract is enriched for maternal cells by microscopic imaging. In some embodiments, the extract is enriched for maternal cells using one or more maternal cell biomarkers. In some embodiments, the extract is enriched for maternal cells by negative immunomagnetic cell separation. In some embodiments, the negative immunomagnetic cell separation enriches for maternal cells using one or more fetal cell biomarkers.

In some embodiments, the extract is enriched for maternal cells using one or more isolation agents. In some embodiments, the extract is enriched for maternal cells using from about one isolation agent to about five isolation agents, or from about five isolation agents to about ten isolation agents. In some embodiments, the extract is enriched for maternal cells using at least about one isolation agent, at least about two isolation agents, at least about three isolation agents, at least about four isolation agents, at least about five isolation agents, at least about six isolation agents, at least about seven isolation agents, at least about eight isolation agents, at least about nine isolation agents, at least about ten isolation agents, or more. In some embodiments, the extract is enriched for maternal cells using at most about ten isolation agents, at most about nine isolation agents, at most about eight isolation agents, at most about seven isolation agents, at most about six isolation agents, at most about five isolation agents, at most about four isolation agents, at most about three isolation agents, at most about two isolation agents, at most about one isolation agent, or less. In some embodiments, the extract is enriched for maternal cells using about one isolation agent, about two isolation agents, about three isolation agents, about four isolation agents, about five isolation agents, about six isolation agents, about seven isolation agents, about eight isolation agents, about nine isolation agents, or about ten isolation agents. In some embodiments, the one or more isolation agents has a second isolation agent. In some embodiments, the extract is enriched for maternal cells using one or more isolation agent. In some embodiments, the one or more isolation agent couples to a second biomarker in the subpopulation of maternal cells.

In some embodiments, the method further comprises sorting the subpopulation of the fetal cells in the extract from the subpopulation of maternal cells. In some embodiments, the sorting of the subpopulation of the cells in the extract provides an enriched sample. In some embodiments, the extract is sorted to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, the extract is enriched for fetal cells by sorting the extract for a cell characteristic. Non-limiting examples of cell characteristics include size, expression of a specific gene, expression of an extracellular protein, and expression of an intracellular protein.

In some embodiments, the extract is enriched for fetal cells by sorting the extract for cells that are from about 6 micrometers (μm) to about 14 μm in diameter. In some embodiments, the extract is enriched for fetal cells by sorting the extract for cells that are at least about 6 μm, at least about 7 μm, at least about 8 μm, at least about 9 μm, at least about 10 μm, at least about 11 μm, at least about 12 μm, at least about 13 μm, at least about 14 μm, or more in diameter. In some embodiments, the extract is enriched for fetal cells by sorting the extract for cells that are at most about 14 μm, at most about 13 μm, at most about 12 μm, at most about 11 μm, at most about 10 μm, at most about 9 μm, at most about 8 μm, at most about 7 μm, at most about 6 μm, or less in diameter. In some embodiments, the extract is enriched for fetal cells by sorting the extract for cells that are about 6 μm, about 7 μm, about 8 μm, about 9 μm, about 10 μm, about 11 μm, about 12 μm, about 13 μm, or about 14 μm in diameter.

In some embodiments, the extract is enriched for fetal cells by sorting the extract for expression of an extracellular biomarker. In some embodiments, the extract is enriched for fetal cells by sorting the extract for the absence of expression of an extracellular biomarker. In some embodiments, the extract is enriched for fetal cell by sorting the extract for expression of an intracellular biomarker. In some embodiments, the extract is enriched for fetal cells by sorting the extract for the absence of expression of an intracellular biomarker. In some embodiments, the extract is enriched for fetal cells by sorting the extract for expression of a specific gene. In some embodiments, the extract is enriched for fetal cells by sorting the extract for absence of expression of a specific gene. In some embodiments, the specific gene is expressed as DNA. In some embodiments, the specific gene is expressed as RNA. In some embodiments, the extract is sorted by immunomagnetic cell separation. In some embodiments, the immunomagnetic cell separation is positive immunomagnetic cell selection. In some embodiments, the extract is enriched for fetal cells by negative immunomagnetic cell separation. In some embodiments, the negative immunomagnetic cell separation enriches for fetal cells using one or more maternal cell biomarkers. In some embodiments, the extract is sorted by FACS. In some embodiments, the extract is sorted by MACS. In some embodiments, the extract is sorted by microscopic microdissection. In some embodiments, the extract is sorted by microscopic imaging. In some embodiments, the extract is sorted for fetal cells using one or more isolation agent. In some embodiments, the one or more isolation agents has a first isolation agent. In some embodiments, the first isolation agent couples to a first biomarker in the subpopulation of fetal cells. In some embodiments, the extract is sorted by microfluidic cell sorting.

In some embodiments, the method further comprises sorting the subpopulation of the maternal cells in the extract from the subpopulation of fetal cells. In some embodiments, the sorting of the subpopulation of the cells in the extract provides an enriched sample. In some embodiments, the extract is sorted to provide an enriched sample. In some embodiments, the enriched sample is enriched in maternal cells. In some embodiments, the enriched sample is depleted in fetal cells. In some embodiments, the enriched sample is enriched in maternal cells and depleted in fetal cells. In some embodiments, the extract is enriched for maternal cells by sorting the extract for a cell characteristic. Non-limiting examples of cell characteristics include size, expression of a specific gene, expression of an extracellular protein, and expression of an intracellular protein.

In some embodiments, the extract is enriched for maternal cells by sorting the extract for cells that are from about 6 micrometers (μm) to about 14 μm in diameter. In some embodiments, the extract is enriched for maternal cells by sorting the extract for cells that are at least about 6 μm, at least about 7 μm, at least about 8 μm, at least about 9 μm, at least about 10 μm, at least about 11 μm, at least about 12 μm, at least about 13 μm, at least about 14 μm, or more in diameter. In some embodiments, the extract is enriched for maternal cells by sorting the extract for cells that are at most about 14 μm, at most about 13 μm, at most about 12 μm, at most about 11 μm, at most about 10 μm, at most about 9 μm, at most about 8 μm, at most about 7 μm, at most about 6 μm, or less in diameter. In some embodiments, the extract is enriched for maternal cells by sorting the extract for cells that are about 6 μm, about 7 μm, about 8 μm, about 9 μm, about 10 μm, about 11 μm, about 12 μm, about 13 μm, or about 14 μm in diameter.

In some embodiments, the extract is enriched for maternal cells by sorting the extract for expression of an extracellular biomarker. In some embodiments, the extract is enriched for maternal cells by sorting the extract for the absence of expression of an extracellular biomarker. In some embodiments, the extract is enriched for maternal cell by sorting the extract for expression of an intracellular biomarker. In some embodiments, the extract is enriched for maternal cells by sorting the extract for the absence of expression of an intracellular biomarker. In some embodiments, the extract is enriched for maternal cells by sorting the extract for expression of a specific gene. In some embodiments, the extract is enriched for maternal cells by sorting the extract for absence of expression of a specific gene. In some embodiments, the specific gene is expressed as DNA. In some embodiments, the specific gene is expressed as RNA. In some embodiments, the extract is sorted by immunomagnetic cell separation. In some embodiments, the immunomagnetic cell separation is positive immunomagnetic cell selection. In some embodiments, the extract is enriched for maternal cells by negative immunomagnetic cell separation. In some embodiments, the negative immunomagnetic cell separation enriches for maternal cells using one or more fetal cell biomarkers. In some embodiments, the extract is sorted by FACS. In some embodiments, the extract is sorted by MACS. In some embodiments, the extract is sorted by microscopic microdissection. In some embodiments, the extract is sorted by microscopic imaging. In some embodiments, the extract is sorted for maternal cells using one or more isolation agents. In some embodiments, the one or more isolation agents has a second isolation agent. In some embodiments, the second isolation agent couples to a second biomarker in the subpopulation of maternal cells.

In some embodiments, the subpopulation of fetal cells is early gestation fetal cells. In some embodiments, the subpopulation of fetal cells is later gestation fetal cells. In some embodiments, the subpopulation of fetal cells is trophoblasts. In some embodiments, the subpopulation of fetal cells is early gestation trophoblasts. In some embodiments, the subpopulation of fetal cells is later gestation trophoblasts. In some embodiments, the subpopulation of fetal cells is erythroblasts. In some embodiments, the subpopulation of fetal cells is megakaryocytes. In some embodiments, the subpopulation of fetal cells is stromal cells. In some embodiments, the subpopulation of fetal cells is endothelial cells. In some embodiments, the subpopulation of fetal cells is lymphocytes. In some embodiments, the subpopulation of fetal cells is nucleated red blood cells. In some embodiments, the subpopulation of fetal cells is hematopoietic progenitor cells. In some embodiments, the subpopulation of fetal cells is mesenchymal progenitor cells. In some embodiments, the subpopulation of fetal cells is vascular endothelial cells.

In some embodiments, the subpopulation of fetal cells is made up for one or more subpopulations of fetal cells. In some embodiments, the second subpopulation of fetal cells is different in cell type from the first subpopulation of fetal cells. In some embodiments, the first subpopulation of fetal cells is early gestation fetal cells and the second subpopulation of fetal cells is later gestation fetal cells. In some embodiments, the first subpopulation of fetal cells is early gestation trophoblasts and the second subpopulation of fetal cells is later gestation trophoblasts. In some embodiments, the first subpopulation of fetal cells is later gestation trophoblasts and the second subpopulation of fetal cells is early gestation trophoblasts.

In some embodiments, the first subpopulation of fetal cells is early gestation trophoblasts and the second subpopulation of fetal cells is erythroblasts. In some embodiments, the first subpopulation of fetal cells is later gestation trophoblasts and the second subpopulation of fetal cells is erythroblasts.

In some embodiments, the first subpopulation of fetal cells is early gestation trophoblasts and the second subpopulation of fetal cells is megakaryocytes. In some embodiments, the first subpopulation of fetal cells is later gestation trophoblasts and the second subpopulation of fetal cells is megakaryocytes.

In some embodiments, the first subpopulation of fetal cells is early gestation trophoblasts and the second subpopulation of fetal cells is stromal cells. In some embodiments, the first subpopulation of fetal cells is later gestation trophoblasts and the second subpopulation of fetal cells is stromal cells.

In some embodiments, the first subpopulation of fetal cells is early gestation trophoblasts and the second subpopulation of fetal cells is endothelial cells. In some embodiments, the first subpopulation of fetal cells is later gestation trophoblasts and the second subpopulation of fetal cells is endothelial cells.

In some embodiments, the first subpopulation of fetal cells is early gestation trophoblasts and the second subpopulation of fetal cells is lymphocytes. In some embodiments, the first subpopulation of fetal cells is later gestation trophoblasts and the second subpopulation of fetal cells is lymphocytes.

In some embodiments, the first subpopulation of fetal cells is early gestation trophoblasts and the second subpopulation of fetal cells is nucleated red blood cells. In some embodiments, the first subpopulation of fetal cells is later gestation trophoblasts and the second subpopulation of fetal cells is nucleated red blood cells.

In some embodiments, the first subpopulation of fetal cells is early gestation trophoblasts and the second subpopulation of fetal cells is hematopoietic progenitor cells. In some embodiments, the first subpopulation of fetal cells is later gestation trophoblasts and the second subpopulation of fetal cells is hematopoietic progenitor cells.

In some embodiments, the first subpopulation of fetal cells is early gestation trophoblasts and the second subpopulation of fetal cells is mesenchymal progenitor cells. In some embodiments, the first subpopulation of fetal cells is later gestation trophoblasts and the second subpopulation of fetal cells is mesenchymal progenitor cells.

In some embodiments, the first subpopulation of fetal cells is early gestation trophoblasts and the second subpopulation of fetal cells is vascular endothelial cells. In some embodiments, the first subpopulation of fetal cells is later gestation trophoblasts and the second subpopulation of fetal cells is vascular endothelial cells.

In some embodiments, the first subpopulation of fetal cells is erythroblasts and the second subpopulation of fetal cells is megakaryocytes. In some embodiments, the first subpopulation of fetal cells is erythroblasts and the second subpopulation of fetal cells is stromal cells. In some embodiments, the first subpopulation of fetal cells is erythroblasts and the second subpopulation of fetal cells are endothelial cells. In some embodiments, the first subpopulation of fetal cells is erythroblasts and the second subpopulation of fetal cells is lymphocytes. In some embodiments, the first subpopulation of fetal cells is erythroblasts and the second subpopulation of fetal cells is nucleated red blood cells. In some embodiments, the first subpopulation of fetal cells is erythroblasts and the second subpopulation of fetal cells is hematopoietic progenitor cells. In some embodiments, the first subpopulation of fetal cells is erythroblasts and the second subpopulation of fetal cells is mesenchymal progenitor cells. In some embodiments, the first subpopulation of fetal cells is erythroblasts and the second subpopulation of fetal cells is vascular endothelial cells.

In some embodiments, the first subpopulation of fetal cells is megakaryocytes and the second population of fetal cells is stromal cells. In some embodiments, the first subpopulation of fetal cells is megakaryocytes and the second population of fetal cells is endothelial cells. In some embodiments, the first subpopulation of fetal cells is megakaryocytes and the second population of fetal cells is lymphocytes. In some embodiments, the first subpopulation of fetal cells is megakaryocytes and the second subpopulation of fetal cells is nucleated red blood cells. In some embodiments, the first subpopulation of fetal cells is megakaryocytes and the second subpopulation of fetal cells is hematopoietic progenitor cells. In some embodiments, the first subpopulation of fetal cells is megakaryocytes and the second subpopulation of fetal cells is mesenchymal progenitor cells. In some embodiments, the first subpopulation of fetal cells is megakaryocytes and the second population of fetal cells is vascular endothelial cells.

In some embodiments, the first subpopulation of fetal cells is stromal cells and the second subpopulation of fetal cells is endothelial cells. In some embodiments, the first subpopulation of fetal cells is stromal cells and the second subpopulation of fetal cells is lymphocytes. In some embodiments, the first subpopulation of fetal cells is stromal cells and the second subpopulation of fetal cells is nucleated red blood cells. In some embodiments, the first subpopulation of fetal cells is stromal cells and the second subpopulation of fetal cells is hematopoietic progenitor cells. In some embodiments, the first subpopulation of fetal cells is stromal cells and the second subpopulation of fetal cells is mesenchymal progenitor cells. In some embodiments, the first subpopulation of fetal cells is stromal cells and the second subpopulation of fetal cells is vascular endothelial cells.

In some embodiments, the first subpopulation of fetal cells is endothelial cells and the second subpopulation of fetal cells is lymphocytes. In some embodiments, the first subpopulation of fetal cells is endothelial cells and the second subpopulation of fetal cells is nucleated red blood cells. In some embodiments, the first subpopulation of fetal cells is endothelial cells and the second subpopulation of fetal cells is hematopoietic progenitor cells. In some embodiments, the first subpopulation of fetal cells is endothelial cells and the second subpopulation of fetal cells is mesenchymal progenitor cells. In some embodiments, the first subpopulation of fetal cells is endothelial cells and the second subpopulation of fetal cells is vascular endothelial cells.

In some embodiments, the first subpopulation of fetal cells is lymphocytes and the second subpopulation of fetal cells is nucleated red blood cells. In some embodiments, the first subpopulation of fetal cells is lymphocytes and the second subpopulation of fetal cells is hematopoietic progenitor cells. In some embodiments, the first subpopulation of fetal cells is lymphocytes and the second subpopulation of fetal cells is mesenchymal progenitor cells. In some embodiments, the first subpopulation of fetal cells is lymphocytes and the second subpopulation of fetal cells is vascular endothelial cells.

In some embodiments, the first subpopulation of fetal cells is nucleated red blood cells and the second subpopulation of fetal cells is hematopoietic progenitor cells. In some embodiments, the first subpopulation of fetal cells is nucleated red blood cells and the second subpopulation of fetal cells is mesenchymal progenitor cells. In some embodiments, the first subpopulation of fetal cells is nucleated red blood cells and the second subpopulation of fetal cells is vascular endothelial cells.

In some embodiments, the first subpopulation of fetal cells is hematopoietic progenitor cells and the second subpopulation of fetal cells is mesenchymal progenitor cells. In some embodiments, the first subpopulation of fetal cells is hematopoietic progenitor cells and the second subpopulation of fetal cells is vascular endothelial cells. In some embodiments, the first subpopulation of fetal cells is mesenchymal progenitor cells and the second subpopulation of fetal cells is hematopoietic progenitor cells. In some embodiments, the first subpopulation of fetal cells is mesenchymal progenitor cells and the second subpopulation of fetal cells is vascular endothelial cells.

In some embodiments, the method further comprises isolating the subpopulation of fetal cells from the maternal biological sample prior to sequencing. In some embodiments, the subpopulation of fetal cells is isolated from the maternal biological sample. In some embodiments, the subpopulation of fetal cells is isolated by contacting the maternal biological sample with a first isolation agent. In some embodiments, the maternal biological sample is contacted with one or more isolation agents. In some embodiments, the one or more isolation agents has a first isolation agent. In some embodiments, the first isolation agent couples to a first biomarker in the subpopulation of fetal cells.

In some embodiments, the method further comprises isolating the subpopulation of maternal cells from the maternal biological sample prior to sequencing. In some embodiments, the subpopulation of maternal cells is isolated from the maternal biological sample. In some embodiments, the subpopulation of maternal cells is isolated by contacting the maternal biological sample with one or more isolation agents. In some embodiments, the isolation agents include a second isolation agent. In some embodiments, the second isolation agent couples to a second biomarker in the subpopulation of maternal cells.

In some embodiments, the method further comprises incubating the maternal biological sample after the contacting. In some embodiments, subsequent to the contacting, the maternal biological sample is incubated with one or more isolation agents specific for one or more fetal biomarkers. In some embodiments, the maternal biological sample is incubated with at least the one or more isolation agents specific for one or more fetal biomarkers from about 0 hours to about 48 hours.

In some embodiments, the maternal biological sample is incubated with the one or more isolation agents specific for one or more fetal biomarkers from about 0 hour to about 1 hour, from about 1 hour to about 2 hour, from about 2 hours to about 3 hours, from about 3 hours to about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, from about 10 hours to about 11 hours, from about 11 hours to about 12 hours, from about 12 hours to about 13 hours, from about 13 hours to about 14 hours, from about 14 hours to about 15 hours, from about 15 hours to about 16 hours, from about 16 hours to about 17 hours, from about 17 hours to about 18 hours, from about 18 hours to about 19 hours, from about 19 hours to about 20 hours, from about 20 hours to about 21 hours, from about 21 hours to about 22 hours, from about 22 hours to about 23 hours, from about 23 hours to about 24 hours, from about 24 hours to about 26 hours, from about 26 hours to about 28 hours, from about 28 hours to about 30 hours, from about 30 hours to about 32 hours, from about 32 hours to about 34 hours, from about 34 hours to about 36 hours, from about 36 hours to about 38 hours, from about 38 hours to about 40 hours, from about 40 hours to about 42 hours, from about 42 hours to about 44 hours, from about 44 hours to about 46 hours, from about 46 hours to about 48 hours.

In some embodiments, the maternal biological sample is incubated with the one or more isolation agents specific for one or more fetal biomarkers for at least about 0 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 1 hour, at least about 1.25 hours, at least about 1.5 hours, at least about 1.75 hours, at least about 2 hours, at least about 2.25 hours, at least about 2.5 hours, at least about 2.75 hours, at least about 3 hours, at least about 3.25 hours, at least about 3.5 hours, at least about 3.75 hours, at least about 4 hours, at least about 4.25 hours, at least about 4.5 hours, at least about 4.75 hours, at least about 5 hours, at least about 5.25 hours, at least about 5.5 hours, at least about 5.75 hours, at least about 6 hours, at least about 6.25 hours, at least about 6.5 hours, at least about 6.75 hours, at least about 7 hours, at least about 7.25 hours, at least about 7.5 hours, at least about 7.75 hours, at least about 8 hours, at least about 8.25 hours, at least about 8.5 hours, at least about 8.75 hours, at least about 9 hours, at least about 9.25 hours, at least about 9.5 hours, at least about 9.75 hours, at least about 10 hours, at least about 10.25 hours, at least about 10.5 hours, at least about 10.75 hours, at least about 11 hours, at least about 11.25 hours, at least about 11.5 hours, at least about 11.75 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, at least about 48 hours, or more.

In some embodiments, the maternal biological sample is incubated with the one or more isolation agents specific for one or more fetal biomarkers for at most about 48 hours, at most about 47 hours, at most about 46 hours, at most about 45 hours, at most about 44 hours, at most about 43 hours, at most about 42 hours, at most about 41 hours, at most about 40 hours, at most about 39 hours, at most about 38 hours, at most about 37 hours, at most about 36 hours, at most about 35 hours, at most about 34 hours, at most about 33 hours, at most about 32 hours, at most about 31 hours, at most about 30 hours, at most about 29 hours, at most about 28 hours, at most about 27 hours, at most about 26 hours, at most about 25 hours, at most about 24 hours, at most about 23 hours, at most about 22 hours, at most about 21 hours, at most about 20 hours, at most about 19 hours, at most about 18 hours, at most about 17 hours, at most about 16 hours, at most about 15 hours, at most about 14 hours, at most about 13 hours, at most about 12 hours, at most about 11.75 hours, at most about 11.5 hours, at most about 11.25 hours, at most about 11 hours, at most about 10.75 hours, at most about 10.5 hours, at most about 10.25 hours, at most about 10 hours, at most about 9.75 hours, at most about 9.5 hours, at most about 9.25 hours, at most about 9 hours, at most about 8.75 hours, at most about 8.5 hours, at most about 8.25 hours, at most about 8 hours, at most about 7.75 hours, at most about 7.5 hours, at most about 7.25 hours, at most about 7 hours, at most about 6.75 hours, at most about 6.5 hours, at most about 6.25 hours, at most about 6 hours, at most about 5.75 hours, at most about 5.5 hours, at most about 5.25 hours, at most about 5 hours, at most about 4.75 hours, at most about 4.5 hours, at most about 4.25 hours, at most about 4 hours, at most about 3.75 hours, at most about 3.5 hours, at most about 3.25 hours, at most about 3 hours, at most about 2.75 hours, at most about 2.5 hours, at most about 2.25 hours, at most about 2 hours, at most about 1.75 hours, at most about 1.5 hours, at most about 1.25 hours, at most about 1 hour, at most about 55 minutes, at most about 50 minutes, at most about 45 minutes, at most about 40 minutes, at most about 35 minutes, at most about 30 minutes, at most about 25 minutes, at most about 20 minutes, at most about 15 minutes, at most about 10 minutes, at most about 5 minutes, at most about 0 minutes, or less.

In some embodiments, the maternal biological sample is incubated with the one or more isolation agents specific for one or more fetal biomarkers for about 0 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, about 5.25 hours, about 5.5 hours, about 5.75 hours, about 6 hours, about 6.25 hours about 6.5 hours, about 6.75 hours, about 7 hours, about 7.25 hours, about 7.5 hours, about 7.75 hours, about 8 hours, about 8.25 hours, about 8.5 hours, about 8.75 hours, about 9 hours, about 9.25 hours, about 9.5 hours, about 9.75 hours, about 10 hours, about 10.25 hours, about 10.5 hours, about 10.75 hours, about 11 hours, about 11.25 hours, about 11.5 hours, about 11.75 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47, or about 48 hours.

In some embodiments, the maternal biological sample is incubated with the at least one isolation agent specific for one or more fetal biomarkers at a temperature between about 20° C. to about 70° C. In some embodiments, the maternal biological sample is incubated with the at least one isolation agent specific for one or more fetal biomarkers at a temperature of at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., or more. In some embodiments, the maternal biological sample is incubated with the at least one isolation agent specific for one or more fetal biomarkers at a temperature of at most about 70° C., at most about 69° C., at most about 68° C., at most about 67° C., at most about 66° C., at most about 65° C., at most about 64° C., at most about 63° C., at most about 62° C., at most about 61° C., most about 60° C., at most about 59° C., at most about 58° C., at most about 57° C., at most about 56° C., at most about 55° C., at most about 54° C., at most about 53° C., at most about 52° C., at most about 51° C., at most about 50° C., at most about 49° C., at most about 48° C., at most about 47° C., at most about 46° C., at most about 45° C., at most about 44° C., at most about 43° C., at most about 42° C., at most about 41° C., at most about 40° C., at most about 39° C., at most about 38° C., at most about 37° C., at most about 36° C., at most about 35° C., at most about 34° C., at most about 33° C., at most about 32° C., at most about 31° C., at most about 30° C., at most about 29° C., at most about 28° C., at most about 27° C., at most about 26° C., at most about 25° C., at most about 24° C., at most about 23° C., at most about 22° C., at most about 21° C., at most about 20° C., or less. In some embodiments, the maternal biological sample is incubated with the at least one isolation agent specific to one or more fetal biomarkers at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

In some embodiments, the maternal biological sample is incubated with one or more isolation agents that couples to one or more fetal biomarkers. In some embodiments, the maternal biological sample is incubated with one or more isolation agents that couples to one or more maternal biomarkers. In some embodiments, the maternal biological sample is incubated with one or more isolation agents that couples to one or more fetal biomarkers, then subsequently incubated with one or more isolation agents that couples to one or more maternal biomarkers. In some embodiments, that maternal biological sample is incubated with one or more isolation agents that couples to one or more maternal biomarkers, then subsequently incubated with one or more isolation agents that couples to one or more fetal biomarkers. In some embodiments, the maternal biological sample is incubated with one or more isolation agents that couples to one or more fetal biomarkers, then subsequently incubated with another one or more isolation agents that couples to another one or more fetal biomarkers. In some embodiments, the maternal biological sample is incubated with one or more isolation agents that couples to one or more maternal biomarkers, then incubated with one or more isolation agents that couples to one or more fetal biomarkers, and then incubated with another one or more isolation agents that couples to another one or more fetal biomarkers.

In some embodiments, the first isolation agent couples to a first biomarker. In some embodiments, the first biomarker is one or more fetal biomarkers. In some embodiments, the one or more isolation agents specific for one or more fetal biomarkers couples to an extracellular biomarker. In some embodiments, the one or more isolation agents specific for one or more fetal biomarkers couples to an extracellular protein. In some embodiments, the one or more isolation agents specific for one or more fetal biomarkers couples to a cell surface protein. In some embodiments, the one or more isolation agents specific for one or more fetal biomarkers couples to a soluble cell surface protein. In some embodiments, the one or more isolation agents specific for one or more fetal biomarkers couples to a cell-surface receptor. In some embodiments, the one or more isolation agents specific for one or more fetal biomarkers couples to a transmembrane protein. In some embodiments, the one or more isolation agents specific for one or more fetal biomarkers couples to a transmembrane channel protein.

In some embodiments, the one or more isolation agents specific for one or more fetal biomarkers couples to an intracellular biomarker. In some embodiments, the one or more isolation agents specific for one or more fetal biomarkers couples to an intracellular protein. In some embodiments, the one or more isolation agents specific for one or more fetal biomarkers couples to an enzyme. In some embodiments, the one or more isolation agents specific for one or more fetal biomarkers couples to a phosphoprotein. In some embodiments, the one or more isolation agents specific for one or more fetal biomarkers couples to a growth factor. In some embodiments, the one or more isolation agents specific for one or more fetal biomarkers couples to a structural protein.

In some embodiments, the one or more isolation agents specific for one or more fetal biomarkers couples to a nucleic acid biomarker. In some embodiments, the one or more isolation agents specific for one or more fetal biomarkers couples to a DNA biomarker. In some embodiments, the one or more isolation agents specific for one or more fetal biomarkers couples to an RNA biomarker. In some embodiments, the one or more isolation agents specific for one or more fetal biomarkers couples to mRNA. In some embodiments, the one or more isolation agents specific for one or more fetal biomarkers couples to rRNA. In some embodiments, the one or more isolation agents specific for one or more fetal biomarkers couples to lncRNA. In some embodiments, the one or more isolation agents specific for one or more fetal biomarkers couples to miRNA. In some embodiments, the one or more isolation agents specific for one or more fetal biomarkers couples to siRNA.

In some embodiments, the first isolation agent couples to one or more fetal biomarkers. In some embodiments, the first biomarker is one or more fetal biomarkers. In some embodiments, the first biomarker is a Sct biomarker, a H19 biomarker, a Tfpi biomarker, or a Fthl17a biomarker. In some embodiments, the first biomarker is a Sct biomarker. In some embodiments, the first biomarker is a H19 biomarker. In some embodiments, the first biomarker is a Tfpi biomarker. In some embodiments, the first biomarker is a Fthl17a biomarker.

In some embodiments, the method further comprises isolating the subpopulation of fetal cells from the maternal biological sample prior to sequencing. In some embodiments, the subpopulation of fetal cells is isolated from the maternal biological sample. In some embodiments, the subpopulation of fetal cells is isolated by contacting the maternal biological sample with one or more isolation agents specific for one or more maternal biomarkers. In some embodiments, the maternal biological sample is contacted with one or more isolation agents specific for one or more maternal biomarkers. In some embodiments, the one or more isolation agents specific for one or more maternal biomarkers to a second biomarker in the subpopulation of maternal cells.

In some embodiments, the method further comprises isolating the subpopulation of maternal cells from the maternal biological sample prior to sequencing. In some embodiments, the subpopulation of maternal cells is isolated from the maternal biological sample. In some embodiments, the subpopulation of maternal cells is isolated by contacting the maternal biological sample with a one or more isolation agents specific for one or more fetal biomarkers. In some embodiments, the one or more isolation agents specific for one or more fetal biomarkers couples to a first biomarker in the subpopulation of fetal cells.

In some embodiments, the method further comprises incubating the maternal biological sample after the contacting. In some embodiments, subsequent to the contacting, the maternal biological sample is incubated with the one or more isolation agents specific for one or more maternal biomarkers. In some embodiments, the maternal biological sample is incubated with the one or more isolation agents specific for one or more maternal biomarkers from about 0 hours to about 48 hours.

In some embodiments, the maternal biological sample is incubated with the second isolation agent from about 0 hour to about 1 hour, from about 1 hour to about 2 hour, from about 2 hours to about 3 hours, from about 3 hours to about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, from about 10 hours to about 11 hours, from about 11 hours to about 12 hours, from about 12 hours to about 13 hours, from about 13 hours to about 14 hours, from about 14 hours to about 15 hours, from about 15 hours to about 16 hours, from about 16 hours to about 17 hours, from about 17 hours to about 18 hours, from about 18 hours to about 19 hours, from about 19 hours to about 20 hours, from about 20 hours to about 21 hours, from about 21 hours to about 22 hours, from about 22 hours to about 23 hours, from about 23 hours to about 24 hours, from about 24 hours to about 26 hours, from about 26 hours to about 28 hours, from about 28 hours to about 30 hours, from about 30 hours to about 32 hours, from about 32 hours to about 34 hours, from about 34 hours to about 36 hours, from about 36 hours to about 38 hours, from about 38 hours to about 40 hours, from about 40 hours to about 42 hours, from about 42 hours to about 44 hours, from about 44 hours to about 46 hours, from about 46 hours to about 48 hours.

In some embodiments, the maternal biological sample is incubated with the one or more isolation agents specific for one or more maternal biomarkers for at least about 0 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 1 hour, at least about 1.25 hours, at least about 1.5 hours, at least about 1.75 hours, at least about 2 hours, at least about 2.25 hours, at least about 2.5 hours, at least about 2.75 hours, at least about 3 hours, at least about 3.25 hours, at least about 3.5 hours, at least about 3.75 hours, at least about 4 hours, at least about 4.25 hours, at least about 4.5 hours, at least about 4.75 hours, at least about 5 hours, at least about 5.25 hours, at least about 5.5 hours, at least about 5.75 hours, at least about 6 hours, at least about 6.25 hours, at least about 6.5 hours, at least about 6.75 hours, at least about 7 hours, at least about 7.25 hours, at least about 7.5 hours, at least about 7.75 hours, at least about 8 hours, at least about 8.25 hours, at least about 8.5 hours, at least about 8.75 hours, at least about 9 hours, at least about 9.25 hours, at least about 9.5 hours, at least about 9.75 hours, at least about 10 hours, at least about 10.25 hours, at least about 10.5 hours, at least about 10.75 hours, at least about 11 hours, at least about 11.25 hours, at least about 11.5 hours, at least about 11.75 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, at least about 48 hours, or more.

In some embodiments, the maternal biological sample is incubated with the one or more isolation agents specific for one or more maternal biomarkers for at most about 48 hours, at most about 47 hours, at most about 46 hours, at most about 45 hours, at most about 44 hours, at most about 43 hours, at most about 42 hours, at most about 41 hours, at most about 40 hours, at most about 39 hours, at most about 38 hours, at most about 37 hours, at most about 36 hours, at most about 35 hours, at most about 34 hours, at most about 33 hours, at most about 32 hours, at most about 31 hours, at most about 30 hours, at most about 29 hours, at most about 28 hours, at most about 27 hours, at most about 26 hours, at most about 25 hours, at most about 24 hours, at most about 23 hours, at most about 22 hours, at most about 21 hours, at most about 20 hours, at most about 19 hours, at most about 18 hours, at most about 17 hours, at most about 16 hours, at most about 15 hours, at most about 14 hours, at most about 13 hours, at most about 12 hours, at most about 11.75 hours, at most about 11.5 hours, at most about 11.25 hours, at most about 11 hours, at most about 10.75 hours, at most about 10.5 hours, at most about 10.25 hours, at most about 10 hours, at most about 9.75 hours, at most about 9.5 hours, at most about 9.25 hours, at most about 9 hours, at most about 8.75 hours, at most about 8.5 hours, at most about 8.25 hours, at most about 8 hours, at most about 7.75 hours, at most about 7.5 hours, at most about 7.25 hours, at most about 7 hours, at most about 6.75 hours, at most about 6.5 hours, at most about 6.25 hours, at most about 6 hours, at most about 5.75 hours, at most about 5.5 hours, at most about 5.25 hours, at most about 5 hours, at most about 4.75 hours, at most about 4.5 hours, at most about 4.25 hours, at most about 4 hours, at most about 3.75 hours, at most about 3.5 hours, at most about 3.25 hours, at most about 3 hours, at most about 2.75 hours, at most about 2.5 hours, at most about 2.25 hours, at most about 2 hours, at most about 1.75 hours, at most about 1.5 hours, at most about 1.25 hours, at most about 1 hour, at most about 55 minutes, at most about 50 minutes, at most about 45 minutes, at most about 40 minutes, at most about 35 minutes, at most about 30 minutes, at most about 25 minutes, at most about 20 minutes, at most about 15 minutes, at most about 10 minutes, at most about 5 minutes, at most about 0 minutes, or less.

In some embodiments, the maternal biological sample is incubated with the one or more isolation agents specific for one or more maternal biomarkers for about 0 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, about 5.25 hours, about 5.5 hours, about 5.75 hours, about 6 hours, about 6.25 hours about 6.5 hours, about 6.75 hours, about 7 hours, about 7.25 hours, about 7.5 hours, about 7.75 hours, about 8 hours, about 8.25 hours, about 8.5 hours, about 8.75 hours, about 9 hours, about 9.25 hours, about 9.5 hours, about 9.75 hours, about 10 hours, about 10.25 hours, about 10.5 hours, about 10.75 hours, about 11 hours, about 11.25 hours, about 11.5 hours, about 11.75 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47, or about 48 hours.

In some embodiments, the maternal biological sample is incubated with the at least one isolation agent specific for one or more maternal biomarkers at a temperature between about 20° C. to about 70° C. In some embodiments, the maternal biological sample is incubated with the at least one isolation agent specific for one or more maternal biomarkers at a temperature of at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., or more. In some embodiments, the maternal biological sample is incubated with the at least one isolation agent specific for one or more maternal biomarkers at a temperature of at most about 70° C., at most about 69° C., at most about 68° C., at most about 67° C., at most about 66° C., at most about 65° C., at most about 64° C., at most about 63° C., at most about 62° C., at most about 61° C., most about 60° C., at most about 59° C., at most about 58° C., at most about 57° C., at most about 56° C., at most about 55° C., at most about 54° C., at most about 53° C., at most about 52° C., at most about 51° C., at most about 50° C., at most about 49° C., at most about 48° C., at most about 47° C., at most about 46° C., at most about 45° C., at most about 44° C., at most about 43° C., at most about 42° C., at most about 41° C., at most about 40° C., at most about 39° C., at most about 38° C., at most about 37° C., at most about 36° C., at most about 35° C., at most about 34° C., at most about 33° C., at most about 32° C., at most about 31° C., at most about 30° C., at most about 29° C., at most about 28° C., at most about 27° C., at most about 26° C., at most about 25° C., at most about 24° C., at most about 23° C., at most about 22° C., at most about 21° C., at most about 20° C., or less. In some embodiments, the maternal biological sample is incubated with the at least one isolation agent specific for one or more maternal biomarkers at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

In some embodiments, the maternal biological sample is incubated with one or more isolation agents that couples to one or more fetal biomarkers. In some embodiments, the maternal biological sample is incubated with one or more isolation agents that couples to one or more maternal biomarkers. In some embodiments, the maternal biological sample is incubated with one or more isolation agents that couples to one or more fetal biomarkers, then subsequently incubated with one or more isolation agents that couples to one or more maternal biomarkers. In some embodiments, the maternal biological sample is incubated with one or more isolation agents that couples to one or more maternal biomarkers, then subsequently incubated with one or more isolation agents that couples to one or more fetal biomarkers. In some embodiments, the maternal biological sample is incubated with one or more isolation agents that couples to one or more fetal biomarkers, then subsequently incubated with another one or most isolation agents that couples to another one or more fetal biomarkers. In some embodiments, the maternal biological sample is incubated with one or more isolation agents that couples to one or more maternal biomarkers, then incubated with one or more isolation agents that couples to one or more fetal biomarkers, and then incubated with another one or more isolation agents that couples to another one or more fetal biomarkers.

In some embodiments, the maternal biological sample undergoes at least one round of enrichment to enrich/isolate fetal cells from the maternal blood sample. In some embodiments, the maternal biological sample undergoes between about one round of enrichment and about ten rounds of enrichment to enrich/isolate fetal cells from the maternal biological sample. In some embodiments, the maternal biological sample undergoes at least about one round, at least about two rounds, at least about three rounds, at least about four rounds, at least about five rounds, at least about six rounds, at least about seven rounds, at least about eight rounds, at least about nine rounds, or at least about ten rounds or more of enrichment to enrich/isolate fetal cells from the maternal biological sample. In some embodiments, the maternal biological sample undergoes at most about ten rounds, at most about nine rounds, at most about eight rounds, at most about seven rounds, at most about six rounds, at most about five rounds, at most about four rounds, at most about three rounds, at most about two rounds, at most about one round, or less of enrichment to enrich/isolate fetal cells from the maternal biological sample. In some embodiments, the maternal biological sample undergoes about one round, about two rounds, about three rounds, about four rounds, about five rounds, about six rounds, about seven rounds, about eight rounds, about nine rounds, or about ten rounds of enrichment to enrich/isolate fetal cells from the maternal biological sample. In some embodiments, the maternal biological sample undergoes about one round of enrichment to enrich/isolate fetal cells from the maternal biological sample. In some embodiments, the maternal biological sample undergoes about two rounds of enrichment to enrich/isolate fetal cells from the maternal biological sample. In some embodiments, the maternal biological sample undergoes about three rounds of enrichment to enrich/isolate fetal cells from the maternal biological sample. In some embodiments, the maternal biological sample undergoes about four rounds of enrichment to enrich/isolate fetal cells from the maternal biological sample. In some embodiments, the maternal biological sample undergoes about five rounds of enrichment to enrich/isolate fetal cells from the maternal biological sample.

In some embodiments, the at least one round of enrichment enriches for at least one fetal biomarker. In some embodiments, the at least one round of enrichment enriches for at least one maternal biomarker. In some embodiments, the at least one round of enrichment enriches for maternal biomarkers, subsequently followed by enriching for at least one fetal biomarker. In some embodiments, the at least one round of enrichment enriches for at least one maternal biomarkers, followed by enriching for at least one fetal biomarkers, and then followed by enriching for at least another fetal biomarker. In some embodiments, the at least one round of enrichment enriches for fetal biomarkers, subsequently followed by enriching for at least another fetal biomarker.

In some embodiments, the at least one fetal biomarker is about one fetal biomarker disclosed herein. In some embodiments, the at least one fetal biomarker is at least two of more fetal biomarkers disclosed herein. In some embodiments, the at least one fetal biomarker is between about two fetal biomarkers to about ten fetal biomarkers. In some embodiments, the at least one fetal biomarker is at least one about fetal biomarker, at least about two fetal biomarkers, at least about three fetal biomarkers, at least about four fetal biomarkers, at least about five fetal biomarkers, at least about six fetal biomarkers, at least about seven fetal biomarkers, at least about eight fetal biomarkers, at least about nine fetal biomarkers, or at least about ten fetal biomarkers. In some embodiments, the at least one fetal biomarker is at most about ten fetal biomarkers, at most about nine fetal biomarkers, at most about eight fetal biomarkers, at most about seven fetal biomarkers, at most about six fetal biomarkers, at most about five fetal biomarkers, at most about four fetal biomarkers, at most about three fetal biomarkers, at most about two fetal biomarkers, at most about one fetal biomarker, or less. In some embodiments, the at least one fetal biomarker is about one fetal biomarker, about two fetal biomarkers, about three fetal biomarkers, about four fetal biomarkers, about five fetal biomarkers, about six fetal biomarkers, about seven fetal biomarkers, about eight fetal biomarkers, about nine fetal biomarkers, or about ten fetal biomarkers. In some embodiments, the at least one fetal biomarker is about two fetal biomarkers. In some embodiments, the at least one fetal biomarker is about three fetal biomarkers. In some embodiments, the at least one fetal biomarker is about four fetal biomarkers. In some embodiments, the at least one fetal biomarker is about five fetal biomarkers. In some embodiments, the at least one fetal biomarker is any combination of fetal biomarkers disclosed within the present disclosure.

In some embodiments, the at least one maternal biomarker is about one maternal biomarker disclosed herein. In some embodiments, the at least one maternal biomarker is at least two of more maternal biomarkers disclosed herein. In some embodiments, the at least one maternal biomarker is between about two maternal biomarkers to about ten maternal biomarkers. In some embodiments, the at least one maternal biomarker is at least one about maternal biomarker, at least about two maternal biomarkers, at least about three maternal biomarkers, at least about four maternal biomarkers, at least about five maternal biomarkers, at least about six maternal biomarkers, at least about seven maternal biomarkers, at least about eight maternal biomarkers, at least about nine maternal biomarkers, or at least about ten maternal biomarkers. In some embodiments, the at least one maternal biomarker is at most about ten maternal biomarkers, at most about nine maternal biomarkers, at most about eight maternal biomarkers, at most about seven maternal biomarkers, at most about six maternal biomarkers, at most about five maternal biomarkers, at most about four maternal biomarkers, at most about three maternal biomarkers, at most about two maternal biomarkers, at most about one maternal biomarker, or less. In some embodiments, the at least one maternal biomarker is about one maternal biomarker, about two maternal biomarkers, about three maternal biomarkers, about four maternal biomarkers, about five maternal biomarkers, about six maternal biomarkers, about seven maternal biomarkers, about eight maternal biomarkers, about nine maternal biomarkers, or about ten maternal biomarkers. In some embodiments, the at least one maternal biomarker is about two maternal biomarkers. In some embodiments, the at least one maternal biomarker is about three maternal biomarkers. In some embodiments, the at least one maternal biomarker is about four maternal biomarkers. In some embodiments, the at least one maternal biomarker is about five maternal biomarkers. In some embodiments, the at least one maternal biomarker is any combination of maternal biomarkers disclosed within the present disclosure.

In some embodiments, the one or more isolation agents specific for one or more maternal biomarkers couples to a second biomarker. In some embodiments, the second biomarker is a maternal biomarker. In some embodiments, the isolation agent specific for a maternal biomarker couples to an extracellular biomarker. In some embodiments, the isolation agent specific for a maternal biomarker couples to an extracellular protein. In some embodiments, the isolation agent specific for a maternal biomarker couples to a cell surface protein. In some embodiments, the isolation agent specific for a maternal biomarker couples to a soluble cell surface protein. In some embodiments, the isolation agent specific for a maternal biomarker couples to a cell-surface receptor. In some embodiments, the isolation agent specific for a maternal biomarker couples to a transmembrane protein. In some embodiments, the isolation agent specific for a maternal biomarker couples to a transmembrane channel protein.

In some embodiments, the isolation agent specific for a maternal biomarker couples to an intracellular biomarker. In some embodiments, the isolation agent specific for a maternal biomarker couples to an intracellular protein. In some embodiments, the isolation agent specific for a maternal biomarker couples to an enzyme. In some embodiments, the isolation agents specific for a maternal biomarker couples to a phosphoprotein. In some embodiments, the isolation agent specific for a maternal biomarker couples to a growth factor. In some embodiments, the isolation agent specific for a maternal biomarker couples to a structural protein.

In some embodiments, the isolation agent specific for a maternal biomarker couples to a nucleic acid biomarker. In some embodiments, the isolation agent specific for a maternal biomarker couples to a DNA biomarker. In some embodiments, the isolation agent specific for a maternal biomarker couples to an RNA biomarker. In some embodiments, the isolation agent specific for a maternal biomarker couples to mRNA. In some embodiments, the isolation agent specific for a maternal biomarker couples to rRNA. In some embodiments, the isolation agent specific for a maternal biomarker couples to lncRNA. In some embodiments, the isolation agent specific for a maternal biomarker couples to miRNA. In some embodiments, the second isolation agent couples to siRNA.

In some embodiments, the second isolation agent couples to a maternal biomarkers. In some embodiments, the second biomarker is one or more maternal biomarkers. In some embodiments, the second biomarker is a Coro1a biomarker, a Cd52 biomarker, a Cyba biomarker, a Rac2 biomarker, a Laptm5 biomarker, or a Ptprc biomarker. In some embodiments, the second biomarker is a Coro1a biomarker. In some embodiments, the second biomarker is a Cd52 biomarker. In some embodiments, the second biomarker is a Cyba biomarker. In some embodiments, the second biomarker is a Rac2 biomarker. In some embodiments, the second biomarker is a Laptm5 biomarker. In some embodiments, the second biomarker is a Ptprc biomarker.

After obtaining the subpopulation of fetal cells and/or the subpopulation of maternal cells, the one or more isolation agents bound to the one or more biomarkers can be removed. In some embodiments, the one or more isolation agents bound to the one or more biomarkers can be removed by one or more washing operations. In some embodiments, the one or more isolation agents bound to the one or more biomarkers can be removed by centrifugation. In some embodiments, the one or more isolation agents bound to the one or more biomarkers can be removed by centrifugation of the subpopulation of fetal cells and/or the subpopulation of maternal cells from between 1200 revolutions per minute (rpm) to about 1800 rpm. In some embodiments, the one or more isolation agents bound to the one or more biomarkers can be removed by centrifugation of the subpopulation of fetal cells and/or the subpopulation of maternal cells at a rate of at least about 1200 rpm, at least about 1250 rpm, at least about 1300 rpm, at least about 1350 rpm, at least about 1400 rpm, at least about 1450 rpm, at least about 1500 rpm, at least about 1550 rpm, at least about 1600 rpm, at least about 1650 rpm, at least about 1700 rpm, at least about 1750 rpm, at least about 1800 rpm, or more. In some embodiments, the one or more isolation agents bound to the one or more biomarkers can be removed by centrifuging the subpopulation of fetal cells and/or the subpopulation of maternal cells at a rate of at most 1800 rpm, at most about 1750 rpm, at most about 1700 rpm, at most about 1650 rpm, at most about 1600 rpm, at most about 1550 rpm, at most about 1500 rpm, at most about 1450 rpm, at most about 1400 rpm, at most about 1350 rpm, at most about 1300 rpm, at most about 1250 rpm, at most about 1200 rpm, or less. In some embodiments, the one or more isolation agents bound to the one or more biomarkers can be removed by centrifugation of the subpopulation of fetal cells and/or the subpopulation of maternal cells at about 1200 rpm, about 1250 rpm, about 1300 rpm, about 1350 rpm, about 1400 rpm, about 1450 rpm, about 1500 rpm, about 1550 rpm, about 1600 rpm, about 1650 rpm, about 1700 rpm, about 1750 rpm, about 1800 rpm, or more. In some embodiments, the one or more isolation agents bound to the one or more biomarkers can be removed by centrifugation of the subpopulation of fetal cells and/or the subpopulation of maternal cells from between about 0 minutes to about 30 minutes. In some embodiments, the one or more isolation agents bound to the one or more biomarkers can be removed by centrifugation of the subpopulation of fetal cells and/or the subpopulation of maternal cells for at least about 0 minutes, at least about 5 minutes, at least about 10 minutes, at least about 15 minutes, at least about 20 minutes, at least about 25 minutes, at least about 30 minutes, or more. In some embodiments, the one or more isolation agents bound to the one or more biomarkers can be removed by centrifugation of the subpopulation of fetal cells and/or the subpopulation of maternal cells for at most about 30 minutes, at most about 25 minutes, at most about 20 minutes, at most about 15 minutes, at most about 10 minutes, at most about 5 minutes, at most about 0 minutes, or less. In some embodiments, the one or more isolation agents bound to the one or more biomarkers can be removed by centrifugation of the subpopulation of fetal cells and/or the subpopulation of maternal cells for about 0 minutes, about 5 minutes, about 10 minutes, about 15 minutes, about 20 minutes, about 25 minutes, or about 30 minutes.

In some embodiments, the one or more isolation agents bound to the one or more biomarkers can be removed by exposing the subpopulation of fetal cells and/or subpopulation of maternal cells to heat. In some embodiments, the one or more isolation agents bound to the one or more biomarkers can be removed by exposing the subpopulation of fetal cells and/or the subpopulation of maternal cells to heat from between about 0 minutes to about 30 minutes. In some embodiments, the one or more isolation agents bound to the one or more biomarkers can be removed by exposing the subpopulation of fetal cells and/or the subpopulation of maternal cells to heat for at least about 0 minutes, at least about 5 minutes, at least about 10 minutes, at least about 15 minutes, at least about 20 minutes, at least about 25 minutes, at least about 30 minutes, or more. In some embodiments, the one or more isolation agents bound to the one or more biomarkers can be removed by exposing the subpopulation of fetal cells and/or the subpopulation of maternal cells to heat for at most about 30 minutes, at most about 25 minutes, at most about 20 minutes, at most about 15 minutes, at most about 10 minutes, at most about 5 minutes, at most about 0 minutes, or less. In some embodiments, the one or more isolation agents bound to the one or more biomarkers can be removed by exposing the subpopulation of fetal cells and/or the subpopulation of maternal cells to heat for about 0 minutes, about 5 minutes, about 10 minutes, about 15 minutes, about 20 minutes, about 25 minutes, or about 30 minutes. In some embodiments, the one or more isolation agents bound to the one or more biomarkers can be removed by exposing the subpopulation of fetal cells and/or the subpopulation of maternal cells to a temperature of between about 50° C. to about 100° C. In some embodiments, the one or more isolation agents bound to the one or more biomarkers can be removed by exposing the subpopulation of fetal cells and/or the subpopulation of maternal cells to a temperature of at least about 50° C., at least about 55° C., at least about 60° C., at least about 65° C., at least about 70° C., at least about 75° C., at least about 80° C., at least about 85° C., at least about 90° C., at least about 95° C., at least about 100° C., or greater. In some embodiments, the one or more isolation agents bound to the one or more biomarkers can be removed by exposing the subpopulation of fetal cells and/or the subpopulation of maternal cells to a temperature of at most about 100° C., at most about 95° C., at most about 90° C., at most about 85° C., at most about 80° C., at most about 75° C., at most about 70° C., at most about 65° C., at most about 60° C., at most about 55° C., at most about 50, or less. In some embodiments, the one or more isolation agents bound to the one or more biomarkers can be removed by exposing the subpopulation of fetal cells and/or the subpopulation of maternal cells to a temperature of about 50° C., about 55° C., about 60° C., about 65° C., about 70° C., about 75° C., about 80° C., about 85° C., about 90° C., about 95° C., or about 100° C.

After obtaining the subpopulation of fetal cells and/or the subpopulation of maternal cells, the fetal cells and/or maternal cells can be processed to generate sequencing data of the genome of the fetal cells. Processing the biological sample obtained from the subject can comprise (i) subjecting the CFCs to conditions that are sufficient to isolate, enrich, or extract a plurality of fetal cell genetic material, and (ii) sequencing the plurality of fetal cell genetic material to generate a plurality of sequencing reads. Processing the biological sample obtained from the subject can comprise (i) subjecting the maternal cells to conditions that are sufficient to isolate, enrich, or extract a plurality of maternal cell genetic material, and (ii) sequencing the plurality of maternal cell genetic material to generate a plurality of sequencing reads. In some embodiments, the plurality of sequencing reads is used to determine whether the fetus (or fetuses) has a genetic disorder. The nucleic acid molecules can comprise ribonucleic acid (RNA) or deoxyribonucleic acid (DNA). The nucleic acid molecules can comprise DNA. The nucleic acid molecules can comprise RNA. The nucleic acid molecules (e.g., RNA or DNA) can be extracted from the subpopulation of fetal cells by a variety of methods, such as a DNeasy Blood and Tissue Kit, a PureLink Genomic DNA kit, or a Wizard® HMW DNA Extraction Kit. The extraction method can extract all RNA or DNA molecules from a sample.

In some embodiments, the genetic material from the subpopulation of fetal cells is genetically analyzed. In some embodiments, genetic material is analyzed via PCR, sequencing, hybridization methods, or any combination thereof. In some embodiments, the genetic material is analyzed via a whole genome scan. In some embodiments, the genetic material is analyzed via short tandem repeat analysis. In some embodiments, the genetic material is analyzed via microsatellite analysis.

In some embodiments, the genetic analysis is a hybridization method. In some embodiments, the hybridization method is a restriction fragment length polymorphism (RFLP), a southern blot analysis, a microarray, a fluorescence in situ hybridization (FISH), a comparative genomic hybridization, in situ hybridization, or any combination thereof.

In some embodiments, the genetic material from the subpopulation of fetal cells is amplified prior to genetic analysis. In some embodiments, the genetic material from the subpopulation of fetal cells is amplified to increase the amount of the genetic material from the subpopulation of fetal cells by between about 0% to about 500%. In some embodiments, the genetic material from the subpopulation of fetal cells is amplified to increase the amount of genetic material from the subpopulation of fetal cell by at least about 0%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 160%, at least about 180%, at least about 200%, at least about 250%, at least about 300%, at least about 350%, at least about 400%, at least about 450%, at least about 500%, or more. In some embodiments, the genetic material from the subpopulation of fetal cells is amplified to increase the amount of genetic material from the subpopulation of fetal cell by at most about 500%, at most about 450%, at most about 400%, at most about 350%, at most about 300%, at most about 250%, at most about 200%, at most about 180%, at most about 160%, at most about 140%, at most about 120%, at most about 100%, at most about 90%, at most about 80%, at most about 70%, at most about 60%, at most about 50%, at most about 40%, at most about 30%, at most about 20%, at most about 10%, at most about 0%, or less. In some embodiments, the genetic material from the subpopulation of fetal cells is amplified to increase the amount of genetic material from the subpopulation of fetal cell by about 0%, about 10%, about 20%, about 30%, about 40%, about 50%, about 60%, about 70%, about 80%, about 90%, about 100%, about 120%, about 140%, about 160%, about 180%, about 200%, about 250%, about 300%, about 350%, about 400%, about 450%, or about 500%.

some embodiments, the genetic material from the subpopulation of maternal cells is amplified prior to genetic analysis. In some embodiments, the genetic material from the subpopulation of maternal cells is amplified to increase the amount of the genetic material from the subpopulation of maternal cells by between about 0% to about 500%. In some embodiments, the genetic material from the subpopulation of maternal cells is amplified to increase the amount of genetic material from the subpopulation of maternal cell by at least about 0%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 160%, at least about 180%, at least about 200%, at least about 250%, at least about 300%, at least about 350%, at least about 400%, at least about 450%, at least about 500%, or more. In some embodiments, the genetic material from the subpopulation of maternal cells is amplified to increase the amount of genetic material from the subpopulation of maternal cell by at most about 500%, at most about 450%, at most about 400%, at most about 350%, at most about 300%, at most about 250%, at most about 200%, at most about 180%, at most about 160%, at most about 140%, at most about 120%, at most about 100%, at most about 90%, at most about 80%, at most about 70%, at most about 60%, at most about 50%, at most about 40%, at most about 30%, at most about 20%, at most about 10%, at most about 0%, or less. In some embodiments, the genetic material from the subpopulation of maternal cells is amplified to increase the amount of genetic material from the subpopulation of maternal cell by about 0%, about 10%, about 20%, about 30%, about 40%, about 50%, about 60%, about 70%, about 80%, about 90%, about 100%, about 120%, about 140%, about 160%, about 180%, about 200%, about 250%, about 300%, about 350%, about 400%, about 450%, or about 500%.

In some embodiments, the genetic analysis is sequencing. In some embodiments, the method further comprises sequencing the nucleic acid molecules from the subpopulation of fetal cells. In some embodiments, the nucleic acid molecules from the subpopulation of fetal cells generate a nucleic acid sequence. In some embodiments, the nucleic acid sequence is from the subpopulation of fetal cells. In some embodiments, the method further comprises sequencing the nucleic acid molecules from the subpopulation of maternal cells. In some embodiments, the nucleic acid molecules from the subpopulation of maternal cells generate another nucleic acid sequence. In some embodiments, the other nucleic acid sequence is from the subpopulation of maternal cells. In some embodiments, the nucleic acid sequence and/or the other nucleic acid sequence is a DNA sequence. In some embodiments, the nucleic acid sequence and/or the other nucleic acid sequence is a RNA sequence.

Non-limiting examples of sequencing platforms include Sanger sequencing, massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina). In some embodiments, the sequencing is performed on a next generation sequencing platform. In some embodiments, the sequencing is performed on a massively parallel sequencing platform. In some embodiments, the sequencing is performed on a paired-end sequencing platform. In some embodiments, the sequencing is performed on a polony sequencing platform. In some embodiments, the sequencing is performed on a high-throughput sequencing platform. In some embodiments, the sequencing is performed on a shotgun sequencing platform. In some embodiments, the sequencing is performed on a single-molecule sequencing platform. In some embodiments, the sequencing is performed on a nanopore sequencing platform. In some embodiments, the sequencing is performed on a semiconductor sequencing platform. In some embodiments, the sequencing is performed on a pyrosequencing platform. In some embodiments, the sequencing is performed on a sequencing-by-synthesis platform. In some embodiments, the sequencing is performed on a sequencing-by-ligation platform. In some embodiments, the sequencing is performed on a sequencing-by-hybridization platform. In some embodiments, the sequencing is performed on an RNA-SEQ (Illumina) platform.

The sequencing can comprise nucleic acid amplification (e.g., of RNA or DNA molecules). In some embodiments, the entire genome is amplified prior to sequencing.

In some embodiments, the nucleic acid amplification is polymerase chain reaction (PCR). A suitable number of rounds of PCR (e.g., PCR, qPCR, reverse-transcriptase PCR, digital PCR, etc.) is performed to amplify an initial amount of nucleic acid (e.g., RNA or DNA) to a desired input quantity for subsequent sequencing. In some embodiments, the PCR is used for global amplification of target nucleic acids. This amplification can comprise using adapter sequences that are first ligated to different molecules followed by PCR amplification using universal primers. PCR can be performed using a kit of all the necessary tools. In some embodiments, only certain target nucleic acids within a population of nucleic acids can be amplified. Specific primers, for example, in conjunction with adapter ligation, can be used to amplify certain targets selectively for downstream sequencing. The PCR can comprise targeted amplification of one or more genomic loci, such as genomic loci associated with pregnancy-related states. The sequencing can comprise use of simultaneous reverse transcription (RT) and polymerase chain reaction (PCR), such as a OneStep RT-PCR kit protocol.

In some embodiments, the genetic analysis of fetal genetic material is PCR. In some embodiments, the PCT is quantitative PCR (qPCR). In some embodiments, the qPCR uses Taqman primer/probes sets. In some embodiments, the PCR is RT-PCR. In some embodiments, the PCR is droplet digital PCR (ddPCR). In some embodiments, the PCR is digital PCR. In some embodiments, the PCR analysis is a PCR-based mutation detection. In some embodiments, the PCR analysis is single nucleotide polymorphisms (SNP) genotyping arrays.

In some embodiments, the method further comprises collecting paternal cells from the father of the fetus. In some embodiments, the paternal cells from the father of the fetus are collected from a biological sample taken from the father. In some examples, the biological sample is a blood sample. In some examples, the biological sample is a tissue and/or biopsy sample. In some examples, the biological sample is a biological fluid sample. In some examples, the biological fluid sample is blood, saliva, interstitial fluid, extracellular fluid, or any combination thereof.

In some embodiments, the paternal cells have from about one paternal cell type to about 10 paternal cell types. In some embodiments, the paternal cells have at least about one paternal cell types, at least about two paternal cell types, at least about three paternal cell types, at least about four paternal cell types, at least about five paternal cell types, at least about six paternal cell types, at least about seven paternal cell types, at least about eight paternal cell types, at least about nine paternal cell types, at least about ten paternal cell types, or more. In some embodiments, the paternal cells have at most about ten paternal cell types, at most about nine paternal cell types, at most about eight paternal cell types, at most about seven paternal cell types, at most about six paternal cell types, at most about five paternal cell types, at most about four paternal cell types, at most about three paternal cell types, at most about two paternal cell types, at most about one paternal cell type, or less. In some embodiments, the paternal cells have about one paternal cell types, about two paternal cell types, about three paternal cell types, about four paternal cell types, about five paternal cell types, about six paternal cell types, about seven paternal cell types, about eight paternal cell types, about nine paternal cell types, or about ten paternal cell types. Non-limiting examples of types of paternal cells include red blood cells, T cells, B cells, dendritic cells, macrophages, monocytes, erythrocytes, neutrophils, and lymphocytes.

In some embodiments, the paternal cells are isolated from the biological sample from the father. In some embodiments, the paternal cells are isolated from the biological sample from the father by removing non-nucleated cells (e.g., red blood cells), extracellular proteins, extracellular nucleic acid molecules, extracellular metabolites, or any combination thereof from the biological sample.

In some embodiments, the paternal cells can have one or more of the maternal biomarkers disclosed herein. In some embodiments, the paternal cells can couple to one or more of the isolation agents specific to maternal biomarkers disclosed herein.

In some embodiments, the paternal cells are quantified. In some embodiments, the paternal cells are quantified by flow cytometry. In some embodiments, the paternal cells are quantified by fluorescence-activated cell sorting (FACS) flow cytometry. In some embodiments, the paternal cells are isolated through magnetic-activated cell sorting (MACS). In some embodiments, the paternal cells are quantified by microscopic microdissection. In some embodiments, the paternal cells are quantified by microscopic imaging. In some embodiments, the paternal cells are quantified by acoustic focusing flow cytometry. In some embodiments, the paternal cells are quantified by a hemocytometer. In some embodiments, the paternal cells are quantified by an automated cell counter. In some embodiments, the paternal cells are quantified by spectrophotometry.

In some embodiments, the paternal cells can be processed to generate sequencing data of the genome of the paternal cells. Processing the biological sample obtained from the subject can comprise (i) subjecting the paternal cells to conditions that are sufficient to isolate, enrich, or extract a plurality of fetal cell genetic material, and (ii) sequencing the plurality of paternal cell genetic material to generate a plurality of sequencing reads. The nucleic acid molecules can comprise ribonucleic acid (RNA) or deoxyribonucleic acid (DNA). The nucleic acid molecules can comprise DNA. The nucleic acid molecules can comprise RNA. The nucleic acid molecules (e.g., RNA or DNA) can be extracted from the paternal cells by a variety of methods, such as a DNeasy Blood and Tissue Kit, a PureLink Genomic DNA kit, or a Wizard® HMW DNA Extraction Kit. The extraction method can extract all RNA or DNA molecules from a sample.

In some embodiments, the genetic material from the paternal cells is genetically analyzed. In some embodiments, genetic material is analyzed via PCR, sequencing, hybridization methods, or any combination thereof. In some embodiments, the genetic material is analyzed via a whole genome scan. In some embodiments, the genetic material is analyzed via short tandem repeat analysis. In some embodiments, the genetic material is analyzed via microsatellite analysis.

In some embodiments, the genetic analysis is a hybridization method. In some embodiments, the hybridization method is a restriction fragment length polymorphism (RFLP), a southern blot analysis, a microarray, a fluorescence in situ hybridization (FISH), a comparative genomic hybridization, in situ hybridization, or any combination thereof.

In some embodiments, the genetic material from the paternal cells is amplified prior to genetic analysis. In some embodiments, the genetic material from the paternal cells is amplified to increase the amount of the genetic material from the paternal cells by between about 0% to about 500%. In some embodiments, the genetic material from the paternal cells is amplified to increase the amount of genetic material from the paternal cells by at least about 0%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 160%, at least about 180%, at least about 200%, at least about 250%, at least about 300%, at least about 350%, at least about 400%, at least about 450%, at least about 500%, or more. In some embodiments, the genetic material from the paternal cells is amplified to increase the amount of genetic material from the paternal cells by at most about 500%, at most about 450%, at most about 400%, at most about 350%, at most about 300%, at most about 250%, at most about 200%, at most about 180%, at most about 160%, at most about 140%, at most about 120%, at most about 100%, at most about 90%, at most about 80%, at most about 70%, at most about 60%, at most about 50%, at most about 40%, at most about 30%, at most about 20%, at most about 10%, at most about 0%, or less. In some embodiments, the genetic material from the paternal cells is amplified to increase the amount of genetic material from the paternal cells by about 0%, about 10%, about 20%, about 30%, about 40%, about 50%, about 60%, about 70%, about 80%, about 90%, about 100%, about 120%, about 140%, about 160%, about 180%, about 200%, about 250%, about 300%, about 350%, about 400%, about 450%, or about 500%.

In some embodiments, the fetal genetic material is analyzed in a genetic analysis. In some embodiments, the maternal genetic material is analyzed in a genetic analysis. In some embodiments, the fetal genetic material and the maternal genetic material are analyzed in a genetic analysis. In some embodiments, the paternal genetic material is analyzed in a genetic analysis. In some embodiments, the fetal genetic material, the maternal genetic material, and the paternal genetic material are analyzed in a genetic analysis.

In some embodiments, the genetic analysis is sequencing. In some embodiments, the method further comprises sequencing the nucleic acid molecules from the subpopulation of fetal cells. In some embodiments, the nucleic acid molecules from the subpopulation of fetal cells generate a nucleic acid sequence. In some embodiments, the nucleic acid sequence is from the subpopulation of fetal cells. In some embodiments, the method further comprises sequencing the nucleic acid molecules from the subpopulation of maternal cells. In some embodiments, the nucleic acid molecules from the subpopulation of maternal cells generate another nucleic acid sequence. In some embodiments, the other nucleic acid sequence is from the subpopulation of maternal cells. In some embodiments, the method further comprises sequencing the nucleic acid molecules from the paternal cells. In some embodiments, the nucleic acid molecules from the paternal cells generate a second nucleic acid sequence. In some embodiments, the second nucleic acid sequence is from the paternal cells. In some embodiments, the nucleic acid sequence, the second nucleic acid sequence, and/or the other nucleic acid sequence is a DNA sequence. In some embodiments, the nucleic acid sequence, the second nucleic acid sequence, and/or the other nucleic acid sequence is a RNA sequence.

Non-limiting examples of sequencing platforms include Sanger sequencing, massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina). In some embodiments, the sequencing is performed on a next generation sequencing platform. In some embodiments, the sequencing is performed on a massively parallel sequencing platform. In some embodiments, the sequencing is performed on a paired-end sequencing platform. In some embodiments, the sequencing is performed on a polony sequencing platform. In some embodiments, the sequencing is performed on a high-throughput sequencing platform. In some embodiments, the sequencing is performed on a shotgun sequencing platform. In some embodiments, the sequencing is performed on a single-molecule sequencing platform. In some embodiments, the sequencing is performed on a nanopore sequencing platform. In some embodiments, the sequencing is performed on a semiconductor sequencing platform. In some embodiments, the sequencing is performed on a pyrosequencing platform. In some embodiments, the sequencing is performed on a sequencing-by-synthesis platform. In some embodiments, the sequencing is performed on a sequencing-by-ligation platform. In some embodiments, the sequencing is performed on a sequencing-by-hybridization platform. In some embodiments, the sequencing is performed on an RNA-SEQ (Illumina) platform.

The sequencing can comprise nucleic acid amplification (e.g., of RNA or DNA molecules). In some embodiments, the entire genome is amplified prior to sequencing.

In some embodiments, the nucleic acid amplification is polymerase chain reaction (PCR). A suitable number of rounds of PCR (e.g., PCR, qPCR, reverse-transcriptase PCR, digital PCR, etc.) is performed to amplify an initial amount of nucleic acid (e.g., RNA or DNA) to a desired input quantity for subsequent sequencing. In some embodiments, the PCR is used for global amplification of target nucleic acids. This amplification can comprise using adapter sequences that are first ligated to different molecules followed by PCR amplification using universal primers. PCR can be performed using a kit of all the necessary tools. In some embodiments, only certain target nucleic acids within a population of nucleic acids can be amplified. Specific primers, for example, in conjunction with adapter ligation, can be used to amplify certain targets selectively for downstream sequencing. The PCR can comprise targeted amplification of one or more genomic loci, such as genomic loci associated with pregnancy-related states. The sequencing can comprise use of simultaneous reverse transcription (RT) and polymerase chain reaction (PCR), such as a OneStep RT-PCR kit protocol.

In some embodiments, the genetic analysis of fetal genetic material, the maternal genetic material, and/or the paternal genetic material is PCR. In some embodiments, the PCT is quantitative PCR (qPCR). In some embodiments, the qPCR uses Taqman primer/probes sets. In some embodiments, the PCR is RT-PCR. In some embodiments, the PCR is droplet digital PCR (ddPCR). In some embodiments, the PCR is digital PCR. In some embodiments, the PCR analysis is a PCR-based mutation detection. In some embodiments, the PCR analysis is single nucleotide polymorphisms (SNP) genotyping arrays.

After subjecting the nucleic acid molecules to sequencing, suitable bioinformatics processes can be performed on the sequence reads to generate the data indicative of the presence or absence of genetic disorders in the fetus (or fetuses). In some embodiments, the sequence reads are aligned to one or more reference genomes (e.g., a genome of one or more species such as a human genome). The aligned sequence reads can be quantified at one or more genomic loci to identify the presence or absence of the genetic disorder.

In some embodiments, the subpopulation of maternal cells is quantified. In some embodiments, the subpopulation of maternal cells is quantified by flow cytometry. In some embodiments, the subpopulation of maternal cells is quantified by fluorescence-activated cell sorting (FACS) flow cytometry. In some embodiments, the ex vivo population of cells is isolated through magnetic-activated cell sorting (MACS). In some embodiments, the subpopulation of maternal cells is quantified by microscopic microdissection. In some embodiments, the subpopulation of maternal cells is quantified by microscopic imaging. In some embodiments, the subpopulation of maternal cells is quantified by acoustic focusing flow cytometry. In some embodiments, the subpopulation of maternal cells is quantified by a hemocytometer. In some embodiments, the subpopulation of maternal cells is quantified by an automated cell counter. In some embodiments, the subpopulation of maternal cells is quantified by spectrophotometry.

In some embodiments, the subpopulation of fetal cells is quantified. In some embodiments, the subpopulation of fetal cells is quantified by flow cytometry. In some embodiments, the subpopulation of fetal cells is quantified by fluorescence-activated cell sorting (FACS) flow cytometry. In some embodiments, the ex vivo population of cells is isolated through magnetic-activated cell sorting (MACS). In some embodiments, the subpopulation of fetal cells is quantified by microscopic microdissection. In some embodiments, the subpopulation of fetal cells is quantified by microscopic imaging. In some embodiments, the subpopulation of fetal cells is quantified by acoustic focusing flow cytometry. In some embodiments, the subpopulation of fetal cells is quantified by a hemocytometer. In some embodiments, the subpopulation of fetal cells is quantified by an automated cell counter. In some embodiments, the subpopulation of fetal cells is quantified by spectrophotometry. In some embodiments, the subpopulation of maternal cells and the subpopulation of fetal cells are quantified. In some embodiments, the subpopulation of fetal cells and the subpopulation of maternal cells are quantified by flow cytometry.

In some embodiments, the subpopulation of fetal cells and the subpopulation of maternal cells are quantified by fluorescence-activated cell sorting (FACS) flow cytometry. In some embodiments, the ex vivo population of cells is isolated through magnetic-activated cell sorting (MACS). In some embodiments, the subpopulation of fetal cells and the subpopulation of maternal cells are quantified by microscopic microdissection. In some embodiments, the subpopulation of fetal cells and the subpopulation of maternal cells are quantified by microscopic imaging. In some embodiments, the subpopulation of fetal cells and the subpopulation of maternal cells are quantified by acoustic focusing flow cytometry. In some embodiments, subpopulation of fetal cells and the subpopulation of maternal cells are quantified by a hemocytometer. In some embodiments, the subpopulation of fetal cells and the subpopulation of maternal cells are quantified by an automated cell counter. In some embodiments, the subpopulation of fetal cells and the subpopulation of maternal cells are quantified by spectrophotometry.

In some embodiments, the present disclosure provides a method of performing an assay on an ex vivo population of cells. In some embodiments, the ex vivo population of cells is isolated from a subject. In some embodiments, the ex vivo population of cells are isolated from a biological sample taken from the subject. In some embodiments, the biological sample is a blood sample. In some embodiments, the blood sample is plasma. In some embodiments, the blood sample is serum. In some embodiments, the blood sample is whole blood. In some embodiments, the blood sample is whole blood depleted of red blood cells. In some embodiments, the blood sample is a buffy coat. In some embodiments, the blood sample is peripheral blood mononuclear cells. In some embodiments, the blood sample is nucleated white blood cells. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof.

In some embodiments, the sample is from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the sample is from a pregnant subject. In some embodiments, the sample from the pregnant subject is a maternal biological sample. In some embodiments, the ex vivo population of cells has a subpopulation of fetal cells. In some embodiments, the ex vivo population of cell has a subpopulation of maternal cells. In some embodiments, the ex vivo population of cells has a subpopulation of fetal cells and a subpopulation of maternal cells.

In some embodiments, the subpopulation of fetal cells is from about 50% to about 100% of cells in the ex vivo population of cells. In some embodiments, the subpopulation of fetal cells is from about 50% to about 60%, about 60% to about 70%, about 70% to about 80%, about 80% to about 90%, or about 90% to about 100% of cells in the ex vivo population of cells. In some embodiments, the subpopulation of fetal cells is at least about 50%, at least about 55%, at least about 60%, at least about 65%, at least about 70%, at least about 75%, at least about 80%, at least about 85%, at least about 90%, at least about 95%, or about 100% of cells in the ex vivo population of cells. In some embodiments, the subpopulation of fetal cells is at most about 100%, at most about 95%, at most about 90%, at most about 85%, at most about 80%, at most about 75%, at most about 70%, at most about 65%, at most about 60%, at most about 55%, at most about 50%, or less of cells in the ex vivo population of cells. In some embodiments, the subpopulation of fetal cells is at least about 50% of cells in the ex vivo population of cells. In some embodiments, the subpopulation of fetal cells is at least about 60% of cells in the ex vivo population of cells. In some embodiments, the subpopulation of fetal cells is at least about 70% of cells in the ex vivo population of cells. In some embodiments, the subpopulation of fetal cells is at least about 80% of cells in the ex vivo population of cells. In some embodiments, the subpopulation of fetal cells is at least about 90% of cells in the ex vivo population of cells.

In some embodiments, the method further comprising depleting red blood cells from the ex vivo population of cells.

In some embodiments, the assay is a cell morphology assay. Non-limiting examples of cell morphology assays include immunofluorescence, scanning electron microscopy, and hematoxylin-Eosin (HE) staining. In some embodiments, the cell morphology assay can determine a size of a cell, a shape of a cell, presence or absence of organelles in a cell, presence or absence of cell membrane of a cell, number of organelles in a cell, or any combination thereof. In some embodiments, the assay is a cell counting assay. Non-limiting examples of cell counting assays include flow cytometry, FACS, MACS, acoustic focusing flow cytometry, hemocytometry, microscopic microdissection, microscopic imaging, and spectrophotometry. In some embodiments, the assay is a cell viability assay. Non-limiting examples of cell viability assays include cell metabolism assays, ATP detection assays, tetrazolium reduction assays, and protease activity assays. In some embodiments, the assay is a cell proliferation assay. Non-limiting examples of cell proliferation assays include DNA synthesis proliferation assays, generational analysis by dye dilution assays, and DNA content quantitation cell proliferation assays. In some embodiments, the assay is a cytotoxicity assay. Non-limiting examples of cytotoxicity assays include lactate dehydrogenase assays, DNA-binding dye assays, and Annexin V staining.

In some embodiments, the assay indicates a likelihood that the ex vivo population of cells is susceptible to a disease or condition. In some embodiments, the assay indicates a likelihood that the subpopulation of maternal cells is susceptible to a disease. In some embodiments, the assay indicates a likelihood that the subpopulation of fetal cells is susceptible to a disease. In some embodiments, the assay indicates that a fetus associated with subpopulation of fetal cells is susceptible to a disease. In some embodiments, the disease is a genetic condition.

In some embodiments, the results of the assay is compared to information from one or more databases. In some embodiments, the one or more databases include information related to fetal disorders, genetic disorders, genetic conditions, fetal or any combination thereof. Non-limiting examples of the information from the one or more databases include genetic sequencing information from fetal cells affected by a genetic disorder or condition, morphological characteristics of fetal cells affected by a genetic disorder or condition, chromosomal make-up of fetal cells affected by a genetic disorder or condition, gene expression information from the subpopulation of fetal cells affected by a genetic disorder or condition, protein expression levels from fetal cells affected by a genetic disorder or condition, functional characteristics of fetal cells affected by a genetic disorder or condition, or any combination thereof. In some embodiments, the one or more databases are preexisting databases. In some embodiments, the one or more databases are databases currently being generated. In some embodiments, the results of the assay are compared to the one or more databases to determine if the fetus has a genetic disorder or a genetic condition. In some embodiments, the comparison of the results of the assay and the one or more databases can be provided to the mother. In some embodiments, the comparison of the results of the assay and the one or more databases can be provided to a clinician. In some embodiments, the comparison of the results of the assay and the one or more databases can be provided to the mother and a clinician. In some embodiments, the comparison of the results of the assay and the one or more databases generates a diagnosis of the one or more genetic conditions or disorders. In some embodiments, if the results from the assay is the same as the information from the one or more databases, then the comparison generates a diagnosis of one or more genetic conditions or disorders. In some embodiments, if the results from the assay is different from the information from the one or more databases, the then comparison may not generate a diagnosis of one or more genetic conditions or disorders.

Non-limiting examples of genetic conditions or disorders include 1p36 deletion syndrome, 1q21.1 deletion syndrome, 2q37 deletion syndrome, 5q deletion, 5, 10-methenyltetrahydrofolate synthestase deficiency, 17q12 microdeletion syndrome, 17q12 microduplication syndrome, 18p deletion syndrome, 21-hydroxylase deficiency, alpha 1-antitrypsin deficiency, achalasia-addisonianism-alacrima (AAA) syndrome, Aarskog-Scott syndrome, ABCD syndrome, Absnece deformity of leg-cataract, Aceruloplasminemia, Acheiropodia, Achondrogenesis type II, achondroplasia, acute intermittent porphyria, adenylosuccinate lyase deficiency, adrenoleukodyst94estatiolagille syndrome, ADULT syndrome, Aicardi-Goutieres syndrome, Albinism, Alexander disease, Alfi's syndrome, alkaptonuria, Alport syndrome, alternating hemiplegia of childhood, aortic arch anomaly, peculiar facies, Amish lethal microcephaly, amyotrophic lateral sclerosis, angel-shaped phalango-epiphyseal dysplasia, Alstrom syndrome, Alzheimer's syndrome, amelogenesis imperfecta, aminolevulinic acid dehydratase deficiency porphyria, androgen insensitivity syndrome, angelman syndrome, aphalangy-syndactyly-microcephaly syndrome, apert syndrome, arthrogryposis, ataxia telangiectasia, axenfeld syndrome, Bainbridge-ropers syndrome, breae-stevenson cutis gyrate syndrome, beckwith-wiedermann syndrome, Benjamin syndrome, biotinidase deficiency, Bjornstad syndrome, Blepharophimosis syndromes, Bloom syndrome, Birt-Hogg-Dube syndrome, Brody myopathy, Brunner syndrome, CADASIL syndrome, Cat eye syndrome, CRASIL syndrome, chronic granulomatous disorder, campomelic dysplasia, captodactyly-taurinuria syndrome, Canavan disease, Carpenter syndrome, CDKL5 deficiency disorder, cerebral dysgenesis-neuropathy-ichthyosis-keratoderma syndrome (CEDNIK), cleft palate short stature vertebral anomalies syndrome, congenital muscular dystrophy-infantile cataract-hypogonadism syndrome, cystic fibrosis, Charcot-Marie-Tooth disease, CHARGE syndrome, Chediak-Higashi syndrome, Grebe-type chonodrodysplasia, cleidocranial dysostosis, Cockayne syndrome, Coffin-Lowry syndrome, Cohen syndrome, type II collagenopathy, type XI collagenopathy, congenital insensitivity to pain with anhidrosis (CIPA), congenital muscular dystrophy, cornelia de Lange syndrome, Cowden syndrome, CPO deficiency, Cranio-lenticulo-sutural dysplasia, Cri du chat, Crohn's disease, Crouzon syndrome, Crouzonodermoskeletal syndrome, Currarino syndrome, Darier's disease, Dent's disease, Denys-Drash syndrome, De Grouchy syndrome, dolichonychia, Down Syndrome, DiGeorge Syndrome, Distal hereditary motor neuropathies, distal muscular dystrophy, Duchenne muscular dystrophy, Dravet syndrome, Ectrodactyly-polydactyly syndrome, Edwards Syndrome, Ehlers-Danlos syndrome, Emanuel syndrome, Emery-Dreifuss syndrome, Epidermolysis bullosa, erythropoietic protoporphyria, Fanconi amenia, Fabry disease, Factor V Leiden thrombophilia, fatal familial insomnia, familial adenomatous polyposis, familial dysautonomia, Familial Creutzfeld-Jakob disease, familial episodic pain syndrome, familial thoracic aortic aneurysm and aortic dissection, Feingold syndrome, FG syndrome, FBXW7 neurodevelopmental syndrome, Fibular aplasia-ectrodactyly syndrome, Fine-Lubinsky syndrome, Fragile X syndrome, Friedreich's ataxia, G6PD deficiency, glactosemia, Gaucher disease, Gerstmann-Straussler-Scheinker syndrome, Gillespie syndrome, type I glutaric aciduria, type II glutaric aciduria, GRACILE syndrome, GRIN2B-related neurodevelopmental disorder, Griscelli syndrome, Gustavson syndrome, Hailey-Hailey disease, Harlequin type ichthyosis, Hemochromatosis type 1, Hemochromatosis type 2A, Hemochromatosis type 2B, Hemochromatosis type 3, Hemochromatosis 4, Hemochromatosis 5, hemophilia, hepatoerythropoietic porphyria, hereditary coproporphyria, hereditary hemorrhagic telangiectasia, hereditary inclusion body myopathy, hereditary multiple exostoses, hereditary spastic paraplegia, Hermansky-Pudiak syndrome, hereditary neuropathy with liability to pressure palsies (HNPP), heterotaxy, homocystinuria, Huntington's disease, Hunter syndrome, Hurler syndrome, Hutchinson-Gilford progeria syndrome, hyperlysinemia, primary hyperoxaluria, hyperphenylalainemia, Tangier disease, hypochondrogenesis, hypochondroplasia, immunodeficiency-centrometric instability-facial anomalies syndrome (ICF), incontinentia pigmenti, infantile cerebral and cerebellar atrophy with postnatal progressive microcephaly, ischiopatellar dysplasia, isodicentric 15, PRICKLE1-related progressive myoclonus epilepsy with ataxia, Jackson-Weiss syndrome, Jacobsen syndrome, Joubert syndrome, Juvenile-onset dystonia, Juvenile primary lateral sclerosis, Keloid disorder, KIF1A-associated neurological disorder, Kleefstra syndrome, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Gridle Muscular Dystrophy, Lynch syndrome, lipoprotein lipase deficiency, malignany hyperthermia, Maple syrup urine disease, Marfan syndrome, Maroteaux-Lamy syndrome, McCune-Albright syndrome, McLeod syndrome, MEDNIK syndrome, familial Mediterranean fever, Mekes disease, methemoglobinemia, methylmalonic acidemia, Micro syndrome, microcephaly, Miller-Dieker syndrome, Morquio syndrome, Mowat-Wilson syndrome, Muenke syndrome, type 1 multiple endocrine neoplasia, type 2 multiple endocrine neoplasia, muscular dystrophy, Becker muscular dystrophy, myostatin-related muscle hypertrophy, myotonic dystrophy, Natowicz syndrome, Nedamss neurodevelopment disorder, type I neurofibromatosis, type II neurofibromatosis, Niemann-Pick disease, Nonketotic hyperglycinemia, nonsyndromic deafness, noonan syndrome, Norman-Roberts syndrome, Ogden syndrome, Omenn syndrome, Osteogenesis imperfecta, Ostravik-Lindemann-Solberg syndrome, Pantothenate kinase-associated neurodegeneration, Patau syndrome, PCC deficiency, porphyria cutanea tarda, Pendred syndrome, Peutz-Jeghers syndrome, Pfeiffer syndrome, Phelan-McDermid syndrome, Phenylketouria, Pipecolic acidemia, Pitt-Hopkins syndrome, Polycystic kidney disease, polycystic ovary syndrome, porphyria, Prader-Willi syndrome, Primary ciliary dyskinesia, primary pulmonary hypertension, protein C deficiency, protein S deficiency, proximal 18q deletion syndrome, Pseudo-Gaucher disease, pseudoxanthoma elasticum, Retinitis pigmentosa, Rett syndrome, Roberts syndrome, Rubinstein-Taybi syndrome, Sandhoff disease, Sanfilippo syndrome, Schwartz-Jampel syndrome, Sjogren-Larsson syndrome, Skin fragility-wooly hair-palmoplantar keratoderma syndrome, Spondyloepiphyseal dysplasia congenita, Shprintzen-Goldbery syndrome, Sickle cell anemia, Siderius X-linked mental retardation syndrome, Sideroblastic anemia, Sly syndrome, Smith-Lemli-Optiz syndrome, Smith-Magenis syndrome, Synder-Robinson syndrome, Spinal muscular atrophy, Spinocerebellar ataxia, split hand split foot-nystagmus syndrome, SSB syndrome, Stargardt disease, Stickler syndrome, Strudwick syndrome, Tay-Sachs disease, tetrahydrobiopterin deficiency, thanatophoric dysplasia, thickened earlobes-conductive deafness syndrome, Treacher Collins syndrome, Tuberous sclerosis complex, Turner syndrome, Usher syndrome, Variegate porphyria, Vijoen-Kallis-Voges syndrome, von Hippel-Lindau disease, von Willebrand disease, Waardenburg syndrome, Warkany syndrome 2, Weissenbacher-Zweymüller syndrome, Weyer's ulnar ray/oligodactyly syndrome, Williams syndrome, Wilson disease, Woodhouse-Sakati syndrome, Wolf-Hirschhorn syndrome, Xeroderma pigmentosum, Fragile X syndrome, X-linked spinal-bulbar muscle atrophy, Xp11.2 duplication syndrome, X-linked severe combined immunodeficiency, X-linked sideroblastic anemia, triple X syndrome, XXXX syndrome, XXXXX syndrome, XXXXY syndrome, XYY syndrome, XXYY syndrome, XYYY syndrome, XXXY syndrome, XYYYY syndrome, and Zellweger syndrome.

In some embodiments, the genetic condition is down syndrome. In some embodiments, the genetic disorder is Fragile X syndrome. In some embodiments, the genetic disorder is triple X syndrome. In some embodiments, the genetic disorder is XXXX syndrome. In some embodiments, the genetic disorder is XXXXX syndrome. In some embodiments, the genetic disorder is XXXXY. In some embodiments, the genetic disorder is XYY syndrome. In some embodiments, the genetic disorder is XYY syndrome. In some embodiments, the genetic disorder is XXYY syndrome. In some embodiments, the genetic disorder is XYYY. In some embodiments, the genetic disorder is XXXY syndrome. In some embodiments, the genetic disorder is XYYYY. In some embodiments, the genetic disorder is Tay-Sachs disease. In some embodiments, the genetic disorder is Angelman syndrome. In some embodiments, the genetic disorder is Cystic fibrosis. In some embodiments, the genetic disorder is DiGeorge syndrome. In some embodiments, the genetic disorder is muscular dystrophy. In some embodiments, the genetic disorder is Duchenne muscular dystrophy. In some embodiments, the genetic disorder is fetal familial insomnia. In some embodiments, the genetic disorder is Creutzfeld-Jakob disease. In some embodiments, the genetic disorder is Gerstmann-Sträussler-Scheinker syndrome. In some embodiments, the genetic disorder is Hailey-Hailey disease. In some embodiments, the genetic disorder is Hemophilia. In some embodiments, the genetic disorder is Huntington's disease. In some embodiments, the genetic disorder is Marfan syndrome. In some embodiments, the genetic disorder is Prader-Willi syndrome. In some embodiments, the genetic disorder is Sickle Cell disease. In some embodiments, the genetic disorder is Turner syndrome. In some embodiments, the genetic disorder is von Hippel-Lindau disease.

In some embodiments, the genetic condition is a chromosomal abnormality. Non-limiting examples of chromosomal abnormalities include aneuploidy, translocation, unbalanced translocation, rearrangement, subtelomeric rearrangement, unbalance chromosomal rearrangement, unbalance subtelomeric rearrangement, deletion, inversions, unbalanced inversions, duplication, telomere instability, telomere shortening, single nucleotide substitution, micro deletion, micro-insertion, short deletions, short insertion, multi-nucleotide changes, DNA methylation and, and loss of imprint (LOI). Non-limiting examples of chromosomal aneuploidy include trisomy 21, trisomy 18, trisomy 13, trisomy 16, polysomies, monosomies, and XXX. In some embodiments, the chromosomal aneuploidy is a complete monosomy. In some embodiments, the complete monosomy is monosomy X, monosomy 21, monosomy 22, monosomy 16, or monosomy 15. In some embodiments, the chromosomal aneuploidy is a partial monosomy. In some embodiments, the partial monosomy is monosomy X, monosomy 21, monosomy 22, monosomy 16, or monosomy 15. In some embodiments, the chromosomal abnormality is trisomy 18. In some embodiments, the chromosomal abnormality is trisomy 13.

In some embodiments, the subpopulation of fetal cells are used to perform a fetal genetic risk analysis. In some embodiments, the fetal genetic risk analysis determines whether an adverse health factor to the pregnancy exists. In some embodiments, the fetal genetic risk analysis determines whether an absence of an adverse health factor to the pregnancy exists. In some embodiments, the fetal genetic risk analysis determines a presence of a genetic disorder or condition in the fetus. In some embodiments, the fetal genetic risk analysis determines an absence of a genetic disorder or condition in the fetus. In some embodiments, the fetal genetic risk analysis determines the current health of the fetus. In some examples, the fetal genetic risk analysis determines whether the fetus has a low risk, a medium risk, or a high risk of developing health complications during the pregnancy. In some embodiments, a low risk of developing health complications during the pregnancy can mean an unlikelihood (e.g., less than about 30% probability) that the fetus develops health complications during the pregnancy. In some embodiments, a medium risk of developing health complications during the pregnancy can mean a possibility (e.g., between about 30% to about 60% probability) that the fetus develops health complications during the pregnancy. In some embodiments, a high risk of developing health complications during the pregnancy can mean a high likelihood (e.g., greater than about 60% probability) that the fetus develops health complications during the pregnancy. In some embodiments, the fetal genetic risk analysis determines the current health of the mother during the pregnancy. In some embodiments, the fetal genetic risk analysis determines whether the mother has a low risk, a medium risk, or a high risk of developing health complications during the pregnancy. In some embodiments, a low risk of developing health complications during the pregnancy can mean an unlikelihood (e.g., less than about 30% probability) that the mother develops health complications during the pregnancy. In some embodiments, a medium risk of developing health complications during the pregnancy can mean that a possibility (e.g., between about 30% to about 60% probability) that the mother develops health complications during the pregnancy. In some embodiments, a high risk of developing health complications during the pregnancy can mean a high likelihood (e.g., greater than about 60% probability) that the mother develops health complications during the pregnancy. In some embodiments, the fetal genetic risk analysis determines the current risk level of a miscarriage in the mother. In some embodiments, the fetal genetic risk analysis is performed via performing one or more of the assays disclosed herein. In some embodiments, the fetal genetic risk analysis performed via a sequencing assay on the subpopulation of the fetal cells.

In some embodiments, the one or more assays performed can be a genetic assay. In some examples, the genetic assay can be PCR, whole genome sequencing (WGS), targeted genomic sequencing, targeted ribonucleic acid sequencing, hybridization methods, short tandem repeat analysis, microsatellite analysis, or any combination thereof. In some embodiments, the one or more assays performed can be a cell morphology assay. In some embodiments, the one or more assays can be a cell counting assay. In some embodiments, the one or more assays can be a cell viability assay. In some embodiments, the one or more assays can be a cell proliferation assay. In some embodiments, the one or more assays can be a cytotoxicity assay. In some embodiments, the one or more assays can be a cell function assay. In some examples, the cell function assay can include a cell migration assay, a cell invasion assay, an angiogenesis assay, a reactive oxygen species assay, a protein assay (e.g., enzyme-linked immunosorbent assay (ELISA), MTT assay), a live cell analysis assay, a gene expression assay, a stimulation assay or any combination thereof. In some examples, a stimulation assay is an assay that measures how a cell reacts to a stimulus. Non-limiting examples of stimuli include cell metabolic or signaling responses to glucose, oxygen, or growth factor levels in the media bathing the purified CFC. In some examples, the response of the cell to the stimulus that is measured can be alterations in (i) CFC metabolism, (ii) signal transduction, (iii) stress response, (iv) apoptosis, (v) cell cycle, or any combination of (i)-(v).

In some embodiments, the fetal genetic risk analysis is a genomic sequencing assay. In some embodiments, the genomic sequencing assay is a whole genome sequencing assay. In some embodiments, the genomic sequencing assay is a targeted genomic sequencing assay. In some examples, the targeted genomic sequencing assay can be a PCR assay, a hybridization assay, a RNAseq assay, a transposon-mediated fragmentation assay, or any combination thereof.

In some embodiments, the fetal genetic risk analysis is a cell phenotype assay. In some embodiments, the cell phenotype assay is a cell metabolism assay. In some examples, the cell metabolism assay measures the metabolism of one or more metabolites within a cell. Non-limiting examples of the one or more metabolites include glucose, cholesterol, ATP, galactose, hydrogen peroxide, lactate, ALT, glycerol, AST, lactate dehydrogenase, superoxide dismutase, GTP, amylase, hemoglobin, lipid, antioxidant, acetylcholinesterase, glutathione, nitrite/nitrate, β-hydroxybutyrate, acetyl-coenzyme A, alkaline phosphatase, bile acid, catalase, urea, glutathione, α-glucosidase, glutamate, phosphate, creatine kinase, ammonia, HMG-COA reductase, pyruvate, ethanol, phospholipid, protein carbonyl, BCG albumin, aldehyde dehydrogenase, α-ketoglutarate, glutathione peroxidase, acetylcholinesterase, NAD, cytochrome C oxidase, β-glucosidase, heme, hyaluronidase inhibit, bilirubin, angiotensin I converting enzyme (ACE), oxalate, citrate, fructose, lactose, pyruvate, starch, glycogen, alcohol, or any combination thereof. In some embodiments, the cell phenotype assay is a cell morphology assay. In some embodiments, the cell phenotype assay is a cell counting assay. In some embodiments, the cell phenotype assay is a cell viability assay. In some embodiments, the cell phenotype assay is a cell proliferation assay. In some embodiments, the cell phenotype assay is a cytotoxicity assay. In some embodiments, the cell phenotype assay is a cell signaling assay. In some embodiments, the cell phenotype assay is an apoptosis assay. In some embodiments, the cell phenotype assay is a cellular stress assay. Non-limiting examples of cellular stress assays include reactive oxygen species or reactive nitrogen species assays, glutathione assays, demethylation assays, DNA break assays, total thiol assay, or any combination thereof.

In some embodiments, the fetal genetic risk analysis is a gene expression assay (e.g., RNAseq). In some embodiments, the gene expression assay can measure a level of at least one gene (e.g., mRNA transcript, regulatory molecule, etc.) compared to the baseline expression level of the at least one gene. In some embodiments, the gene expression assay measures the level of at least one gene. In some embodiments, the at least one gene can comprise at least about one gene, at least about two genes, at least about three genes, at least about four genes, at least about five genes, at least about six genes, at least about seven genes, at least about eight genes, at least about nine genes, at least about 10 genes, at least about 15 genes, at least about 20 genes, at least about 25 genes, at least about 30 genes, at least about 35 genes, at least about 40 genes, at least about 45 genes, at least about 50 genes, or more. In some embodiments, the at least one gene can comprise at most about 50 genes, at most about 45 genes, at most about 40 genes, at most about 35 genes, at most about 30 genes, at most about 25 genes, at most about 20 genes, at most about 15 genes, at most about 10 genes, at most about nine genes, at most about eight genes, at most about seven genes, at most about six genes, at most about five genes, at most about four genes, at most about three genes, at most about two genes, at most about one gene, or less. In some embodiments, the at least one gene can comprise about one gene, about two genes, about three genes, about four genes, about five genes, about six genes, about seven genes, about eight genes, about nine genes, about 10 genes, about 15 genes, about 20 genes, about 25 genes, about 30 genes, about 35 genes, about 40 genes, about 45 genes, or about 50 genes. In some embodiments, the gene expression assay can determine the genotype of a cell. In some embodiments, the gene expression assay can be a whole genome sequencing assay. In some embodiments, the gene expression assay can be a chromosomal assay. In some examples, the chromosomal assay identifies one or more chromosomal assays.

In some embodiments, the genomic sequencing assay can be a whole genome sequencing assay. In some embodiments, the whole genome sequence of the fetal cell can be compared to the whole genome sequence of a maternal cell. In some embodiments, the whole genome sequence of the fetal cell can be compared to the whole genome sequence of the maternal cell to determine the genetic information (e.g., sequence variants, single nucleotide variants, alleles, genes) that the fetus inherited from the mother. In some embodiments, the whole genome sequence of the fetal cell can be compared to the whole genome sequence of a paternal cell. In some embodiments, the whole genome sequence of the fetal cell can be compared to the whole genome sequence of the maternal cell to determine the genetic information (e.g., sequence variants, single nucleotide variants, alleles, genes, copy number variants, insertions, deletions) that the fetus inherited from the father. In some embodiments, the whole genome sequence of the fetal cell can be compared to the whole genome sequence of a maternal cell and the whole genome sequence of a paternal cell. In some embodiments, the whole genome sequence of the fetal cell can be compared to the whole genome sequence of the maternal cell to determine the genetic information (e.g., sequence variants, single nucleotide variants, alleles, genes) that the fetus inherited from the mother and the father. In some embodiments, the comparison of the whole genome sequence of the fetal cell, the whole genome sequence of the maternal cell, and the whole genome sequence of the paternal cells can be used to determine the genetic information of the fetal cell that was inherited from the father and the genetic information of the fetal cell that was inherited from the mother. In some embodiments, the comparison of the whole genome sequence of the fetal cell, the whole genome sequence of the maternal cell, and the whole genome sequence of the paternal cells to determine de novo somatic mutations in the whole genome sequence of the fetal cell that are absent from the whole genome sequence of the maternal cell and the whole genome sequence of the paternal cell.

In some embodiments, the whole genome sequencing assay further comprises whole genome amplification. In some examples, the whole genome amplification occurs prior to the whole genome sequencing. In some embodiments, the whole genome amplification is multiple displacement amplification (MDA), degenerate oligonucleotide PCR (DOP-PCR), Primer Extensions Preamplification (PEP), tagged random primer PCT (T-PCR), long and accurate PCR (LA-PCR), ligation-mediated PCR (LM-PCR), interspersed repetitive sequence PCR (IRS-PCR), multiple annealing and looping-based amplification cycles, (MALBAC), linear amplification via transposon insertion (LIANTI), microfluidic amplification, Primary Template-directed Amplification, or any combination thereof.

In some embodiments, the gene sequencing (e.g., targeted DNA sequencing) assay can generate genetic sequences of a targeted nucleic acid sequence (e.g., nucleotide sequences of genes, alleles, sequence variations in the gene sequence, single nucleotide variants in the gene sequence, insertions, deletions). In some embodiments, the genetic sequences of the fetal cell can be compared to the genetic sequences of a maternal cell. In some embodiments, the genetic sequences of the fetal cell can be compared to the genetic sequences of a paternal cell. In some embodiments, the genetic sequences of the fetal cell can be compared to the genetic sequences of a maternal cell and the genetic sequences of a paternal cell. In some embodiments, the genetic sequences of the fetal cell can be compared to the genetic sequences of a maternal cell. In some embodiments, the genetic sequences of the fetal cell can be compared to the genetic sequences of the maternal cell to determine the genetic sequences that the fetus inherited from the mother. In some embodiments, the genetic sequences of the fetal cell can be compared to the genetic sequences of a paternal cell. In some embodiments, the genetic sequences of the fetal cell can be compared to the genetic sequences of the maternal cell to determine the genetic sequences that the fetus inherited from the father. In some embodiments, the genetic sequences of the fetal cell can be compared to the genetic sequences of a maternal cell and the genetic sequences of a paternal cell. In some embodiments, the genetic sequences of the fetal cell can be compared to the genetic sequences of the maternal cell to determine the genetic sequences that the fetus inherited from the mother and the father. In some embodiments, the comparison of the genetic sequences of the fetal cell, the genetic sequences of the maternal cell, and the genetic sequences of the paternal cells can be used to determine the genetic sequences of the fetal cell that was inherited from the father and the genetic sequences of the fetal cell that was inherited from the mother. In some embodiments, the comparison of the genetic sequences of the fetal cell, the genetic sequences of the maternal cell, and the genetic sequences of the paternal cells to determine de novo somatic mutations in the genetic sequences of the fetal cell that are absent from the genetic sequences of the maternal cell and the genetic sequences of the paternal cell.

In some embodiments, the fetal genetic risk analysis is a protein assay. In some embodiments, the protein assay determines the level of at least one protein in a cell. In some embodiments, the at least one protein can comprise at least about one protein, at least about two proteins, at least about three proteins, at least about four proteins, at least about five proteins, at least about six proteins, at least about seven proteins, at least about eight proteins, at least about nine proteins, at least about 10 proteins, at least about 15 proteins, at least about 20 proteins, at least about 25 proteins, at least about 30 proteins, at least about 35 proteins, at least about 40 proteins, at least about 45 proteins, at least about 50 proteins, or more. In some embodiments, the at least one protein can comprise at most about 50 proteins, at most about 45 proteins, at most about 40 proteins, at most about 35 proteins, at most about 30 proteins, at most about 25 proteins, at most about 20 proteins, at most about 15 proteins, at most about 10 proteins, at most about nine proteins, at most about eight proteins, at most about seven proteins, at most about six proteins, at most about five proteins, at most about four proteins, at most about three proteins, at most about two proteins, at most about one protein, or less. In some embodiments, the at least one protein can comprise about one protein, about two proteins, about three proteins, about four proteins, about five proteins, about six proteins, about seven proteins, about eight proteins, about nine proteins, about 10 proteins, about 15 proteins, about 20 proteins, about 25 proteins, about 30 proteins, about 35 proteins, about 40 proteins, about 45 proteins, or about 50 proteins. Non-limiting examples of protein assays can include ELISA, MTT assay, real-time PCT, immunoblotting, western blot, immunohistochemistry, Warburg-Christian assay, Lowry assay, Bradford assay, bicinchoninic acid assay (BCA), high-performance liquid-based chromatography, immunoprecipitation, SDS-PAGE, and any combination thereof.

In some embodiments, the fetal genetic risk analysis can produce results. In some embodiments, the results of the fetal genetic risk analysis are the results of the one or more assays performed. Non-limiting examples of results of the fetal genetic risk analysis include genetic sequencing information from the subpopulation of fetal cells, morphological characteristics of the subpopulation of fetal cells, chromosomal make-up of the subpopulation of fetal cells, gene expression information from the subpopulation of fetal cells, protein expression levels from the subpopulation of fetal cells, functional characteristics of the subpopulation of fetal cells, or any combination thereof. In some embodiments, the results of the fetal genetic risk analysis are compared one or more databases. In some embodiments, the one or more databases include information related to fetal disorders, genetic disorders, genetic conditions, fetal health in utereo, miscarriage risks, fetal health risks, maternal health risks, therapeutics, clinical outcomes, possible therapeutic treatments, or any combination thereof. Non-limiting examples of the information from the one or more databases include genetic sequencing information from fetal cells affected by a genetic disorder or condition, genetic sequencing information from maternal and/or fetal cells where the fetus experienced fetus health risks during pregnancy, genetic sequencing information from maternal cells and/or fetal cells where the mother experienced maternal health risks during pregnancy, morphological characteristics of fetal cells affected by a genetic disorder or condition, morphological characteristics of fetal cells from a fetus that experienced fetal health risks during pregnancy, morphological characteristics of maternal cells and/or fetal cells where the mother experienced maternal health risks during pregnancy, chromosomal make-up of fetal cells affected by a genetic disorder or condition, chromosomal make-up of the maternal cells and/or fetus cells wherein the mother experienced maternal health risks during pregnancy, chromosomal make-up of the fetus cells where the fetus experienced fetus health risks during pregnancy, gene expression information from the subpopulation of fetal cells affected by a genetic disorder or condition, gene expression information from the subpopulation of fetal cells affected where the fetus experienced fetus health risks during pregnancy, gene expression information from the subpopulation of fetal cells and/or maternal cells where the mother experienced maternal health risks during pregnancy, protein expression levels from fetal cells affected by a genetic disorder or condition, protein expression levels from fetal cells from a fetus that experienced fetal health risks during pregnancy, protein expression levels from fetal cells and/or maternal cells where the mother experienced maternal health risks during pregnancy, functional characteristics of fetal cells from a fetus that experienced fetal health risks during pregnancy, functional characteristics of fetal cells affected by a genetic disorder or condition, functional characteristics of fetal cells and/or maternal cells where the method experienced maternal health risk during pregnancy, or any combination thereof. In some embodiments, the one or more databases are preexisting databases. In some embodiments, the one or more databases are databases currently being generated. In some embodiments, the results of the fetal genetic risk analysis are compared to the one or more databases to determine if the fetus has a fetal disorder, a genetic disorder, a genetic condition, fetal health risks, possible clinical outcomes of the fetus, possible therapeutic treatments for the fetus, or any combination thereof. In some embodiments, the results of the fetal genetic risk analysis are compared to the one or more databases to determine if the mother has a risk of miscarriage, maternal health risks, possible therapeutic treatments for the pregnancy, possible clinical outcomes of the pregnancy, or any combination thereof. In some embodiments, the comparison of the results of the fetal genetic risk analysis and the one or more databases can be provided to the mother. In some embodiments, the comparison of the results of the fetal genetic risk analysis and the one or more databases can further include one or more recommended clinical treatments to mitigate current and/or future health risks to the mother and/or fetus. In some embodiments, the comparison of the results of the fetal genetic risk analysis and the one or more databases can be provided to a clinician. In some embodiments, based on the comparison of the results of the fetal genetic risk analysis and the one or more databases, a clinician can recommend one or more clinical treatments to mitigate current and/or future health risks to the fetus and/or the mother. In some embodiments, the comparison of the results of the fetal genetic risk analysis and the one or more databases can be provided to the mother and a clinician. In some embodiments, the comparison of the results of the fetal genetic risk analysis and the one or more databases generates a diagnosis of the one or more genetic conditions or disorders. In some embodiments, if the results from the fetal genetic risk analysis is the same as the information from the one or more databases, then the comparison generates a diagnosis of one or more genetic conditions or disorders. In some embodiments, if the results from the fetal genetic risk analysis is different from the information from the one or more databases, the then comparison may not generate a diagnosis of one or more genetic conditions or disorders. In some embodiments, the comparison of the results of the fetal genetic risk analysis and the one or more databases generates a diagnosis of one or more fetal health risks. In some embodiments, if the results from the fetal genetic risk analysis is the same as the information from the one or more databases, then the comparison generates a diagnosis of one or more fetal health risks. In some embodiments, if the results from the fetal genetic risk analysis is different from the information from the one or more databases, then comparison may not generate a diagnosis of one or more fetal health risks. In some embodiments, the comparison of the results of the fetal genetic risk analysis and the one or more databases generates a diagnosis of one or more maternal health risks. In some embodiments, if the results from the fetal genetic risk analysis is the same as the information from the one or more databases, then the comparison generates a diagnosis of one or more maternal health risks. In some embodiments, if the results from the fetal genetic risk analysis is different from the information from the one or more databases, then comparison may not generate a diagnosis of one or more maternal health risks.

In some embodiments, the comparison of the results of the fetal genetic risk analysis and the one or more databases can be used to determine current health risks to the fetus. In some embodiments, if the results from the fetal genetic risk analysis is the same as the information from the one or more databases, then the comparison determines one or more current health risks to the fetus. In some embodiments, if the results from the fetal genetic risk analysis is different from the information from the one or more databases, the then comparison may not determine one or more current health risks to the fetus. In some embodiments, the comparison of the results of the fetal genetic risk analysis and the one or more databases can be used to determine current health risks to the mother. In some embodiments, if the results from the fetal genetic risk analysis is the same as the information from the one or more databases, then the comparison determines one or more current health risks to the mother. In some embodiments, if the results from the fetal genetic risk analysis is different from the information from the one or more databases, the then comparison may not determine one or more current health risks to the mother. In some embodiments, the comparison of the results of the fetal genetic risk analysis and the one or more databases can be used to determine current health risks to the fetus and the mother. In some embodiments, if the results from the fetal genetic risk analysis is the same as the information from the one or more databases, then the comparison determines one or more current health risks to the fetus and the mother. In some embodiments, if the results from the fetal genetic risk analysis is different from the information from the one or more databases, the then comparison may not determine one or more current health risks to the fetus and the mother.

In some embodiments, the current and/or future health risks can be any condition or disorder that adversely impacts the mother and/or the fetus during pregnancy. In some examples, the current and/or future health risks for the mother can include high blood pressure, gestational diabetes, preeclampsia, preterm labor, pregnancy loss/miscarriage, stillbirth, vomiting, nausea, iron-deficiency anemia, amniotic fluid complications, placental abruption, placenta previa, eclampsia, or any combination thereof. In some embodiments, the current and/or future health risks for the fetus can include preterm labor, miscarriage, low birth weight, blindness, deafness, bone deformities, intellectual disability, genetic conditions or disorders, birth defects, poor growth in the uterus, or any combination thereof.

In some embodiments, the comparison of the results of the fetal genetic risk analysis and the one or more databases can be used to determine future health risks to the fetus. In some embodiments, if the results from the fetal genetic risk analysis is the same as the information from the one or more databases, then the comparison determines one or more future health risks to the fetus. In some embodiments, if the results from the fetal genetic risk analysis is different from the information from the one or more databases, the then comparison may not determine one or more future health risks to the fetus. In some embodiments, the comparison of the results of the fetal genetic risk analysis and the one or more databases can be used to determine future health risks to the mother. In some embodiments, if the results from the fetal genetic risk analysis is the same as the information from the one or more databases, then the comparison determines one or more future health risks to the mother. In some embodiments, if the results from the fetal genetic risk analysis is different from the information from the one or more databases, then the comparison may not determine one or more future health risks to the mother. In some embodiments, the comparison of the results of the fetal genetic risk analysis and the one or more databases can be used to determine future health risks to the fetus and the mother. In some embodiments, if the results from the fetal genetic risk analysis is the same as the information from the one or more databases, then the comparison determines one or more future health risks to the fetus and the mother. In some embodiments, if the results from the fetal genetic risk analysis is different from the information from the one or more databases, the then comparison may not determine one or more future health risks to the fetus and the mother.

In some embodiments, the comparison of the results of the fetal genetic risk analysis and the one or more databases can be used to determine clinical treatments to mitigate the health risks to the fetus. In some embodiments, if the results from the fetal genetic risk analysis is the same as the information from the one or more databases, then the comparison determines one or more clinical treatments to mitigate the health risks to the fetus. In some embodiments, if the results from the fetal genetic risk analysis is different from the information from the one or more databases, then the comparison may not determine one or more clinical treatments to mitigate the health risks to the fetus.

In some embodiments, the comparison of the results of the fetal genetic risk analysis and the one or more databases can be used to determine clinical treatments to mitigate the health risks to the mother. In some embodiments, if the results from the fetal genetic risk analysis is the same as the information from the one or more databases, then the comparison determines one or more clinical treatments to mitigate the health risks to the mother. In some embodiments, if the results from the fetal genetic risk analysis is different from the information from the one or more databases, then the comparison may not determine one or more clinical treatments to mitigate the health risks to the mother.

In some embodiments, the comparison of the results of the fetal genetic risk analysis and the one or more databases can be used to determine clinical treatments to mitigate the health risks to the mother and the fetus. In some embodiments, if the results from the fetal genetic risk analysis is the same as the information from the one or more databases, then the comparison determines one or more clinical treatments to mitigate the health risks to the mother and the fetus. In some embodiments, if the results from the fetal genetic risk analysis is different from the information from the one or more databases, the then comparison may not determine one or more clinical treatments to mitigate the health risks to the mother and the fetus.

In some embodiments, the clinical treatments can be treatments to mitigate health risks to the fetus and/or the mother. Non-limiting examples of the clinical treatments include prenatal vitamins, iron supplements, insulin, blood pressure lowering medications, induction of labor, bed rest, blood transfusion, intravenous fluids, nausea medication, antibiotics, vaccination for the fetus following birth (e.g., Hepatitis B vaccination), vaccinations for the mother during the pregnancy (e.g., Flu or Covid vaccination), cesarean delivery, surgery, observation in a hospital, external cephalic version (ECV), or any combination thereof. In some embodiments, the comparison of the results of the fetal genetic risk analysis and the one or more databases can be used to determine clinical treatments to mitigate the health risks to the fetus and the mother.

In some embodiments, the subpopulation of maternal cells is quantified. In some embodiments, the subpopulation of maternal cells is quantified by flow cytometry. In some embodiments, the subpopulation of maternal cells is quantified by fluorescence-activated cell sorting (FACS) flow cytometry. In some embodiments, the subpopulation of maternal cells is quantified by magnetic-activated cell sorting (MACS) flow cytometry. In some embodiments, the subpopulation of maternal cells is quantified by microscopic microdissection. In some embodiments, the subpopulation of maternal cells is quantified by microscopic imaging. In some embodiments, the subpopulation of maternal cells is quantified by acoustic focusing flow cytometry. In some embodiments, the subpopulation of maternal cells is quantified by a hemocytometer. In some embodiments, the subpopulation of maternal cells is quantified by an automated cell counter. In some embodiments, the subpopulation of maternal cells is quantified by spectrophotometry.

In some embodiments, the subpopulation of fetal cells is quantified. In some embodiments, the subpopulation of fetal cells is quantified by flow cytometry. In some embodiments, the subpopulation of fetal cells is quantified by fluorescence-activated cell sorting (FACS) flow cytometry. In some embodiments, the subpopulation of fetal cells is quantified by magnetic-activated cell sorting (MACS) flow cytometry. In some embodiments, the subpopulation of fetal cells is quantified by microscopic microdissection. In some embodiments, the subpopulation of fetal cells is quantified by microscopic imaging. In some embodiments, the subpopulation of fetal cells is quantified by acoustic focusing flow cytometry. In some embodiments, the subpopulation of fetal cells is quantified by a hemocytometer. In some embodiments, the subpopulation of fetal cells is quantified by an automated cell counter. In some embodiments, the subpopulation of fetal cells is quantified by spectrophotometry.

In some embodiments, the subpopulation of maternal cells and the subpopulation of fetal cells are quantified. In some embodiments, the subpopulation of fetal cells and the subpopulation of maternal cells are quantified by flow cytometry. In some embodiments, the subpopulation of fetal cells and the subpopulation of maternal cells are quantified by fluorescence-activated cell sorting (FACS) flow cytometry. In some embodiments, the subpopulation of fetal cells and the subpopulation of maternal cells are quantified by magnetic-activated cell sorting (MACS). In some embodiments, the subpopulation of fetal cells and the subpopulation of maternal cells are quantified by microscopic microdissection. In some embodiments, the subpopulation of fetal cells and the subpopulation of fetal cells are quantified by microscopic imaging. In some embodiments, the subpopulation of fetal cells and the subpopulation of maternal cells are quantified by acoustic focusing flow cytometry. In some embodiments, subpopulation of fetal cells and the subpopulation of maternal cells are quantified by a hemocytometer. In some embodiments, the subpopulation of fetal cells and the subpopulation of maternal cells are quantified by an automated cell counter. In some embodiments, the subpopulation of fetal cells and the subpopulation of maternal cells are quantified by spectrophotometry.

In some embodiments, the present disclosure provides a method of sequencing one or more nucleic acid sequences from an ex vivo population of cells. In some embodiments, the ex vivo population of cells is an ex vivo population of fetal cells. In some embodiments, the ex vivo population of fetal cells has multiple fetal cell types (e.g., early gestation trophoblasts, late gestation trophoblasts, erythroblasts, megakaryocytes, stromal cells, endothelial cells, lymphocytes, nucleated red blood cells, hematopoietic progenitor cells, vascular endothelial cells, and mesenchymal progenitor cells). In some embodiments, the ex vivo population of fetal cells has from about 1 to about 10 fetal cell types. In some embodiments, the ex vivo population of fetal cells has from about 1 to about 2, from about 2 to about 3, from about 3 to about 4, from about 4 to about 5, from about 5 to about 6, from about 6 to about 7, from about 7 to about 8, from about 8 to about 9, or from about 9 to about 10 fetal cell types. In some embodiments, the ex vivo population of fetal cell from has at least about 1, at least about 2, at least about 3, at least about 4, at least about 5, at least about 6, at least about 7, at least about 8, at least about 9, at least about 10, or more fetal cell types. In some embodiments, the ex vivo population of fetal cells has at most about 10, at most about 9, at most about 8, at most about 7, at most about 6, at most about 5, at most about 4, at most about 3, at most about 2, or at most about 1, fetal cell types. In some embodiments, the ex vivo population of fetal cells has about 1, about 2, about 3, about 4, about 5, about 6, about 7, about 8, about 9, or about 10 fetal cell types. In some embodiments, each fetal cell type in the ex vivo population of fetal cells is different.

In some embodiments, the method comprises sequencing a first nucleic acid sequence. In some embodiments, the first nucleic acid sequence is from a first fetal cell type from the ex vivo population of fetal cells. In some embodiments, the method further comprises sequencing a second nucleic acid sequence. In some embodiments, the second nucleic acid sequence is from a second fetal cell type from the ex vivo population of fetal cells. In some embodiments, the method further comprises sequencing a third nucleic acid sequence. In some embodiments, the third nucleic acid sequence is from a third fetal cell type from the ex vivo population of fetal cells. In some embodiments, the method further comprises sequencing a fourth nucleic acid sequence. In some embodiments, the fourth nucleic acid sequence is from a fourth fetal cell type from the ex vivo population of fetal cells. In some embodiments, the method further comprises sequencing a fifth nucleic acid sequence. In some embodiments, the fifth nucleic acid sequence is from a fifth fetal cell types from the ex vivo population of fetal cells. In some embodiments, the first fetal cell type, the second fetal cell type, the third fetal cell type, the fourth fetal cell type, and the fifth fetal cell type are different.

In some embodiments, the method further comprises analyzing at least one nucleic acid sequence. In some embodiments, the first nucleic acid sequence is analyzed. In some embodiments, the second nucleic acid sequence is analyzed. In some embodiments, the third nucleic acid sequence is analyzed. In some embodiments, the fourth nucleic acid sequence is analyzed. In some embodiments, the fifth nucleic acid sequence is analyzed.

In some embodiments, the first nucleic acid sequence and the second nucleic acid molecule are analyzed. In some embodiments, the first nucleic acid sequence and third nucleic acid molecule are analyzed. In some embodiments, the first nucleic acid sequence and the fourth nucleic acid molecule are analyzed. In some embodiments, the first nucleic acid sequence and the fifth nucleic acid molecule are analyzed.

In some embodiments, the second nucleic acid sequence and the third nucleic acid molecule are analyzed. In some embodiments, the second nucleic acid sequence and the fourth nucleic acid molecule are analyzed. In some embodiments, the second nucleic acid sequence and the fifth nucleic acid molecules are analyzed.

In some embodiments, the third nucleic acid sequence and the fourth nucleic acid sequence are analyzed. In some embodiments, the third nucleic acid sequence and the fifth nucleic acid sequence are analyzed. In some embodiments, the fourth nucleic acid sequence and the fifth nucleic acid sequence are analyzed.

In some embodiments, the first nucleic acid sequence, the second nucleic acid sequence, and the third nucleic acid sequence are analyzed. In some embodiments, the first nucleic acid sequence, the second nucleic acid sequence, and the fourth nucleic acid sequence are analyzed. In some embodiments, the first nucleic acid sequence, the second nucleic acid sequence, and the fifth nucleic acid sequence are analyzed.

In some embodiments, the second nucleic acid sequence, the third nucleic acid sequence, and the fourth nucleic acid sequence are analyzed. In some embodiments, the second nucleic acid sequence, the third nucleic acid sequence, and the fifth nucleic acid sequence are analyzed. In some embodiments, the third nucleic acid sequence, the fourth nucleic acid sequence, and the fifth nucleic acid sequence are analyzed.

In some embodiments, the first nucleic acid sequence, the third nucleic acid sequence, the fourth nucleic acid sequence, and the fifth nucleic acid sequence are analyzed. In some embodiments, the first nucleic acid sequence, the second nucleic acid sequence, the third nucleic acid sequence, and the fourth nucleic acid sequence are analyzed. In some embodiments, the second nucleic acid sequence, the third nucleic acid sequence, the fourth nucleic acid sequence, and the fifth nucleic acid sequence are analyzed. In some embodiments, the first nucleic acid sequence, the second nucleic acid sequence, the fourth nucleic acid sequence, and the fifth nucleic acid sequence are analyzed. In some embodiments, the first nucleic acid sequence, the second nucleic acid sequence, the third nucleic acid sequence, and the fifth nucleic acid sequence are analyzed.

In some embodiments, the first nucleic acid sequence, the second nucleic acid sequence, the third nucleic acid sequence, the fourth nucleic acid sequence, and the fifth nucleic acid sequence are analyzed.

In some embodiments, the analyzing determines a likelihood that the ex vivo population of cells is susceptible to a disease or condition. In some embodiments, the disease or condition is a genetic condition or disease. In some embodiments, the ex vivo population of cells is associated with a fetus.

In some embodiments, at least one of the first nucleic acid sequence, the second nucleic acid sequence, the third nucleic acid sequence, the fourth nucleic acid sequence, or the fifth nucleic acid sequence is a DNA sequence. In some embodiments, at least the first nucleic acid sequence is a DNA sequence. In some embodiments, at least the second nucleic acid sequence is a DNA sequence. In some embodiments, at least the third nucleic acid sequence is a DNA sequence. In some embodiments, at least the fourth nucleic acid sequence is a DNA sequence. In some embodiments, at least the fifth nucleic acid sequence is a DNA sequence.

In some embodiments, at least one of the first nucleic acid sequence, the second nucleic acid sequence, the third nucleic acid sequence, the fourth nucleic acid sequence, or the fifth nucleic acid sequence is an RNA sequence. In some embodiments, at least the first nucleic acid sequence is an RNA sequence. In some embodiments, at least the second nucleic acid sequence is an RNA sequence. In some embodiments, at least the third nucleic acid sequence is an RNA sequence. In some embodiments, at least the fourth nucleic acid sequence is an RNA sequence. In some embodiments, at least the fifth nucleic acid sequence is an RNA sequence.

In some embodiments, the ex vivo population of fetal cells is in a solution with a Artiodactyla serum. In some embodiments, the Artiodactyla serum is a porcine serum. In some embodiments, the Artiodactyla serum is an ovine serum. In some embodiments, the Artiodactyla serum is a Caprine serum. In some embodiments, the Artiodactyla serum is a bovine serum.

In some embodiments, the method further comprises providing an extract prior to sequencing. In some embodiments, the extract is made by extracting populations of cells from the maternal biological sample. In some embodiments, the population of cells is extracted from the maternal biological sample. In some embodiments, the ex vivo population of fetal cells is extracted from the maternal biological sample. In some embodiments, an ex vivo population of maternal cells is extracted from the maternal biological sample. In some embodiments, the ex vivo population of maternal cells and the ex vivo population of fetal cells are extracted from the maternal biological sample. In some embodiments, the population of cells extracted from the maternal biological sample are used to provide an extract. In some embodiments, the population of cells is extracted from the maternal biological sample using density gradient centrifugation. In some embodiments, the population of cells is extracted from the maternal biological sample using magnetic isolation. In some embodiments, the population of cells is extracted from the maternal biological sample using shear-induced diffusion. In some embodiments, the extract is made up of the ex vivo population of fetal cells. In some embodiments, the extract is made up of the ex vivo population of maternal cells. In some embodiments, the extract is made up of the ex vivo population of fetal cells and the ex vivo population of maternal cells.

In some embodiments, the method further comprises enriching the ex vivo population of fetal cells in the extract to provide an enriched sample. In some embodiments, the extract is enriched to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, extract is enriched for fetal cells by immunomagnetic cell separation. In some embodiments, the immunomagnetic cell separation is positive immunomagnetic cell selection. In some embodiments, the extract is enriched for fetal cells by fluorescence-activated cell sorting (FACS). In some embodiments, the extract is enriched for fetal cells by magnetic-activated cell sorting (MACS). In some embodiments, the extract is enriched for fetal cells by microscopic microdissection. In some embodiments, the extract is enriched for fetal cell by microscopic imaging. In some embodiments, the extract is enriched for fetal cells using one or more fetal cell biomarkers. In some embodiments, the extract is enriched for fetal cells by negative immunomagnetic cell separation. In some embodiments, the negative immunomagnetic cell separation enriches for fetal cells using one or more maternal cell biomarkers. In some embodiments, the extract is enriched for fetal cells using negative selection using one or more maternal biomarkers. In some embodiments, the extract is enriched for fetal cells using positive selection using one or more fetal biomarkers. In some embodiments, the extract is enriched for fetal cells using negative selection using one or more maternal biomarkers, then subsequently positive selection using one or more fetal biomarkers. In some embodiments, the extract is for enriched for fetal cells using positive selection using one or more fetal biomarkers, then subsequently negative selection using one or more maternal biomarkers. In some embodiments, the extract is enriched for fetal cells using positive selection using one or more fetal biomarkers, then subsequentially enriched for fetal cells using positive selection using another one or more fetal biomarkers. In some embodiments, the extract is enriched for fetal cells using negative selection using one or more maternal biomarkers, then enriched for fetal cells using positive selection using one or more fetal biomarkers, and then the extract is enriched for fetal cells using positive selection using another one or more fetal biomarkers. In some embodiments, the extract is enriched for fetal cells using one or more isolation agents specific for one or more fetal cell biomarkers. In some embodiments, the isolation agent specific for a fetal cell biomarker has a first isolation agent. In some embodiments, the first isolation agent couples to a first fetal biomarker. In some embodiments, the extract is enriched for fetal cells using a first isolation agent.

In some embodiments, the ex vivo population of fetal cells is extracted from a biological sample. In some embodiments, the biological sample is a blood sample. In some embodiments, the blood sample is blood plasma. In some embodiments, the blood sample is blood serum. In some embodiments, the blood sample is a buffy coat. In some embodiments, the blood sample is peripheral blood mononuclear cells. In some embodiments, the blood sample is nucleated white blood cells. In some embodiments, the blood sample is whole blood. In some embodiments, the blood sample is whole blood depleted of red blood cells. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof. In some embodiments, the biological sample is from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the sample is from a pregnant subject. In some embodiments, the sample is a maternal biological sample. In some embodiments, the maternal biological sample is from a pregnant subject.

In some embodiments, the method further comprises enriching the ex vivo population of maternal cells in the extract to provide an enriched sample. In some embodiments, the extract is enriched to provide an enriched sample. In some embodiments, the enriched sample is enriched in maternal cells. In some embodiments, the enriched sample is depleted in fetal cells. In some embodiments, the enriched sample is enriched in maternal cells and depleted in fetal cells. In some embodiments, extract is enriched for maternal cells by immunomagnetic cell separation. In some embodiments, the immunomagnetic cell separation is positive immunomagnetic cell selection. In some embodiments, the extract is enriched for maternal cells by fluorescence-activated cell sorting (FACS). In some embodiments, the extract is enriched for maternal cells by magnetic-activated cell sorting (MACS). In some embodiments, the extract is enriched for maternal cells by microscopic microdissection. In some embodiments, the extract is enriched for maternal cells by microscopic imaging. In some embodiments, the extract is enriched for maternal cells using one or more maternal cell biomarkers. In some embodiments, the extract is enriched for maternal cells by negative immunomagnetic cell separation. In some embodiments, the negative immunomagnetic cell separation enriches for maternal cells using one or more fetal cell biomarkers. In some embodiments, the extract is enriched for maternal cells using negative selection using one or more fetal biomarkers then subsequently positive selection using one or more maternal biomarkers. In some embodiments, the extract is enriched for maternal cells using positive selection using one or more maternal biomarkers, then subsequently negative selecting using one or more fetal biomarkers. In some embodiments, the extract is enriched for maternal cells using one or more isolation agent specific for one or more maternal cell biomarkers. In some embodiments, the one or more isolation agents specific for one or more maternal cell biomarkers has a second isolation agent. In some embodiments, the second isolation agent couples to a second biomarker in the ex vivo population of maternal cells.

In some embodiments, the method further comprises sorting the ex vivo population of the fetal cells in the extract from the ex vivo population of maternal cells. In some embodiments, the sorting of the ex vivo population of the cells in the extract provides an enriched sample. In some embodiments, the extract is sorted to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, the extract is enriched for fetal cells by sorting the extract for a cell characteristic. Non-limiting examples of cell characteristics include size, expression of a specific gene, expression of an extracellular protein, and expression of an intracellular protein. In some embodiments, the extract is sorted by immunomagnetic cell separation. In some embodiments, the immunomagnetic cell separation is positive immunomagnetic cell selection. In some embodiments, the extract is enriched for fetal cells by negative immunomagnetic cell separation. In some embodiments, the extract is enriched for fetal cells by positive selection. In some embodiments, the extract is enriched for fetal cells by negative selection (e.g., removing maternal cells). In some embodiments, the extract is enriched for fetal cells by positive selection and negative selection. In some embodiments, the extract is enriched for fetal cells by positive selection prior to negative selection. In some embodiments, the extract is enriched for fetal cells by negative selection followed by positive selection. In some embodiments, the extract is enriched for fetal cells using positive selection using one or more fetal biomarkers, then subsequentially enriched for fetal cells using positive selection using another one or more fetal biomarkers. In some embodiments, the extract is enriched for fetal cells using negative selection using one or more maternal biomarkers, then enriched for fetal cells using positive selection using one or more fetal biomarkers, and then the extract is enriched for fetal cells using positive selection using another one or more fetal biomarkers. In some embodiments, the negative immunomagnetic cell separation enriches for fetal cells using one or more maternal cell biomarkers. In some embodiments, the extract is sorted by FACS. In some embodiments, the extract is sorted by MACS. In some embodiments, the extract is sorted by microscopic microdissection. In some embodiments, the extract is sorted by microscopic imaging. In some embodiments, the extract is sorted for fetal cells using a one or more isolation agents specific to one or more fetal cell biomarkers in the ex vivo population of fetal cells. In some embodiments, the extract is sorted by microfluidic cell sorting.

In some embodiments, the method further comprises sorting the ex vivo population of the maternal cells in the extract from the ex vivo population of fetal cells. In some embodiments, the sorting of the ex vivo population of the cells in the extract provides an enriched sample. In some embodiments, the extract is sorted to provide an enriched sample. In some embodiments, the enriched sample is enriched in maternal cells. In some embodiments, the enriched sample is depleted in fetal cells. In some embodiments, the enriched sample is enriched in maternal cells and depleted in fetal cells. In some embodiments, the extract is enriched for maternal cells by sorting the extract for a cell characteristic. Non-limiting examples of cell characteristics include size, expression of a specific gene, expression of an extracellular protein, and expression of an intracellular protein. In some embodiments, the extract is sorted by immunomagnetic cell separation. In some embodiments, the immunomagnetic cell separation is positive immunomagnetic cell selection. In some embodiments, the extract is enriched for maternal cells by negative immunomagnetic cell separation. In some embodiments, the extract is enriched for maternal cells by positive selection (e.g., enriching for maternal cells). In some embodiments, the extract is enriched for maternal cells by negative selection (e.g., removing fetal cells). In some embodiments, the extract is enriched for maternal cells by positive selection and negative selection. In some embodiments, the extract is enriched for maternal cells by positive selection prior to negative selection. In some embodiments, the extract is enriched for maternal cells by negative selection followed by positive selection. In some embodiments, the negative immunomagnetic cell separation enriches for maternal cells using one or more fetal cell biomarkers. In some embodiments, the extract is sorted by FACS. In some embodiments, the extract is sorted by MACS. In some embodiments, the extract is sorted by microscopic microdissection. In some embodiments, the extract is sorted by microscopic imaging. In some embodiments, the extract is sorted for maternal cells using one or more isolation agents specific for one or more maternal biomarkers in the ex vivo population of maternal cells. In some embodiments, the extract is sorted by microfluidic cell sorting.

In some embodiments, the method further comprises incubating the biological sample. In some embodiments, the biological sample has the ex vivo population of fetal cells. In some embodiments, the biological sample has the ex vivo population of maternal cells. In some embodiments, the biological sample has the ex vivo population of maternal cells and the ex vivo population of fetal cells. In some embodiments, the biological sample is incubated with at least one isolation agent. In some embodiments, the biological sample is incubated with a first isolation agent. In some embodiments, the biological sample is incubated with a second isolation agent. In some embodiments, the biological sample is incubated with the first isolation agent and the second isolation agent.

In some embodiments, the ex vivo population of cells is fixed prior to incubation with the at least one isolation agent. In some embodiments, the ex vivo population of cells is an ex vivo population of fetal cells. In some embodiments, the ex vivo population of cells is an ex vivo population of maternal cells. In some embodiments, the ex vivo population of cells is an ex vivo population of fetal cells and an ex vivo population of maternal cells. In some embodiments, the ex vivo population of cells is fixed via a fixation agent. Non-limiting examples of a fixation agent include formaldehyde, glutaraldehyde, acrolein, glyoxal, paraformaldehyde, Lormant's Reagent (dithiobis(succinimidyl propionate) (DSP)), osmium tetroxide, carbodiimides, diimidoesters, chloro-s-triazides, diisocyanates, diethylpyrocarbonate, maleimides, benzoquinone, mercuric chloride, zinc sulphate, zinc chloride, picric acid, potassium dichromate, ethanol, methanol, acetone, acetic acid, and any combination thereof. In some embodiments, the ex vivo population of cells is permeabilized prior to incubation with the at least one isolation agent. In some embodiments, the ex vivo population of cells is permeabilized via a permeabilization agent. Non-limiting examples of permeabilization agents include methanol, acetone, saponin, Triton X-100, Tween-20, EDTA, nitrilotriacetic acid, sodium hexametaphosphate, digitonin, and any combination thereof. In some embodiments, the ex vivo population of cells is fixed and permeabilized prior to incubation with the at least one isolated agent.

Alternatively, in some embodiments, the ex vivo population of cells is not fixed or permeabilized prior to incubation with the at least one isolated agent.

In some embodiments, the fixation of the ex vivo population of cells is reversed prior to analysis of the fetal cells. In some embodiments, the fixation of the ex vivo population of cells is reversed by removing the cross-links between the molecules in and/or on the cell. In some embodiments, between about 0% to about 100% of the fixation of the ex vivo population of cells is reversed. In some embodiments, at least about 0%, at least about 10%, at least about 20%, at least about 30%, at least about 40%, at least about 50%, at least about 60%, at least about 70%, at least about 80%, at least about 90%, at least about 100%, or more of the fixation of the ex vivo population of cells is reversed. In some embodiments, at most about 100%, at most about 90%, at most about 80%, at most about 70%, at most about 60%, at most about 50%, at most about 40%, at most about 30%, at most about 20%, at most about 10%, at most about 0%, or less of the fixation of the ex vivo population of cells is reversed. In some embodiments, about 0%, about 10%, about 20%, about 30%, about 40%, about 50%, about 60%, about 70%, about 80%, about 90%, or about 100% of the fixation of the ex vivo population of cells is reversed. In some embodiments, the fixation of the ex vivo population of cells is reversed via treatment with citraconic anhydride, Tris buffer, phosphate buffer, sodium borohydride, Sarkosyl, gloxal, low concentration of paraformaldehyde, Lomant's reagent, dithiobis(succinimidly propionate), paraformaldehyde, dithiothreitol, heat, lithium aluminium hydride, Red-Al, sodium amalgam, sodium-lead alloy, zinc amalgam, diborane, sodium borohydride, sulfur dioxide, hydrazine, diisobutylaluminium hydride, oxalic acid, formic acid, ascorbic acid, or any combination thereof.

In some embodiments, the maternal biological sample is incubated with one or more isolation agents that couples to one or more fetal biomarkers. In some embodiments, the maternal biological sample is incubated with one or more isolation agents that couples to one or more maternal biomarkers. In some embodiments, the maternal biological sample is incubated with one or more isolation agents that couples to one or more fetal biomarkers, then subsequently incubated with one or more isolation agents that couples to one or more maternal biomarkers. In some embodiments, that maternal biological sample is incubated with one or more isolation agents that couples to one or more maternal biomarkers, then subsequently incubated with one or more isolation agents that couples to one or more fetal biomarkers. In some embodiments, the maternal biological sample is incubated with one or more isolation agents that couples to one or more fetal biomarkers, then subsequently incubated with another one or more isolation agents that couples to another one or more fetal biomarkers. In some embodiments, the maternal biological sample is incubated with one or more isolation agents that couples to one or more maternal biomarkers, then incubated with one or more isolation agents that couples to one or more fetal biomarkers, and then incubated with another one or more isolation agents that couples to another one or more fetal biomarkers.

In some embodiments, the first isolation agent couples to a first biomarker. In some embodiments, the first biomarker is one or more fetal biomarkers. In some embodiments, the second isolation agent couples to a second biomarker. In some embodiments, the second biomarker is one or more maternal biomarkers. In some embodiments, the isolation agent couples to an extracellular biomarker. In some embodiments, the isolation agent couples to an extracellular protein. In some embodiments, the isolation agent couples to a cell surface protein. In some embodiments, the isolation agent couples to a soluble cell surface protein. In some embodiments, the isolation agent couples to a cell-surface receptor. In some embodiments, the isolation agent couples to a transmembrane protein. In some embodiments, the isolation agent couples to a transmembrane channel protein.

In some embodiments, the isolation agent couples to an intracellular biomarker. In some embodiments, the isolation agent couples to an intracellular protein. In some embodiments, the isolation agent couples to an enzyme. In some embodiments, the isolation agent couples to a phosphoprotein. In some embodiments, the isolation agent couples to a growth factor. In some embodiments, the isolation agent couples to a structural protein.

In some embodiments, the isolation agent couples to a nucleic acid biomarker. In some embodiments, the isolation agent couples to a DNA biomarker. In some embodiments, the isolation agent couples to an RNA biomarker. In some embodiments, the isolation agent couples to mRNA. In some embodiments, the isolation agent couples to rRNA. In some embodiments, the isolation agent couples to lncRNA. In some embodiments, the isolation agent couples to miRNA. In some embodiments, the isolation agent couples to siRNA.

In some embodiments, the incubating further comprises sorting the population of cells from the sample. In some embodiments, the ex vivo population of fetal cells is sorted. In some embodiments, the ex vivo population of maternal cells is sorted. In some embodiments, the ex vivo population of fetal cells is sorted based on the presence of the first isolation agent. In some embodiments, the ex vivo population of fetal cells is sorted based on the absence of the second isolation agent. In some embodiments, the ex vivo population of fetal cells is sorted based on the presence of the first isolation agent and the absence of the second isolation agent. In some embodiments, the ex vivo population of maternal cells is sorted based on the presence of the second isolation agent. In some embodiments, the ex vivo population of maternal cells is sorted based on the absence of the first isolation agent. In some embodiments, the ex vivo population of maternal cells is sorted based on the presence of the second isolation agent and the absence of the first isolation agent.

In some embodiments, the ex vivo population of fetal cells is sorted from the sample. In some embodiments, the ex vivo population of fetal cells is sorted based on a presence of a first isolation agent. In some embodiments, the first isolation agent is specific for one or more fetal biomarkers. In some embodiments, the first isolation agent couples to the one or more fetal biomarkers. In some embodiments, the ex vivo population of fetal cells is sorted based on detection of a presence of the first isolation agent coupled to the one or more fetal biomarkers. In some embodiments, cells from the sample that are coupled to the first isolation agent are separated from other cells from the sample that are not coupled to the first isolation agent.

In some embodiments, the ex vivo population of fetal cells is sorted from the sample. In some embodiments, the ex vivo population of fetal cells is sorted based on an absence of a second isolation agent. In some embodiments, the second isolation agent is specific for one or more maternal biomarkers. In some embodiments, the second isolation agent couples to the one or more maternal biomarkers. In some embodiments, the ex vivo population of fetal cells is sorted based on detection of an absence of the second isolation agent coupled to the one or more maternal biomarkers. In some embodiments, cells from the sample that are coupled to the second isolation agent are separated from other cells from the sample that are not coupled to the second isolation agent.

In some embodiments, the ex vivo population of fetal cells is sorted from the sample. In some embodiments, the ex vivo population of fetal cells is sorted based on a presence of a first isolation agent and an absence of a second isolation marker. In some embodiments, the first isolation agent is specific for one or more fetal biomarkers. In some embodiments, the first isolation agent couples to the one or more fetal biomarkers. In some embodiments, the second isolation agent is specific for one or more maternal biomarkers. In some embodiments, the second isolation couples to the one or more maternal biomarkers. In some embodiments, the ex vivo population of fetal cells is sorted based on detection of a presence of the first isolation agent coupled to the one or more fetal biomarkers and an absence of the second isolation agent coupled to the one or more maternal biomarkers. In some embodiments, cells from the sample that are coupled to the first isolation agent and are not coupled to the second isolation agent are separated from other cells from the sample that are not coupled to the first isolation agent and are coupled to the second isolation agent.

In some embodiments, the ex vivo population of maternal cells is sorted from the sample. In some embodiments, the ex vivo population of maternal cells is sorted based on a presence of a second isolation agent. In some embodiments, the second isolation agent is specific for one or more maternal biomarkers. In some embodiments, the second isolation agent couples to the one or more maternal biomarkers. In some embodiments, the ex vivo population of maternal cells is sorted based on detection of a presence of the second isolation agent coupled to the one or more maternal biomarkers. In some embodiments, cells from the sample that are coupled to the second isolation agent are separated from other cells from the sample that are not coupled to the second isolation agent.

In some embodiments, the ex vivo population of maternal cells is sorted from the sample. In some embodiments, the ex vivo population of maternal cells is sorted based on an absence of a first isolation agent. In some embodiments, the first isolation agent is specific for one or more fetal biomarkers. In some embodiments, the first isolation agent couples to the one or more fetal biomarkers. In some embodiments, the ex vivo population of maternal cells is sorted based on detection of an absence of the first isolation agent coupled to the one or more fetal biomarkers. In some embodiments, cells from the sample that are coupled to the first isolation agent are separated from other cells from the sample that are not coupled to the first isolation agent.

In some embodiments, the ex vivo population of maternal cells is sorted from the sample. In some embodiments, the ex vivo population of maternal cells is sorted based on a presence of a second isolation agent and an absence of a first isolation marker. In some embodiments, the first isolation agent is specific for one or more fetal biomarkers. In some embodiments, the first isolation agent couples to the one or more fetal biomarkers. In some embodiments, the second isolation agent is specific for one or more maternal biomarkers. In some embodiments, the second isolation couples to the one or more maternal biomarkers. In some embodiments, the ex vivo population of maternal cells is sorted based on detection of a presence of the second isolation agent coupled to the one or more maternal biomarkers and an absence of the first isolation agent coupled to the one or more fetal biomarkers. In some embodiments, cells from the sample that are coupled to the first isolation agent and are not coupled to the second isolation agent are separated from other cells from the sample that are not coupled to the first isolation agent and are coupled to the second isolation agent.

In some embodiments, the one or more isolation agents directly couple to the one or more biomarkers. In some embodiments, the one or more isolation agents couples to the one or more biomarkers via a covalent bond. In some embodiments, the covalent bond is a peptide bond. In some embodiments, the one or more isolation agents couples to the one or more biomarkers via a non-covalent interaction. In some embodiments, the non-covalent interaction is an ionic interaction. In some embodiments, the non-covalent interaction is a hydrogen bond. In some embodiments, the non-covalent interaction is van der Waals interaction. In some embodiments, the non-covalent interaction is a hydrophobic interaction.

In some embodiments, the one or more isolation agents indirectly couple to the one or more biomarkers. In some embodiments, the one or more isolation agents couple to the one or more biomarkers via a linker. In some embodiments, the linker is a cleavable linker. In some embodiments, the cleavable linker is a chemical labile linker. Non-limiting examples of chemical labile linkers include 6-(maleimidocaproyl)hydrazone, (4-(4-acetylphenoxy)butanoic acid) hydrazone, N-acetyl-y-calicheamicin dimethyl hydrazide, or any combination thereof. In some embodiments, the cleavable linker is a reducible linker. In some embodiments, the reducible linker is a disulfide linker or a glutathione-sensitive disulfide linker. In some embodiments, the cleavable linker is an enzyme-cleavable linker. Non-limiting examples of enzyme-cleavable linkers include peptide-based linkers, valine-citrulline, phenylalanine-lysine, valine-alanine, para-aminobenzyl carbamate, PEG linkers, β-glucuronide linker, β-galactoside linkers, or any combination thereof. In some embodiments, the cleavable linker is a phosphatase-cleavable linker. In some embodiments, the phosphatase-cleavable linker is a phosphate diester. In some embodiments, the linker is a non-cleavable linker. Non-limiting examples of non-cleavable linkers include thioethers, maleimidocapropy, N-succinimidyl-4-(maleimidomethyl)cyclohexane-1-carboxylate linker, and any combination thereof.

In some embodiments, the first isolation agent couples to an extracellular biomarker. In some embodiments, the first isolation agent couples to an extracellular protein. In some embodiments, the first isolation agent couples to a cell surface protein. In some embodiments, the first isolation agent couples to a soluble cell surface protein. In some embodiments, the first isolation agent couples to a cell-surface receptor. In some embodiments, the first isolation agent couples to a transmembrane protein. In some embodiments, the first isolation agent couples to a transmembrane channel protein.

In some embodiments, the first isolation agent couples to an intracellular biomarker. In some embodiments, the first isolation agent couples to an intracellular protein. In some embodiments, the first isolation agent couples to an enzyme. In some embodiments, the first isolation agent couples to a phosphoprotein. In some embodiments, the first isolation agent couples to a growth factor. In some embodiments, the first isolation agent couples to a structural protein.

In some embodiments, the first isolation agent couples to a nucleic acid biomarker. In some embodiments, the first isolation agent couples to a DNA biomarker. In some embodiments, the first isolation agent couples to an RNA biomarker. In some embodiments, the first isolation agent couples to mRNA. In some embodiments, the first isolation agent couples to rRNA. In some embodiments, the first isolation agent couples to lncRNA. In some embodiments, the first isolation agent couples to miRNA. In some embodiments, the first isolation agent couples to siRNA.

In some embodiments, the isolation agent couples to a fetal biomarker. In some embodiments, the first biomarker is a fetal biomarker. In some embodiments, the biomarker is a Sct biomarker, a H19 biomarker, a Tfpi biomarker, or a Fthl17a biomarker. In some embodiments, the biomarker is a Sct biomarker. In some embodiments, the biomarker is a H19 biomarker. In some embodiments, the biomarker is a Tfpi biomarker. In some embodiments, the biomarker is a Fthl17a biomarker.

In some embodiments, the first isolation agent couples to one or more fetal biomarkers. In some embodiments, the first biomarker is a fetal biomarker. In some embodiments, the first biomarker is a Sct biomarker, a H19 biomarker, a Tfpi biomarker, or a Fthl17a biomarker. In some embodiments, the first biomarker is a Sct biomarker. In some embodiments, the first biomarker is a H19 biomarker. In some embodiments, the first biomarker is a Tfpi biomarker. In some embodiments, the first biomarker is a Fthl17a biomarker.

In some embodiments, the second isolation agent couples to a second biomarker. In some embodiments, the second biomarker is a maternal biomarker. In some embodiments, the second isolation agent couples to an extracellular biomarker. In some embodiments, the second isolation agent couples to an extracellular protein. In some embodiments, the second isolation agent couples to a cell surface protein. In some embodiments, the second isolation agent couples to a soluble cell surface protein. In some embodiments, the second isolation agent couples to a cell-surface receptor. In some embodiments, the second isolation agent couples to a transmembrane protein. In some embodiments, the second isolation agent couples to a transmembrane channel protein.

In some embodiments, the second isolation agent couples to an intracellular biomarker. In some embodiments, the second isolation agent couples to an intracellular protein. In some embodiments, the second isolation agent couples to an enzyme. In some embodiments, the second isolation agent couples to a phosphoprotein. In some embodiments, the second isolation agent couples to a growth factor. In some embodiments, the second isolation agent couples to a structural protein.

In some embodiments, the second isolation agent couples to a nucleic acid biomarker. In some embodiments, the second isolation agent couples to a DNA biomarker. In some embodiments, the second isolation agent couples to an RNA biomarker. In some embodiments, the second isolation agent couples to mRNA. In some embodiments, the second isolation agent couples to rRNA. In some embodiments, the second isolation agent couples to lncRNA. In some embodiments, the second isolation agent couples to miRNA. In some embodiments, the second isolation agent couples to siRNA.

In some embodiments, the isolation agent couples to one or more maternal biomarkers. In some embodiments, the second biomarker is one or more maternal biomarkers. In some embodiments, the biomarker is a Coro1a biomarker, a Cd52 biomarker, a Cyba biomarker, a Rac2 biomarker, a Laptm5 biomarker, or a Ptprc biomarker. In some embodiments, the biomarker is a Coro1a biomarker. In some embodiments, the biomarker is a Cd52 biomarker. In some embodiments, the biomarker is a Cyba biomarker. In some embodiments, the biomarker is a Rac2 biomarker. In some embodiments, the biomarker is a Laptm5 biomarker. In some embodiments, the biomarker is a Ptprc biomarker.

In some embodiments, the second isolation agent couples to one or more maternal biomarkers. In some embodiments, the second biomarker is a maternal biomarker. In some embodiments, the second biomarker is a Coro1a biomarker, a Cd52 biomarker, a Cyba biomarker, a Rac2 biomarker, a Laptm5 biomarker, or a Ptprc biomarker. In some embodiments, the second biomarker is a Coro1a biomarker. In some embodiments, the second biomarker is a Cd52 biomarker. In some embodiments, the second biomarker is a Cyba biomarker. In some embodiments, the second biomarker is a Rac2 biomarker. In some embodiments, the second biomarker is a Laptm5 biomarker. In some embodiments, the second biomarker is a Ptprc biomarker.

In some embodiments, the ex vivo population of fetal cells is early gestation fetal cells. In some embodiments, the ex vivo population of fetal cells is later gestation fetal cells. In some embodiments, the ex vivo population of fetal cells is early gestation fetal cells and later gestation fetal cells. In some embodiments, the ex vivo population of fetal cells is early gestation trophoblasts, later gestation trophoblasts, erythroblasts, megakaryocytes, stromal cells, endothelial cells, lymphocytes, nucleated red blood cells, hematopoietic progenitor cells, or mesenchymal progenitor cells. In some embodiments, the ex vivo population of fetal cells is early gestation trophoblasts. In some embodiments, the ex vivo population of fetal cells is later gestation trophoblasts. In some embodiments, the ex vivo population of fetal cells is erythroblasts. In some embodiments, the ex vivo population of fetal cells is megakaryocytes. In some embodiments, the ex vivo population of fetal cells is stromal cells. In some embodiments, the ex vivo population of fetal cells is endothelial cells. In some embodiments, the ex vivo population of fetal cells is lymphocytes. In some embodiments, the ex vivo population of fetal cells is nucleated red blood cells. In some embodiments, the ex vivo population of fetal cells is hematopoietic progenitor cells. In some embodiments, the ex vivo population of fetal cells is mesenchymal progenitor cells. In some embodiments, the ex vivo population of fetal cells is vascular endothelial cells.

In some embodiments, the first fetal cell type is early gestation trophoblasts, later gestation trophoblasts, erythroblasts, megakaryocytes, stromal cells, endothelial cells, lymphocytes, nucleated red blood cells, hematopoietic progenitor cells, mesenchymal progenitor cells, or vascular endothelial cells. In some embodiments, the first fetal cell type is early gestation trophoblasts. In some embodiments, the first fetal cell type is later gestation trophoblasts. In some embodiments, the first fetal cell type is erythroblasts. In some embodiments, the first fetal cell type is megakaryocytes. In some embodiments, the first fetal cell type is stromal cells. In some embodiments, the first fetal cell type is endothelial cells. In some embodiments, the first fetal cell type is lymphocytes. In some embodiments, the first fetal cell type is nucleated red blood cells. In some embodiments, the first fetal cell type is hematopoietic progenitor cells. In some embodiments, the first fetal cell type is mesenchymal progenitor cells. In some embodiments, the first fetal cell type is vascular endothelial cells.

In some embodiments, the second fetal cell type is early gestation trophoblasts, later gestation trophoblasts, erythroblasts, megakaryocytes, stromal cells, endothelial cells, lymphocytes, nucleated red blood cells, hematopoietic progenitor cells, mesenchymal progenitor cells, or vascular endothelial cells. In some embodiments, the second fetal cell type is early gestation trophoblasts. In some embodiments, the second fetal cell type is later gestation trophoblasts. In some embodiments, the second fetal cell type is erythroblasts. In some embodiments, the second fetal cell type is megakaryocytes. In some embodiments, the second fetal cell type is stromal cells. In some embodiments, the second fetal cell type is endothelial cells. In some embodiments, the second fetal cell type is lymphocytes. In some embodiments, the second fetal cell type is nucleated red blood cells. In some embodiments, the second fetal cell type is hematopoietic progenitor cells. In some embodiments, the second fetal cell type is mesenchymal progenitor cells. In some embodiments, the second fetal cell type is vascular endothelial cells.

In some embodiments, the third fetal cell type is early gestation trophoblasts, later gestation trophoblasts, erythroblasts, megakaryocytes, stromal cells, endothelial cells, lymphocytes, nucleated red blood cells, hematopoietic progenitor cells, mesenchymal progenitor cells, or vascular endothelial cells. In some embodiments, the third fetal cell type is early gestation trophoblasts. In some embodiments, the third fetal cell type is later gestation trophoblasts. In some embodiments, the third fetal cell type is erythroblasts. In some embodiments, the third fetal cell type is megakaryocytes. In some embodiments, the third fetal cell type is stromal cells. In some embodiments, the third fetal cell type is endothelial cells. In some embodiments, the third fetal cell type is lymphocytes. In some embodiments, the third fetal cell type is nucleated red blood cells. In some embodiments, the third fetal cell type is hematopoietic progenitor cells. In some embodiments, the third fetal cell type is mesenchymal progenitor cells. In some embodiments, the third fetal cell type is vascular endothelial cells.

In some embodiments, the fourth fetal cell type is early gestation trophoblasts, later gestation trophoblasts, erythroblasts, megakaryocytes, stromal cells, endothelial cells, lymphocytes, nucleated red blood cells, hematopoietic progenitor cells, mesenchymal progenitor cells, or vascular endothelial cells. In some embodiments, the fourth fetal cell type is early gestation trophoblasts. In some embodiments, the fourth fetal cell type is later gestation trophoblasts. In some embodiments, the fourth fetal cell type is erythroblasts. In some embodiments, the fourth fetal cell type is megakaryocytes. In some embodiments, the fourth fetal cell type is stromal cells. In some embodiments, the fourth fetal cell type is endothelial cells. In some embodiments, the fourth fetal cell type is lymphocytes. In some embodiments, the fourth fetal cell type is nucleated red blood cells. In some embodiments, the fourth fetal cell type is hematopoietic progenitor cells. In some embodiments, the fourth fetal cell type is mesenchymal progenitor cells. In some embodiments, the fourth fetal cell type is vascular endothelial cells.

In some embodiments, the fifth fetal cell type is early gestation trophoblasts, later gestation trophoblasts, erythroblasts, megakaryocytes, stromal cells, endothelial cells, lymphocytes, nucleated red blood cells, hematopoietic progenitor cells, mesenchymal progenitor cells, or vascular endothelial cells. In some embodiments, the fifth fetal cell type is early gestation trophoblasts. In some embodiments, the fifth fetal cell type is later gestation trophoblasts. In some embodiments, the fifth fetal cell type is erythroblasts. In some embodiments, the fifth fetal cell type is megakaryocytes. In some embodiments, the fifth fetal cell type is stromal cells. In some embodiments, the fifth fetal cell type is endothelial cells. In some embodiments, the fifth fetal cell type is lymphocytes. In some embodiments, the fifth fetal cell type is nucleated red blood cells. In some embodiments, the fifth fetal cell type is hematopoietic progenitor cells. In some embodiments, the fifth fetal cell type is mesenchymal progenitor cells. In some embodiments, the fifth fetal cell type is vascular endothelial cells.

In some embodiments, the present disclosure provides a method of performing a single round of enrichment on an ex vivo population of cells. In some embodiments, the single round of enrichment is performed to enrich a population of fetal cells in a sample. In some embodiments, the single round of enrichment is performed to enrich a population of maternal cells in a sample. In some embodiments, the sample is a cell sample. In some embodiments, the single round of enrichment is performed to enrich a population of fetal cells from a population of maternal cells. In some embodiments, the single round of enrichment is performed to enrich a population of maternal cells from a population of fetal cells. In some embodiments, the single round of enrichment enriches the population of fetal cell from the population of maternal cells. In some embodiments, the single round of enrichment enriches the population of fetal cells from the population of maternal cells to a level that provides a quantity of fetal nucleic acid that is sufficient for characterization as fetal nucleic acid rather than maternal nucleic acid.

In some embodiments, the single round of enrichment produces a population of cells that is composed of at least about 30% fetal cells. In some embodiments, the single round of enrichment produces a population of cells that is composed of at least about 30% fetal cells, at least about 35% fetal cells, at least about 40% fetal cells, at least about 45% fetal cells, at least about 50% fetal cells, at least about 55% fetal cells, at least about 60% fetal cells, at least about 65% fetal cells, at least about 70% fetal cells, at least about 75% fetal cells, at least about 80% fetal cells, at least about 85% fetal cells, at least about 90% fetal cells, at least about 95% fetal cells, at least about 100% fetal cells, or more. In some embodiments, the single round of enrichment produces a population of cells that is composed of about 30% fetal cells, about 35% fetal cells, about 40% fetal cells, about 45% fetal cells, about 50% fetal cells, about 55% fetal cells, about 60% fetal cells, about 65% fetal cells, about 70% fetal cells, about 75% fetal cells, about 80% fetal cells, about 85% fetal cells, about 90% fetal cells, about 95% fetal cells, or about 100% fetal cells.

In some embodiments the single round of enrichment produces a population of cells that is composed of at most about 70% maternal cells. In some embodiments, the single round of enrichment produces a population of cells that is composed of at most about 70% maternal cells, at most about 65% maternal cells, at most about 60% maternal cells, at most about 55% maternal cells, at most about 50% maternal cells, at most about 45% maternal cells, at most about 40% maternal cells, at most about 35% maternal cells, at most about 30% maternal cells, at most about 25% maternal cells, at most about 20% maternal cells, at most about 15% maternal cells, at most about 10% maternal cells, at most about 5% maternal cells, at most about 0% maternal cells, or less. In some embodiments, the single round of enrichment produces a population of cells that is composed of about 0% maternal cells, about 5% maternal cells, about 10% maternal cells, about 15% maternal cells, about 20% maternal cells, about 25% maternal cells, about 30% maternal cells, about 35% maternal cells, about 40% maternal cells, about 45% maternal cells, about 50% maternal cells, about 55% maternal cells, about 60% maternal cells, about 65% maternal cells, about 70% maternal cells, about 75% maternal cells, about 80% maternal cells, about 85% maternal cells, about 90% maternal cells, about 95% maternal cells, or about 100% maternal cells.

In some embodiments, the single round of enrichment produces a population of cells that is composed of at least about 30% maternal cells. In some embodiments, the single round of enrichment produces a population of cells that is composed of at least about 30% maternal cells, at least about 35% maternal cells, at least about 40% maternal cells, at least about 45% maternal cells, at least about 50% maternal cells, at least about 55% maternal cells, at least about 60% maternal cells, at least about 65% maternal cells, at least about 70% maternal cells, at least about 75% maternal cells, at least about 80% maternal cells, at least about 85% maternal cells, at least about 90% maternal cells, at least about 95% maternal cells, at least about 100% maternal cells, or more. In some embodiments, the single round of enrichment produces a population of cells that is composed of about 30% maternal cells, about 35% maternal cells, about 40% maternal cells, about 45% maternal cells, about Pan50% maternal cells, about 55% maternal cells, about 60% maternal cells, about 65% maternal cells, about 70% maternal cells, about 75% maternal cells, about 80% maternal cells, about 85% maternal cells, about 90% maternal cells, about 95% maternal cells, or about 100% maternal cells.

In some embodiments the single round of enrichment produces a population of cells that is composed of at most about 70% fetal cells. In some embodiments, the single round of enrichment produces a population of cells that is composed of at most about 70% fetal cells, at most about 65% fetal cells, at most about 60% fetal cells, at most about 55% fetal cells, at most about 50% fetal cells, at most about 45% fetal cells, at most about 40% fetal cells, at most about 35% fetal cells, at most about 30% fetal cells, at most about 25% fetal cells, at most about 20% fetal cells, at most about 15% fetal cells, at most about 10% fetal cells, at most about 5% fetal cells, at most about 0% fetal cells, or less. In some embodiments, the single round of enrichment produces a population of cells that is composed of about 0% fetal cells, about 5% fetal cells, about 10% fetal cells, about 15% fetal cells, about 20% fetal cells, about 25% fetal cells, about 30% fetal cells, about 35% fetal cells, about 40% fetal cells, about 45% fetal cells, about 50% fetal cells, about 55% fetal cells, about 60% fetal cells, about 65% fetal cells, about 70% fetal cells, or more fetal cells.

In some embodiments, the level that provides the quantity of fetal nucleic acid that is sufficient for characterization as fetal nucleic acid is from about 25 nanograms (ng) of nucleic acid to about 500 ng of nucleic acid. In some embodiments, the level of fetal nucleic acid is from about 25 ng to about 50 ng, from about 50 ng to about 100 ng, from about 100 ng to about 150 ng, from about 150 ng to about 200 ng, from about 200 ng to about 250 ng, from about 250 ng to about 300 ng, from about 300 ng to about 350 ng, from about 350 ng to about 400 ng, from about 400 ng to about 450 ng, or from about 450 ng to about 500 ng.

In some embodiments, the level of fetal nucleic acid is at least about 25 ng, at least about 30 ng, at least about 35 ng, at least about 40 ng, at least about 45 ng, at least about 50 ng, at least about 55 ng, at least about 60 ng, at least about 65 ng, at least about 70 ng, at least about 75 ng, at least about 80 ng, at least about 85 ng, at least about 90 ng, at least about 95 ng, at least about 100 ng, at least about 110 ng, at least about 120 ng, at least about 130 ng, at least about 140 ng, at least about 150 ng, at least about 160 ng, at least about 170 ng, at least about 180 ng, at least about 190 ng, at least about 200 ng, at least about 210 ng, at least about 220 ng, at least about 230 ng, at least about 240 ng, at least about 250 ng, at least about 260 ng, at least about 270 ng, at least about 280 ng, at least about 290 ng, at least about 300 ng, at least about 310 ng, at least about 320 ng, at least about 330 ng, at least about 340 ng, at least about 350 ng, at least about 360, at least about 370 ng, at least about 380 ng, at least about 390 ng, at least about 400 ng, at least about 410 ng, at least about 420 ng, at least about 430 ng, at least about 440 ng, at least about 450 ng, at least about 460 ng, at least about 470 ng, at least about 480 ng, at least about 490 ng, at least about 500 ng, or more.

In some embodiments, the level of fetal nucleic acid is at most about 500 ng, at most about 490 ng, at most about 480 ng, at most about 470 ng, at most about 460 ng, at most about 450 ng, at most about 440 ng, at most about 430 ng, at most about 420 ng, at most about 410 ng, at most about 400 ng, at most about 390 ng, at most about 380 ng, at most about 370 ng, at most about 360 ng, at most about 350 ng, at most about 340 ng, at most about 330 ng, at most about 320 ng, at most about 310 ng, at most about 300 ng, at most about 290 ng, at most about 280 ng, at most about 270 ng, at most about 260 ng, at most about 250 ng, at most about 240 ng, at most about 230 ng, at most about 220 ng, at most about 210 ng, at most about 200 ng, at most about 190 ng, at most about 180 ng, at most about 170 ng, at most about 160 ng, at most about 150 ng, at most about 140 ng, at most about 130 ng, at most about 120 ng, at most about 110 ng, at most about 100 ng, at most about 95 ng, at most about 90 ng, at most about 85 ng, at most about 80 ng, at most about 75 ng, at most about 70 ng, at most about 65 ng, at most about 60 ng, at most about 55 ng, at most about 50 ng, at most about 45 ng, at most about 40 ng, at most about 35 ng, at most about 30 ng, at most about 25 ng, or less.

In some embodiments, the level of fetal nucleic acid is about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 110 ng, about 120 ng, about 130 ng, about 140 ng, about 150 ng, about 160 ng, about 170 ng, about 180 ng, about 190 ng, about 200 ng, about 210 ng, about 220 ng, about 230 ng, about 240 ng, about 250 ng, about 260 ng, about 270 ng, about 280 ng, about 290 ng, about 300 ng, about 310 ng, about 320 ng, about 330 ng, about 340 ng, about 350 ng, about 360 ng, about 370 ng, about 380 ng, about 390 ng, about 400 ng, about 410 ng, about 420 ng, about 430 ng, about 440 ng, about 450 ng, about 460 ng, about 470 ng, about 480 ng, about 490 ng, or about 500 ng.

In some embodiments, the level that provides the quantity of fetal nucleic acid that is sufficient for characterization as fetal nucleic acid rather than maternal nucleic acid is at least about 50% of fetal cells of the enriched sample. In some embodiments, the level that provides the quantity of fetal nucleic acid is at least about 50%, at least about 55%, at least about 60%, at least about 65%, at least about 70%, at least about 75%, at least about 80%, at least about 85%, at least about 90%, at least about 95%, or about 100% of fetal cells of the enriched sample. In some embodiments, the level that provides the quantity of fetal nucleic acid is about 50%, about 55%, about 60%, about 65%, about 70%, about 75%, about 80%, about 85%, about 90%, about 95%, or about 100% of fetal cells of the enriched sample.

In some embodiments, the fetal nucleic acid is characterized by the presence of an allele not found in the maternal nucleic acid. In some embodiments, the fetal nucleic acid is characterized by the absence of an allele found in the maternal nucleic acid. In some embodiments, the fetal nucleic acid is characterized by the presence of an allele not found in the maternal nucleic acid and by the absence of an allele found in the maternal nucleic acid.

In some embodiments, the method further comprising depleting red blood cells from the ex vivo population of cells. In some embodiments, red blood cells (RBCs) are depleted from the ex vivo population of cells via ammonium chloride lysis, density gradient centrifugation, hypotonic lysis, immunomagnetic cell separation, sedimentation, or any combination thereof.

In some embodiments, the method comprises performing about one round of enrichment. In some embodiments, the method comprises performing no greater than one round of enrichment. In some embodiments, the method comprises performing one and no greater than about one round of enrichment. In some embodiments, the method comprises performing from one round of enrichment to five rounds of enrichment. In some embodiments, the method comprises performing at least one round of enrichment, at least about two rounds of enrichment, at least about three rounds of enrichment, at least about four rounds of enrichment, at least about five rounds of enrichment, or more. In some embodiments, the method comprises performing at most about five rounds of enrichment, about four rounds of enrichment, about three rounds of enrichment, about two rounds of enrichments, about two rounds of enrichment, or less. In some embodiments, the method comprises performing about one round of enrichment, about two rounds of enrichment, about three rounds of enrichment, about four rounds of enrichment, or about five rounds of enrichment.

In some embodiments, one or more rounds of enrichment is positive selection. In some embodiments, the positive selection involves isolation and/or purification of cells with one or more isolation agents coupled to one or more biomarkers. In some embodiments, the one or more isolation agents are coupled to one or more fetal biomarkers. In some examples, the positive selection involves the isolation and/or purification of fetal cells. In some embodiments, the one or more isolation agents are coupled to one or more maternal biomarkers. In some examples, the positive selection involves the isolation and/or purification of maternal cells. In some embodiments, one or more rounds of enrichment is negative selection. In some embodiments, the negative selection involves isolation and/or purification of cells with an absence of one or more isolation agents coupled to one or more biomarkers. In some embodiments, the one or more isolation agents are coupled to one or more fetal biomarkers. In some examples, the negative selection involves the isolation and/or purification of maternal cells. In some embodiments, the one or more isolation agents are coupled to one or more maternal biomarkers. In some examples, the negative selection involves the isolation and/or purification of fetal cells. In some embodiments, the one or more rounds of enrichment are negative selection and positive selection. In some embodiments, the one or more isolation agents are coupled to one or more fetal biomarkers in a first round of enrichment and then subsequently another one or more isolation agents are coupled to one or more maternal biomarkers in a second round of enrichment. In some embodiments, the first round of enrichment is positive selection for isolation and/or purification of fetal cells and the second round of enrichment is negative selection for isolation and/or purification of fetal cells. In some embodiments, the first round of enrichment is negative selection for the isolation and/or purification of maternal cells and the second round of enrichment is positive selection for the isolation and/or purification of maternal cells. In some embodiments, the one or more isolation agents are coupled to one or more maternal biomarkers in a first round of enrichment and then subsequently another one or more isolation agents are coupled to one or more fetal biomarkers in a second round of enrichment. In some examples, the first round of enrichment is positive selection for isolation and/or purification of maternal cells and the second round of enrichment is negative selection for isolation and/or purification of maternal cells. In some examples, the first round of enrichment is negative selection for the isolation and/or purification of fetal cells and the second round of enrichment is positive selection for the isolation and/or purification of fetal cells. Tables 1-5 depicts possible biomarkers or biomarker combinations used in enrichment protocols to isolate different fetal cell types.

TABLE 1
Biomarkers and Biomarker combination to enrich
CFCs from maternal biological samples.

| | | % yield_All | % purity_All |
|---|---|---|---|
| Biomarker Set #1 | Biomarker Set #2 | CFC | CFC |
| Krt18 + Prl3d1 + Sct + Tfpi | H19 + Hspb1 + Prl3b1 + Sct | 83.46 | 98.23 |
| Krt18 + Prl3d1 + Sct + Tfpi | H19 + Hspb1 + Rhox9 + Sct | 83.46 | 98.23 |
| Krt18 + Prl3d1 + Sct + Tfpi | Ctsj + H19 + Hspb1 + Sct | 83.46 | 98.23 |
| Krt18 + Prl3d1 + Sct + Tfpi | H19 + Hspb1 + Krt8 + Sct | 83.46 | 98.23 |
| Krt18 + Prl3d1 + Sct + Tfpi | Fthl17a + H19 + Hspb1 + Sct | 83.46 | 98.23 |
| Krt18 + Prl3d1 + Sct + Tfpi | H19 + Hspb1 + Sct + Sparc | 83.46 | 98.23 |
| Krt18 + Prl3d1 + Sct + Tfpi | H19 + Hspb1 + Krt19 + Sct | 83.46 | 98.23 |
| Krt18 + Krt19 + Sct + Tfpi | H19 + Hspb1 + Prl3b1 + Sct | 83.46 | 98.23 |
| Krt18 + Krt19 + Sct + Tfpi | H19 + Hspb1 + Sct + Sparc | 83.46 | 98.23 |
| Krt18 + Krt19 + Sct + Tfpi | H19 + Hspb1 + Prl3d1 + Sct | 83.46 | 98.23 |
| Krt18 + Krt19 + Sct + Tfpi | Ctsj + H19 + Hspb1 + Sct | 83.46 | 98.23 |
| Krt18 + Krt19 + Sct + Tfpi | H19 + Hspb1 + Krt8 + Sct | 83.46 | 98.23 |
| Krt18 + Krt19 + Sct + Tfpi | H19 + Hspb1 + Rhox9 + Sct | 83.46 | 98.23 |
| Krt18 + Krt19 + Sct + Tfpi | Fthl17a + H19 + Hspb1 + Sct | 83.46 | 98.23 |
| Krt18 + Krt19 + Sct + Tfpi | H19 + Hspb1 + Krt19 + Sct | 83.46 | 98.23 |
| Krt18 + Krt19 + Sct + Tfpi | H19 + Hspb1 + Sct | 83.46 | 98.23 |
| Krt18 + Prl3d1 + Sct + Tfpi | H19 + Hspb1 + Sct | 83.46 | 98.23 |
| Krt18 + Krt19 + Sct + Tfpi | H19 + Hspb1 + Krt7 | 83.46 | 99.11 |
| Krt18 + Prl3d1 + Sct + Tfpi | H19 + Hspb1 + Krt7 | 83.46 | 99.11 |
| Krt18 + Krt19 + Sct + Tfpi | Cryab + H19 + Rhox9 | 83.46 | 100.00 |
| Krt18 + Krt19 + Sct + Tfpi | H19 + Rhox9 + Sbsn | 83.46 | 100.00 |
| Krt18 + Krt19 + Sct + Tfpi | H19 + Prl8a2 + Rhox9 | 83.46 | 100.00 |
| Krt18 + Krt19 + Sct + Tfpi | Gpx3 + H19 + Rhox9 | 83.46 | 100.00 |
| Krt18 + Krt19 + Sct + Tfpi | Dmkn + H19 + Rhox9 | 83.46 | 100.00 |
| Krt18 + Prl3d1 + Sct + Tfpi | Gpx3 + H19 + Rhox9 | 83.46 | 100.00 |
| Cdh5 + Sct + Tfpi | Gm7120 + Hspb1 + Sct | 80.45 | 98.17 |
| Map1b + Sct + Tfpi | Cdh5 + Hspb1 + Sct | 80.45 | 98.17 |
| Cdh5 + Sct + Tfpi | Hspb1 + Map1b + Sct | 80.45 | 98.17 |
| Fthl17a + Sct + Tfpi | Hspb1 + Plac1 + Sct | 79.70 | 98.15 |
| Sct + Slc38a4 + Tfpi | Gm7120 + Hspb1 + Sct | 79.70 | 98.15 |
| Hspg2 + Sct + Tfpi | Gm7120 + Hspb1 + Sct | 79.70 | 98.15 |
| Plac1 + Sct + Tfpi | Gm7120 + Hspb1 + Sct | 79.70 | 98.15 |
| Lepr + Sct + Tfpi | Gm7120 + Hspb1 + Sct | 79.70 | 98.15 |
| Fnd3c2 + Sct + Tfpi | Cdh5 + Hspb1 + Sct | 79.70 | 98.15 |
| Gm7120 + Sct + Tfpi | Cdh5 + Hspb1 + Sct | 79.70 | 98.15 |
| Igf2bp1 + Sct + Tfpi | Cdh5 + Hspb1 + Sct | 79.70 | 98.15 |
| Crip2 + Sct + Tfpi | Cdh5 + Hspb1 + Sct | 79.70 | 98.15 |
| Col4a1 + Sct + Tfpi | Cdh5 + Hspb1 + Sct | 79.70 | 98.15 |
| Lamb2 + Sct + Tfpi | Cdh5 + Hspb1 + Sct | 79.70 | 98.15 |
| Adgrf5 + Sct + Tfpi | Gm7120 + Hspb1 + Sct | 79.70 | 98.15 |
| Mt2 + Sct + Tfpi | Hspb1 + Plac1 + Sct | 79.70 | 98.15 |
| Rbp1 + Sct + Tfpi | Hspb1 + Plac1 + Sct | 79.70 | 98.15 |
| Cryab + Sct + Tfpi | Gm7120 + Hspb1 + Sct | 79.70 | 98.15 |
| Pfpl + Sct + Tfpi | Hspb1 + Plac1 + Sct | 79.70 | 98.15 |
| Plac1 + Sct + Tfpi | Hspb1 + Plac1 + Sct | 79.70 | 98.15 |
| Procr + Sct + Tfpi | Gm7120 + Hspb1 + Sct | 79.70 | 98.15 |
| Fthl17a + Sct + Tfpi | Cdh5 + Hspb1 + Sct | 79.70 | 98.15 |
| Cdh5 + Hspb1 + Sct | Cdh5 + Fthl17a + Sct | 79.70 | 98.15 |
| Mt2 + Sct + Tfpi | Cryab + Hspb1 + Sct | 79.70 | 98.15 |
| Cdh5 + Fthl17a + Sct | — | 79.70 | 98.15 |
| Dmkn + Sct + Tfpi | Cryab + Hspb1 + Sct | 79.70 | 98.15 |
| Fthl17a + Sct + Tfpi | Cryab + Hspb1 + Sct | 79.70 | 98.15 |
| Dmkn + Sct + Tfpi | Cdh5 + Hspb1 + Sct | 79.70 | 98.15 |
| Rbp1 + Sct + Tfpi | Cryab + Hspb1 + Sct | 79.70 | 98.15 |
| Dmkn + Sct + Tfpi | Hspb1 + Plac1 + Sct | 79.70 | 98.15 |
| Rbp1 + Sct + Tfpi | Cdh5 + Hspb1 + Sct | 79.70 | 98.15 |
| Plet1 + Sct + Tfpi | Gm7120 + Hspb1 + Sct | 79.70 | 98.15 |
| Mt2 + Sct + Tfpi | Cdh5 + Hspb1 + Sct | 79.70 | 98.15 |
| Cdh5 + Sct + Tfpi | Fnd3c2 + Hspb1 + Sct | 79.70 | 98.15 |
| Sct + Sdc1 + Tfpi | Gm7120 + Hspb1 + Sct | 79.70 | 98.15 |
| Igf2 + Sct + Tfpi | Cdh5 + Hspb1 + Sct | 79.70 | 98.15 |
| Sct + Tfpi + Zfp36l3 | Hspb1 + Plac1 + Sct | 78.95 | 98.13 |
| Igf2bp1 + Sct + Tfpi | Hspb1 + Plac1 + Sct | 78.95 | 98.13 |
| Fnd3c2 + Sct + Tfpi | Hspb1 + Plac1 + Sct | 78.95 | 98.13 |
| Cdh5 + Sct + Tfpi | Cdh5 + Sct + Sparc | 78.95 | 98.13 |
| Map1b + Sct + Tfpi | Cdh5 + Sct + Sparc | 78.95 | 99.06 |
| Cdh5 + Hspb1 + Sct | Cdh5 + Sct + Sparc | 78.95 | 98.13 |
| Mt2 + Sct + Tfpi | Cryab + Fthl17a + Sct | 78.95 | 99.06 |
| Rbp1 + Sct + Tfpi | Cryab + Fthl17a + Sct | 78.95 | 99.06 |
| Fthl17a + Sct + Tfpi | Cryab + Fthl17a + Sct | 78.95 | 99.06 |
| Cdh5 + Sct + Tfpi | Hspb1 + Sbsn + Sct | 78.95 | 98.13 |
| Lepr + Sct + Tfpi | Hspb1 + Sbsn + Sct | 78.95 | 98.13 |
| Map1b + Sct + Tfpi | Hspb1 + Plac1 + Sct | 78.95 | 98.13 |
| Hspb1 + Sct + Tfpi | Cryab + Fthl17a + Sct | 78.95 | 99.06 |
| Dusp9 + Sct + Tfpi | Hspb1 + Plac1 + Sct | 78.95 | 98.13 |
| Lama5 + Sct + Tfpi | Hspb1 + Plac1 + Sct | 78.95 | 98.13 |
| Procr + Sct + Tfpi | Hspb1 + Sbsn + Sct | 78.95 | 98.13 |
| Cryab + Sct + Tfpi | Hspb1 + Sbsn + Sct | 78.95 | 98.13 |
| Sct + Sdc1 + Tfpi | Hspb1 + Sbsn + Sct | 78.95 | 98.13 |
| Cdh3 + Sct + Tfpi | Hspb1 + Plac1 + Sct | 78.95 | 98.13 |
| Map1b + Sct + Tfpi | Cdh5 + Fthl17a + Sct | 78.95 | 99.06 |
| Sct + Tfpi | Hspb1 + Plac1 + Sct | 78.95 | 98.13 |
| Rarres2 + Sct + Tfpi | Hspb1 + Plac1 + Sct | 78.95 | 98.13 |
| Cdh5 + Sct + Sparc | Cdh5 + Fthl17a + Sct | 78.95 | 98.13 |
| Cited1 + Sct + Tfpi | Hspb1 + Plac1 + Sct | 78.95 | 98.13 |
| Sct + Serpinb9c + Tfpi | Hspb1 + Plac1 + Sct | 78.95 | 98.13 |
| Cdh5 + Sct + Tfpi | Cdh5 + Sct | 78.95 | 98.13 |
| Map1b + Sct + Tfpi | Cdh5 + Sct | 78.95 | 99.06 |
| Cdh5 + Hspb1 + Sct | Cdh5 + Sct | 78.95 | 98.13 |
| Nppc + Sct + Tfpi | Hspb1 + Plac1 + Sct | 78.95 | 98.13 |
| Cdh5 + Sct + Sparc | Cdh5 + Sct | 78.95 | 98.13 |
| Sct + Serpinb9e + Tfpi | Hspb1 + Plac1 + Sct | 78.95 | 98.13 |
| Sct + Slc30a2 + Tfpi | Hspb1 + Plac1 + Sct | 78.95 | 98.13 |
| Fermt2 + Sct + Tfpi | Hspb1 + Plac1 + Sct | 78.95 | 98.13 |
| Cdh5 + Fthl17a + Sct | Cdh5 + Sct | 78.95 | 98.13 |
| Cdh5 + Sct | — | 78.95 | 98.13 |
| Sct + Slc38a4 + Tfpi | Hspb1 + Plac1 + Sct | 78.95 | 98.13 |
| Sct + Tfap2c + Tfpi | Hspb1 + Plac1 + Sct | 78.95 | 98.13 |
| Sct + Tfpi + Tpbpa | Hspb1 + Plac1 + Sct | 78.95 | 98.13 |
| Nrk + Sct + Tfpi | Hspb1 + Plac1 + Sct | 78.95 | 98.13 |
| Cyp17a1 + Sct + Tfpi | Hspb1 + Plac1 + Sct | 78.95 | 98.13 |
| Adgrf5 + Sct + Tfpi | Hspb1 + Plac1 + Sct | 78.95 | 98.13 |
| Sct + Serpinb9g + Tfpi | Hspb1 + Plac1 + Sct | 78.95 | 98.13 |
| Hand1 + Sct + Tfpi | Hspb1 + Plac1 + Sct | 78.95 | 98.13 |
| Nup62cl + Sct + Tfpi | Hspb1 + Plac1 + Sct | 78.95 | 98.13 |
| Sct + Shroom1 + Tfpi | Hspb1 + Plac1 + Sct | 78.95 | 98.13 |
| Parva + Sct + Tfpi | Hspb1 + Plac1 + Sct | 78.95 | 98.13 |
| Nxf7 + Sct + Tfpi | Hspb1 + Plac1 + Sct | 78.95 | 98.13 |
| Htra1 + Sct + Tfpi | Hspb1 + Plac1 + Sct | 78.95 | 98.13 |
| Sct + Tfpi + Trap1a | Hspb1 + Plac1 + Sct | 78.95 | 98.13 |
| Hspg2 + Sct + Tfpi | Hspb1 + Plac1 + Sct | 78.95 | 98.13 |
| Igf2 + Sct + Tfpi | Hspb1 + Plac1 + Sct | 78.95 | 98.13 |
| Plet1 + Sct + Tfpi | Hspb1 + Plac1 + Sct | 78.95 | 98.13 |
| Pramef12 + Sct + Tfpi | Hspb1 + Plac1 + Sct | 78.95 | 98.13 |
| Sct + Sult1e1 + Tfpi | Hspb1 + Plac1 + Sct | 78.95 | 98.13 |
| 1700011M02Rik + Sct + Tfpi | Hspb1 + Plac1 + Sct | 78.95 | 98.13 |
| Sct + Serpinb9f + Tfpi | Hspb1 + Plac1 + Sct | 78.95 | 98.13 |
| Cdh5 + Sct + Tfpi | Hspb1 + Plac1 + Sct | 78.95 | 98.13 |
| Plet1 + Sct + Tfpi | Hspb1 + Sbsn + Sct | 78.95 | 98.13 |
| Cdh5 + Sct + Tfpi | Cdh5 + Fthl17a + Sct | 78.95 | 98.13 |
| Cryab + Fthl17a + Sct | — | 78.95 | 99.06 |
| Cdh5 + Hspb1 + Sct | Cryab + Fthl17a + Sct | 78.95 | 99.06 |
| Cryab + Hspb1 + Sct | Cryab + Fthl17a + Sct | 78.95 | 99.06 |
| Pawr + Sct + Tfpi | Hspb1 + Plac1 + Sct | 78.95 | 98.13 |
| Crip2 + Hspb1 + Sct | Cdh5 + Sct + Tfpi | 78.95 | 98.13 |

TABLE 2
Biomarkers and Biomarker combination to enrich Early
Trophoblasts from maternal biological samples.

| | | % yield_Early | % purity_Early |
|---|---|---|---|
| Biomarker Set #1 | Biomarker Set #2 | Trophoblast | Trophoblast |
| Dst + H19 + Map1b | Prl3d1 + Prl5a1 | 71.93 | 91.11 |
| Gm7120 + H19 + Dst | Prl3d1 + Prl5a1 | 71.93 | 91.11 |
| H19 + Map1b + Rere | Prl3d1 + Prl5a1 | 71.93 | 91.11 |
| Gm7120 + H19 + Rere | Prl3d1 + Prl5a1 | 70.18 | 90.91 |
| Prl3d1 + Prl5a1 | H19 | 70.18 | 90.91 |
| Prl3d1 + Prl5a1 | H19 + Krt14 | 70.18 | 90.91 |
| Prl3d1 + Prl5a1 | 1700011M02Rik + H19 | 70.18 | 90.91 |
| Prl3d1 + Prl5a1 | Cdh3 + H19 | 70.18 | 90.91 |
| Prl3d1 + Prl5a1 | Fnd3c2 + H19 | 70.18 | 90.91 |
| Prl3d1 + Prl5a1 | 3830417A13Rik + H19 | 70.18 | 90.91 |
| Prl3d1 + Prl5a1 | H19 + Prl3d2 | 70.18 | 90.91 |
| Prl3d1 + Prl5a1 | Gm9112 + H19 | 70.18 | 90.91 |
| Prl3d1 + Prl5a1 | H19 + Ldoc1 | 70.18 | 90.91 |
| Prl3d1 + Prl5a1 | H19 + Igf2bp1 | 70.18 | 90.91 |
| Prl3d1 + Prl5a1 | H19 + Pramef12 | 70.18 | 90.91 |
| Prl3d1 + Prl5a1 | H19 + Plac1 | 70.18 | 90.91 |
| Prl3d1 + Prl5a1 | Adgrf5 + H19 | 70.18 | 90.91 |
| Prl3d1 + Prl5a1 | H19 + Slc38a4 | 70.18 | 90.91 |
| Prl3d1 + Prl5a1 | H19 + Psg29 | 70.18 | 90.91 |
| Prl3d1 + Prl5a1 | H19 + Prl3d3 | 70.18 | 90.91 |
| Krt18 + Krt7 | Prl3d1 + Psg22 | 70.18 | 90.91 |
| Prl3d1 + Prl5a1 | H19 + Prl8a1 | 70.18 | 90.91 |
| Prl3d1 + Prl5a1 | H19 + Psg22 | 70.18 | 90.91 |
| Prl3d1 + Prl5a1 | H19 + Parva | 70.18 | 90.91 |
| Prl3d1 + Prl5a1 | H19 + Slc30a2 | 70.18 | 90.91 |
| Prl3d1 + Prl5a1 | Ghrh + H19 | 70.18 | 90.91 |
| Prl3d1 + Prl5al | H19 + Mmp1a | 70.18 | 90.91 |
| Prl3d1 + Prl5a1 | H19 + Hsd3b6 | 70.18 | 90.91 |
| H19 + Prl8a2 | Prl3d1 + Prl5a1 | 70.18 | 90.91 |
| H19 + Prl7a1 | Prl3d1 + Prl5a1 | 70.18 | 90.91 |
| H19 + Rbp1 | Prl3d1 + Prl5al | 70.18 | 90.91 |
| H19 + Serpine1 | Prl3d1 + Prl5a1 | 70.18 | 90.91 |
| H19 + Procr | Prl3d1 + Prl5a1 | 70.18 | 90.91 |
| H19 + Serpinb9b | Prl3d1 + Prl5a1 | 70.18 | 90.91 |
| H19 + Sdc1 | Prl3d1 + Prl5a1 | 70.18 | 90.91 |
| H19 + Prnp | Prl3d1 + Prl5a1 | 70.18 | 90.91 |
| Fscn1 + H19 | Prl3d1 + Prl5a1 | 70.18 | 90.91 |
| H19 + Gm26870 | Prl3d1 + Prl5a1 | 70.18 | 90.91 |
| H19 + Rere | Prl3d1 + Prl5a1 | 70.18 | 90.91 |
| Prl3d1 + Psg22 | Krt18 + Krt7 | 70.18 | 90.91 |
| Krt18 + Krt7 | Prl3d1 + Prl5al | 70.18 | 90.91 |
| Fthl17a + H19 + Krt14 | Prl3d1 + Prl3d2 | 68.42 | 90.70 |
| Cdh3 + Fthl17a + H19 | Prl3d1 + Prl3d2 | 68.42 | 90.70 |
| Fnd3c2 + Fthl17a + H19 | Prl3d1 + Prl3d2 | 68.42 | 90.70 |
| 1700011M02Rik + Fthl17a + H19 | Prl3d1 + Prl3d2 | 68.42 | 90.70 |
| Fthl17a + H19 + Prl3d2 | Prl3d1 + Prl3d2 | 68.42 | 90.70 |
| Fthl17a + Gm9112 + H19 | Prl3d1 + Prl3d2 | 68.42 | 90.70 |
| Fthl17a + H19 + Ldoc1 | Prl3d1 + Prl3d2 | 68.42 | 90.70 |
| 3830417A13Rik + Fthl17a + H19 | Prl3d1 + Prl3d2 | 68.42 | 90.70 |
| Fthl17a + H19 + Serpinb9f | Prl3d1 + Prl3d2 | 68.42 | 90.70 |
| Fthl17a + H19 + Igf2bp1 | Prl3d1 + Prl3d2 | 68.42 | 90.70 |
| Fthl17a + H19 + Sult1e1 | Prl3d1 + Prl3d2 | 68.42 | 90.70 |
| Fthl17a + H19 + Pramef12 | Prl3d1 + Prl3d2 | 68.42 | 90.70 |
| Fthl17a + H19 + Zfp36l3 | Prl3d1 + Prl3d2 | 68.42 | 90.70 |
| Fthl17a + H19 + Rhox5 | Prl3d1 + Prl3d2 | 68.42 | 90.70 |
| Fthl17a + H19 + Rhox12 | Prl3d1 + Prl3d2 | 68.42 | 90.70 |
| Fthl17a + H19 + Plac1 | Prl3d1 + Prl3d2 | 68.42 | 90.70 |
| 1600025M17Rik + Fthl17a + H19 | Prl3d1 + Prl3d2 | 68.42 | 90.70 |
| Fthl17a + H19 + Serpinb9g | Prl3d1 + Prl3d2 | 68.42 | 90.70 |
| Fthl17a + H19 + Prl2c1 | Prl3d1 + Prl3d2 | 68.42 | 90.70 |
| Adgrf5 + Fthl17a + H19 | Prl3d1 + Prl3d2 | 68.42 | 90.70 |
| Fthl17a + H19 + Slc38a4 | Prl3d1 + Prl3d2 | 68.42 | 90.70 |
| Fthl17a + H19 + Tpbpa | Prl3d1 + Prl3d2 | 68.42 | 90.70 |
| Fthl17a + H19 + Psg29 | Prl3d1 + Prl3d2 | 68.42 | 90.70 |
| Rrm2 + Cryab + Htra1 | Adgrf5 + Trap1a | 35.09 | 90.91 |
| Rrm2 + Cryab + Htra1 | Plac1 + Trap1a | 35.09 | 90.91 |
| Rrm2 + Cryab + Htra1 | Nrk + Plet1 | 35.09 | 90.91 |
| Rrm2 + Cryab + Htra1 | Fthl17a + Plet1 | 33.33 | 90.48 |
| Rrm2 + Cryab + Des | Adgrf5 + Trap1a | 31.58 | 94.74 |
| Rrm2 + Cryab + Mfap5 | Adgrf5 + Trap1a | 31.58 | 94.74 |
| Rrm2 + Cryab + Dmkn | Adgrf5 + Trap1a | 31.58 | 94.74 |
| Rrm2 + Cryab + Des | Plac1 + Trap1a | 31.58 | 90.00 |
| Rrm2 + Cryab + Mfap5 | Plac1 + Trap1a | 31.58 | 90.00 |
| Rrm2 + Cryab + Dmkn | Plac1 + Trap1a | 31.58 | 90.00 |
| Rrm2 + Cryab + Des | Nrk + Plet1 | 31.58 | 90.00 |
| Rrm2 + Cryab + Mfap5 | Nrk + Plet1 | 31.58 | 90.00 |
| Rrm2 + Cryab + Dmkn | Nrk + Plet1 | 31.58 | 90.00 |
| Ces1a + 1600015I10Rik | Plac1 + Sct + Tfpi | 28.07 | 94.12 |
| Ces1a + 1600015I10Rik | Adgrf5 + Sct + Tfpi | 28.07 | 94.12 |
| Ces1a + 1600015I10Rik | Sct + Slc38a4 + Tfpi | 28.07 | 94.12 |
| Ces1a + 1600015I10Rik | Hspg2 + Sct + Tfpi | 28.07 | 94.12 |
| Ces1a + 1600015I10Rik | Plet1 + Sct + Tfpi | 28.07 | 94.12 |
| Ces1a + 1600015I10Rik | Cdh5 + Sct + Tfpi | 28.07 | 94.12 |
| Ces1a + 1600015I10Rik | Lepr + Sct + Tfpi | 28.07 | 94.12 |
| Ces1a + 1600015I10Rik | Procr + Sct + Tfpi | 28.07 | 94.12 |
| Ces1a + 1600015I10Rik | Cryab + Sct + Tfpi | 28.07 | 94.12 |
| Ces1a + 1600015I10Rik | Sct + Sdc1 + Tfpi | 28.07 | 94.12 |
| Plac1 + Sct + Tfpi | Hsd3b6 | 26.32 | 93.75 |
| Adgrf5 + Sct + Tfpi | Hsd3b6 | 26.32 | 93.75 |
| Sct + Slc38a4 + Tfpi | Hsd3b6 | 26.32 | 93.75 |
| Hspg2 + Sct + Tfpi | Hsd3b6 | 26.32 | 93.75 |
| Plet1 + Sct + Tfpi | Hsd3b6 | 26.32 | 93.75 |
| Cdh5 + Sct + Tfpi | Hsd3b6 | 26.32 | 93.75 |
| Lepr + Sct + Tfpi | Hsd3b6 | 26.32 | 93.75 |
| Procr + Sct + Tfpi | Hsd3b6 | 26.32 | 93.75 |
| Cryab + Sct + Tfpi | Hsd3b6 | 26.32 | 93.75 |
| Sct + Sdc1 + Tfpi | Hsd3b6 | 26.32 | 93.75 |
| Ces1a + Chrdl2 | Plac1 + Sct + Tfpi | 26.32 | 93.75 |
| Chrdl2 + 1600015I10Rik | Plac1 + Sct + Tfpi | 26.32 | 93.75 |
| Ces1a + Chrdl2 | Adgrf5 + Sct + Tfpi | 26.32 | 93.75 |
| Chrdl2 + 1600015I10Rik | Adgrf5 + Sct + Tfpi | 26.32 | 93.75 |
| Ces1a + Chrdl2 | Sct + Slc38a4 + Tfpi | 26.32 | 93.75 |
| Chrdl2 + 1600015I10Rik | Sct + Slc38a4 + Tfpi | 26.32 | 93.75 |
| Ces1a + Chrdl2 | Hspg2 + Sct + Tfpi | 26.32 | 93.75 |
| Chrdl2 + 1600015I10Rik | Hspg2 + Sct + Tfpi | 26.32 | 93.75 |
| Ces1a + Chrdl2 | Plet1 + Sct + Tfpi | 26.32 | 93.75 |
| Chrdl2 + 1600015I10Rik | Plet1 + Sct + Tfpi | 26.32 | 93.75 |
| Ces1a + Chrdl2 | Cdh5 + Sct + Tfpi | 26.32 | 93.75 |
| Chrdl2 + 1600015I10Rik | Cdh5 + Sct + Tfpi | 26.32 | 93.75 |
| Ces1a + Chrdl2 | Lepr + Sct + Tfpi | 26.32 | 93.75 |
| Chrdl2 + 1600015110Rik | Lepr + Sct + Tfpi | 26.32 | 93.75 |
| Ces1a + Chrdl2 | Procr + Sct + Tfpi | 26.32 | 93.75 |
| Chrdl2 + 1600015110Rik | Procr + Sct + Tfpi | 26.32 | 93.75 |
| Ces1a + Chrdl2 | Cryab + Sct + Tfpi | 26.32 | 93.75 |
| Chrdl2 + 1600015I10Rik | Cryab + Sct + Tfpi | 26.32 | 93.75 |
| Ces1a + Chrdl2 | Sct + Sdc1 + Tfpi | 26.32 | 93.75 |
| Chrdl2 + 1600015I10Rik | Sct + Sdc1 + Tfpi | 26.32 | 93.75 |
| Ces1a + Chrdl2 | Tfpi + Cryab + Des | 26.32 | 93.75 |
| Ces1a + 1600015I10Rik | Tfpi + Cryab + Des | 26.32 | 93.75 |
| Ces1a + Chrdl2 | Tfpi + Cryab + Mfap5 | 26.32 | 93.75 |
| Ces1a + 1600015I10Rik | Tfpi + Cryab + Mfap5 | 26.32 | 93.75 |
| Ces1a + Chrdl2 | Tfpi + Cryab + Htra1 | 26.32 | 93.75 |
| Ces1a + 1600015I10Rik | Tfpi + Cryab + Htra1 | 26.32 | 93.75 |
| Ces1a + Chrdl2 | Tfpi + Cryab + Dmkn | 26.32 | 93.75 |
| Ces1a + 1600015I10Rik | Tfpi + Cryab + Dmkn | 26.32 | 93.75 |

TABLE 3
Biomarkers and Biomarker combination to enrich Late
Trophoblasts from maternal biological samples.

| | | % yield_Late | % purity_Late |
|---|---|---|---|
| Biomarker Set #1 | Biomarker Set #2 | Trophoblast | Trophoblast |
| Cts6 | Prl2b1 | 37.78 | 94.44 |
| Krt19 + Prl2b1 | Cts6 | 37.78 | 94.44 |
| Cited1 + Prl2b1 | Cts6 | 37.78 | 94.44 |
| Igf2 + Prl2b1 | Cts6 | 37.78 | 94.44 |
| Prl2b1 + Prl3d1 | Cts6 | 37.78 | 94.44 |
| Prl3d1 + Prl2b1 + Lepr | Cts6 | 37.78 | 94.44 |
| Prl2c2 + Prl2b1 + Lepr | Cts6 | 37.78 | 94.44 |
| Psg29 + Prl2b1 + Lepr | Cts6 | 37.78 | 94.44 |
| Sparc | Cts6 | 35.56 | 94.12 |
| Igf2 + Sparc | Cts6 | 35.56 | 94.12 |
| Cyp11a1 + Sparc | Cts6 | 35.56 | 94.12 |
| Krt7 + Sparc | Cts6 | 35.56 | 94.12 |
| Lepr | Cts6 | 31.11 | 93.33 |
| Hsd17b2 | Cts6 | 28.89 | 92.86 |
| Igf2 + Nrk | Cts6 | 28.89 | 92.86 |
| Igf2 + Procr | Cts6 | 28.89 | 92.86 |
| Nrk + Prl2c2 | Cts6 | 26.67 | 92.31 |
| Cited1 + Nrk | Cts6 | 26.67 | 92.31 |
| Igf2 + Krt19 | Cts6 | 26.67 | 92.31 |
| Cdh5 + Igf2 | Cts6 | 26.67 | 92.31 |
| Cts6 | Cited1 | 24.44 | 91.67 |
| Hand1 + Prl2c3 | 1700011M02Rik | 24.44 | 91.67 |
| Crip2 + Prl2c3 | 1700011M02Rik | 24.44 | 91.67 |
| Syngr1 | Cts6 | 24.44 | 91.67 |
| Cdh3 + Gm9112 | Cts6 | 24.44 | 91.67 |
| 1600025M17Rik + Gm9112 | Cts6 | 24.44 | 91.67 |
| Gm9112 + Nrk | Cts6 | 24.44 | 91.67 |
| 1700011M02Rik + Nrk | Cts6 | 24.44 | 91.67 |
| Gm9112 + Krt19 | Cts6 | 24.44 | 91.67 |
| 1600025M17Rik + Nrk | Cts6 | 24.44 | 91.67 |
| Gm9112 + Prl2c2 | Cts6 | 24.44 | 91.67 |
| Cited1 + Hand1 | Cts6 | 24.44 | 91.67 |
| Cited1 + Rhox5 | Cts6 | 24.44 | 91.67 |
| Cited1 + Fermt2 | Cts6 | 24.44 | 91.67 |
| Cited1 + Krt19 | Cts6 | 24.44 | 91.67 |
| Igf2 + Rhox5 | Cts6 | 24.44 | 91.67 |
| Cdh5 + Nrk | Cts6 | 24.44 | 91.67 |
| Krt19 + Procr | Cts6 | 24.44 | 91.67 |
| Procr | Cts6 | 22.22 | 90.91 |
| Gm9112 + Serpinb9d | Cts6 | 22.22 | 90.91 |
| Gm9112 + Serpinb9f | Cts6 | 22.22 | 90.91 |
| 3830417A13Rik + Gm9112 | Cts6 | 22.22 | 90.91 |
| Fnd3c2 + Gm9112 | Cts6 | 22.22 | 90.91 |
| Gm9112 + Pramef12 | Cts6 | 22.22 | 90.91 |
| Gm9112 + Hand1 | Cts6 | 22.22 | 90.91 |
| Gm9112 + Serpinb9g | Cts6 | 22.22 | 90.91 |
| Gm9112 + Nup62cl | Cts6 | 22.22 | 90.91 |
| Gm9112 + Prl2c1 | Cts6 | 22.22 | 90.91 |
| Gm9112 + Rhox5 | Cts6 | 22.22 | 90.91 |
| Fnd3c2 + Nrk | Cts6 | 22.22 | 90.91 |
| Nrk + Serpinb9f | Cts6 | 22.22 | 90.91 |
| Cdh3 + Nrk | Cts6 | 22.22 | 90.91 |
| Hand1 + Nrk | Cts6 | 22.22 | 90.91 |
| 1700011M02Rik + Krt19 | Cts6 | 22.22 | 90.91 |
| Nrk + Serpinb9g | Cts6 | 22.22 | 90.91 |
| Nrk + Rhox5 | Cts6 | 22.22 | 90.91 |
| Nrk + Slc38a4 | Cts6 | 22.22 | 90.91 |
| Krt19 + Slc38a4 | Cts6 | 22.22 | 90.91 |
| Krt19 + Nrk | Cts6 | 22.22 | 90.91 |
| Prl2c2 + Slc38a4 | Cts6 | 22.22 | 90.91 |
| Nrk + Plet1 | Cts6 | 22.22 | 90.91 |
| Nrk + Prl7a1 | Cts6 | 22.22 | 90.91 |
| Igf2 + Prl7a1 | Cts6 | 22.22 | 90.91 |
| Hand1 + Procr | Cts6 | 22.22 | 90.91 |
| Procr + Rhox5 | Cts6 | 22.22 | 90.91 |
| Nup62cl + Prl7d1 | Zfp36l3 | 20.00 | 100.00 |
| Crip2 | 1700011M02Rik | 20.00 | 100.00 |
| Ctsj + Nup62cl | Zfp36l3 | 20.00 | 90.00 |
| Ctsj + Sbsn | Zfp36l3 | 20.00 | 90.00 |
| Prpf4b | Fnd3c2 + Krt19 | 20.00 | 90.00 |
| Cts6 | Nrk | 20.00 | 90.00 |
| Cts6 | Gm9112 | 20.00 | 90.00 |
| Erdr1 + Gm7120 | 1700011M02Rik | 20.00 | 90.00 |
| Fnd3c2 + Fthl17a | Zfp36l3 | 20.00 | 90.00 |
| Fthl17a + Rhox12 | Zfp36l3 | 20.00 | 90.00 |
| Rhox12 + Rhox5 | Zfp36l3 | 20.00 | 90.00 |
| Adgrf5 + Fthl17a | Zfp36l3 | 20.00 | 90.00 |
| Fermt2 + Fthl17a | Zfp36l3 | 20.00 | 90.00 |
| Fthl17a + Prl7d1 | Zfp36l3 | 20.00 | 90.00 |
| Prpf4b | Rhox6 + Sparc | 20.00 | 90.00 |
| Prpf4b | Prl2c5 + Rhox6 + Sparc | 20.00 | 90.00 |
| Ctsl | Cts6 | 20.00 | 90.00 |
| Gm9112 + Prl3d2 | Cts6 | 20.00 | 90.00 |
| Gm9112 + Igf2bp1 | Cts6 | 20.00 | 90.00 |
| Gm9112 + Ldoc1 | Cts6 | 20.00 | 90.00 |
| Gm9112 + Sult1e1 | Cts6 | 20.00 | 90.00 |
| Gm9112 + Krt14 | Cts6 | 20.00 | 90.00 |
| 1700011M02Rik + Hand1 | Cts6 | 20.00 | 90.00 |
| 1700011M02Rik + Serpinb9g | Cts6 | 20.00 | 90.00 |
| 1600025M17Rik + 1700011M02Rik | Cts6 | 20.00 | 90.00 |
| 1700011M02Rik + Rhox5 | Cts6 | 20.00 | 90.00 |
| Nrk + Serpinb9d | Cts6 | 20.00 | 90.00 |
| Nrk + Prl3d2 | Cts6 | 20.00 | 90.00 |
| Ldoc1 + Nrk | Cts6 | 20.00 | 90.00 |
| Nrk + Sult1e1 | Cts6 | 20.00 | 90.00 |
| Krt14 + Nrk | Cts6 | 20.00 | 90.00 |
| 3830417A13Rik + Nrk | Cts6 | 20.00 | 90.00 |
| Cdh3 + Krt19 | Cts6 | 20.00 | 90.00 |
| Krt19 + Nup62cl | Cts6 | 20.00 | 90.00 |
| Adgrf5 + Nrk | Cts6 | 20.00 | 90.00 |
| Nrk + Plac1 | Cts6 | 20.00 | 90.00 |
| Nrk + Tpbpa | Cts6 | 20.00 | 90.00 |
| Nrk + Prl3d3 | Cts6 | 20.00 | 90.00 |
| Nrk + Tfap2c | Cts6 | 20.00 | 90.00 |
| Fermt2 + Nrk | Cts6 | 20.00 | 90.00 |
| Nrk + Serpinb9e | Cts6 | 20.00 | 90.00 |
| Nrk + Psg29 | Cts6 | 20.00 | 90.00 |
| Nrk + Parva | Cts6 | 20.00 | 90.00 |
| Nrk + Serpinb9c | Cts6 | 20.00 | 90.00 |
| 1700011M02Rik + Prl2c2 | Cts6 | 20.00 | 90.00 |
| Nrk + Prl8a9 | Cts6 | 20.00 | 90.00 |
| Nrk + Psg22 | Cts6 | 20.00 | 90.00 |
| Nrk + Slc30a2 | Cts6 | 20.00 | 90.00 |
| Hsd3b6 + Nrk | Cts6 | 20.00 | 90.00 |
| Nrk + Prl8a1 | Cts6 | 20.00 | 90.00 |
| Nrk + Nxf7 | Cts6 | 20.00 | 90.00 |
| Prl2c2 + Rhox5 | Cts6 | 20.00 | 90.00 |
| Nrk + Prl2a1 | Cts6 | 20.00 | 90.00 |
| Htra1 + Nrk | Cts6 | 20.00 | 90.00 |
| Krt19 + Prl2c2 | Cts6 | 20.00 | 90.00 |
| Nrk + Prl4a1 | Cts6 | 20.00 | 90.00 |
| Dusp9 + Nrk | Cts6 | 20.00 | 90.00 |
| Cdh5 + Krt19 | Cts6 | 20.00 | 90.00 |
| Dusp9 + Igf2 | Cts6 | 20.00 | 90.00 |
| Krt19 + Tinagl1 | Cts6 | 20.00 | 90.00 |

TABLE 4
Biomarkers and Biomarker combination to enrich
Trophoblasts from maternal biological samples.

| | | % | % |
|---|---|---|---|
| Biomarker Set #1 | Biomarker Set #2 | yield_Trophoblast | purity_Trophoblast |
| Map1b + Prl2c5 + Rhox9 | Nup62cl + Rhox9 + Sct | 95.10 | 97.00 |
| Map1b + Prl2c5 + Rhox9 | Ctsr + Rhox9 + Sct | 95.10 | 97.00 |
| Map1b + Prl2c5 + Rhox9 | Sct + Krt18 | 95.10 | 97.00 |
| Map1b + Prl2c5 + Rhox9 | Rhox9 + Sct + Zfp36l3 | 95.10 | 97.00 |
| Krt18 + Sct + Serpinb9d | Map1b + Prl2c5 + Rhox9 | 95.10 | 97.00 |
| Krt14 + Krt18 + Sct | Map1b + Prl2c5 + Rhox9 | 95.10 | 97.00 |
| Hand1 + Krt18 + Sct | Map1b + Prl2c5 + Rhox9 | 95.10 | 97.00 |
| Krt18 + Sct + Serpinb9g | Map1b + Prl2c5 + Rhox9 | 95.10 | 97.00 |
| Krt18 + Sct + Slc38a4 | Map1b + Prl2c5 + Rhox9 | 95.10 | 97.00 |
| Cyp17a1 + Krt18 + Sct | Map1b + Prl2c5 + Rhox9 | 95.10 | 97.00 |
| Krt18 + Nppc + Sct | Map1b + Prl2c5 + Rhox9 | 95.10 | 97.00 |
| Hsd3b6 + Krt18 + Sct | Map1b + Prl2c5 + Rhox9 | 95.10 | 97.00 |
| Krt18 + Prl7a1 + Sct | Map1b + Prl2c5 + Rhox9 | 95.10 | 97.00 |
| Krt18 + Prl3d1 + Sct | Map1b + Prl2c5 + Rhox9 | 95.10 | 97.00 |
| Krt18 + Rbp1 + Sct | Map1b + Prl2c5 + Rhox9 | 95.10 | 97.00 |
| Bcat1 + Krt18 + Sct | Map1b + Prl2c5 + Rhox9 | 95.10 | 97.00 |
| Krt18 + Prl3b1 + Sct | Map1b + Prl2c5 + Rhox9 | 95.10 | 97.00 |
| Ctsj + Fthl17a + Krt18 + Sct | Map1b + Prl2c5 + Rhox9 | 95.10 | 97.00 |
| Hsd17b2 + Krt18 + Sct | Map1b + Prl2c5 + Rhox9 | 95.10 | 97.00 |
| Krt7 + Rhox9 + Sct | Map1b + Prl2c5 + Rhox9 | 95.10 | 97.00 |
| Cdkn1c + Rhox9 + Sct | Map1b + Prl2c5 + Rhox9 | 95.10 | 97.00 |
| Cdkn1c + Krt19 + Sct | Map1b + Prl2c5 + Rhox9 | 95.10 | 97.00 |
| Cdkn1c + Krt8 + Sct | Map1b + Prl2c5 + Rhox9 | 95.10 | 97.00 |
| Krt18 + Sct + Syngr1 | Map1b + Prl2c5 + Rhox9 | 95.10 | 97.00 |
| Ctsj + Krt18 + Prl3d1 + Sct | Map1b + Prl2c5 + Rhox9 | 95.10 | 97.00 |
| Cdh5 + Sct + Tfpi | Igf2 + Trap1a | 79.41 | 96.43 |
| Sct + Sdc1 + Tfpi | Igf2 + Trap1a | 78.43 | 96.39 |
| Hspg2 + Sct + Tfpi | Igf2 + Trap1a | 78.43 | 96.39 |
| Lepr + Sct + Tfpi | Igf2 + Trap1a | 78.43 | 96.39 |
| Plac1 + Sct + Tfpi | Igf2 + Trap1a | 78.43 | 96.39 |
| Cryab + Sct + Tfpi | Igf2 + Trap1a | 78.43 | 96.39 |
| Procr + Sct + Tfpi | Igf2 + Trap1a | 78.43 | 96.39 |
| Plet1 + Sct + Tfpi | Igf2 + Trap1a | 78.43 | 96.39 |
| Adgrf5 + Sct + Tfpi | Igf2 + Trap1a | 78.43 | 96.39 |
| Sct + Slc38a4 + Tfpi | Igf2 + Trap1a | 78.43 | 96.39 |
| Cdh5 + Sct + Tfpi | Igf2 + Nrk | 74.51 | 96.20 |
| Sct + Sdc1 + Tfpi | Igf2 + Nrk | 73.53 | 96.15 |
| Plet1 + Sct + Tfpi | Igf2 + Nrk | 73.53 | 96.15 |
| Cdh5 + Sct + Tfpi | Fthl17a + Igf2 | 73.53 | 96.15 |
| Plac1 + Sct + Tfpi | Igf2 + Nrk | 73.53 | 96.15 |
| Adgrf5 + Sct + Tfpi | Igf2 + Nrk | 73.53 | 96.15 |
| Hspg2 + Sct + Tfpi | Igf2 + Nrk | 73.53 | 96.15 |
| Sct + Slc38a4 + Tfpi | Igf2 + Nrk | 73.53 | 96.15 |
| Cryab + Sct + Tfpi | Igf2 + Nrk | 73.53 | 96.15 |
| Procr + Sct + Tfpi | Igf2 + Nrk | 73.53 | 96.15 |
| Lepr + Sct + Tfpi | Igf2 + Nrk | 73.53 | 96.15 |
| Cryab + Sct + Tfpi | Nrk + Trap1a | 72.55 | 98.67 |
| Sct + Sdc1 + Tfpi | Fthl17a + Igf2 | 72.55 | 96.10 |
| Lepr + Sct + Tfpi | Nrk + Trap1a | 72.55 | 98.67 |
| Plet1 + Sct + Tfpi | Nrk + Trap1a | 72.55 | 98.67 |
| Sct + Slc38a4 + Tfpi | Nrk + Trap1a | 72.55 | 98.67 |
| Sct + Sdc1 + Tfpi | Nrk + Trap1a | 72.55 | 98.67 |
| Adgrf5 + Sct + Tfpi | Nrk + Trap1a | 72.55 | 98.67 |
| Cdh5 + Sct + Tfpi | Nrk + Trap1a | 72.55 | 98.67 |
| Procr + Sct + Tfpi | Nrk + Trap1a | 72.55 | 98.67 |
| Plac1 + Sct + Tfpi | Nrk + Trap1a | 72.55 | 98.67 |
| Procr + Sct + Tfpi | Fthl17a + Igf2 | 72.55 | 96.10 |
| Plet1 + Sct + Tfpi | Fthl17a + Igf2 | 72.55 | 96.10 |
| Hspg2 + Sct + Tfpi | Fthl17a + Igf2 | 72.55 | 96.10 |
| Hspg2 + Sct + Tfpi | Nrk + Trap1a | 72.55 | 98.67 |
| Cryab + Sct + Tfpi | Fthl17a + Igf2 | 72.55 | 96.10 |
| Sct + Slc38a4 + Tfpi | Fthl17a + Igf2 | 72.55 | 96.10 |
| Plac1 + Sct + Tfpi | Fthl17a + Igf2 | 72.55 | 96.10 |
| Lepr + Sct + Tfpi | Fthl17a + Igf2 | 72.55 | 96.10 |
| Adgrf5 + Sct + Tfpi | Fthl17a + Igf2 | 72.55 | 96.10 |
| Hspb1 + Lepr | Fthl17a + Procr + Sct | 71.57 | 97.33 |
| Procr + Sct + Tfpi | Fthl17a + Trap1a | 71.57 | 98.65 |
| Plac1 + Sct + Tfpi | Fthl17a + Trap1a | 71.57 | 98.65 |
| Hspb1 + Slc38a4 | Fthl17a + Procr + Sct | 71.57 | 97.33 |
| Hspb1 + Lepr | Fthl17a + Plet1 + Sct | 71.57 | 97.33 |
| Cdh5 + Hspb1 | Fthl17a + Plet1 + Sct | 71.57 | 97.33 |
| Sct + Sdc1 + Tfpi | Fthl17a + Trap1a | 71.57 | 98.65 |
| Cdh5 + Sct + Tfpi | Fthl17a + Trap1a | 71.57 | 98.65 |
| Plet1 + Sct + Tfpi | Fthl17a + Trap1a | 71.57 | 98.65 |
| Hspg2 + Sct + Tfpi | Fthl17a + Trap1a | 71.57 | 98.65 |
| Cdh5 + Hspb1 | Fthl17a + Procr + Sct | 71.57 | 97.33 |
| Cryab + Sct + Tfpi | Fthl17a + Trap1a | 71.57 | 98.65 |
| Lepr + Sct + Tfpi | Fthl17a + Trap1a | 71.57 | 98.65 |
| Hspb1 + Slc38a4 | Fthl17a + Plet1 + Sct | 71.57 | 97.33 |
| Sct + Slc38a4 + Tfpi | Fthl17a + Trap1a | 71.57 | 98.65 |
| Adgrf5 + Sct + Tfpi | Fthl17a + Trap1a | 71.57 | 98.65 |
| Hspb1 + Lepr | Adgrf5 + Fthl17a + Sct | 70.59 | 97.30 |
| Hspb1 + Slc38a4 | Fthl17a + Hspg2 + Sct | 70.59 | 97.30 |
| Cdh5 + Hspb1 | Adgrf5 + Fthl17a + Sct | 70.59 | 97.30 |
| Hspb1 + Slc38a4 | Adgrf5 + Fthl17a + Sct | 70.59 | 97.30 |
| Hspb1 + Lepr | Fthl17a + Plac1 + Sct | 70.59 | 97.30 |
| Cdh5 + Hspb1 | Fthl17a + Sct + Tnfrsf9 | 70.59 | 97.30 |
| Cdh5 + Hspb1 | Fthl17a + Plac1 + Sct | 70.59 | 97.30 |
| Hspb1 + Slc38a4 | Fthl17a + Sct + Sdc1 | 70.59 | 97.30 |
| Hspb1 + Lepr | Fthl17a + Sct + Tnfrsf9 | 70.59 | 97.30 |
| Hspb1 + Lepr | Fthl17a + Hspg2 + Sct | 70.59 | 97.30 |
| Hspb1 + Lepr | Fthl17a + Sct + Sdc1 | 70.59 | 97.30 |
| Cdh5 + Hspb1 | Fthl17a + Sct + Sdcl | 70.59 | 97.30 |
| Cdh5 + Hspb1 | Fthl17a + Hspg2 + Sct | 70.59 | 97.30 |
| Hspb1 + Slc38a4 | Fthl17a + Sct + Tnfrsf9 | 70.59 | 97.30 |
| Hspb1 + Lepr | Procr + Sct | 70.59 | 97.30 |
| Cdh5 + Hspb1 | Procr + Sct | 70.59 | 97.30 |
| Hspb1 + Slc38a4 | Procr + Sct | 70.59 | 97.30 |
| Hspb1 + Lepr | Plet1 + Sct | 70.59 | 97.30 |
| Cdh5 + Hspb1 | Plet1 + Sct | 70.59 | 97.30 |
| Hspb1 + Slc38a4 | Plet1 + Sct | 70.59 | 97.30 |
| Hspb1 + Slc38a4 | Fthl17a + Plac1 + Sct | 70.59 | 97.30 |
| Hspb1 + Slc38a4 | Sct + Tnfrsf9 | 69.61 | 97.26 |
| Hspb1 + Slc38a4 | Sct + Sdc1 | 69.61 | 97.26 |
| Adgrf5 + Sct + Tfpi | Hand1 + Trap1a | 69.61 | 98.61 |
| Cdh5 + Sct + Tfpi | Hand1 + Trap1a | 69.61 | 98.61 |
| Lepr + Sct + Tfpi | Hand1 + Trap1a | 69.61 | 98.61 |
| Procr + Sct + Tfpi | Hand1 + Trap1a | 69.61 | 98.61 |
| Hspb1 + Sdc1 | Fthl17a + Procr + Sct | 69.61 | 97.26 |
| Cdh5 + Hspb1 | Fthl17a + Lepr + Sct | 69.61 | 97.26 |
| Hspb1 + Slc38a4 | Fthl17a + Lepr + Sct | 69.61 | 97.26 |
| Hspb1 + Tnfrsf9 | Fthl17a + Plet1 + Sct | 69.61 | 97.26 |
| Hspb1 + Sdc1 | Fthl17a + Plet1 + Sct | 69.61 | 97.26 |
| Hspb1 + Lepr | Hspg2 + Sct | 69.61 | 97.26 |
| Cdh5 + Hspb1 | Hspg2 + Sct | 69.61 | 97.26 |
| Hspb1 + Tnfrsf9 | Fthl17a + Procr + Sct | 69.61 | 97.26 |
| Hspb1 + Slc38a4 | Hspg2 + Sct | 69.61 | 97.26 |
| Hspb1 + Lepr | Fthl17a + Sct + Slc38a4 | 69.61 | 97.26 |
| Cdh5 + Hspb1 | Fthl17a + Sct + Slc38a4 | 69.61 | 97.26 |
| Hspb1 + Slc38a4 | Fthl17a + Sct + Slc38a4 | 69.61 | 97.26 |
| Plac1 + Sct + Tfpi | Hand1 + Trap1a | 69.61 | 98.61 |
| Sct + Slc38a4 + Tfpi | Hand1 + Trap1a | 69.61 | 98.61 |
| Hspg2 + Sct + Tfpi | Hand1 + Trap1a | 69.61 | 98.61 |
| Sct + Sdc1 + Tfpi | Hand1 + Trap1a | 69.61 | 98.61 |
| Cryab + Sct + Tfpi | Hand1 + Trap1a | 69.61 | 98.61 |

TABLE 5
Biomarkers and Biomarker combination to enrich
Stromal cells from maternal biological samples.

| | | % yield_Stromal | % purity_Stromal |
|---|---|---|---|
| Biomarker Set #1 | Biomarker Set #2 | Cell | Cell |
| Gpx3 + Dmkn + Prl8a2 | H19 + Krt19 + Prl8a2 | 100.00 | 90.91 |
| Mt1 + Gpx3 + Dmkn | H19 + Krt19 + Prl8a2 | 100.00 | 90.91 |
| 4930486L24Rik + Gpx3 + Dmkn | H19 + Krt19 + Prl8a2 | 100.00 | 90.91 |
| Ly6c1 + Mt2 + Dmkn | H19 + Krt19 + Prl8a2 | 100.00 | 90.91 |
| Rrm2 + Gpx3 + Dmkn | H19 + Krt19 + Prl8a2 | 100.00 | 90.91 |
| Gpx3 + Dmkn + Prl8a2 | Krt19 + Prl8a2 + Sct | 100.00 | 90.91 |
| Mt1 + Mt2 + Dmkn | Krt19 + Prl8a2 + Sct | 100.00 | 90.91 |
| 4930486L24Rik + Gpx3 + Dmkn | Krt19 + Prl8a2 + Sct | 100.00 | 90.91 |
| Ly6c1 + Mt2 + Dmkn | Krt19 + Prl8a2 + Sct | 100.00 | 90.91 |
| Rrm2 + Gpx3 + Dmkn | Krt19 + Prl8a2 + Sct | 100.00 | 90.91 |
| Gpx3 + Dmkn + Prl8a2 | Cryab + H19 + Krt19 | 100.00 | 90.91 |
| Mt1 + Gpx3 + Dmkn | Cryab + H19 + Krt19 | 100.00 | 90.91 |
| 4930486L24Rik + Gpx3 + Dmkn | Cryab + H19 + Krt19 | 100.00 | 90.91 |
| Ly6c1 + Mt2 + Dmkn | Cryab + H19 + Krt19 | 100.00 | 90.91 |
| Rrm2 + Gpx3 + Dmkn | Cryab + H19 + Krt19 | 100.00 | 90.91 |
| Gpx3 + Dmkn + Prl8a2 | Cryab + Krt19 + Sct | 100.00 | 90.91 |
| Mt1 + Mt2 + Dmkn | Cryab + Krt19 + Sct | 100.00 | 90.91 |
| 4930486L24Rik + Gpx3 + Dmkn | Cryab + Krt19 + Sct | 100.00 | 90.91 |
| Ly6c1 + Mt2 + Dmkn | Cryab + Krt19 + Sct | 100.00 | 90.91 |
| Rrm2 + Gpx3 + Dmkn | Cryab + Krt19 + Sct | 100.00 | 90.91 |
| Gpx3 + Dmkn + Prl8a2 | H19 + Krt18 + Prl8a2 | 100.00 | 90.91 |
| Mt1 + Gpx3 + Dmkn | H19 + Krt18 + Prl8a2 | 100.00 | 90.91 |
| 4930486L24Rik + Gpx3 + Dmkn | H19 + Krt18 + Prl8a2 | 100.00 | 90.91 |
| Ly6c1 + Mt2 + Dmkn | H19 + Krt18 + Prl8a2 | 100.00 | 90.91 |
| Rrm2 + Gpx3 + Dmkn | H19 + Krt18 + Prl8a2 | 100.00 | 90.91 |
| Tfpi + Cryab + Dmkn | Mt1 + Gpx3 + Dmkn | 100.00 | 90.91 |
| Rrm2 + Mt2 + Dmkn | Tfpi + Cryab + Dmkn | 100.00 | 90.91 |
| Rrm2 + Gpx3 + Dmkn | Tfpi + Cryab + Dmkn | 100.00 | 100.00 |
| 4930486L24Rik + Gpx3 + Dmkn | Tfpi + Cryab + Dmkn | 100.00 | 90.91 |
| Ly6c1 + Mt2 + Dmkn | Tfpi + Cryab + Dmkn | 100.00 | 90.91 |
| Ly6c1 + Gpx3 + Dmkn | Tfpi + Cryab + Dmkn | 100.00 | 100.00 |
| Tfpi + Cryab + Dmkn | Gpx3 + Dmkn | 100.00 | 100.00 |
| Des + Mfap5 | Plac1 + Sct + Tfpi | 90.00 | 100.00 |
| Des + Mfap5 + Htra1 | Plac1 + Sct + Tfpi | 90.00 | 90.00 |
| Rrm2 + Mt2 + Mfap5 | Tfpi + Cryab + Dmkn | 90.00 | 90.00 |
| Ly6c1 + Mt2 + Mfap5 | Plac1 + Sct + Tfpi | 90.00 | 90.00 |
| Ly6c1 | Plac1 + Sct + Tfpi | 90.00 | 100.00 |
| Mt1 | Plac1 + Sct + Tfpi | 90.00 | 90.00 |
| Gpx3 | Plac1 + Sct + Tfpi | 90.00 | 100.00 |
| Ly6c1 + Gpx3 + Dmkn | Plac1 + Sct + Tfpi | 90.00 | 100.00 |
| Rrm2 + Mt2 + Des | Tfpi + Cryab + Dmkn | 90.00 | 90.00 |
| Rrm2 + Gpx3 + Des | Plac1 + Sct + Tfpi | 90.00 | 90.00 |
| Htra1 + Mfap5 | Plac1 + Sct + Tfpi | 90.00 | 90.00 |
| Gpx3 | Adgrf5 + Sct + Tfpi | 90.00 | 100.00 |
| Gpx3 + Des + Mfap5 | Plac1 + Sct + Tfpi | 90.00 | 100.00 |
| Gpx3 + Htra1 + Des | Plac1 + Sct + Tfpi | 90.00 | 90.00 |
| Gpx3 + Htra1 + Mfap5 | Plac1 + Sct + Tfpi | 90.00 | 90.00 |
| Mt1 + Gpx3 + Dmkn | Plac1 + Sct + Tfpi | 90.00 | 90.00 |
| Mt2 + Mfap5 | Plac1 + Sct + Tfpi | 90.00 | 90.00 |
| Mt1 + Gpx3 + Des | Plac1 + Sct + Tfpi | 90.00 | 90.00 |
| Mt1 + Gpx3 + Mfap5 | Plac1 + Sct + Tfpi | 90.00 | 90.00 |
| Ly6c1 + Gpx3 + Htra1 | Adgrf5 + Sct + Tfpi | 90.00 | 90.00 |
| 4930486L24Rik + Gpx3 + Des | Plac1 + Sct + Tfpi | 90.00 | 90.00 |
| 4930486L24Rik + Gpx3 + Mfap5 | Plac1 + Sct + Tfpi | 90.00 | 90.00 |
| 4930486L24Rik + Gpx3 + Htra1 | Plac1 + Sct + Tfpi | 90.00 | 90.00 |
| 4930486L24Rik + Gpx3 + Dmkn | Plac1 + Sct + Tfpi | 90.00 | 90.00 |
| Ly6c1 + Gpx3 + Des | Plac1 + Sct + Tfpi | 90.00 | 100.00 |
| Ly6c1 + Gpx3 + Mfap5 | Plac1 + Sct + Tfpi | 90.00 | 100.00 |
| Ly6c1 + Gpx3 + Htra1 | Plac1 + Sct + Tfpi | 90.00 | 90.00 |
| Gpx3 + Htra1 + Des | Sct + Slc38a4 + Tfpi | 90.00 | 90.00 |
| Ly6c1 + Mt2 + Des | Plac1 + Sct + Tfpi | 90.00 | 90.00 |
| Rrm2 + Gpx3 + Htra1 | Tfpi + Cryab + Dmkn | 90.00 | 90.00 |
| Ly6c1 + Mt2 + Dmkn | Plac1 + Sct + Tfpi | 90.00 | 90.00 |
| Mt1 + Gpx3 + Mfap5 | Adgrf5 + Sct + Tfpi | 90.00 | 90.00 |
| Mt2 + Des + Mfap5 | Plac1 + Sct + Tfpi | 90.00 | 90.00 |
| Rrm2 + Gpx3 + Mfap5 | Plac1 + Sct + Tfpi | 90.00 | 90.00 |
| Rrm2 + Gpx3 + Dmkn | Plac1 + Sct + Tfpi | 90.00 | 90.00 |
| Mt1 | Adgrf5 + Sct + Tfpi | 90.00 | 90.00 |
| 4930486L24Rik | Adgrf5 + Sct + Tfpi | 90.00 | 90.00 |
| Ly6c1 | Adgrf5 + Sct + Tfpi | 90.00 | 100.00 |
| Des + Mfap5 | Adgrf5 + Sct + Tfpi | 90.00 | 100.00 |
| Htra1 + Mfap5 | Adgrf5 + Sct + Tfpi | 90.00 | 90.00 |
| Des + Mfap5 + Htra1 | Adgrf5 + Sct + Tfpi | 90.00 | 90.00 |
| Gpx3 + Des + Mfap5 | Adgrf5 + Sct + Tfpi | 90.00 | 100.00 |
| Gpx3 + Htra1 + Des | Adgrf5 + Sct + Tfpi | 90.00 | 90.00 |
| Gpx3 + Dmkn | Plac1 + Sct + Tfpi | 90.00 | 100.00 |
| Gpx3 + Dmkn | Adgrf5 + Sct + Tfpi | 90.00 | 100.00 |
| Gpx3 + Htra1 | Plac1 + Sct + Tfpi | 90.00 | 90.00 |
| Mt2 + Des + Mfap5 | Adgrf5 + Sct + Tfpi | 90.00 | 90.00 |
| Mt2 + Mfap5 | Adgrf5 + Sct + Tfpi | 90.00 | 90.00 |
| Mt1 + Gpx3 + Des | Adgrf5 + Sct + Tfpi | 90.00 | 90.00 |
| Gpx3 + Htra1 | Adgrf5 + Sct + Tfpi | 90.00 | 90.00 |
| Mt1 + Gpx3 + Dmkn | Adgrf5 + Sct + Tfpi | 90.00 | 90.00 |
| 4930486L24Rik + Gpx3 + Des | Adgrf5 + Sct + Tfpi | 90.00 | 90.00 |
| 4930486L24Rik + Gpx3 + Mfap5 | Adgrf5 + Sct + Tfpi | 90.00 | 90.00 |
| 4930486L24Rik + Gpx3 + Htra1 | Adgrf5 + Sct + Tfpi | 90.00 | 90.00 |
| 4930486L24Rik + Gpx3 + Dmkn | Adgrf5 + Sct + Tfpi | 90.00 | 90.00 |
| Ly6c1 + Gpx3 + Des | Adgrf5 + Sct + Tfpi | 90.00 | 100.00 |
| Ly6c1 + Gpx3 + Mfap5 | Adgrf5 + Sct + Tfpi | 90.00 | 100.00 |
| Gpx3 + Mfap5 | Adgrf5 + Sct + Tfpi | 90.00 | 100.00 |
| Gpx3 + Mfap5 | Plac1 + Sct + Tfpi | 90.00 | 100.00 |
| Ly6c1 + Gpx3 + Dmkn | Adgrf5 + Sct + Tfpi | 90.00 | 100.00 |
| Ly6c1 + Mt2 + Des | Adgrf5 + Sct + Tfpi | 90.00 | 90.00 |
| Ly6c1 + Mt2 + Mfap5 | Adgrf5 + Sct + Tfpi | 90.00 | 90.00 |
| Ly6c1 + Mt2 + Dmkn | Adgrf5 + Sct + Tfpi | 90.00 | 90.00 |
| Rrm2 + Gpx3 + Des | Adgrf5 + Sct + Tfpi | 90.00 | 90.00 |
| Rrm2 + Gpx3 + Mfap5 | Adgrf5 + Sct + Tfpi | 90.00 | 90.00 |
| Rrm2 + Gpx3 + Dmkn | Adgrf5 + Sct + Tfpi | 90.00 | 90.00 |
| Mt1 | Sct + Slc38a4 + Tfpi | 90.00 | 90.00 |
| Gpx3 | Sct + Slc38a4 + Tfpi | 90.00 | 100.00 |
| 4930486L24Rik | Sct + Slc38a4 + Tfpi | 90.00 | 90.00 |
| Ly6c1 | Sct + Slc38a4 + Tfpi | 90.00 | 100.00 |
| Des + Mfap5 | Sct + Slc38a4 + Tfpi | 90.00 | 100.00 |
| Htra1 + Mfap5 | Sct + Slc38a4 + Tfpi | 90.00 | 90.00 |
| Des + Mfap5 + Htra1 | Sct + Slc38a4 + Tfpi | 90.00 | 90.00 |
| Gpx3 + Des + Mfap5 | Sct + Slc38a4 + Tfpi | 90.00 | 100.00 |
| Gpx3 + Htra1 + Mfap5 | Sct + Slc38a4 + Tfpi | 90.00 | 90.00 |
| Gpx3 + Des | Sct + Slc38a4 + Tfpi | 90.00 | 100.00 |
| Gpx3 + Mfap5 | Sct + Slc38a4 + Tfpi | 90.00 | 100.00 |
| Gpx3 + Htra1 | Sct + Slc38a4 + Tfpi | 90.00 | 90.00 |
| Rrm2 + Gpx3 + Mfap5 | Tfpi + Cryab + Dmkn | 90.00 | 100.00 |
| Rrm2 + Gpx3 + Des | Tfpi + Cryab + Dmkn | 90.00 | 100.00 |
| Ly6c1 + Mt2 + Mfap5 | Tfpi + Cryab + Dmkn | 90.00 | 90.00 |
| Ly6c1 + Mt2 + Des | Tfpi + Cryab + Dmkn | 90.00 | 90.00 |
| 4930486L24Rik | Plac1 + Sct + Tfpi | 90.00 | 90.00 |
| Lepr + Sct + Tfpi | Gpx3 | 90.00 | 100.00 |
| Procr + Sct + Tfpi | Gpx3 | 90.00 | 100.00 |
| Hspg2 + Sct + Tfpi | Gpx3 | 90.00 | 100.00 |
| Tfpi + Cryab + Dmkn | Gpx3 | 90.00 | 100.00 |
| Tfpi + Cryab + Htra1 | Gpx3 | 90.00 | 100.00 |
| Sct + Slc38a4 + Tfpi | Gpx3 | 90.00 | 100.00 |
| Tfpi + Cryab + Mfap5 | Gpx3 | 90.00 | 100.00 |
| Tfpi + Cryab + Des | Gpx3 | 90.00 | 100.00 |
| Plet1 + Sct + Tfpi | Gpx3 | 90.00 | 100.00 |
| Cdh5 + Sct + Tfpi | Gpx3 | 90.00 | 100.00 |

In some embodiments, the performing enriches the cell sample for the population of fetal cells. In some embodiments, the performing enriches the cell sample for the population of fetal cells with a yield of between about 0% to about 100% fetal cells. In some embodiments, the performing enriches the cell sample for the population of fetal cells with a yield of at least about 0% fetal cells, at least about 5% fetal cells, at least about 10% fetal cells, at least about 15% fetal cells, at least about 20% fetal cells, at least about 25% fetal cells, at least about 30% fetal cells, at least about 35% fetal cells, at least about 40% fetal cells, at least about 45% fetal cells, at least about 50% fetal cells, at least about 55% fetal cells, at least about 60% fetal cells, at least about 65% fetal cells, at least about 70% fetal cells, at least about 75% fetal cells, at least about 80% fetal cells, at least about 85% fetal cells, at least about 90% fetal cells, at least about 95% fetal cells, at least about 100% fetal cells, or more. In some embodiments, the performing enriches the cell sample for the population of fetal cells with a yield of at most about 100% fetal cells, at most about 95% fetal cells, at most about 90% fetal cells, at most about 85% fetal cells, at most about 80% fetal cells, at most about 75% fetal cells, at most about 70% fetal cells, at most about 65% fetal cells, at most about 60% fetal cells, at most about 55% fetal cells, at most about 50% fetal cells, at most about 45% fetal cells, at most about 40% fetal cells, at most about 35% fetal cells, at most about 30% fetal cells, at most about 25% fetal cells, at most about 20% fetal cells, at most about 15% fetal cells, at most about 10% fetal cells, at most about 5% fetal cells, at most about 0% fetal cells, or less. In some embodiments, the performing enriches the cell sample for the population of fetal cells with a yield of about 0% fetal cells, about 5% fetal cells, about 10% fetal cells, about 15% fetal cells, about 20% fetal cells, about 25% fetal cells, about 30% fetal cells, about 35% fetal cells, about 40% fetal cells, about 45% fetal cells, about 50% fetal cells, about 55% fetal cells, about 60% fetal cells, about 65% fetal cells, about 70% fetal cells, about 75% fetal cells, about 80% fetal cells, about 85% fetal cells, about 90% fetal cells, about 95% fetal cells, or about 100% fetal cells.

In some embodiments, the performing enriches the cell sample for the population of fetal cells. In some embodiments, the performing enriches the cell sample for the population of fetal cells with a purity of between about 0% to about 100% fetal cells. In some embodiments, the performing enriches the cell sample for the population of fetal cells with a purity of at least about 0% fetal cells, at least about 5% fetal cells, at least about 10% fetal cells, at least about 15% fetal cells, at least about 20% fetal cells, at least about 25% fetal cells, at least about 30% fetal cells, at least about 35% fetal cells, at least about 40% fetal cells, at least about 45% fetal cells, at least about 50% fetal cells, at least about 55% fetal cells, at least about 60% fetal cells, at least about 65% fetal cells, at least about 70% fetal cells, at least about 75% fetal cells, at least about 80% fetal cells, at least about 85% fetal cells, at least about 90% fetal cells, at least about 95% fetal cells, at least about 100% fetal cells, or more. In some embodiments, the performing enriches the cell sample for the population of fetal cells with a purity of at most about 100% fetal cells, at most about 95% fetal cells, at most about 90% fetal cells, at most about 85% fetal cells, at most about 80% fetal cells, at most about 75% fetal cells, at most about 70% fetal cells, at most about 65% fetal cells, at most about 60% fetal cells, at most about 55% fetal cells, at most about 50% fetal cells, at most about 45% fetal cells, at most about 40% fetal cells, at most about 35% fetal cells, at most about 30% fetal cells, at most about 25% fetal cells, at most about 20% fetal cells, at most about 15% fetal cells, at most about 10% fetal cells, at most about 5% fetal cells, at most about 0% fetal cells, or fewer. In some embodiments, the performing enriches the cell sample for the population of fetal cells with a purity of about 0% fetal cells, about 5% fetal cells, about 10% fetal cells, about 15% fetal cells, about 20% fetal cells, about 25% fetal cells, about 30% fetal cells, about 35% fetal cells, about 40% fetal cells, about 45% fetal cells, about 50% fetal cells, about 55% fetal cells, about 60% fetal cells, about 65% fetal cells, about 70% fetal cells, about 75% fetal cells, about 80% fetal cells, about 85% fetal cells, about 90% fetal cells, about 95% fetal cells, or about 100% fetal cells.

In some embodiments, the performing enriches the cell sample for the population of maternal cells. In some embodiments, the performing enriches the cell sample for the population of maternal cells with a yield of between about 0% to about 100% fetal cells. In some embodiments, the performing enriches the cell sample for the population of maternal cells with a yield of at least about 0% maternal cells, at least about 5% maternal cells, at least about 10% maternal cells, at least about 15% maternal cells, at least about 20% maternal cells, at least about 25% maternal cells, at least about 30% maternal cells, at least about 35% maternal cells, at least about 40% maternal cells, at least about 45% maternal cells, at least about 50% maternal cells, at least about 55% maternal cells, at least about 60% maternal cells, at least about 65% maternal cells, at least about 70% maternal cells, at least about 75% maternal cells, at least about 80% maternal cells, at least about 85% maternal cells, at least about 90% maternal cells, at least about 95% maternal cells, at least about 100% maternal cells, or more. In some embodiments, the performing enriches the cell sample for the population of maternal cells with a yield of at most about 100% maternal cells, at most about 95% maternal cells, at most about 90% maternal cells, at most about 85% maternal cells, at most about 80% maternal cells, at most about 75% maternal cells, at most about 70% maternal cells, at most about 65% maternal cells, at most about 60% maternal cells, at most about 55% maternal cells, at most about 50% maternal cells, at most about 45% maternal cells, at most about 40% maternal cells, at most about 35% maternal cells, at most about 30% maternal cells, at most about 25% maternal cells, at most about 20% maternal cells, at most about 15% maternal cells, at most about 10% maternal cells, at most about 5% maternal cells, at most about 0% maternal cells, or fewer. In some embodiments, the performing enriches the cell sample for the population of maternal cells with a yield of about 0% maternal cells, about 5% maternal cells, about 10% maternal cells, about 15% maternal cells, about 20% maternal cells, about 25% maternal cells, about 30% maternal cells, about 35% maternal cells, about 40% maternal cells, about 45% maternal cells, about 50% maternal cells, about 55% maternal cells, about 60% maternal cells, about 65% maternal cells, about 70% maternal cells, about 75% maternal cells, about 80% maternal cells, about 85% maternal cells, about 90% maternal cells, about 95% maternal cells, or about 100% maternal cells.

In some embodiments, the performing enriches the cell sample for the population of maternal cells. In some embodiments, the performing enriches the cell sample for the population of maternal cells with a purity of between about 0% to about 100% maternal cells. In some embodiments, the performing enriches the cell sample for the population of maternal cells with a purity of at least about 0% maternal cells, at least about 5% maternal cells, at least about 10% maternal cells, at least about 15% maternal cells, at least about 20% maternal cells, at least about 25% maternal cells, at least about 30% maternal cells, at least about 35% maternal cells, at least about 40% maternal cells, at least about 45% maternal cells, at least about 50% maternal cells, at least about 55% maternal cells, at least about 60% maternal cells, at least about 65% maternal cells, at least about 70% maternal cells, at least about 75% maternal cells, at least about 80% maternal cells, at least about 85% maternal cells, at least about 90% maternal cells, at least about 95% maternal cells, at least about 100% maternal cells, or more. In some embodiments, the performing enriches the cell sample for the population of fetal cells with a purity of at most about 100% maternal cells, at most about 95% maternal cells, at most about 90% maternal cells, at most about 85% maternal cells, at most about 80% maternal cells, at most about 75% maternal cells, at most about 70% maternal cells, at most about 65% maternal cells, at most about 60% maternal cells, at most about 55% maternal cells, at most about 50% maternal cells, at most about 45% maternal cells, at most about 40% maternal cells, at most about 35% maternal cells, at most about 30% maternal cells, at most about 25% maternal cells, at most about 20% maternal cells, at most about 15% maternal cells, at most about 10% maternal cells, at most about 5% maternal cells, at most about 0% maternal cells, or fewer. In some embodiments, the performing enriches the cell sample for the population of fetal cells with a purity of about 0% maternal cells, about 5% maternal cells, about 10% maternal cells, about 15% maternal cells, about 20% maternal cells, about 25% maternal cells, about 30% maternal cells, about 35% maternal cells, about 40% maternal cells, about 45% maternal cells, about 50% maternal cells, about 55% maternal cells, about 60% maternal cells, about 65% maternal cells, about 70% maternal cells, about 75% maternal cells, about 80% maternal cells, about 85% maternal cells, about 90% maternal cells, about 95% maternal cells, or about 100% maternal cells.

In some embodiments, the performing enriches the cell sample for the population of fetal cells. In some embodiments, the performing enriches the cell sample for the population of maternal cells. In some embodiments, the cell sample is enriched in fetal cells and depleted in maternal cells. In some embodiments, cell sample is enriched for fetal cells by immunomagnetic cell separation. In some embodiments, the immunomagnetic cell separation is positive immunomagnetic cell selection. In some embodiments, the cell sample is enriched for fetal cells by fluorescence-activated cell sorting (FACS). In some embodiments, the cell sample is enriched for fetal cells by microscopic microdissection. In some embodiments, the cell samples is enriched for fetal cells by microscopic imaging. In some embodiments, the cell sample is enriched for fetal cells using one or more fetal cell biomarkers. In some embodiments, the cell sample is enriched for fetal cells by negative immunomagnetic cell separation. In some embodiments, the negative immunomagnetic cell separation enriches for fetal cells using one or more maternal cell biomarkers. In some embodiments, the cell sample is enriched for fetal cells using one or more isolation agents specific for one or more fetal cell biomarkers. In some embodiments, the one or more isolation agents specific for one or more fetal cell biomarkers has a first isolation agent. In some embodiments, the first isolation agent couples to a first biomarker in the population of fetal cells. In some embodiments, the cell sample is enriched for fetal cells by density gradient centrifugation. In some embodiments, the cell sample is enriched for fetal cells by immunodensity cell isolation. In some embodiments, the cell sample is enriched for fetal cells by microfluidic cell sorting.

In some embodiments, the population of fetal cells and the population of maternal cells are from a biological sample. In some embodiments, the biological sample is a cell sample. In some embodiments, the cell sample is a blood sample. In some embodiments, the blood sample is blood plasma. In some embodiments, the blood sample is blood serum. In some embodiments, the blood sample is a buffy coat. In some embodiments, the blood sample is peripheral blood mononuclear cells. In some embodiments, the blood sample is nucleated white blood cells. In some embodiments, the blood sample is whole blood. In some embodiments, the blood sample is whole blood depleted of red blood cells. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof. In some embodiments, the sample is from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the sample is from a pregnant subject. In some embodiments, the sample is a maternal biological sample. In some embodiments, the maternal biological sample is from a pregnant subject.

In some embodiments, the method further comprises providing an extract prior to performing to the enrichment. In some embodiments, the extract is made by extracting cells from the cell sample. In some embodiments, the extract is composed of cells from the cell sample. In some embodiments, the extract is composed of nucleated cells from the cell sample. In some embodiments, non-nucleated cells are removed from the cell sample to generate the extract. In some embodiments, extracellular proteins in the cell sample are removed from the cell sample to generate the extract. In some embodiments, extracellular nucleic acids in the cell sample are removed from the cell sample to generate the extract. In some embodiments, extracellular metabolites in the cell sample are removed from the cell sample to generate the extract. In some embodiments, the extract is generated via gradient centrifugation. In some examples, the gradient centrifugation is sediment gradient centrifugation. In some embodiments, the cells are extracted from the cell sample. In some embodiments, the population of fetal cells is extracted from the cell sample. In some embodiments, the population of maternal cells is extracted from the cell sample. In some embodiments, the population of maternal cells and the population of fetal cells are extracted from the cell sample. In some embodiments, the cells extracted from the cell sample are used to provide an extract. In some embodiments, the cells are extracted from the cell sample using density gradient centrifugation. In some embodiments, the cells are extracted from the cell sample using magnetic isolation. In some embodiments, the cells are extracted from the cell sample using shear-induced diffusion. In some embodiments, the extract is made up of the population of fetal cells. In some embodiments, the extract is made up of the population of maternal cells. In some embodiments, the extract is made up of the population of fetal cells and the population of maternal cells.

In some embodiments, the method further comprises sorting the population of the fetal cells in the extract from the population of maternal cells prior to the performing. In some embodiments, the sorting of the cells in the extract provides an enriched sample. In some embodiments, the extract is sorted to provide an enriched sample. In some embodiments, the enriched sample is enriched in fetal cells. In some embodiments, the enriched sample enriched in fetal cells has a purity of fetal cells in the enriched sample between 0% to about 100% of total cells in the enriched sample. In some embodiments, the enriched sample enriched in fetal cells has a purity of fetal cells in the enriched sample of at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more of total cells in the enriched sample. In some embodiments, the enriched sample enriched in fetal cells has a purity of fetal cells in the enriched sample between 0% to about 100% of total cells in the enriched sample of at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less of total cells in the enriched sample. In some embodiments, the enriched sample enriched in fetal cells has a purity of fetal cells in the enriched sample between 0% to about 100% of total cells in the enriched sample of about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of total cells in the enriched sample. In some embodiments, the enriched sample is depleted in maternal cells. In some embodiments, the enriched sample enriched in maternal cells has a purity of fetal cells in the enriched sample between 0% to about 100% of total cells in the enriched sample. In some embodiments, the enriched sample enriched in maternal cells has a purity of fetal cells in the enriched sample of at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more of total cells in the enriched sample. In some embodiments, the enriched sample enriched in maternal cells has a purity of fetal cells in the enriched sample between 0% to about 100% of total cells in the enriched sample of at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 0%, or less of total cells in the enriched sample. In some embodiments, the enriched sample enriched in fetal cells has a purity of maternal cells in the enriched sample between 0% to about 100% of total cells in the enriched sample of about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of total cells in the enriched sample. In some embodiments, the enriched sample is enriched in fetal cells and depleted in maternal cells. In some embodiments, the extract is enriched for fetal cells by sorting the extract for a cell characteristic. Non-limiting examples of cell characteristics include size, expression of a specific gene, expression of an extracellular protein, and expression of an intracellular protein. In some embodiments, the extract is sorted by immunomagnetic cell separation. In some embodiments, the immunomagnetic cell separation is positive immunomagnetic cell selection. In some embodiments, the extract is enriched for fetal cells by negative immunomagnetic cell separation. In some embodiments, the negative immunomagnetic cell separation enriches for fetal cells using one or more maternal cell biomarkers. In some embodiments, the extract is sorted by FACS. In some embodiments, the extract is sorted by MACS. In some embodiments, the extract is sorted by microscopic microdissection. In some embodiments, the extract is sorted by microscopic imaging. In some embodiments, the extract is sorted for fetal cells using one or more isolation agents specific for one or more fetal cell biomarkers.

In some embodiments, the method further comprises incubating (e.g., contacting) the maternal biological sample prior to the performing the enrichment. In some embodiments, subsequent to the performing, the cell sample is incubated with the one or more isolation agents specific for one or more fetal cell biomarkers.

In some embodiments, the one or more isolation agents specific for one or more fetal cell biomarkers has a first isolation agent. In some embodiments, the first isolation agent couples to a first biomarker. In some embodiments, the first biomarker is one or more fetal biomarkers. In some embodiments, the isolation agent specific for one or more fetal cell biomarkers couples to an extracellular biomarker. In some embodiments, the isolation agent specific for one or more fetal cell biomarkers couples to an extracellular protein. In some embodiments, the isolation agent specific for one or more fetal cell biomarkers couples to a cell surface protein. In some embodiments, the isolation agent specific for a fetal cell biomarker couples to a soluble cell surface protein. In some embodiments, the isolation agent specific for a fetal cell biomarker couples to a cell-surface receptor. In some embodiments, the isolation agent specific for a fetal cell biomarker couples to a transmembrane protein. In some embodiments, the isolation agent specific for a fetal cell biomarker couples to a transmembrane channel protein.

In some embodiments, the isolation agent specific for a fetal cell biomarker couples to an intracellular biomarker. In some embodiments, the isolation agent specific for a fetal cell biomarker couples to an intracellular protein. In some embodiments, the isolation agent specific for a fetal cell biomarker couples to an enzyme. In some embodiments, the isolation agent specific for a fetal cell biomarker couples to a phosphoprotein. In some embodiments, the isolation agent specific for a fetal cell biomarker couples to a growth factor. In some embodiments, the isolation agent specific for a fetal cell biomarker couples to a structural protein.

In some embodiments, the isolation agents specific for a fetal cell biomarker couples to a nucleic acid marker. In some embodiments, the isolation agent specific for a fetal cell biomarker couples to a DNA biomarker. In some embodiments, the isolation agent specific for a fetal cell biomarker couples to an RNA biomarker. In some embodiments, the isolation agent specific for a fetal cell biomarker couples to mRNA. In some embodiments, the isolation agent specific for a fetal cell biomarkers couples to rRNA. In some embodiments, the isolation agent specific for a fetal cell biomarker couples to lncRNA. In some embodiments, the isolation agent specific for a fetal cell biomarker couples to miRNA. In some embodiments, the isolation agent specific for a fetal cell biomarker couples to siRNA.

In some embodiments, the isolation agent specific for a fetal cell biomarker couples to a first biomarker in the population of fetal cells. In some embodiments, the isolation agent specific for a fetal cell biomarker is an affinity reagent. In some embodiments, the affinity reagent is a ligand. In some embodiments, the affinity reagent is a protein. In some embodiments, the isolation agent specific for a fetal cell biomarkers is an antibody. In some embodiments, the isolation agent specific for a fetal cell biomarker is labeled. In some embodiments, the isolation agent specific for a fetal cell biomarker is fluorescently labeled. In some embodiments, the isolation agent specific for a fetal cell biomarker is coupled to a fluorescent protein or peptide. In some embodiments, the affinity reagent is a nucleic acid molecule. In some embodiments, the isolation agent specific for a fetal cell biomarker is an aptamer. In some embodiments, the isolation agent specific for a fetal cell biomarker is a nucleic acid probe. In some embodiments, the nucleic acid probe is DNA. In some embodiments, the nucleic acid probe is RNA.

In some embodiments, the isolation agent can comprise one or more isolation agents. In some embodiments, the one or more isolation agents can be at least about one isolation agent, at least about two isolation agents, at least about three isolation agents, at least about four isolation agents, at least about five isolation agents, at least about six isolation agents, at least about seven isolation agents, at least about eight isolation agents, at least about nine isolation agents, at least about 10 isolation agents, at least about 15 isolation agents, at least about 20 isolation agents, at least about 25 isolation agents, at least about 30 isolation agents, at least about 35 isolation agents, at least about 40 isolation agents, at least about 45 isolation agents, at least about 50 isolation agents, or more. In some embodiments, the one or more isolation agents can be at most about 50 isolation agents, at most about 45 isolation agents, at most about 40 isolation agents, at most about 35 isolation agents, at most about 30 isolation agents, at most about 25 isolation agents, at most about 20 isolation agents, at most about 15 isolation agents, at most about 10 isolation agents, at most about nine isolation agents, at most about eight isolation agents, at most about seven isolation agents, at most about six isolation agents, at most about five isolation agents, at most about four isolation agents, at most about three isolation agents, at most about two isolation agents, at most about one isolation agent, or fewer. In some embodiments, the one or more isolation agents can be about one isolation agent, about two isolation agents, about three isolation agents, about four isolation agents, about five isolation agents, about six isolation agents, about seven isolation agents, about eight isolation agents, about nine isolation agents, about 10 isolation agents, about 15 isolation agents, about 20 isolation agents, about 25 isolation agents, about 30 isolation agents, about 35 isolation agents, about 40 isolation agents, about 45 isolation agents, or about 50 isolation agents. In some embodiments, the one or more isolation agents can be specific to fetal cell biomarkers. In some embodiments, the one or more isolation agents can be specific to maternal cell biomarkers. In some embodiments, the one or more isolation agents can be specific to fetal cell biomarkers and one or more isolation agents specific maternal cell biomarkers. In some embodiments, the one or more isolation agents can be nucleic acid probes. In some embodiments, the one or more isolation agents can be affinity reagents. In some embodiments, the one or more isolation agents can be nucleic acid probes and affinity reagents.

In some embodiments, the isolation agent specific for a fetal cell biomarker couples to one or more fetal biomarkers. In some embodiments, the fetal cell biomarker is a Sct biomarker, a Krt8 biomarker, a Krt18 biomarker, a H19 biomarker, a Tfpi biomarker, or a Fthl17a biomarker. In some embodiments, the fetal cell biomarker is a Sct biomarker, a H19 biomarker, a Tfpi biomarker, or a Fthl17a biomarker. In some embodiments, the one or more fetal cell biomarkers is a Sct biomarker. In some embodiments, the one or more fetal cell biomarkers is a H19 biomarker. In some embodiments, the one or more fetal cell biomarkers is a Tfpi biomarker. In some embodiments, the one or more fetal cell biomarkers is a Fthl17a biomarker.

In some embodiments, the method further comprises incubating the maternal biological sample prior to the performing the enrichment. In some embodiments, prior to the performing, the cell sample is incubated with one or more isolation agents specific for one or more maternal cell biomarkers.

In some embodiments, the isolation agent specific for a maternal cell biomarkers has a second isolation agent. In some embodiments, the second isolation agent couples to a second biomarker. In some embodiments, the second biomarker is one or more maternal biomarkers. In some embodiments, the isolation agent specific for a maternal cell biomarker couples to an extracellular biomarker. In some embodiments, the isolation agent specific for a maternal cell biomarker couples to an extracellular protein. In some embodiments, the isolation agent specific for a maternal cell biomarker couples to a cell surface protein. In some embodiments, the isolation agent specific for a maternal cell biomarker couples to a soluble cell surface protein. In some embodiments, the isolation agent specific for a maternal cell biomarker couples to a cell-surface receptor. In some embodiments, the isolation agent specific for a maternal cell biomarker couples to a transmembrane protein. In some embodiments, the isolation agent specific for a maternal cell biomarker couples to a transmembrane channel protein.

In some embodiments, the isolation agent specific for a maternal cell biomarker couples to an intracellular biomarker. In some embodiments, the isolation agent specific for a maternal cell biomarker couples to an intracellular protein. In some embodiments, the isolation agent specific for a maternal cell biomarker couples to an enzyme. In some embodiments, the isolation agent specific for a maternal cell biomarker couples to a phosphoprotein. In some embodiments, the isolation agent specific for a maternal cell biomarkers couples to a growth factor. In some embodiments, the isolation agent specific for a maternal cell biomarker couples to a structural protein.

In some embodiments, the isolation agent specific for a maternal cell biomarker couples to a nucleic acid marker. In some embodiments, the isolation agent specific for a maternal cell biomarker couples to a DNA biomarker. In some embodiments, the isolation agent specific for a maternal cell biomarker couples to a RNA biomarker. In some embodiments, the isolation agent specific for a maternal cell biomarker couples to mRNA. In some embodiments, the isolation agent specific for a maternal cell biomarker couples to rRNA. In some embodiments, the isolation agent specific for a maternal cell biomarker couples to lncRNA. In some embodiments, the isolation agent specific for a maternal cell biomarkers couples to miRNA. In some embodiments, the isolation agent specific for a maternal cell biomarker couples to siRNA.

In some embodiments, the isolation agent specific for a maternal cell biomarker couples to a second biomarker in the population of maternal cells. In some embodiments, the isolation agents specific for a maternal cell biomarker is an affinity reagent. In some embodiments, the affinity reagent is a ligand. In some embodiments, the affinity reagent is a protein. In some embodiments, the isolation agent specific for a maternal cell biomarker is an antibody. In some embodiments, the isolation agent specific for a maternal cell biomarker is labeled. In some embodiments, the isolation agent specific for a maternal cell biomarker is fluorescently labeled. In some embodiments, the isolation agent specific for a maternal cell biomarker is coupled to a fluorescent protein or peptide. In some embodiments, the affinity reagent is a nucleic acid molecule. In some embodiments, the isolation agent specific a maternal cell biomarkers is an aptamer. In some embodiments, the isolation agent specific for a maternal cell biomarker is a nucleic acid probe. In some embodiments, the nucleic acid probe is DNA. In some embodiments, the nucleic acid probe is RNA.

In some embodiments, the isolation agent specific for one or more maternal cell biomarkers couples to one or more maternal biomarkers. In some embodiments, the maternal cell biomarker is a Coro1a biomarker, a Cd52 biomarker, a Cyba biomarker, a Rac2 biomarker, a Laptm5 biomarker, or a Ptpre biomarker. In some embodiments, the maternal cell biomarker is a Coro1a biomarker, a Cd52 biomarker, a Cyba biomarker, a Rac2 biomarker, a Laptm5 biomarker, or a Ptprc biomarker. In some embodiments, the maternal cell biomarker is a Coro1a biomarker. In some embodiments, the maternal cell biomarker biomarker is a Cd52 biomarker. In some embodiments, the maternal cell biomarker biomarker is a Cyba biomarker. In some embodiments, the maternal cell biomarker biomarker is a Rac2 biomarker. In some embodiments, the maternal cell biomarker biomarker is a Laptm5 biomarker. In some embodiments, the maternal cell biomarker is a Ptprc biomarker.

In some embodiments, the maternal biological sample is incubated with one or more isolation agents specific for maternal biomarkers. In some embodiments, the maternal biological sample is incubated with one or more isolation agents specific for fetal biomarkers. In some embodiments, the maternal biological sample is incubated with one or more isolation agents specific for fetal biomarkers, then subsequently incubated with one or more isolation agents specific for maternal biomarkers. In some embodiments, the maternal biological sample is incubated with one or more isolation agents specific for maternal biomarkers, then subsequently incubated with one or more isolation agents specific for fetal biomarkers. In some embodiments, the maternal biological sample is incubated with one or more isolation agents specific for fetal biomarkers, then incubated with another one or more isolation agents for another fetal biomarkers. In some embodiments, the maternal biological sample is incubated with one or more isolation agents specific for maternal biomarkers, then incubated with one or more isolation agents specific for fetal biomarkers, and then incubated with another one or more isolation agents specific for another fetal biomarkers.

In some embodiments, the method further comprises sequencing a nucleic acid sequence. In some embodiments, the nucleic acid sequence is from the population of fetal cells. In some embodiments, the nucleic acid sequence is from the population of maternal cells.

In some embodiments, the nucleic acid sequence is a DNA sequence. In some embodiments, the nucleic acid sequence is a RNA sequence. Non-limiting examples of sequencing platforms include massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

In some embodiments, the present disclosure provides a method of extracting cells from a maternal biological sample. In some embodiments, the method comprises extracting an ex vivo subpopulation of maternal cells and an ex vivo subpopulation of fetal cells from a maternal biological sample. In some embodiments, the method further comprises, subsequent to the extracting, enriching the ex vivo subpopulation of fetal cells from the ex vivo subpopulation of maternal cells. In some embodiments, the method further comprises, subsequent to the enriching, sorting the ex vivo subpopulation of fetal cells from the ex vivo subpopulation of maternal cells to provide a sample enriched in fetal cells and depleted in maternal cells. In some embodiments, the method further comprises, subsequent to the sorting, sequencing a nucleic acid from the ex vivo subpopulation of fetal cells.

In some embodiments, the method further comprising depleting red blood cells from the maternal biological sample.

In some embodiments, the present disclosure provides a method of contacting a maternal biological sample. In some embodiments, the method comprises contacting a maternal biological sample with at least one isolation agent. In some embodiments, the maternal biological sample has an ex vivo subpopulation of fetal cells. In some embodiments, the maternal biological sample has an ex vivo subpopulation of maternal cells. In some embodiments, the biological blood sample has an ex vivo subpopulation of fetal cells and an ex vivo subpopulation of maternal cells. In some embodiments, the maternal biological sample is contacted with at least one isolation agent. In some embodiments, the at least one isolation agent couples to a biomarker in the ex vivo subpopulation of fetal cells. In some embodiments, the method further comprises, subsequent to the contacting, incubating the at least one isolation agent with the maternal biological sample. In some embodiments, the method further comprises, subsequent to the incubating, isolating the ex vivo subpopulation of fetal cells in the maternal biological sample from the ex vivo subpopulation of maternal cells in the maternal biological sample. In some embodiments, the method further comprises, subsequent to the isolating, sequencing a nucleic acid from the ex vivo subpopulation of fetal cells.

In some embodiments, the method further comprising depleting red blood cells from the maternal biological sample.

In some embodiments, the present disclosure provides a method of contacting a maternal biological sample. In some embodiments, the method comprises contacting a maternal biological sample with an at least one another isolation agent. In some embodiments, the maternal biological sample has an ex vivo subpopulation of fetal cells. In some embodiments, the maternal biological sample has an ex vivo subpopulation of maternal cells. In some embodiments, the maternal biological sample has an ex vivo subpopulation of maternal cells and an ex vivo population of fetal cells. In some embodiments, the method further comprises, subsequent to the contacting, incubating an isolation agent with the maternal biological sample. In some embodiments, the method further comprises, subsequent to the incubating, isolating the ex vivo subpopulation of fetal cells in the maternal biological sample from the ex vivo subpopulation of maternal cells in the maternal biological sample. In some embodiments, the method further comprises, subsequent to the isolating, sorting the ex vivo subpopulation of fetal cells from the ex vivo subpopulation of maternal cells. In some embodiments, the method further comprises, subsequent to the sorting, sequencing a nucleic acid from the ex vivo subpopulation of fetal cells.

In some embodiments, the method further comprising depleting red blood cells from the maternal biological sample.

In some embodiments, the present disclosure provides a method of contacting a maternal biological sample. In some embodiments, the method comprises contacting a maternal biological sample with an at least a first isolation agent and at least a second isolation agent. In some embodiments, the at least the first isolation agent couples to a fetal biomarker. In some embodiments, the at least the second isolation agent couples to a maternal biomarker. In some embodiments, the maternal biological sample has an ex vivo subpopulation of fetal cells. In some embodiments, the maternal biological sample has an ex vivo subpopulation of maternal cells. In some embodiments, the maternal biological sample has an ex vivo subpopulation of maternal cells and an ex vivo population of fetal cells. In some embodiments, the method further comprises, subsequent to the contacting, incubating an isolation agent with the maternal biological sample. In some embodiments, the method further comprises, subsequent to the incubating, isolating the ex vivo subpopulation of fetal cells in the maternal biological sample from the ex vivo subpopulation of maternal cells in the maternal biological sample. In some embodiments, the method further comprises, subsequent to the isolating, sorting the ex vivo subpopulation of fetal cells from the ex vivo subpopulation of maternal cells. In some embodiments, the method further comprises, subsequent to the sorting, genetically analyzing a nucleic acid from the ex vivo subpopulation of fetal cells.

In some embodiments, the method further comprising depleting red blood cells from the maternal biological sample.

In some embodiments, the present disclosure provides a method of incubating a population of cells from a sample of a subject. In some embodiments, the population of cells comprises a subpopulation of fetal cells. In some embodiments, the population of cells comprises a subpopulation of maternal cells. In some embodiments, the population of cells comprises a subpopulation of fetal cells and a subpopulation of maternal cells. In some embodiments, the sample of the subject is incubated with from about one isolation agent to about ten isolation agents. In some embodiments, the sample of the subject is incubated with at least about one isolation agent, at least about two isolation agents at least about three isolation agents, at least about four isolation agents, at least about five isolation agents, at least about six isolation agents, at least about seven isolation agents, at least about eight isolation agents, at least about nine isolation agents, at least about ten isolation agents, or more. In some embodiments, the sample of the subject is incubated with at most about ten isolation agents, at most about nine isolation agents, at most about eight isolation agents, at most about seven isolation agents, at most about six isolation agents, at most about five isolation agents, at most about four isolation agents, at most about three isolation agents, at most about two isolation agents, at most about one isolation agent, or less. In some embodiments, the sample of the subject is incubated with about one isolation agent, about two isolation agents, about three isolation agents, about four isolation agents, about five isolation agents, about six isolation agents, about seven isolation agents, about eight isolation agents, about nine isolation agents, or about ten isolation agents.

In some embodiments, a first isolation agent is specific for a fetal cell biomarker and/or a maternal cell biomarker. In some embodiments, a second isolation agent is specific for a fetal cell biomarker and/or a maternal cell biomarker. In some embodiments, a third isolation agent is specific for a fetal cell biomarker and/or a maternal cell biomarker. In some embodiments, a fourth isolation agent is specific for a fetal cell biomarker and/or a maternal cell biomarker. In some embodiments, a fifth isolation agent is specific for a fetal cell biomarker and/or a maternal cell biomarker. In some embodiments, a sixth isolation agent is specific for a fetal cell biomarker and/or a maternal cell biomarker. In some embodiments, a seventh isolation agent is specific for a fetal cell biomarker and/or a maternal cell biomarker. In some embodiments, an eighth isolation agent is specific for a fetal cell biomarker and/or a maternal cell biomarker. In some embodiments, a ninth isolation agent is specific for a fetal cell biomarker and/or a maternal cell biomarker. In some embodiments, a tenth isolation agent is specific for a fetal cell biomarker and/or a maternal cell biomarker.

In some embodiments, the sample of the subject is incubated with six isolation agents. In some embodiments, the six isolation agents are a first isolation agent, a second isolation agent, a third isolation agent, a fourth isolation agent, a fifth isolation agent, and a sixth isolation agent. In some embodiments, the first isolation agent is specific for a first biomarker in the subpopulation of fetal cells. In some embodiments, the second isolation agent is specific for a second biomarker in the subpopulation of fetal cells. In some embodiments, the third isolation agent is specific for a third biomarker in the subpopulation of fetal cells. In some embodiments, the fourth isolation agent is specific for a fourth biomarker in the subpopulation of fetal cells. In some embodiments, the fifth isolation agent is specific for a fifth biomarker in the subpopulation of fetal cells. In some embodiments, the sixth isolation agent is specific for a sixth biomarker in the subpopulation of maternal cells.

In some embodiments, the biological sample is a blood sample. In some embodiments, the blood sample is plasma. In some embodiments, the blood sample is serum. In some embodiments, the blood sample is whole blood. In some embodiments, the blood sample is whole blood depleted of red blood cells. In some embodiments, the blood sample is buffy coat. In some embodiments, the blood sample is peripheral blood mononuclear cells. In some embodiments, the blood sample is nucleated white blood cells. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof. In some embodiments, the sample is from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the sample is from a pregnant subject. In some embodiments, the sample from the pregnant subject is a maternal biological sample.

In some embodiments, the maternal biological sample is incubated with at least one isolation agent from about 0 hours to about 48 hours. In some embodiments, the maternal biological sample is incubated with the second isolation agent from about 0 hour to about 1 hour, from about 1 hour to about 2 hour, from about 2 hours to about 3 hours, from about 3 hours to about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, from about 10 hours to about 11 hours, from about 11 hours to about 12 hours, from about 12 hours to about 13 hours, from about 13 hours to about 14 hours, from about 14 hours to about 15 hours, from about 15 hours to about 16 hours, from about 16 hours to about 17 hours, from about 17 hours to about 18 hours, from about 18 hours to about 19 hours, from about 19 hours to about 20 hours, from about 20 hours to about 21 hours, from about 21 hours to about 22 hours, from about 22 hours to about 23 hours, from about 23 hours to about 24 hours, from about 24 hours to about 26 hours, from about 26 hours to about 28 hours, from about 28 hours to about 30 hours, from about 30 hours to about 32 hours, from about 32 hours to about 34 hours, from about 34 hours to about 36 hours, from about 36 hours to about 38 hours, from about 38 hours to about 40 hours, from about 40 hours to about 42 hours, from about 42 hours to about 44 hours, from about 44 hours to about 46 hours, from about 46 hours to about 48 hours.

In some embodiments, the maternal biological sample is incubated with the at least one isolation agent for at least about 0 minutes, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 1 hour, at least about 1.25 hours, at least about 1.5 hours, at least about 1.75 hours, at least about 2 hours, at least about 2.25 hours, at least about 2.5 hours, at least about 2.75 hours, at least about 3 hours, at least about 3.25 hours, at least about 3.5 hours, at least about 3.75 hours, at least about 4 hours, at least about 4.25 hours, at least about 4.5 hours, at least about 4.75 hours, at least about 5 hours, at least about 5.25 hours, at least about 5.5 hours, at least about 5.75 hours, at least about 6 hours, at least about 6.25 hours, at least about 6.5 hours, at least about 6.75 hours, at least about 7 hours, at least about 7.25 hours, at least about 7.5 hours, at least about 7.75 hours, at least about 8 hours, at least about 8.25 hours, at least about 8.5 hours, at least about 8.75 hours, at least about 9 hours, at least about 9.25 hours, at least about 9.5 hours, at least about 9.75 hours, at least about 10 hours, at least about 10.25 hours, at least about 10.5 hours, at least about 10.75 hours, at least about 11 hours, at least about 11.25 hours, at least about 11.5 hours, at least about 11.75 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, at least about 48 hours, or more.

In some embodiments, the maternal biological sample is incubated with the at least one isolation agent for at most about 48 hours, at most about 47 hours, at most about 46 hours, at most about 45 hours, at most about 44 hours, at most about 43 hours, at most about 42 hours, at most about 41 hours, at most about 40 hours, at most about 39 hours, at most about 38 hours, at most about 37 hours, at most about 36 hours, at most about 35 hours, at most about 34 hours, at most about 33 hours, at most about 32 hours, at most about 31 hours, at most about 30 hours, at most about 29 hours, at most about 28 hours, at most about 27 hours, at most about 26 hours, at most about 25 hours, at most about 24 hours, at most about 23 hours, at most about 22 hours, at most about 21 hours, at most about 20 hours, at most about 19 hours, at most about 18 hours, at most about 17 hours, at most about 16 hours, at most about 15 hours, at most about 14 hours, at most about 13 hours, at most about 12 hours, at most about 11.75 hours, at most about 11.5 hours, at most about 11.25 hours, at most about 11 hours, at most about 10.75 hours, at most about 10.5 hours, at most about 10.25 hours, at most about 10 hours, at most about 9.75 hours, at most about 9.5 hours, at most about 9.25 hours, at most about 9 hours, at most about 8.75 hours, at most about 8.5 hours, at most about 8.25 hours, at most about 8 hours, at most about 7.75 hours, at most about 7.5 hours, at most about 7.25 hours, at most about 7 hours, at most about 6.75 hours, at most about 6.5 hours, at most about 6.25 hours, at most about 6 hours, at most about 5.75 hours, at most about 5.5 hours, at most about 5.25 hours, at most about 5 hours, at most about 4.75 hours, at most about 4.5 hours, at most about 4.25 hours, at most about 4 hours, at most about 3.75 hours, at most about 3.5 hours, at most about 3.25 hours, at most about 3 hours, at most about 2.75 hours, at most about 2.5 hours, at most about 2.25 hours, at most about 2 hours, at most about 1.75 hours, at most about 1.5 hours, at most about 1.25 hours, at most about 1 hour, at most about 55 minutes, at most about 50 minutes, at most about 45 minutes, at most about 40 minutes, at most about 35 minutes, at most about 30 minutes, at most about 25 minutes, at most about 20 minutes, at most about 15 minutes, at most about 10 minutes, at most about 5 minutes, at most about 0 minutes, or less.

In some embodiments, the maternal biological sample is incubated with the at least one isolation agent for about 0 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, about 5.25 hours, about 5.5 hours, about 5.75 hours, about 6 hours, about 6.25 hours about 6.5 hours, about 6.75 hours, about 7 hours, about 7.25 hours, about 7.5 hours, about 7.75 hours, about 8 hours, about 8.25 hours, about 8.5 hours, about 8.75 hours, about 9 hours, about 9.25 hours, about 9.5 hours, about 9.75 hours, about 10 hours, about 10.25 hours, about 10.5 hours, about 10.75 hours, about 11 hours, about 11.25 hours, about 11.5 hours, about 11.75 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47, or about 48 hours.

In some embodiments, the maternal biological sample is incubated with the at least one isolation agent at a temperature between about 20° C. to about 70° C. In some embodiments, the maternal biological sample is incubated with the at least one isolation agent at a temperature of at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., or more. In some embodiments, the maternal biological sample is incubated with the at least one isolation agent at a temperature of at most about 70° C., at most about 69° C., at most about 68° C., at most about 67° C., at most about 66° C., at most about 65° C., at most about 64° C., at most about 63° C., at most about 62° C., at most about 61° C., most about 60° C., at most about 59° C., at most about 58° C., at most about 57° C., at most about 56° C., at most about 55° C., at most about 54° C., at most about 53° C., at most about 52° C., at most about 51° C., at most about 50° C., at most about 49° C., at most about 48° C., at most about 47° C., at most about 46° C., at most about 45° C., at most about 44° C., at most about 43° C., at most about 42° C., at most about 41° C., at most about 40° C., at most about 39° C., at most about 38° C., at most about 37° C., at most about 36° C., at most about 35° C., at most about 34° C., at most about 33° C., at most about 32° C., at most about 31° C., at most about 30° C., at most about 29° C., at most about 28° C., at most about 27° C., at most about 26° C., at most about 25° C., at most about 24° C., at most about 23° C., at most about 22° C., at most about 21° C., at most about 20° C., or less. In some embodiments, the maternal biological sample is incubated with the at least one isolation agent at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

In some embodiments, the maternal biological sample is incubated with one or more isolation agents that couples to one or more fetal biomarkers. In some embodiments, the maternal biological sample is incubated with one or more isolation agents that couples to one or more maternal biomarkers. In some embodiments, the maternal biological sample is incubated with one or more isolation agents that couples to one or more fetal biomarkers, then subsequently incubated with one or more isolation agents that couples to one or more maternal biomarkers. In some embodiments, that maternal biological sample is incubated with one or more isolation agents that couples to one or more maternal biomarkers, then subsequently incubated with one or more isolation agents that couples to one or more fetal biomarkers. In some embodiments, the maternal biological sample is incubated with one or more isolation agents that couples to one or more fetal biomarkers, then incubated with another one or more isolation agents that couples to another one or more fetal biomarkers. In some embodiments, the maternal biological sample is incubated with one or more isolation agents that couples to one or more maternal biomarkers, then incubated with one or more isolation agents that couples to one or more fetal biomarkers, and then subsequently incubated with another one or more isolation agents that couples to another one or more fetal biomarkers.

In some embodiments, the isolation agent is an affinity reagent. In some embodiments, the affinity reagent is a ligand. In some embodiments, the affinity reagent is a protein. In some embodiments, the isolation agent is an antibody. In some embodiments, the isolation agent is labeled. In some embodiments, the isolation agent is fluorescently labeled. In some embodiments, the isolation agent is coupled to a fluorescent protein or peptide. In some embodiments, the affinity reagent is a nucleic acid molecule. In some embodiments, the isolation agent is an aptamer. In some embodiments, the isolation agent is a nucleic acid probe. In some embodiments, the nucleic acid probe is DNA. In some embodiments, the nucleic acid probe is RNA.

In some embodiments, one of the first isolation agent, the second isolation agent, the third isolation agent, the fourth isolation agent, the fifth isolation agent, or the sixth isolation agent is nucleic acid probe. In some embodiments, the first isolation agent is a nucleic acid probe. In some embodiments, the second isolation agent is a nucleic acid probe. In some embodiments, the third isolation agent is a nucleic acid probe. In some embodiments, the fourth isolation agent is a nucleic acid probe. In some embodiments, the fifth isolation agent is nucleic acid probe. In some embodiments, the sixth isolation agent is a nucleic acid probe. In some embodiments, the first isolation agent, the second isolation agent, the third isolation agent, the fourth isolation agent, the fifth isolation agent, and the sixth isolation agent are a nucleic acid probe. In some embodiments, the nucleic acid probe couples to the one or more biomarkers. In some embodiments, the nucleic acid probe is configured to couple to a biomarker. In some embodiments, the nucleic acid probe is configured to directly couple to one or more biomarkers. In some embodiments, the nucleic acid probe couples with one or more biomarkers via a covalent bond. In some embodiments, the covalent bond is a peptide bond. In some embodiments, the nucleic acid probe couples to one or more biomarkers via a non-covalent interaction. In some embodiments, the non-covalent interaction is an ionic interaction. In some embodiments, the non-covalent interaction is a hydrogen bond. In some embodiments, the non-covalent interaction is van der Waals interaction. In some embodiments, the non-covalent interaction is a hydrophobic interaction.

In some embodiments, the nucleic acid probe is configured to couple indirectly to one or more biomarkers. In some embodiments, the nucleic acid probe couple to the one or more biomarkers via a linker. In some embodiments, the linker is a cleavable linker. In some embodiments, the cleavable linker is a chemical labile linker. Non-limiting examples of chemical labile linkers include 6-(maleimidocaproyl) hydrazone, (4-(4-acetylphenoxy) butanoic acid) hydrazone, N-acetyl-γ-calicheamicin dimethyl hydrazide, or any combination thereof. In some embodiments, the cleavable linker is a reducible linker. In some embodiments, the reducible linker is a disulfide linker or a glutathione-sensitive disulfide linker. In some embodiments, the cleavable linker is an enzyme-cleavable linker. Non-limiting examples of enzyme-cleavable linkers include peptide-based linkers, valine-citrulline, phenylalanine-lysine, valine-alanine, para-aminobenzyl carbamate, PEG linkers, β-glucuronide linker, β-galactoside linkers, and any combination thereof. In some embodiments, the cleavable linker is a phosphatase-cleavable linker. In some embodiments, the phosphatase-cleavable linker is a phosphate diester. In some embodiments, the linker is a non-cleavable linker. Non-limiting examples of non-cleavable linkers include thioethers, maleimidocapropy, N-succinimidyl-4-(maleimidomethyl)cyclohexane-1-carboxylate linker, and any combination thereof.

In some embodiments, one of the first isolation agent, the second isolation agent, the third isolation agent, the fourth isolation agent, the fifth isolation agent, or the sixth isolation agent is a ligand. In some embodiments, the first isolation agent is a ligand. In some embodiments, the second isolation agent is a ligand. In some embodiments, the third isolation agent is a ligand. In some embodiments, the fourth isolation agent is a ligand. In some embodiments, the fourth isolation agent is a ligand. In some embodiments, the fifth isolation agent is a ligand. In some embodiments, the sixth isolation agent is a ligand. In some embodiments, the first isolation agent, the second isolation agent, the third isolation agent, the fourth isolation agent, the fifth isolation agent, and the sixth isolation agent are a ligand. In some embodiments, the ligand couples to the one or more biomarkers. In some embodiments, the ligand couples to the one or more biomarkers. In some embodiments, the ligand is configured to couple to a biomarker. In some embodiments, the ligand is configured to directly couple to one or more biomarkers. In some embodiments, the ligand couples with one or more biomarkers via a covalent bond. In some embodiments, the covalent bond is a peptide bond. In some embodiments, the ligand couples to one or more biomarkers via a non-covalent interaction. In some embodiments, the non-covalent interaction is an ionic interaction. In some embodiments, the non-covalent interaction is a hydrogen bond. In some embodiments, the non-covalent interaction is van der Waals interaction. In some embodiments, the non-covalent interaction is a hydrophobic interaction.

In some embodiments, the ligand is configured to indirectly couple to one or more biomarkers. In some embodiments, the ligand couples to the one or more biomarkers via a linker. In some embodiments, the linker is a cleavable linker. In some embodiments, the cleavable linker is a chemical labile linker. Non-limiting examples of chemical labile linkers include 6-(maleimidocaproyl)hydrazone, (4-(4-acetylphenoxy)butanoic acid) hydrazone, N-acetyl-γ-calicheamicin dimethyl hydrazide, or any combination thereof. In some embodiments, the cleavable linker is a reducible linker. In some embodiments, the reducible linker is a disulfide linker or a glutathione-sensitive disulfide linker. In some embodiments, the cleavable linker is an enzyme-cleavable linker. Non-limiting examples of enzyme-cleavable linkers include peptide-based linkers, valine-citrulline, phenylalanine-lysine, valine-alanine, para-aminobenzyl carbamate, PEG linkers, β-glucuronide linker, β-galactoside linkers, and any combination thereof. In some embodiments, the cleavable linker is a phosphatase-cleavable linker. In some embodiments, the phosphatase-cleavable linker is a phosphate diester. In some embodiments, the linker is a non-cleavable linker. Non-limiting examples of non-cleavable linkers include thioethers, maleimidocapropy, N-succinimidyl-4-(maleimidomethyl) cyclohexane-1-carboxylate linker, and any combination thereof.

In some embodiments, the isolation agent couples to an extracellular biomarker. In some embodiments, the isolation agent couples to an extracellular protein. In some embodiments, the isolation agent couples to a cell surface protein. In some embodiments, the isolation agent couples to a soluble cell surface protein. In some embodiments, the isolation agent couples to a cell-surface receptor. In some embodiments, the isolation agent couples to a transmembrane protein. In some embodiments, the isolation agent couples to a transmembrane channel protein.

In some embodiments, the isolation agent couples to an intracellular biomarker. In some embodiments, the isolation agent couples to an intracellular protein. In some embodiments, the isolation agent couples to an enzyme. In some embodiments, the isolation agent couples to a phosphoprotein. In some embodiments, the isolation agent couples to a growth factor. In some embodiments, the isolation agent couples to a structural protein.

In some embodiments, the isolation agent couples to a nucleic acid biomarker. In some embodiments, the isolation agent couples to a DNA biomarker. In some embodiments, the isolation agent couples to an RNA biomarker. In some embodiments, the isolation agent couples to mRNA. In some embodiments, the isolation agent couples to rRNA. In some embodiments, the isolation agent couples to lncRNA. In some embodiments, the second isolation agent couples to miRNA. In some embodiments, the isolation agent couples to siRNA.

In some embodiments, the first isolation agent, the second isolation agent, the third isolation agent, the fourth isolation agent, the fifth isolation agent, or the sixth isolation agent is specific for a cell surface marker. In some embodiments, if the at least one isolation agent is specific for a cell surface marker, then the at least one isolation agent couples to the cell surface marker. In some embodiments, if the at least one isolation agent is specific for a cell surface marker, then the at least one isolation agent may not couple to another cell surface marker that is different from the cell surface marker. In some embodiments, if the at least one isolation agent is specific for a cell surface marker, then the at least one isolation agent may not couple to a soluble cell surface marker or protein, a nucleic acid marker, or any combination thereof.

Alternatively, in some embodiments, if the at least one isolation agent is specific for a cell surface marker, then the at least one isolation agent may couple to another cell surface marker that is different from the cell surface marker. In some examples, if the at least one isolation agent is specific for a cell surface marker, then the at least one isolation agent may couple to a soluble cell surface marker or protein, a nucleic acid marker, or any combination thereof.

In some embodiments, the first isolation agent is specific for a cell surface marker or protein. In some embodiments, the second isolation agent is specific for a cell surface marker or protein. In some embodiments, the third isolation agent is specific for a cell surface marker or protein. In some embodiments, the fourth isolation agent is specific for a cell surface marker or protein. In some embodiments, the fifth isolation agent is specific for a cell surface marker or protein. In some embodiments, the sixth isolation agent is specific for a cell surface marker or protein. In some embodiments, the first isolation agent, the second isolation agent, the third isolation agent, the fourth isolation agent, the fifth isolation agent, and the sixth isolation agent are specific for a cell surface marker or protein.

In some embodiments, the first isolation agent, the second isolation agent, the third isolation agent, the fourth isolation agent, the fifth isolation agent, or the sixth isolation agent is specific for a soluble cell surface marker or protein. In some embodiments, if the at least one isolation agent is specific for a soluble cell surface marker or protein, then the at least one isolation agent couples to the soluble cell surface marker or protein. In some embodiments, if the at least one isolation agent is specific for a soluble cell surface marker or protein, then the at least one isolation agent may not couple to another soluble cell surface marker or protein that is different from the soluble cell surface marker or protein. In some embodiments, if the at least one isolation agent is specific for a soluble cell surface marker or protein, then the at least one isolation agent may not couple to a cell surface marker, a nucleic acid marker, or any combination thereof.

Alternatively, in some embodiments, if the at least one isolation agent is specific for a soluble cell surface marker or protein, then the at least one isolation agent may couple to another soluble cell surface marker or protein that is different from the soluble cell surface marker or protein. In some embodiments, if the at least one isolation agent is specific for a soluble cell surface marker or protein, then the at least one isolation agent may couple to a cell surface marker, a nucleic acid marker, or any combination thereof.

In some embodiments, the first isolation agent is specific for a soluble cell surface marker or protein. In some embodiments, the second isolation agent is specific for a soluble cell surface marker or protein. In some embodiments, the third isolation agent is specific for a soluble cell surface marker or protein. In some embodiments, the fourth isolation agent is specific for a soluble cell surface marker or protein. In some embodiments, the fifth isolation agent is specific for a soluble cell surface marker or protein. In some embodiments, the sixth isolation agent is specific for a soluble cell surface marker or protein. In some embodiments, the first isolation agent, the second isolation agent, the third isolation agent, the fourth isolation agent, the fifth isolation agent, and the sixth isolation agent are specific for a soluble cell surface marker or protein.

In some embodiments, the first isolation agent, the second isolation agent, the third isolation agent, the fourth isolation agent, the fifth isolation agent, or the sixth isolation agent is specific for a nucleic acid marker. In some embodiments, if the at least one isolation agent is specific for a nucleic acid marker, then the at least one isolation agent couples to the nucleic acid marker. In some examples, if the at least one isolation agent is specific for a nucleic acid marker, then the at least one isolation agent may not couple to another nucleic acid marker that is different from the cell surface marker. In some embodiments, if the at least one isolation agent is specific for a nucleic acid marker, then the at least one isolation agent may not couple to a soluble cell surface marker or protein, a cell surface marker, or any combination thereof.

Alternatively, in some embodiments, if the at least one isolation agent is specific for a nucleic acid marker, then the at least one isolation agent may couple to another nucleic acid marker that is different from the nucleic acid marker. In some embodiments, if the at least one isolation agent is specific for a nucleic acid marker, then the at least one isolation agent may couple to a soluble cell surface marker or protein, a cell surface marker, or any combination thereof.

In some embodiments, the first isolation agent is specific for a nucleic acid marker. In some embodiments, the second isolation agent is specific for a nucleic acid marker. In some embodiments, the third isolation agent is specific for a nucleic acid marker. In some embodiments, the fourth isolation agent is specific for a nucleic acid marker. In some embodiments, the fifth isolation agent is specific for a nucleic acid marker. In some embodiments, the sixth isolation agent is specific for a nucleic acid marker. In some embodiments, the first isolation agent, the second isolation agent, the third isolation agent, the fourth isolation agent, the fifth isolation agent, and the sixth isolation agent are specific for a nucleic acid marker.

In some embodiments, the method further comprises enriching the subpopulation of fetal cells after incubating. In some embodiments, the sample is enriched to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, the subpopulation of fetal cells is enriched from the subpopulation of maternal cells. In some embodiments, the enriched sample is enriched in fetal cells by positive selection using one or more isolation markers specific for one or more fetal biomarkers. In some embodiments, the enriched sample is enriched in fetal cells by negative selection using one or more isolation markers specific for one or more maternal biomarkers. In some embodiments, the enriched sample is enriched in fetal cells by positive selection using one or more isolation markers specific for one or more fetal biomarkers. In some embodiments, the enriched sample is enriched in fetal cells by negative selection by negative selection using one or more maternal biomarkers. In some embodiments, the enriched sample is enriched in fetal cells by positive selection and then subsequently negative selection. In some embodiments, the enriched sample is enriched in fetal cells by negative selection and then subsequently by positive selection. In some embodiments, sample is enriched for fetal cells by immunomagnetic cell separation. In some embodiments, the immunomagnetic cell separation is positive immunomagnetic cell selection. In some embodiments, the sample is enriched for fetal cells by fluorescence-activated cell sorting (FACS). In some embodiments, the ex vivo population of cells is isolated through magnetic-activated cell sorting (MACS). In some embodiments, the sample is enriched for fetal cells by microscopic microdissection. In some embodiments, the sample is enriched for fetal cells by microscopic imaging. In some embodiments, the sample is enriched for fetal cells using one or more fetal cell biomarkers. In some embodiments, the sample is enriched for fetal cells by negative immunomagnetic cell separation. In some embodiments, the negative immunomagnetic cell separation enriches for fetal cells using one or more maternal cell biomarkers.

In some embodiments, the sample is enriched for fetal cells using the first isolation agent, the second isolation agent, the third isolation agent, the fourth isolation agent, or the fifth isolation agent. In some embodiments, the sample is enriched for fetal cells using the first isolation agent. In some embodiments, the sample is enriched for fetal cells using the second isolation agent. In some embodiments, the sample is enriched for fetal cells using the third isolation agent. In some embodiments, the sample is enriched for fetal cells using the fourth isolation agent. In some embodiments, the sample is enriched for fetal cells using the fifth isolation agent. In some embodiments, the sample is enriched for fetal cells using the sixth isolation agent.

In some embodiments, the sample is enriched for fetal cells using the first isolation agent and the second isolation agent. In some embodiments, the sample is enriched for fetal cells using the first isolation agent and the third isolation agent. In some embodiments, the sample is enriched for fetal cells using the first isolation agent and the fourth isolation agent. In some embodiments, the sample is enriched for fetal cells using the first isolation agent the fifth isolation agent. In some embodiments, the sample is enriched for fetal cells using the first isolation agent and the sixth isolation agent.

In some embodiments, the sample is enriched for fetal cells using the second isolation agent and the third isolation agent. In some embodiments, the sample is enriched for fetal cells using the second isolation agent and the fourth isolation agent. In some embodiments, the sample is enriched for fetal cells using the second isolation agent and the fifth isolation agent. In some embodiments, the sample is enriched for fetal cells using the second isolation agent and the sixth isolation agent.

In some embodiments, the sample is enriched for fetal cells using the third isolation agent and the fourth isolation agent. In some embodiments, the sample is enriched for fetal cells using the third isolation agent and the fifth isolation agent. In some embodiments, the sample is enriched for fetal cells using the third isolation agent and the sixth isolation agent. In some embodiments, the sample is enriched for fetal cells using the fourth isolation agent and the fifth isolation agent. In some embodiments, the sample is enriched for fetal cells using the fourth isolation agent and the sixth isolation agent. In some embodiments, the sample is enriched for fetal cells using the fifth isolation agent and the sixth isolation agent.

In some embodiments, the sample is enriched for fetal cells using the first isolation agent, the second isolation agent, and the third isolation agent. In some embodiments, the sample is enriched for fetal cells using the first isolation agent, the second isolation agent, and the fourth isolation agent. In some embodiments, the sample is enriched for fetal cells using the first isolation agent, the second isolation agent, and the fifth isolation agent. In some embodiments, the sample is enriched for fetal cells using the first isolation agent, the second isolation agent, and the sixth isolation agent. In some embodiments, the sample is enriched for fetal cells using the first isolation agent, the third isolation agent, and the fourth isolation agent. In some embodiments, the sample is enriched for fetal cells using the first isolation agent, the third isolation agent, and the fifth isolation agent. In some embodiments, the sample is enriched for fetal cells using the first isolation agent, the third isolation agent, and the sixth isolation agent. In some embodiments, the sample is enriched for fetal cells using the first isolation agent, the fourth isolation agent, and the fifth isolation agent. In some embodiments, the sample is enriched for fetal cells using the first isolation agent, the fourth isolation agent, and the sixth isolation agent. In some embodiments, the sample is enriched for fetal cells using the first isolation agent, the fifth isolation agent, and the sixth isolation agent.

In some embodiments, the sample is enriched for fetal cells using the second isolation agent, the third isolation agent, and the fourth isolation agent. In some embodiments, the sample is enriched for fetal cells using the second isolation agent, the third isolation agent, and the fifth isolation agent. In some embodiments, the sample is enriched for fetal cells using the second isolation agent, the third isolation agent, and the sixth isolation agent. In some embodiments, the sample is enriched for fetal cells using the second isolation agent, the fourth isolation agent, and the fifth isolation agent. In some embodiments, the sample is enriched for fetal cells using the second isolation agent, the fourth isolation agent, and the sixth isolation agent. In some embodiments, the sample is enriched for fetal cells using the second isolation agent, the fifth isolation agent, and the sixth isolation agent.

In some embodiments, the sample is enriched for fetal cells using the third isolation agent, the fourth isolation agent, and the fifth isolation agent. In some embodiments, the sample is enriched for fetal cells using the third isolation agent, the fourth isolation agent, and the sixth isolation agent. In some embodiments, the sample is enriched for fetal cells using the fourth isolation agent, the fifth isolation agent, and the sixth isolation agent.

In some embodiments, the sample is enriched for fetal cells using the first isolation agent, the second isolation agent, the third isolation agent, and the fourth isolation agent. In some embodiments, the sample is enriched for fetal cells using the first isolation agent, the second isolation agent, the third isolation agent, and fifth isolation agent. In some embodiments, the sample is enriched for fetal cells using the first isolation agent, the second isolation agent, the third isolation agent, and the sixth isolation agent. In some embodiments, the sample is enriched for fetal cells using the first isolation agent, the second isolation agent, the fourth isolation agent, and the fifth isolation agent. In some embodiments, the sample is enriched for fetal cells using the first isolation agent, the second isolation agent, the fourth isolation agent, and the sixth isolation agent. In some embodiments, the sample is enriched for fetal cells using the first isolation agent, the fourth isolation agent, the fifth isolation agent, and the sixth isolation agent.

In some embodiments, the sample is enriched for fetal cells using the second isolation agent, the third isolation agent, the fourth isolation agent, and the fifth isolation agent. In some embodiments, the sample is enriched for fetal cells using the second isolation agent, the third isolation agent, the fourth isolation agent, and the sixth isolation agent. In some embodiments, the sample is enriched for fetal cells using the second isolation agent, the third isolation agent, the fifth isolation agent, and the sixth isolation agent. In some embodiments, the sample is enriched for fetal cells using the second isolation agent, the fourth isolation agent, the fifth isolation agent, and the sixth isolation agent.

In some embodiments, the sample is enriched for fetal cells using the third isolation agent, the fourth isolation agent, the fifth isolation agent, and the sixth isolation agent.

In some embodiments, the sample is enriched for fetal cells using the first isolation agent, the second isolation agent, the third isolation agent, the fourth isolation agent, and the firth isolation agent. In some embodiments, the sample is enriched for fetal cells using the first isolation agent, the third isolation agent, the fourth isolation agent, the fifth isolation agent, and the sixth isolation agent. In some embodiments, the sample is enriched for fetal cells using the first isolation agent, the second isolation agent, the fourth isolation agent, the fifth isolation agent, and the sixth isolation agent. In some embodiments, the sample is enriched for fetal cells using the first isolation agent, the second isolation agent, the third isolation agent, the fifth isolation agent, and the sixth isolation agent. In some embodiments, the sample is enriched for fetal cells using the first isolation agent, the second isolation agent, the third isolation agent, the fourth isolation agent, and the sixth isolation agent.

In some embodiments, the sample is enriched for fetal cells using the second isolation agent, the third isolation agent, the fourth isolation agent, the fifth isolation agent, and the sixth isolation agent. In some embodiments, the sample is enriched for fetal cells using the first isolation agent, the second isolation agent, the third isolation agent, the fourth isolation agent, the fifth isolation agent, and the sixth isolation agent.

In some embodiments, one of the first biomarker, the second biomarker, the third biomarker, the fourth biomarker, or the fifth biomarker is a Sct biomarker. In some embodiments, the first biomarker is a Sct biomarker. In some embodiments, the second biomarker is a Sct biomarker. In some embodiments, the third biomarker is a Sct biomarker. In some embodiments, the fourth biomarker is a Sct biomarker. In some embodiments, the fifth biomarker is a Sct biomarker.

In some embodiments, one of the first biomarker, the second biomarker, the third biomarker, the fourth biomarker, or the fifth biomarker is a H19 biomarker. In some embodiments, the first biomarker is a H19 biomarker. In some embodiments, the second biomarker is a H19 biomarker. In some embodiments, the third biomarker is a H19 biomarker. In some embodiments, the fourth biomarker is a H19 biomarker. In some embodiments, the fifth biomarker is a H19 biomarker.

In some embodiments, one of the first biomarker, the second biomarker, the third biomarker, the fourth biomarker, or the fifth biomarker is a Krt8 biomarker. In some embodiments, the first biomarker is a Krt8 biomarker. In some embodiments, the second biomarker is a Krt8 biomarker. In some embodiments, the third biomarker is a Krt8 biomarker. In some embodiments, the fourth biomarker is a Krt8 biomarker. In some embodiments, the fifth biomarker is a Krt8 biomarker.

In some embodiments, one of the first biomarker, the second biomarker, the third biomarker, the fourth biomarker, or the fifth biomarker is a Krt18 biomarker. In some embodiments, the first biomarker is a Krt18 biomarker. In some embodiments, the second biomarker is a Krt18 biomarker. In some embodiments, the third biomarker is a Krt18 biomarker. In some embodiments, the fourth biomarker is a Krt18 biomarker. In some embodiments, the fifth biomarker is a Krt18 biomarker.

In some embodiments, one of the first biomarker, the second biomarker, the third biomarker, the fourth biomarker, or the fifth biomarker is a Tfpi biomarker. In some embodiments, the first biomarker is a Tfpi biomarker. In some embodiments, the second biomarker is a Tfpi biomarker. In some embodiments, the third biomarker is a Tfpi biomarker. In some embodiments, the fourth biomarker is a Tfpi biomarker. In some embodiments, the fifth biomarker is a Tfpi biomarker.

In some embodiments, one of the first biomarker, the second biomarker, the third biomarker, the fourth biomarker, or the fifth biomarker is a Fthl17a biomarker. In some embodiments, the first biomarker is a Fthl17a biomarker. In some embodiments, the second biomarker is a Fthl17a biomarker. In some embodiments, the third biomarker is a Fthl17a biomarker. In some embodiments, the fourth biomarker is a Fthl17a biomarker. In some embodiments, the fifth biomarker is a Fthl17a biomarker.

In some embodiments, the sixth biomarker is Coro1a, Cd52, Rac2, Laptm5, or Ptprc. In some embodiments, the sixth biomarker is Coro1a, Cd52, Cyba, Rac2, Laptm5, or Ptprc. In some embodiments, the sixth biomarker is Coro1a. In some embodiments, the sixth biomarker is Cd52. In some embodiments, the sixth biomarker is Cyba. In some embodiments, the sixth biomarker is Rac2. In some embodiments, the sixth biomarker is Laptm5. In some embodiments, the sixth biomarker is Ptprc.

In some embodiments, the present disclosure provides a method of incubating a population of cells from a sample of a subject. In some embodiments, the population of cells comprises a subpopulation of fetal cells. In some embodiments, the population of cells comprises a subpopulation of maternal cells. In some embodiments, the population of cells comprises a subpopulation of fetal cells and a subpopulation of maternal cells. In some embodiments, the sample of the subject is incubated with from about one isolation agent to about ten isolation agents. In some embodiments, the sample of the subject is incubated with at least about one isolation agent, at least about two isolation agents at least about three isolation agents, at least about four isolation agents, at least about five isolation agents, at least about six isolation agents, at least about seven isolation agents, at least about eight isolation agents, at least about nine isolation agents, at least about ten isolation agents, or more. In some embodiments, the sample of the subject is incubated with at most about ten isolation agents, at most about nine isolation agents, at most about eight isolation agents, at most about seven isolation agents, at most about six isolation agents, at most about five isolation agents, at most about four isolation agents, at most about three isolation agents, at most about two isolation agents, at most about one isolation agent, or less. In some embodiments, the sample of the subject is incubated with about one isolation agent, about two isolation agents, about three isolation agents, about four isolation agents, about five isolation agents, about six isolation agents, about seven isolation agents, about eight isolation agents, about nine isolation agents, or about ten isolation agents.

In some embodiments, a first isolation agent is specific for a fetal cell biomarker and/or a maternal cell biomarker. In some embodiments, a second isolation agent is specific for a fetal cell biomarker and/or a maternal cell biomarker. In some embodiments, a third isolation agent is specific for a fetal cell biomarker and/or a maternal cell biomarker. In some embodiments, a fourth isolation agent is specific for a fetal cell biomarker and/or a maternal cell biomarker. In some embodiments, a fifth isolation agent is specific for a fetal cell biomarker and/or a maternal cell biomarker. In some embodiments, a sixth isolation agent is specific for a fetal cell biomarker and/or a maternal cell biomarker. In some embodiments, a seventh isolation agent is specific for a fetal cell biomarker and/or a maternal cell biomarker. In some embodiments, an eighth isolation agent is specific for a fetal cell biomarker and/or a maternal cell biomarker. In some embodiments, a ninth isolation agent is specific for a fetal cell biomarker and/or a maternal cell biomarker. In some embodiments, a tenth isolation agent is specific for a fetal cell biomarker and/or a maternal cell biomarker.

In some embodiments, the sample of the subject is incubated with six isolation agents. In some embodiments, the six isolation agents are a first isolation agent, a second isolation agent, a third isolation agent, a fourth isolation agent, a fifth isolation agent, and a sixth isolation agent. In some embodiments, the first isolation agent is specific for a first biomarker in the subpopulation of fetal cells. In some embodiments, the second isolation agent is specific for a second biomarker in the subpopulation of fetal cells. In some embodiments, the third isolation agent is specific for a third biomarker in the subpopulation of fetal cells. In some embodiments, the fourth isolation agent is specific for a fourth biomarker in the subpopulation of maternal cells. In some embodiments, the fifth isolation agent is specific for a fifth biomarker in the subpopulation of maternal cells. In some embodiments, the sixth isolation agent is specific for a sixth biomarker in the subpopulation of maternal cells.

In some embodiments, the maternal biological sample is incubated with at least one isolation agent.

In some embodiments, the method further comprises enriching the subpopulation of fetal cells after incubating. In some embodiments, the sample is enriched to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, the subpopulation of fetal cells is enriched from the subpopulation of maternal cells. In some embodiments, the enriched sample is enriched in fetal cells by positive selection using one or more isolation markers specific for one or more fetal biomarkers. In some embodiments, the enriched sample is enriched in fetal cells by negative selection by negative selection using one or more maternal biomarkers. In some embodiments, the enriched sample is enriched in fetal cells by positive selection and then subsequently negative selection. In some embodiments, the enriched sample is enriched in fetal cells by negative selection and then subsequently by positive selection. In some embodiments, sample is enriched for fetal cells by immunomagnetic cell separation. In some embodiments, the immunomagnetic cell separation is positive immunomagnetic cell selection. In some embodiments, the sample is enriched for fetal cells by fluorescence-activated cell sorting (FACS). In some embodiments, the ex vivo population of cells is isolated through magnetic-activated cell sorting (MACS). In some embodiments, the sample is enriched for fetal cells by microscopic microdissection. In some embodiments, the sample is enriched for fetal cells by microscopic imaging. In some embodiments, the sample is enriched for fetal cells using one or more fetal cell biomarkers. In some embodiments, the sample is enriched for fetal cells by negative immunomagnetic cell separation. In some embodiments, the negative immunomagnetic cell separation enriches for fetal cells using one or more maternal cell biomarkers.

In some embodiments, the sample is enriched for fetal cells using the first isolation agent, the second isolation agent, the third isolation agent, the fourth isolation agent, or the fifth isolation agent. In some embodiments, the sample is enriched for fetal cells using the first isolation agent. In some embodiments, the sample is enriched for fetal cells using the second isolation agent. In some embodiments, the sample is enriched for fetal cells using the third isolation agent. In some embodiments, the sample is enriched for fetal cells using the fourth isolation agent. In some embodiments, the sample is enriched for fetal cells using the fifth isolation agent. In some embodiments, the sample is enriched for fetal cells using the sixth isolation agent.

In some embodiments, the sample is enriched for fetal cells using the first isolation agent and the second isolation agent. In some embodiments, the sample is enriched for fetal cells using the first isolation agent and the third isolation agent. In some embodiments, the sample is enriched for fetal cells using the first isolation agent and the fourth isolation agent. In some embodiments, the sample is enriched for fetal cells using the first isolation agent the fifth isolation agent. In some embodiments, the sample is enriched for fetal cells using the first isolation agent and the sixth isolation agent.

In some embodiments, the sample is enriched for fetal cells using the second isolation agent and the third isolation agent. In some embodiments, the sample is enriched for fetal cells using the second isolation agent and the fourth isolation agent. In some embodiments, the sample is enriched for fetal cells using the second isolation agent and the fifth isolation agent. In some embodiments, the sample is enriched for fetal cells using the second isolation agent and the sixth isolation agent.

In some embodiments, the sample is enriched for fetal cells using the third isolation agent and the fourth isolation agent. In some embodiments, the sample is enriched for fetal cells using the third isolation agent and the fifth isolation agent. In some embodiments, the sample is enriched for fetal cells using the third isolation agent and the sixth isolation agent. In some embodiments, the sample is enriched for fetal cells using the fourth isolation agent and the fifth isolation agent. In some embodiments, the sample is enriched for fetal cells using the fourth isolation agent and the sixth isolation agent. In some embodiments, the sample is enriched for fetal cells using the fifth isolation agent and the sixth isolation agent.

In some embodiments, the sample is enriched for fetal cells using the first isolation agent, the second isolation agent, and the third isolation agent. In some embodiments, the sample is enriched for fetal cells using the first isolation agent, the second isolation agent, and the fourth isolation agent. In some embodiments, the sample is enriched for fetal cells using the first isolation agent, the second isolation agent, and the fifth isolation agent. In some embodiments, the sample is enriched for fetal cells using the first isolation agent, the second isolation agent, and the sixth isolation agent. In some embodiments, the sample is enriched for fetal cells using the first isolation agent, the third isolation agent, and the fourth isolation agent. In some embodiments, the sample is enriched for fetal cells using the first isolation agent, the third isolation agent, and the fifth isolation agent. In some embodiments, the sample is enriched for fetal cells using the first isolation agent, the third isolation agent, and the sixth isolation agent. In some embodiments, the sample is enriched for fetal cells using the first isolation agent, the fourth isolation agent, and the fifth isolation agent. In some embodiments, the sample is enriched for fetal cells using the first isolation agent, the fourth isolation agent, and the sixth isolation agent. In some embodiments, the sample is enriched for fetal cells using the first isolation agent, the fifth isolation agent, and the sixth isolation agent.

In some embodiments, the sample is enriched for fetal cells using the second isolation agent, the third isolation agent, and the fourth isolation agent. In some embodiments, the sample is enriched for fetal cells using the second isolation agent, the third isolation agent, and the fifth isolation agent. In some embodiments, the sample is enriched for fetal cells using the second isolation agent, the third isolation agent, and the sixth isolation agent. In some embodiments, the sample is enriched for fetal cells using the second isolation agent, the fourth isolation agent, and the fifth isolation agent. In some embodiments, the sample is enriched for fetal cells using the second isolation agent, the fourth isolation agent, and the sixth isolation agent. In some embodiments, the sample is enriched for fetal cells using the second isolation agent, the fifth isolation agent, and the sixth isolation agent.

In some embodiments, the sample is enriched for fetal cells using the third isolation agent, the fourth isolation agent, and the fifth isolation agent. In some embodiments, the sample is enriched for fetal cells using the third isolation agent, the fourth isolation agent, and the sixth isolation agent. In some embodiments, the sample is enriched for fetal cells using the fourth isolation agent, the fifth isolation agent, and the sixth isolation agent.

In some embodiments, the sample is enriched for fetal cells using the first isolation agent, the second isolation agent, the third isolation agent, and the fourth isolation agent. In some embodiments, the sample is enriched for fetal cells using the first isolation agent, the second isolation agent, the third isolation agent, and fifth isolation agent. In some embodiments, the sample is enriched for fetal cells using the first isolation agent, the second isolation agent, the third isolation agent, and the sixth isolation agent. In some embodiments, the sample is enriched for fetal cells using the first isolation agent, the second isolation agent, the fourth isolation agent, and the fifth isolation agent. In some embodiments, the sample is enriched for fetal cells using the first isolation agent, the second isolation agent, the fourth isolation agent, and the sixth isolation agent. In some embodiments, the sample is enriched for fetal cells using the first isolation agent, the fourth isolation agent, the fifth isolation agent, and the sixth isolation agent.

In some embodiments, the sample is enriched for fetal cells using the second isolation agent, the third isolation agent, the fourth isolation agent, and the fifth isolation agent. In some embodiments, the sample is enriched for fetal cells using the second isolation agent, the third isolation agent, the fourth isolation agent, and the sixth isolation agent. In some embodiments, the sample is enriched for fetal cells using the second isolation agent, the third isolation agent, the fifth isolation agent, and the sixth isolation agent. In some embodiments, the sample is enriched for fetal cells using the second isolation agent, the fourth isolation agent, the fifth isolation agent, and the sixth isolation agent.

In some embodiments, the sample is enriched for fetal cells using the third isolation agent, the fourth isolation agent, the fifth isolation agent, and the sixth isolation agent.

In some embodiments, the sample is enriched for fetal cells using the first isolation agent, the second isolation agent, the third isolation agent, the fourth isolation agent, and the firth isolation agent. In some embodiments, the sample is enriched for fetal cells using the first isolation agent, the third isolation agent, the fourth isolation agent, the fifth isolation agent, and the sixth isolation agent. In some embodiments, the sample is enriched for fetal cells using the first isolation agent, the second isolation agent, the fourth isolation agent, the fifth isolation agent, and the sixth isolation agent. In some embodiments, the sample is enriched for fetal cells using the first isolation agent, the second isolation agent, the third isolation agent, the fifth isolation agent, and the sixth isolation agent. In some embodiments, the sample is enriched for fetal cells using the first isolation agent, the second isolation agent, the third isolation agent, the fourth isolation agent, and the sixth isolation agent.

In some embodiments, the sample is enriched for fetal cells using the second isolation agent, the third isolation agent, the fourth isolation agent, the fifth isolation agent, and the sixth isolation agent. In some embodiments, the sample is enriched for fetal cells using the first isolation agent, the second isolation agent, the third isolation agent, the fourth isolation agent, the fifth isolation agent, and the sixth isolation agent.

In some embodiments, the method further comprises enriching the subpopulation of maternal cells after incubating. In some embodiments, the sample is enriched to provide an enriched sample. In some embodiments, the enriched sample is enriched in maternal cells. In some embodiments, the enriched sample is depleted in fetal cells. In some embodiments, the enriched sample is enriched in maternal cells and depleted in fetal cells. In some embodiments, the subpopulation of maternal cells is enriched from the subpopulation of fetal cells. In some embodiments, the enriched sample is enriched in maternal cells by positive selection using one or more isolation markers specific for one or more maternal biomarkers. In some embodiments, the enriched sample is enriched in maternal cells by negative selection by negative selection using one or more fetal biomarkers. In some embodiments, the enriched sample is enriched in maternal cells by positive selection and then subsequently negative selection. In some embodiments, the enriched sample is enriched in maternal cells by negative selection and then subsequently by positive selection. In some embodiments, sample is enriched for fetal cells by immunomagnetic cell separation. In some embodiments, the immunomagnetic cell separation is positive immunomagnetic cell selection. In some embodiments, the sample is enriched for maternal cells by fluorescence-activated cell sorting (FACS). In some embodiments, the ex vivo population of cells is isolated through magnetic-activated cell sorting (MACS). In some embodiments, the sample is enriched for maternal cells by microscopic microdissection. In some embodiments, the sample is enriched for maternal cells by microscopic imaging. In some embodiments, the sample is enriched for maternal cells using one or more fetal cell biomarkers. In some embodiments, the sample is enriched for maternal cells by negative immunomagnetic cell separation. In some embodiments, the negative immunomagnetic cell separation enriches for maternal cells using one or more fetal cell biomarkers.

In some embodiments, the sample is enriched for maternal cells using the first isolation agent, the second isolation agent, the third isolation agent, the fourth isolation agent, the fifth isolation agent, or the sixth isolation agent. In some embodiments, the sample is enriched for maternal cells using the first isolation agent. In some embodiments, the sample is enriched for maternal cells using the second isolation agent. In some embodiments, the sample is enriched for maternal cells using the third isolation agent. In some embodiments, the sample is enriched for maternal cells using the fourth isolation agent. In some embodiments, the sample is enriched for maternal cells using the fifth isolation agent. In some embodiments, the sample is enriched for maternal cells using the sixth isolation agent.

In some embodiments, the sample is enriched for maternal cells using the first isolation agent and the second isolation agent. In some embodiments, the sample is enriched for maternal cells using the first isolation agent and the third isolation agent. In some embodiments, the sample is enriched for maternal cells using the first isolation agent and the fourth isolation agent. In some embodiments, the sample is enriched for maternal cells using the first isolation agent and fifth isolation agent. In some embodiments, the sample is enriched for maternal cells using the first isolation agent and sixth isolation agent.

In some embodiments, the sample is enriched for maternal cells using the second isolation agent and the third isolation agent. In some embodiments, the sample is enriched for maternal cells using the second isolation agent and the fourth isolation agent. In some embodiments, the sample is enriched for maternal cells using the second isolation agent and the fifth isolation agent. In some embodiments, the sample is enriched for maternal cells using the second isolation agent and the sixth isolation agent.

In some embodiments, the sample is enriched for maternal cells using the third isolation agent and the fourth isolation agent. In some embodiments, the sample is enriched for maternal cells using the third isolation agent and the fifth isolation agent. In some embodiments, the sample is enriched for maternal cells using the third isolation agent and the sixth isolation agent. In some embodiments, the sample is enriched for maternal cells using the fourth isolation agent and the fifth isolation agent. In some embodiments, the sample is enriched for maternal cells using the fourth isolation agent and the sixth isolation agent. In some embodiments, the sample is enriched for maternal cells using the fifth isolation agent and the sixth isolation agent.

In some embodiments, the sample is enriched for maternal cells using the first isolation agent, the second isolation agent, and the third isolation agent. In some embodiments, the sample is enriched for maternal cells using the first isolation agent, the second isolation agent, and the fourth isolation agent. In some embodiments, the sample is enriched for maternal cells using the first isolation agent, the second isolation agent, and the fifth isolation agent. In some embodiments, the sample is enriched for maternal cells using the first isolation agent, the second isolation agent, and the sixth isolation agent. In some embodiments, the sample is enriched for maternal cells using the first isolation agent, the third isolation agent, and the fourth isolation agent. In some embodiments, the sample is enriched for maternal cells using the first isolation agent, the third isolation agent, and the fifth isolation agent. In some embodiments, the sample is enriched for maternal cells using the first isolation agent, the third isolation agent, and the sixth isolation agent. In some embodiments, the sample is enriched for maternal cells using the first isolation agent, the fourth isolation agent, and the fifth isolation agent. In some embodiments, the sample is enriched for maternal cells using the first isolation agent, the fourth isolation agent, and the sixth isolation agent. In some embodiments, the sample is enriched for maternal cells using the first isolation agent, the fifth isolation agent, and the sixth isolation agent.

In some embodiments, the sample is enriched for maternal cells using the second isolation agent, the third isolation agent, and the fourth isolation agent. In some embodiments, the sample is enriched for maternal cells using the second isolation agent, the third isolation agent, and the fifth isolation agent. In some embodiments, the sample is enriched for maternal cells using the second isolation agent, the third isolation agent, and the sixth isolation agent. In some embodiments, the sample is enriched for maternal cells using the second isolation agent, the fourth isolation agent, and the fifth isolation agent. In some embodiments, the sample is enriched for maternal cells using the second isolation agent, the fourth isolation agent, and the sixth isolation agent. In some embodiments, the sample is enriched for maternal cells using the second isolation agent, the fifth isolation agent, and the sixth isolation agent.

In some embodiments, the sample is enriched for maternal cells using the third isolation agent, the fourth isolation agent, and the fifth isolation agent. In some embodiments, the sample is enriched for maternal cells using the third isolation agent, the fourth isolation agent, and the sixth isolation agent. In some embodiments, the sample is enriched for maternal cells using the fourth isolation agent, the fifth isolation agent, and the sixth isolation agent.

In some embodiments, the sample is enriched for maternal cells using the first isolation agent, the second isolation agent, the third isolation agent, and the fourth isolation agent. In some embodiments, the sample is enriched for maternal cells using the first isolation agent, the second isolation agent, the third isolation agent, and fifth isolation agent. In some embodiments, the sample is enriched for maternal cells using the first isolation agent, the second isolation agent, the third isolation agent, and the sixth isolation agent. In some embodiments, the sample is enriched for maternal cells using the first isolation agent, the second isolation agent, the fourth isolation agent, and the fifth isolation agent. In some embodiments, the sample is enriched for maternal cells using the first isolation agent, the second isolation agent, the fourth isolation agent, and the sixth isolation agent. In some embodiments, the sample is enriched for maternal cells using the first isolation agent, the fourth isolation agent, the fifth isolation agent, and the sixth isolation agent.

In some embodiments, the sample is enriched for maternal cells using the second isolation agent, the third isolation agent, the fourth isolation agent, and the fifth isolation agent. In some embodiments, the sample is enriched for maternal cells using the second isolation agent, the third isolation agent, the fourth isolation agent, and the sixth isolation agent. In some embodiments, the sample is enriched for maternal cells using the second isolation agent, the third isolation agent, the fifth isolation agent, and the sixth isolation agent. In some embodiments, the sample is enriched for maternal cells using the second isolation agent, the fourth isolation agent, the fifth isolation agent, and the sixth isolation agent.

In some embodiments, the sample is enriched for maternal cells using the third isolation agent, the fourth isolation agent, the fifth isolation agent, and the sixth isolation agent.

In some embodiments, the sample is enriched for maternal cells using the first isolation agent, the second isolation agent, the third isolation agent, the fourth isolation agent, and the firth isolation agent. In some embodiments, the sample is enriched for maternal cells using the first isolation agent, the third isolation agent, the fourth isolation agent, the fifth isolation agent, and the sixth isolation agent. In some embodiments, the sample is enriched for maternal cells using the first isolation agent, the second isolation agent, the fourth isolation agent, the fifth isolation agent, and the sixth isolation agent. In some embodiments, the sample is enriched for maternal cells using the first isolation agent, the second isolation agent, the third isolation agent, the fifth isolation agent, and the sixth isolation agent. In some embodiments, the sample is enriched for maternal cells using the first isolation agent, the second isolation agent, the third isolation agent, the fourth isolation agent, and the sixth isolation agent.

In some embodiments, the sample is enriched for maternal cells using the second isolation agent, the third isolation agent, the fourth isolation agent, the fifth isolation agent, and the sixth isolation agent. In some embodiments, the sample is enriched for maternal cells using the first isolation agent, the second isolation agent, the third isolation agent, the fourth isolation agent, the fifth isolation agent, and the sixth isolation agent.

In some embodiments, one of the first biomarker, the second biomarker, or the third biomarker is a Sct biomarker. In some embodiments, the first biomarker is a Sct biomarker. In some embodiments, the second biomarker is a Sct biomarker. In some embodiments, the third biomarker is a Sct biomarker.

In some embodiments, one of the first biomarker, the second biomarker, or the third biomarker is a H19 biomarker. In some embodiments, the first biomarker is a H19 biomarker. In some embodiments, the second biomarker is a H19 biomarker. In some embodiments, the third biomarker is a H19 biomarker.

In some embodiments, one of the first biomarker, the second biomarker, or the third biomarker is a Krt8 biomarker. In some embodiments, the first biomarker is a Krt8 biomarker. In some embodiments, the second biomarker is a Krt8 biomarker. In some embodiments, the third biomarker is a Krt8 biomarker.

In some embodiments, one of the first biomarker, the second biomarker, or the third biomarker is a Krt18 biomarker. In some embodiments, the first biomarker is a Krt18 biomarker. In some embodiments, the second biomarker is a Krt18 biomarker. In some embodiments, the third biomarker is a Krt18 biomarker.

In some embodiments, one of the first biomarker, the second biomarker, or the third biomarker is a Tfpi biomarker. In some embodiments, the first biomarker is a Tfpi biomarker. In some embodiments, the second biomarker is a Tfpi biomarker. In some embodiments, the third biomarker is a Tfpi biomarker.

In some embodiments, one of the first biomarker, the second biomarker, or the third biomarker is a Fthl17a biomarker. In some embodiments, the first biomarker is a Fthl17a biomarker. In some embodiments, the second biomarker is a Fthl17a biomarker. In some embodiments, the third biomarker is a Fthl17a biomarker.

In some embodiments, the fourth biomarker, the fifth biomarker, or the sixth biomarker is Coro1a, Cd52, Cyba, Rac2, Laptm5, or Ptprc. In some embodiments, the fourth biomarker is a Coro1a biomarker. In some embodiments, the fifth biomarker is a Coro1a biomarker. In some embodiments, the sixth biomarker is a Coro1a biomarker.

In some embodiments, the fourth biomarker is a Cd52 biomarker. In some embodiments, the fifth biomarker is a Cd52 biomarker. In some embodiments, the sixth biomarker is a Cd52 biomarker. In some embodiments, the fourth biomarker is a Cyba biomarker. In some embodiments, the fifth biomarker is a Cyba biomarker. In some embodiments, the sixth biomarker is a Cyba biomarker.

In some embodiments, the fourth biomarker is a Rac2 biomarker. In some embodiments, the fifth biomarker is a Rac2. In some embodiments, the sixth biomarker is a Rac2 biomarker.

In some embodiments, the fourth biomarker is a Laptm5 biomarker. In some embodiments, the fifth biomarker is a Laptm5 biomarker. In some embodiments, the sixth biomarker is a Laptm5 biomarker.

In some embodiments, the fourth biomarker is a Ptprc biomarker. In some embodiments, the fifth biomarker is a Ptprc biomarker. In some embodiments, the sixth biomarker is a Ptprc biomarker.

In some embodiments, the present disclosure provides a method of incubating a population of cells from a sample of a subject. In some embodiments, the population of cells comprises a subpopulation of fetal cells. In some embodiments, the population of cells comprises a subpopulation of maternal cells. In some embodiments, the population of cells comprises a subpopulation of fetal cells and a subpopulation of maternal cells. In some embodiments, the sample of the subject is incubated with from about one isolation agent to about ten isolation agents.

In some embodiments, the sample of the subject is incubated with at least about one isolation agent, at least about two isolation agents at least about three isolation agents, at least about four isolation agents, at least about five isolation agents, at least about six isolation agents, at least about seven isolation agents, at least about eight isolation agents, at least about nine isolation agents, at least about ten isolation agents, or more. In some embodiments, the sample of the subject is incubated with at most about ten isolation agents, at most about nine isolation agents, at most about eight isolation agents, at most about seven isolation agents, at most about six isolation agents, at most about five isolation agents, at most about four isolation agents, at most about three isolation agents, at most about two isolation agents, at most about one isolation agent, or less. In some embodiments, the sample of the subject is incubated with about one isolation agent, about two isolation agents, about three isolation agents, about four isolation agents, about five isolation agents, about six isolation agents, about seven isolation agents, about eight isolation agents, about nine isolation agents, or about ten isolation agents.

In some embodiments, the sample of the subject is incubated with six isolation agents. In some embodiments, the six isolation agents are a first isolation agent, a second isolation agent, a third isolation agent, a fourth isolation agent, a fifth isolation agent, a sixth isolation agent, and a seventh isolation agent. In some embodiments, the first isolation agent is specific for a first biomarker in the subpopulation of maternal cells. In some embodiments, the second isolation agent is specific for a second biomarker in the subpopulation of maternal cells. In some embodiments, the third isolation agent is specific for a third biomarker in the subpopulation of maternal cells. In some embodiments, the fourth isolation agent is specific for a fourth biomarker in the subpopulation of maternal cells. In some embodiments, the fifth isolation agent is specific for a fifth biomarker in the subpopulation of maternal cells. In some embodiments, the sixth isolation agent is specific for a sixth biomarker in the subpopulation of maternal cells. In some embodiments, the seventh isolation agent is specific for a seventh biomarker in the subpopulation of fetal cells.

In some embodiments, a first isolation agent is specific for a fetal cell biomarker and/or a maternal cell biomarker. In some embodiments, a second isolation agent is specific for a fetal cell biomarker and/or a maternal cell biomarker. In some embodiments, a third isolation agent is specific for a fetal cell biomarker and/or a maternal cell biomarker. In some embodiments, a fourth isolation agent is specific for a fetal cell biomarker and/or a maternal cell biomarker. In some embodiments, a fifth isolation agent is specific for a fetal cell biomarker and/or a maternal cell biomarker. In some embodiments, a sixth isolation agent is specific for a fetal cell biomarker and/or a maternal cell biomarker. In some embodiments, a seventh isolation agent is specific for a fetal cell biomarker and/or a maternal cell biomarker. In some embodiments, an eighth isolation agent is specific for a fetal cell biomarker and/or a maternal cell biomarker. In some embodiments, a ninth isolation agent is specific for a fetal cell biomarker and/or a maternal cell biomarker. In some embodiments, a tenth isolation agent is specific for a fetal cell biomarker and/or a maternal cell biomarker.

In some embodiments, the maternal biological sample is incubated with at least one isolation agent.

In some embodiments, the at least one isolation agent is an affinity reagent. In some embodiments, the affinity reagent is a ligand. In some embodiments, the affinity reagent is a protein. In some embodiments, the at least one isolation agent is an antibody. In some embodiments, the at least one isolation agent is labeled. In some embodiments, the at least one isolation agent is fluorescently labeled. In some embodiments, the at least one isolation agent is coupled to a fluorescent protein or peptide. In some embodiments, the affinity reagent is a nucleic acid molecule. In some embodiments, the at least one isolation agent is an aptamer. In some embodiments, the at least one isolation agent is a nucleic acid probe. In some embodiments, the nucleic acid probe is DNA. In some embodiments, the nucleic acid probe is RNA.

In some embodiments, one of the first isolation agent, the second isolation agent, the third isolation agent, the fourth isolation agent, the fifth isolation agent, the sixth isolation agent, or the seventh isolation agent is nucleic acid probe. In some embodiments, the first isolation agent is a nucleic acid probe. In some embodiments, the second isolation agent is a nucleic acid probe. In some embodiments, the third isolation agent is a nucleic acid probe. In some embodiments, the fourth isolation agent is a nucleic acid probe. In some embodiments, the fifth isolation agent is nucleic acid probe. In some embodiments, the sixth isolation agent is a nucleic acid probe. In some embodiments, the seventh isolation agent is a nucleic acid probe. In some embodiments, the first isolation agent, the second isolation agent, the third isolation agent, the fourth isolation agent, the fifth isolation agent, the sixth isolation agent, and the seventh isolation agent are a nucleic acid probe.

In some embodiments, one of the first isolation agent, the second isolation agent, the third isolation agent, the fourth isolation agent, the fifth isolation agent, the sixth isolation agent, or the seventh isolation agent is a ligand. In some embodiments, the first isolation agent is a ligand. In some embodiments, the second isolation agent is a ligand. In some embodiments, the third isolation agent is a ligand. In some embodiments, the fourth isolation agent is a ligand. In some embodiments, the fourth isolation agent is a ligand. In some embodiments, the fifth isolation agent is a ligand. In some embodiments, the sixth isolation agent is a ligand. In some embodiments, the seventh isolation agent is a ligand. In some embodiments, the first isolation agent, the second isolation agent, the third isolation agent, the fourth isolation agent, the fifth isolation agent, the sixth isolation agent, and the seventh isolation agent are a ligand.

In some embodiments, the isolation agent couples to an extracellular biomarker. In some embodiments, the isolation agent couples to an extracellular protein. In some embodiments, the isolation agent couples to a cell surface protein. In some embodiments, the isolation agent couples to a soluble cell surface protein. In some embodiments, the isolation agent couples to a cell-surface receptor. In some embodiments, the isolation agent couples to a transmembrane protein. In some embodiments, the isolation agent couples to a transmembrane channel protein.

In some embodiments, the isolation agent couples to an intracellular biomarker. In some embodiments, the isolation agent couples to an intracellular protein. In some embodiments, the isolation agent couples to an enzyme. In some embodiments, the isolation agent couples to a phosphoprotein. In some embodiments, the isolation agent couples to a growth factor. In some embodiments, the isolation agent couples to a structural protein.

In some embodiments, the isolation agent couples to a nucleic acid biomarker. In some embodiments, the isolation agent couples to a DNA biomarker. In some embodiments, the isolation agent couples to an RNA biomarker. In some embodiments, the isolation agent couples to mRNA. In some embodiments, the isolation agent couples to rRNA. In some embodiments, the isolation agent couples to lncRNA. In some embodiments, the second isolation agent couples to miRNA. In some embodiments, the isolation agent couples to siRNA.

In some embodiments, each of the first isolation agent, the second isolation agent, the third isolation agent, the fourth isolation agent, the fifth isolation agent, the sixth isolation agent, or the seventh isolation agent is independently specific for a cell surface marker or protein. In some embodiments, the first isolation agent is specific for a cell surface marker or protein. In some embodiments, the second isolation agent is specific for a cell surface marker or protein. In some embodiments, the third isolation agent is specific for a cell surface marker or protein. In some embodiments, the fourth isolation agent is specific for a cell surface marker or protein. In some embodiments, the fifth isolation agent is specific for a cell surface marker or protein. In some embodiments, the sixth isolation agent is specific for a cell surface marker or protein. In some embodiments, the seventh isolation agent is specific for a cell surface marker or protein. In some embodiments, the first isolation agent, the second isolation agent, the third isolation agent, the fourth isolation agent, the fifth isolation agent, the sixth isolation agent, and the seventh isolation agent are specific for a cell surface marker or protein.

In some embodiments, the first isolation agent, the second isolation agent, the third isolation agent, the fourth isolation agent, the fifth isolation agent, the sixth isolation agent, or the seventh isolation agent is specific for a soluble cell surface marker or protein. In some embodiments, the first isolation agent is specific for a soluble cell surface marker or protein. In some embodiments, the second isolation agent is specific for a soluble cell surface marker or protein. In some embodiments, the third isolation agent is specific for a soluble cell surface marker or protein. In some embodiments, the fourth isolation agent is specific for a soluble cell surface marker or protein. In some embodiments, the fifth isolation agent is specific for a soluble cell surface marker or protein. In some embodiments, the sixth isolation agent is specific for a soluble cell surface marker or protein. In some embodiments, the seventh isolation agent is specific for a soluble cell surface marker or protein. In some embodiments, the first isolation agent, the second isolation agent, the third isolation agent, the fourth isolation agent, the fifth isolation agent, the sixth isolation agent, and the seventh isolation agent are specific for a soluble cell surface marker or protein.

In some embodiments, the first isolation agent, the second isolation agent, the third isolation agent, the fourth isolation agent, the fifth isolation agent, the sixth isolation agent, or the seventh isolation agent is specific for a nucleic acid marker. In some embodiments, the first isolation agent is specific for a nucleic acid marker. In some embodiments, the second isolation agent is specific for a nucleic acid marker. In some embodiments, the third isolation agent is specific for a nucleic acid marker. In some embodiments, the fourth isolation agent is specific for a nucleic acid marker. In some embodiments, the fifth isolation agent is specific for a nucleic acid marker. In some embodiments, the sixth isolation agent is specific for a nucleic acid marker. In some embodiments, the seventh isolation agent is specific for a nucleic acid marker. In some embodiments, each of the first isolation agent, the second isolation agent, the third isolation agent, the fourth isolation agent, the fifth isolation agent, the sixth isolation agent, and the seventh isolation agent is independently specific for a nucleic acid marker.

In some embodiments, the method further comprises enriching the subpopulation of fetal cells after incubating. In some embodiments, the sample is enriched to provide an enriched sample. In some embodiments, the enriched sample is enriched in fetal cells. In some embodiments, the enriched sample is depleted in maternal cells. In some embodiments, the enriched sample is enriched in fetal cells and depleted in maternal cells. In some embodiments, the subpopulation of fetal cells is enriched from the subpopulation of maternal cells. In some embodiments, the enriched sample is enriched in fetal cells by positive selection using one or more isolation markers specific for one or more fetal biomarkers. In some embodiments, the enriched sample is enriched in fetal cells by negative selection by negative selection using one or more maternal biomarkers. In some embodiments, the enriched sample is enriched in fetal cells by positive selection and then subsequently negative selection. In some embodiments, the enriched sample is enriched in fetal cells by negative selection and then subsequently by positive selection. In some embodiments, a sample is enriched for fetal cells by immunomagnetic cell separation. In some embodiments, the immunomagnetic cell separation is positive immunomagnetic cell selection. In some embodiments, the sample is enriched for fetal cells by fluorescence-activated cell sorting (FACS). In some embodiments, the ex vivo population of cells is isolated through magnetic-activated cell sorting (MACS). In some embodiments, the sample is enriched for fetal cells by microscopic microdissection. In some embodiments, the sample is enriched for fetal cells by microscopic imaging. In some embodiments, the sample is enriched for fetal cells using one or more fetal cell biomarkers. In some embodiments, the sample is enriched for fetal cells by negative immunomagnetic cell separation. In some embodiments, the negative immunomagnetic cell separation enriches for fetal cells using one or more maternal cell biomarkers.

In some embodiments, the sample is enriched for fetal cells using the first isolation agent, the second isolation agent, the third isolation agent, the fourth isolation agent, the fifth isolation agent, the sixth isolation agent, or the seventh isolation agent. In some embodiments, the sample is enriched for fetal cells using the first isolation agent. In some embodiments, the sample is enriched for fetal cells using the second isolation agent. In some embodiments, the sample is enriched for fetal cells using the third isolation agent. In some embodiments, the sample is enriched for fetal cells using the fourth isolation agent. In some embodiments, the sample is enriched for fetal cells using the fifth isolation agent. In some embodiments, the sample is enriched for fetal cells using the sixth isolation agent. In some embodiments, the sample is enriched for fetal cells using the seventh isolation agent.

In some embodiments, the sample is enriched for fetal cells using the first isolation agent and the second isolation agent. In some embodiments, the sample is enriched for fetal cells using the first isolation agent and the third isolation agent. In some embodiments, the sample is enriched for fetal cells using the first isolation agent and the fourth isolation agent. In some embodiments, the sample is enriched for fetal cells using the first isolation agent the fifth isolation agent. In some embodiments, the sample is enriched for fetal cells using the first isolation agent and the sixth isolation agent. In some embodiments, the sample is enriched for fetal cells using the first isolation agent and the seventh isolation agent.

In some embodiments, the sample is enriched for fetal cells using the second isolation agent and the third isolation agent. In some embodiments, the sample is enriched for fetal cells using the second isolation agent and the fourth isolation agent. In some embodiments, the sample is enriched for fetal cells using the second isolation agent and the fifth isolation agent. In some embodiments, the sample is enriched for fetal cells using the second isolation agent and the sixth isolation agent. In some embodiments, the sample is enriched for fetal cells using the second isolation agent and the seventh isolation agent.

In some embodiments, the sample is enriched for fetal cells using the third isolation agent and the fourth isolation agent. In some embodiments, the sample is enriched for fetal cells using the third isolation agent and the fifth isolation agent. In some embodiments, the sample is enriched for fetal cells using the third isolation agent and the sixth isolation agent. In some embodiments, the sample is enriched for fetal cells using the third isolation agent and the seventh isolation agent.

In some embodiments, the sample is enriched for fetal cells using the fourth isolation agent and the fifth isolation agent. In some embodiments, the sample is enriched for fetal cells using the fourth isolation agent and the sixth isolation agent. In some embodiments, the sample is enriched for fetal cells using the fourth isolation agent and the seventh isolation agent. In some embodiments, the sample is enriched for fetal cells using the fifth isolation agent and the sixth isolation agent. In some embodiments, the sample is enriched for fetal cells using the fifth isolation agent the seventh isolation agent. In some embodiments, the sample is enriched for fetal cells using the sixth isolation agent and the seventh isolation agent.

In some embodiments, the sample is enriched for fetal cells using the first isolation agent, the second isolation agent, and the third isolation agent. In some embodiments, the sample is enriched for fetal cells using the first isolation agent, the second isolation agent, and the fourth isolation agent. In some embodiments, the sample is enriched for fetal cells using the first isolation agent, the second isolation agent, and the fifth isolation agent. In some embodiments, the sample is enriched for fetal cells using the first isolation agent, the second isolation agent, and the sixth isolation agent. In some embodiments, the sample is enriched for fetal cells using the first isolation agent, the second isolation agent, the seventh isolation agent.

In some embodiments, the sample is enriched for fetal cells using the first isolation agent, the third isolation agent, and the fourth isolation agent. In some embodiments, the sample is enriched for fetal cells using the first isolation agent, the third isolation agent, and the fifth isolation agent. In some embodiments, the sample is enriched for fetal cells using the first isolation agent, the third isolation agent, and the sixth isolation agent. In some embodiments, the sample is enriched for fetal cells using the first isolation agent, the third isolation agent, and the seventh isolation agent. In some embodiments, the sample is enriched for fetal cells using the first isolation agent, the fourth isolation agent, and the fifth isolation agent. In some embodiments, the sample is enriched for fetal cells using the first isolation agent, the fourth isolation agent, and the sixth isolation agent. In some embodiments, the sample is enriched for fetal cells using the first isolation agent, the fourth isolation agent, and the seventh isolation agent. In some embodiments, the sample is enriched for fetal cells using the first isolation agent, the fifth isolation agent, and the sixth isolation agent. In some embodiments, the sample is enriched for fetal cells using the first isolation agent, the fifth isolation agent, and the seventh isolation agent. In some embodiments, the sample is enriched for fetal cells using the first isolation agent, the sixth isolation agent, and the seventh isolation agent.

In some embodiments, the sample is enriched for fetal cells using the second isolation agent, the third isolation agent, and the fourth isolation agent. In some embodiments, the sample is enriched for fetal cells using the second isolation agent, the third isolation agent, and the fifth isolation agent. In some embodiments, the sample is enriched for fetal cells using the second isolation agent, the third isolation agent, and the sixth isolation agent. In some embodiments, the sample is enriched for fetal cells using the second isolation agent, the third isolation agent, and the seventh isolation agent. In some embodiments, the sample is enriched for fetal cells using the second isolation agent, the fourth isolation agent, and the fifth isolation agent. In some embodiments, the sample is enriched for fetal cells using the second isolation agent, the fourth isolation agent, and the sixth isolation agent. In some embodiments, the sample is enriched for fetal cells using the second isolation agent, the fourth isolation agent, and the seventh isolation agent. In some embodiments, the sample is enriched for fetal cells using the second isolation agent, the fifth isolation agent, and the sixth isolation agent. In some embodiments, the sample is enriched for fetal cells using the second isolation agent, the fifth isolation agent, and the seventh isolation agent. In some embodiments, the sample is enriched for fetal cells using the second isolation agent, the sixth isolation agent, and the seventh isolation agent.

In some embodiments, the sample is enriched for fetal cells using the third isolation agent, the fourth isolation agent, and the fifth isolation agent. In some embodiments, the sample is enriched for fetal cells using the third isolation agent, the fourth isolation agent, and the sixth isolation agent. In some embodiments, the sample is enriched for fetal cells using the third isolation agent, the fourth isolation agent, and the seventh isolation agent. In some embodiments, the sample is enriched for fetal cells using the third isolation agent, the fifth isolation agent, and the seventh isolation agent. In some embodiments, the sample is enriched in fetal cells using the third isolation agent, the fifth isolation agent, and the sixth isolation agent. In some embodiments, the sample is enriched in fetal cells using the third isolation agent, the sixth isolation agent, and the seventh isolation agent.

In some embodiments, the sample is enriched for fetal cells using the fourth isolation agent, the fifth isolation agent, and the sixth isolation agent. In some embodiments, the sample is enriched for fetal cells using the fourth isolation agent, the fifth isolation agent, and the seventh isolation agent. In some embodiments, the sample is enriched for fetal cells using the fourth isolation agent, the sixth isolation agent, and the seventh isolation agent. In some embodiments, the sample is enriched in fetal cells using the fifth isolation agent, the sixth isolation agent, and the seventh isolation agent.

In some embodiments, the sample is enriched for fetal cells using the first isolation agent, the second isolation agent, the third isolation agent, and the fourth isolation agent. In some embodiments, the sample is enriched for fetal cells using the first isolation agent, the second isolation agent, the third isolation agent, and fifth isolation agent. In some embodiments, the sample is enriched for fetal cells using the first isolation agent, the second isolation agent, the third isolation agent, and the sixth isolation agent. In some embodiments, the sample is enriched for fetal cells using the first isolation agent, the second isolation agent, the third isolation agent, and the seventh isolation agent. In some embodiments, the sample is enriched for fetal cells using the first isolation agent, the second isolation agent, the fourth isolation agent, and the fifth isolation agent. In some embodiments, the sample is enriched for fetal cells using the first isolation agent, the second isolation agent, the fourth isolation agent, and the sixth isolation agent. In some embodiments, the sample is enriched for fetal cells using the first isolation agent, the second isolation agent, the fourth isolation agent, and the seventh isolation agent. In some embodiments, the sample is enriched for fetal cells using the first isolation agent, the fourth isolation agent, the fifth isolation agent, and the sixth isolation agent. In some embodiments, the sample is enriched for fetal cells using the first isolation agent, the fourth isolation agent, the sixth isolation agent, and the seventh isolation agent. In some embodiments, the sample is enriched for fetal cells using the first isolation agent, the fifth isolation agent, the sixth isolation agent, and the seventh isolation agent.

In some embodiments, the sample is enriched for fetal cells using the second isolation agent, the third isolation agent, the fourth isolation agent, and the fifth isolation agent. In some embodiments, the sample is enriched for fetal cells using the second isolation agent, the third isolation agent, the fourth isolation agent, and the sixth isolation agent. In some embodiments, the sample is enriched for fetal cells using the second isolation agent, the third isolation agent, the fourth isolation agent, and the seventh isolation agent. In some embodiments, the sample is enriched for fetal cells using the second isolation agent, the third isolation agent, the fifth isolation agent, and the sixth isolation agent. In some embodiments, the sample is enriched for fetal cells using the second isolation agent, the third isolation agent, the fifth isolation agent, and the seventh isolation agent. In some embodiments, the sample is enriched for fetal cells using the second isolation agent, the fourth isolation agent, the fifth isolation agent, and the sixth isolation agent. In some embodiments, the sample is enriched for fetal cells using the second isolation agent, the fourth isolation agent, the fifth isolation agent, and the seventh isolation agent. In some embodiments, the sample is enriched for fetal cells using the second isolation agent, the fifth isolation agent, the sixth isolation agent, and the seventh isolation agent.

In some embodiments, the sample is enriched for fetal cells using the third isolation agent, the fourth isolation agent, the fifth isolation agent, and the sixth isolation agent. In some embodiments, the sample is enriched for fetal cells using the fourth isolation agent, the fifth isolation agent, the sixth isolation agent, and the seventh isolation agent.

In some embodiments, the sample is enriched for fetal cells using the first isolation agent, the second isolation agent, the third isolation agent, the fourth isolation agent, and the fifth isolation agent. In some embodiments, the sample is enriched for fetal cells using the first isolation agent, the third isolation agent, the fourth isolation agent, the fifth isolation agent, and the sixth isolation agent. In some embodiments, the sample is enriched for fetal cells using the first isolation agent, the second isolation agent, the fourth isolation agent, the fifth isolation agent, and the sixth isolation agent. In some embodiments, the sample is enriched for fetal cells using the first isolation agent, the second isolation agent, the third isolation agent, the fifth isolation agent, and the sixth isolation agent. In some embodiments, the sample is enriched for fetal cells using the first isolation agent, the second isolation agent, the third isolation agent, the fourth isolation agent, and the sixth isolation agent.

In some embodiments, the sample is enriched for fetal cells using the second isolation agent, the third isolation agent, the fourth isolation agent, the fifth isolation agent, and the sixth isolation agent. In some embodiments, the sample is enriched for fetal cells using the second isolation agent, the third isolation agent, the fourth isolation agent, the fifth isolation agent, and the seventh isolation agent.

In some embodiments, the sample is enriched for fetal cells using the first isolation agent, the second isolation agent, the fourth isolation agent, the fifth isolation agent, and the seventh isolation agent. In some embodiments, the sample is enriched for fetal cells using the first isolation agent, the second isolation agent, the third isolation agent, the fifth isolation agent, and the seventh isolation agent. In some embodiments, the sample is enriched in fetal cells using the first isolation agent, the second isolation agent, the third isolation agent, the fourth isolation agent, and the seventh isolation agent. In some embodiments, the sample is enriched in fetal cells using the first isolation agent, the second isolation agent, the third isolation agent, the fifth isolation agent, and the sixth isolation agent. In some embodiments, the sample is enriched in fetal cells using the first isolation agent, the second isolation unit, the third isolation unit, the fifth isolation agent, and the seventh isolation agent. In some embodiments, the sample is enriched in fetal cells using the first isolation agent, the second isolation agent, the third isolation agent, the sixth isolation agent, and the seventh isolation agent.

In some embodiments, the sample is enriched for fetal cells using the second isolation agent, the fourth isolation agent, the fifth isolation agent, the sixth isolation agent, and the seventh isolation agent. In some embodiments, the sample is enriched for fetal cells using the second isolation agent, the third isolation agent, the fifth isolation agent, the sixth isolation agent, and the seventh isolation agent. In some embodiments, the sample is enriched for fetal cells using the second isolation agent, the third isolation agent, the fourth isolation agent, the sixth isolation agent, and seventh isolation agent. In some embodiments, the sample is enriched for fetal cells using the second isolation agent, the third isolation agent, the fourth isolation agent, the fifth isolation agent, and the seventh isolation agent.

In some embodiments, the sample is enriched for fetal cells using the third isolation agent, the fourth isolation agent, the fifth isolation agent, the sixth isolation agent, and the seventh isolation agent.

In some embodiments, the sample is enriched for fetal cells using the first isolation agent, the second isolation agent, the third isolation agent, the fourth isolation agent, the fifth isolation agent, and the sixth isolation agent. In some embodiments, the sample is enriched for fetal cells using the first isolation agent, the third isolation agent, the fourth isolation agent, the fifth isolation agent, the sixth isolation agent, and the seventh isolation agent. In some embodiments, the sample is enriched for fetal cells using the first isolation agent, the second isolation agent, the fourth isolation agent, the fifth isolation agent, the sixth isolation agent, and the seventh isolation agent. In some embodiments, the sample is enriched for fetal cells using the first isolation agent, the second isolation, the third isolation agent, the fifth isolation agent, the sixth isolation agent, and the seventh isolation agent. In some embodiments, the sample is enriched for fetal cells using the first isolation agent, the second isolation agent, the third isolation agent, the fourth isolation agent, the sixth isolation agent, and the seventh isolation agent. In some embodiments, the sample is enriched for fetal cells using the first isolation agent, the second isolation agent, the third isolation agent, the fourth isolation agent, the fifth isolation, and the seventh isolation agent.

In some embodiments, the sample is enriched for fetal cells using the second isolation agent, the third isolation agent, the fourth isolation agent, the fifth isolation agent, the sixth isolation agent, and the seventh isolation agent. In some embodiments, the sample is enriched for fetal cells using the first isolation agent, the second isolation agent, the third isolation agent, the fourth isolation agent, the fifth isolation agent, the sixth isolation agent, and the seventh isolation agent.

In some embodiments, the method further comprises enriching the subpopulation of maternal cells after incubating. In some embodiments, the sample is enriched to provide an enriched sample. In some embodiments, the enriched sample is enriched in maternal cells. In some embodiments, the enriched sample is depleted in fetal cells. In some embodiments, the enriched sample is enriched in maternal cells and depleted in fetal cells. In some embodiments, the subpopulation of maternal cells is enriched from the subpopulation of fetal cells. In some embodiments, the enriched sample is enriched in maternal cells by positive selection using one or more isolation markers specific for one or more maternal biomarkers. In some embodiments, the enriched sample is enriched in maternal cells by negative selection by negative selection using one or more fetal biomarkers. In some embodiments, the enriched sample is enriched in maternal cells by positive selection and then subsequently negative selection. In some embodiments, the enriched sample is enriched in maternal cells by negative selection and then subsequently by positive selection. In some embodiments, sample is enriched for fetal cells by immunomagnetic cell separation. In some embodiments, the immunomagnetic cell separation is positive immunomagnetic cell selection. In some embodiments, the sample is enriched for maternal cells by fluorescence-activated cell sorting (FACS). In some embodiments, the sample is enriched for maternal cells by microscopic microdissection. In some embodiments, the sample is enriched for maternal cells by microscopic imaging. In some embodiments, the sample is enriched for maternal cells using one or more fetal cell biomarkers. In some embodiments, the sample is enriched for maternal cells by negative immunomagnetic cell separation. In some embodiments, the negative immunomagnetic cell separation enriches for maternal cells using one or more fetal cell biomarkers.

In some embodiments, the sample is enriched for maternal cells using the first isolation agent, the second isolation agent, the third isolation agent, the fourth isolation agent, the fifth isolation agent, the sixth isolation agent, or the seventh isolation agent. In some embodiments, the sample is enriched for maternal cells using the first isolation agent. In some embodiments, the sample is enriched for maternal cells using the second isolation agent. In some embodiments, the sample is enriched for maternal cells using the third isolation agent. In some embodiments, the sample is enriched for maternal cells using the fourth isolation agent. In some embodiments, the sample is enriched for maternal cells using the fifth isolation agent. In some embodiments, the sample is enriched for maternal cells using the sixth isolation agent. In some embodiments, the sample is enriched for maternal cells using the seventh isolation agent.

In some embodiments, the sample is enriched for maternal cells using the first isolation agent and the second isolation agent. In some embodiments, the sample is enriched for maternal cells using the first isolation agent and the third isolation agent. In some embodiments, the sample is enriched for maternal cells using the first isolation agent and the fourth isolation agent. In some embodiments, the sample is enriched for maternal cells using the first isolation agent the fifth isolation agent. In some embodiments, the sample is enriched for maternal cells using the first isolation agent and the sixth isolation agent. In some embodiments, the sample is enriched for maternal cells using the first isolation agent and the seventh isolation agent.

In some embodiments, the sample is enriched for maternal cells using the second isolation agent and the third isolation agent. In some embodiments, the sample is enriched for maternal cells using the second isolation agent and the fourth isolation agent. In some embodiments, the sample is enriched for maternal cells using the second isolation agent and the fifth isolation agent. In some embodiments, the sample is enriched for maternal cells using the second isolation agent and the sixth isolation agent. In some embodiments, the sample is enriched for maternal cells using the second isolation agent and the seventh isolation agent.

In some embodiments, the sample is enriched for maternal cells using the third isolation agent and the fourth isolation agent. In some embodiments, the sample is enriched for maternal cells using the third isolation agent and the fifth isolation agent. In some embodiments, the sample is enriched for maternal cells using the third isolation agent and the sixth isolation agent. In some embodiments, the sample is enriched for maternal cells using the third isolation agent and the seventh isolation agent.

In some embodiments, the sample is enriched for maternal cells using the fourth isolation agent and the fifth isolation agent. In some embodiments, the sample is enriched for maternal cells using the fourth isolation agent and the sixth isolation agent. In some embodiments, the sample is enriched for maternal cells using the fourth isolation agent and the seventh isolation agent.

In some embodiments, the sample is enriched for maternal cells using the fifth isolation agent and the sixth isolation agent. In some embodiments, the sample is enriched for maternal cells using the fifth isolation agent the seventh isolation agent. In some embodiments, the sample is enriched for maternal cells using the sixth isolation agent and the seventh isolation agent.

In some embodiments, the sample is enriched for maternal cells using the first isolation agent, the second isolation agent, and the third isolation agent. In some embodiments, the sample is enriched for maternal cells using the first isolation agent, the second isolation agent, and the fourth isolation agent. In some embodiments, the sample is enriched for maternal cells using the first isolation agent, the second isolation agent, and the fifth isolation agent. In some embodiments, the sample is enriched for maternal cells using the first isolation agent, the second isolation agent, and the sixth isolation agent. In some embodiments, the sample is enriched for maternal cells using the first isolation agent, the second isolation agent, the seventh isolation agent. In some embodiments, the sample is enriched for maternal cells using the first isolation agent, the third isolation agent, and the fourth isolation agent. In some embodiments, the sample is enriched for maternal cells using the first isolation agent, the third isolation agent, and the fifth isolation agent. In some embodiments, the sample is enriched for maternal cells using the first isolation agent, the third isolation agent, and the sixth isolation agent. In some embodiments, the sample is enriched for maternal cells using the first isolation agent, the third isolation agent, and the seventh isolation agent. In some embodiments, the sample is enriched for maternal cells using the first isolation agent, the fourth isolation agent, and the fifth isolation agent. In some embodiments, the sample is enriched for maternal cells using the first isolation agent, the fourth isolation agent, and the sixth isolation agent. In some embodiments, the sample is enriched for maternal cells using the first isolation agent, the fourth isolation agent, and the seventh isolation agent. In some embodiments, the sample is enriched for maternal cells using the first isolation agent, the fifth isolation agent, and the sixth isolation agent. In some embodiments, the sample is enriched for maternal cells using the first isolation agent, the fifth isolation agent, and the seventh isolation agent. In some embodiments, the sample is enriched for maternal cells using the first isolation agent, the sixth isolation agent, and the seventh isolation agent.

In some embodiments, the sample is enriched for maternal cells using the second isolation agent, the third isolation agent, and the fourth isolation agent. In some embodiments, the sample is enriched for maternal cells using the second isolation agent, the third isolation agent, and the fifth isolation agent. In some embodiments, the sample is enriched for maternal cells using the second isolation agent, the third isolation agent, and the sixth isolation agent. In some embodiments, the sample is enriched for maternal cells using the second isolation agent, the third isolation agent, and the seventh isolation agent. In some embodiments, the sample is enriched for maternal cells using the second isolation agent, the fourth isolation agent, and the fifth isolation agent. In some embodiments, the sample is enriched for maternal cells using the second isolation agent, the fourth isolation agent, and the sixth isolation agent. In some embodiments, the sample is enriched for maternal cells using the second isolation agent, the fourth isolation agent, and the seventh isolation agent. In some embodiments, the sample is enriched for maternal cells using the second isolation agent, the fifth isolation agent, and the sixth isolation agent. In some embodiments, the sample is enriched for maternal cells using the second isolation agent, the fifth isolation agent, and the seventh isolation agent. In some embodiments, the sample is enriched for maternal cells using the second isolation agent, the sixth isolation agent, and the seventh isolation agent.

In some embodiments, the sample is enriched for maternal cells using the third isolation agent, the fourth isolation agent, and the fifth isolation agent. In some embodiments, the sample is enriched for maternal cells using the third isolation agent, the fourth isolation agent, and the sixth isolation agent. In some embodiments, the sample is enriched for maternal cells using the third isolation agent, the fourth isolation agent, and the seventh isolation agent. In some embodiments, the sample is enriched for maternal cells using the third isolation agent, the fifth isolation agent, and the seventh isolation agent. In some embodiments, the sample is enriched in maternal cells using the third isolation agent, the fifth isolation agent, and the sixth isolation agent. In some embodiments, the sample is enriched in maternal cells using the third isolation agent, the sixth isolation agent, and the seventh isolation agent.

In some embodiments, the sample is enriched for maternal cells using the fourth isolation agent, the fifth isolation agent, and the sixth isolation agent. In some embodiments, the sample is enriched for maternal cells using the fourth isolation agent, the fifth isolation agent, and the seventh isolation agent. In some embodiments, the sample is enriched for maternal cells using the fourth isolation agent, the sixth isolation agent, and the seventh isolation agent. In some embodiments, the sample is enriched in maternal cells using the fifth isolation agent, the sixth isolation agent, and the seventh isolation agent.

In some embodiments, the sample is enriched for maternal cells using the first isolation agent, the second isolation agent, the third isolation agent, and the fourth isolation agent. In some embodiments, the sample is enriched for maternal cells using the first isolation agent, the second isolation agent, the third isolation agent, and fifth isolation agent. In some embodiments, the sample is enriched for maternal cells using the first isolation agent, the second isolation agent, the third isolation agent, and the sixth isolation agent. In some embodiments, the sample is enriched for maternal cells using the first isolation agent, the second isolation agent, the third isolation agent, and the seventh isolation agent. In some embodiments, the sample is enriched for maternal cells using the first isolation agent, the second isolation agent, the fourth isolation agent, and the fifth isolation agent. In some embodiments, the sample is enriched for maternal cells using the first isolation agent, the second isolation agent, the fourth isolation agent, and the sixth isolation agent. In some embodiments, the sample is enriched for maternal cells using the first isolation agent, the second isolation agent, the fourth isolation agent, and the seventh isolation agent. In some embodiments, the sample is enriched for maternal cells using the first isolation agent, the fourth isolation agent, the fifth isolation agent, and the sixth isolation agent. In some embodiments, the sample is enriched for maternal cells using the first isolation agent, the fourth isolation agent, the sixth isolation agent, and the seventh isolation agent. In some embodiments, the sample is enriched for maternal cells using the first isolation agent, the fifth isolation agent, the sixth isolation agent, and the seventh isolation agent.

In some embodiments, the sample is enriched for maternal cells using the second isolation agent, the third isolation agent, the fourth isolation agent, and the fifth isolation agent. In some embodiments, the sample is enriched for maternal cells using the second isolation agent, the third isolation agent, the fourth isolation agent, and the sixth isolation agent. In some embodiments, the sample is enriched for maternal cells using the second isolation agent, the third isolation agent, the fourth isolation agent, and the seventh isolation agent. In some embodiments, the sample is enriched for maternal cells using the second isolation agent, the third isolation agent, the fifth isolation agent, and the sixth isolation agent. In some embodiments, the sample is enriched for maternal cells using the second isolation agent, the third isolation agent, the fifth isolation agent, and the seventh isolation agent. In some embodiments, the sample is enriched for maternal cells using the second isolation agent, the fourth isolation agent, the fifth isolation agent, and the sixth isolation agent. In some embodiments, the sample is enriched for maternal cells using the second isolation agent, the fourth isolation agent, the fifth isolation agent, and the seventh isolation agent. In some embodiments, the sample is enriched for maternal cells using the second isolation agent, the fifth isolation agent, the sixth isolation agent, and the seventh isolation agent.

In some embodiments, the sample is enriched for maternal cells using the third isolation agent, the fourth isolation agent, the fifth isolation agent, and the sixth isolation agent. In some embodiments, the sample is enriched for maternal cells using the fourth isolation agent, the fifth isolation agent, the sixth isolation agent, and the seventh isolation agent.

In some embodiments, the sample is enriched for maternal cells using the first isolation agent, the second isolation agent, the third isolation agent, the fourth isolation agent, and the fifth isolation agent. In some embodiments, the sample is enriched for maternal cells using the first isolation agent, the third isolation agent, the fourth isolation agent, the fifth isolation agent, and the sixth isolation agent. In some embodiments, the sample is enriched for maternal cells using the first isolation agent, the second isolation agent, the fourth isolation agent, the fifth isolation agent, and the sixth isolation agent. In some embodiments, the sample is enriched for maternal cells using the first isolation agent, the second isolation agent, the third isolation agent, the fifth isolation agent, and the sixth isolation agent. In some embodiments, the sample is enriched for maternal cells using the first isolation agent, the second isolation agent, the third isolation agent, the fourth isolation agent, and the sixth isolation agent.

In some embodiments, the sample is enriched for maternal cells using the second isolation agent, the third isolation agent, the fourth isolation agent, the fifth isolation agent, and the sixth isolation agent.

In some embodiments, the sample is enriched for maternal cells using the first isolation agent, the third isolation agent, the fourth isolation agent, the fifth isolation agent, and the seventh isolation agent. In some embodiments, the sample is enriched for maternal cells using the first isolation agent, the second isolation agent, the fourth isolation agent, the fifth isolation agent, and the seventh isolation agent. In some embodiments, the sample is enriched for maternal cells using the first isolation agent, the second isolation agent, the third isolation agent, the fifth isolation agent, and the seventh isolation agent. In some embodiments, the sample is enriched in maternal cells using the first isolation agent, the second isolation agent, the third isolation agent, the fourth isolation agent, and the seventh isolation agent. In some embodiments, the sample is enriched in maternal cells using the first isolation agent, the second isolation agent, the third isolation agent, the fifth isolation agent, and the sixth isolation agent. In some embodiments, the sample is enriched in maternal cells using the first isolation agent, the second isolation unit, the third isolation unit, the fifth isolation agent, and the seventh isolation agent. In some embodiments, the sample is enriched in maternal cells using the first isolation agent, the second isolation agent, the third isolation agent, the sixth isolation agent, and the seventh isolation agent.

In some embodiments, the sample is enriched for maternal cells using the second isolation agent, the fourth isolation agent, the fifth isolation agent, the sixth isolation agent, and the seventh isolation agent. In some embodiments, the sample is enriched for maternal cells using the second isolation agent, the third isolation agent, the fifth isolation agent, the sixth isolation agent, and the seventh isolation agent. In some embodiments, the sample is enriched for maternal cells using the second isolation agent, the third isolation agent, the fourth isolation agent, the sixth isolation agent, and seventh isolation agent. In some embodiments, the sample is enriched for maternal cells using the second isolation agent, the third isolation agent, the fourth isolation agent, the fifth isolation agent, and the seventh isolation agent.

In some embodiments, the sample is enriched for maternal cells using the third isolation agent, the fourth isolation agent, the fifth isolation agent, the sixth isolation agent, and the seventh isolation agent.

In some embodiments, the sample is enriched for maternal cells using the first isolation agent, the second isolation agent, the third isolation agent, the fourth isolation agent, the fifth isolation agent, and the sixth isolation agent. In some embodiments, the sample is enriched for maternal cells using the first isolation agent, the third isolation agent, the fourth isolation agent, the fifth isolation agent, the sixth isolation agent, and the seventh isolation agent. In some embodiments, the sample is enriched for maternal cells using the first isolation agent, the second isolation agent, the fourth isolation agent, the fifth isolation agent, the sixth isolation agent, and the seventh isolation agent. In some embodiments, the sample is enriched for maternal cells using the first isolation agent, the second isolation, the third isolation agent, the fifth isolation agent, the sixth isolation agent, and the seventh isolation agent. In some embodiments, the sample is enriched for maternal cells using the first isolation agent, the second isolation agent, the third isolation agent, the fourth isolation agent, the sixth isolation agent, and the seventh isolation agent. In some embodiments, the sample is enriched for maternal cells using the first isolation agent, the second isolation agent, the third isolation agent, the fourth isolation agent, the fifth isolation, and the seventh isolation agent.

In some embodiments, the sample is enriched for maternal cells using the second isolation agent, the third isolation agent, the fourth isolation agent, the fifth isolation agent, the sixth isolation agent, and the seventh isolation agent. In some embodiments, the sample is enriched for maternal cells using the first isolation agent, the second isolation agent, the third isolation agent, the fourth isolation agent, the fifth isolation agent, the sixth isolation agent, and the seventh isolation agent.

In some embodiments, the seventh biomarker is a Sct biomarker, a H19 biomarker, a Krt8 biomarker, a Krt18 biomarker, a Tfpi biomarker, or a Fthl17a biomarker. In some embodiments, the seventh biomarker is a Sct biomarker. In some embodiments, the seventh biomarker is a H19 biomarker. In some embodiments, the seventh biomarker is a Krt8 biomarker. In some embodiments, the seventh biomarker is a Krt18 biomarker. In some embodiments, the seventh biomarker is a Tfpi biomarker. In some embodiments, the seventh biomarker is a Fthl17a biomarker.

In some embodiments, the first biomarker, the second biomarker, the third biomarker, the fourth biomarker, the fifth biomarker, or the sixth biomarker, is Coro1a, Cd52, Cyba, Rac2, Laptm5, or Ptprc. In some embodiments, the first biomarker is a Coro1a biomarker. In some embodiments, the second biomarker is a Coro1a biomarker. In some embodiments, the third biomarker is an Coro1a biomarker. In some embodiments, the fourth biomarker is a Coro1a biomarker. In some embodiments, the fifth biomarker is a Coro1a biomarker. In some embodiments, the sixth biomarker is a Coro1a biomarker.

In some embodiments, the first biomarker is a Cd52 biomarker. In some embodiments, the second biomarker is a Cd52 biomarker. In some embodiments, the third biomarker is a Cd52 biomarker. In some embodiments, the fourth biomarker is a Cd52 biomarker. In some embodiments, the fifth biomarker is a Cd52 biomarker. In some embodiments, the sixth biomarker is a Cd52 biomarker.

In some embodiments, the first biomarker is a Cyba biomarker. In some embodiments, the second biomarker is a Cyba biomarker. In some embodiments, the third biomarker is a Cyba biomarker. In some embodiments, the fourth biomarker is a Cyba biomarker. In some embodiments, the fifth biomarker is a Cyba biomarker. In some embodiments, the sixth biomarker is a Cyba biomarker.

In some embodiments, the first biomarker is a Rac2 biomarker. In some embodiments, the second biomarker is a Rac2 biomarker. In some embodiments, the third biomarker is a Rac2 biomarker. In some embodiments, the fourth biomarker is a Rac2 biomarker. In some embodiments, the fifth biomarker is a Rac2. In some embodiments, the sixth biomarker is a Rac2 biomarker.

In some embodiments, the first biomarker is a Laptm5 biomarker. In some embodiments, the second biomarker is a Laptm5 biomarker. In some embodiments, the third biomarker is Laptm5 biomarker. In some embodiments, the fourth biomarker is a Laptm5 biomarker. In some embodiments, the fifth biomarker is a Laptm5 biomarker. In some embodiments, the sixth biomarker is a Laptm5 biomarker.

In some embodiments, the first biomarker is a Ptprc biomarker. In some embodiments, the second biomarker is a Ptprc biomarker. In some embodiments, the third biomarker is a Ptprc biomarker. In some embodiments, the fourth biomarker is a Ptprc biomarker. In some embodiments, the fifth biomarker is a Ptprc biomarker. In some embodiments, the sixth biomarker is a Ptprc biomarker.

In some embodiments, the present disclosure provides a method of incubating a population of cells with at least one agent. In some embodiments, the agent recognizes a human orthologue of a Sct biomarker. In some embodiments, the population of cells have a subpopulation of fetal cells. In some embodiments, the population of cells have a subpopulation of maternal cells. In some embodiments, the population of cells have a subpopulation of fetal cells and a subpopulation of maternal cells. In some embodiments, the population of cells is from a biological sample of a subject. In some embodiments, the biological sample is a blood sample. In some embodiments, the blood sample is plasma. In some embodiments, the blood sample is serum. In some embodiments, the blood sample is whole blood. In some embodiments, the blood sample is whole blood depleted of red blood cells. In some embodiments, the blood sample is peripheral blood mononuclear cells. In some embodiments, the blood sample is nucleated white blood cells. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof. In some embodiments, the biological sample is from a subject. In some embodiments, the subject is a mammal. In some embodiments, the subject is a mouse. In some embodiments, the subject is a rat. In some embodiments, the subject is a non-human primate. In some embodiments, the subject is a human. In some embodiments, the biological sample is from a pregnant subject. In some embodiments, the biological sample from the pregnant subject is a maternal biological sample.

In some embodiments, the present disclosure provides a method of incubating a population of cells with an agent. In some embodiments, the agent recognizes a human orthologue of a H19 biomarker.

In some embodiments, the present disclosure provides a method of incubating a population of cells with an agent. In some embodiments, the agent recognizes a human orthologue of a Tfpi biomarker.

In some embodiments, the present disclosure provides a method of incubating a population of cells with an agent. In some embodiments, the agent recognizes a human orthologue of a Fthl17a biomarker.

In some embodiments, the present disclosure provides a method of incubating a population of cells with an agent. In some embodiments, the agent recognizes one or more of the fetal biomarkers disclosed herein.

In some embodiments, the present disclosure provides a method of incubating a population of cells with an agent. In some embodiments, the agent recognizes one or more of the maternal biomarkers disclosed herein.

In some embodiments, the present disclosure provides a method of incubating a population of cells with an agent. In some embodiments, the agent recognizes a human orthologue of a Coro1a biomarker.

In some embodiments, the present disclosure provides a method of incubating a population of cells with an agent. In some embodiments, the agent recognizes a human orthologue of a Cd52 biomarker.

In some embodiments, the present disclosure provides a method of incubating a population of cells with an agent. In some embodiments, the agent recognizes a human orthologue of a Cyba biomarker.

In some embodiments, the present disclosure provides a method of incubating a population of cells with an agent. In some embodiments, the agent recognizes a human orthologue of a Rac2 biomarker.

In some embodiments, the present disclosure provides a method of incubating a population of cells with an agent. In some embodiments, the agent recognizes a human orthologue of a Laptm5 biomarker.

In some embodiments, the present disclosure provides a method of incubating a population of cells with an agent. In some embodiments, the agent recognizes a human orthologue of a Ptprc biomarker.

### Kits

In some embodiments, the present disclosure provides kits for isolating fetal cells from a maternal biological sample. In some embodiments, the kit has one or more biological agents that recognize one or more fetal biomarker. In some embodiments, the kit has one or more biological agents that recognize one or more maternal biomarkers. In some embodiments, the kit has one or more biological agents that recognize one or more fetal biomarkers and one or more maternal biomarkers. In some embodiments, the kit has instructions for using the biological agents to process maternal sample to generate fetal cell enriched sample. In some embodiments, the kit has instructions according to one or more methods disclosed herein. In some embodiments, the kit further supplies reference sequences for prenatal testing of genetic conditions or disorders.

In some embodiments, the present disclosure provides kits for collecting maternal blood samples. In some embodiments, the kit provides a lancet. In some embodiments, the lancet is a single-use lancet. In some embodiments, the kit provides a blood collection kit. In some embodiments, the blood collection kit has a single use wing cannula. In some embodiments, the blood collection kit has a flexible tube collected to the single use wing cannula. In some embodiments, the blood collection kit has a holder. In some embodiments, the holder has a luer adaptor. In some embodiments, the kit further provides a blood collection vessel. In some embodiments, the blood collection vessel is a blood tube. In some embodiments, the blood tube has an anticoagulant in the tube. In some embodiments, the blood tube has a preservative in the tube. In some embodiments, the blood tube attaches to the holder in the blood collection kit. In some embodiments, the blood collection vessel is a microcapillary tube. In some embodiments, the blood collection vessel is a blood spot card. In some embodiments, the blood collection vessel is paper. In some embodiments, the kit further provides written instructions on how to collect the maternal blood sample. In some embodiments, the kit further provides a packaging container. In some embodiments, the packaging container has a primary packaging container. In some embodiments, the primary packaging container holds the blood collection vessel. Non-limiting examples of primary packaging containers include plastic bags, cardboard box, plastic containers, Styrofoam container, and conical tubes. In some embodiments, the packaging contained has a second packing container. In some embodiments, the secondary packing container is a mailing package. In some embodiments, the mailing package is preaddressed to a laboratory service.

### Trained Algorithms

In some embodiments, the present disclosure provides a system, method, or kit having data analysis realized in software application, computing hardware, or both. In some embodiments, the analysis application or system includes at least a data receiving module, a data pre-processing module, a data analysis module, a data interpretation module, or a data visualization module. In some embodiments, the data receiving module can comprise computer systems that connect laboratory hardware or instrumentation with computer systems that process laboratory data. In some embodiments, the data pre-processing module can comprise hardware systems or computer software that performs operations on the data in preparation for analysis. Examples of operations that can be applied to the data in the pre-processing module include affine transformations, denoising operations, data cleaning, reformatting, and subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

Feature sets can be generated from datasets obtained using one or more assays of a biological sample, and an algorithm can be used to process one or more of the feature sets to identify novel fetal cell or maternal cell features. For example, the algorithm can be used to apply a machine learning classifier to a plurality of features that are associated with fetal cells and maternal cells to classify a cell being a fetal cell or a maternal cell. For example, an algorithm can be used to apply a machine learning classifier to a plurality of biomarkers isolated from fetal cells and a plurality of biomarkers isolated from maternal cells to classify a feature, such as a biomarker, as being associated with the fetal cell or maternal cell.

The algorithm can be configured to identify the presence or absence of fetal cells or maternal cells with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy can be achieved, for example, for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The algorithm can comprise a classifier configured to accept as input a plurality of input variables or features (e.g., fetal cell features or maternal cell features) and to produce or output one or more output values based on the plurality of input variables or features. The plurality of input variables or features can comprise one or more datasets indicative of the presence or absence of fetal cells or maternal cells. For example, an input variable or feature can comprise a number of sequences corresponding to each of the plurality of fetal biomarkers or maternal biomarkers.

Novel feature sets can be generated from datasets obtained using one or more assays of a biological sample, and an algorithm can be used to process features from fetal cells to identify biomarkers associated with fetal cells. For example, the algorithm can be used to apply a machine learning classifier to a plurality of features from fetal cells that are associated with two or more classes of features inputted into a machine learning model to classify a feature into one of the two or more classes of features. For example, the algorithm can be used to apply a machine learning classifier to a plurality of features from fetal cells and maternal cells to classify a biomarker as being associated with fetal cells or maternal cells.

Novel feature sets can be generated from datasets obtained using one or more assays of a biological sample, and an algorithm can be used to process features from maternal cells to identify biomarkers associated with maternal cells. For example, the algorithm can be used to apply a machine learning classifier to a plurality of features from maternal cells that are associated with two or more classes of features inputted into a machine learning model to classify a feature into one of the two or more classes of features. For example, the algorithm can be used to apply a machine learning classifier to a plurality of features from maternal cells and fetal cells to classify a biomarker as being associated with maternal cells or fetal cells.

The algorithm can be configured to identify novel features associated with fetal cells or maternal cells with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy can be achieved, for example, for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The algorithm can comprise a classifier configured to accept as input a plurality of input variables or features (e.g., features of fetal cells or maternal cells) and to produce or output one or more output values based on the plurality of input variables or features. The plurality of input variables or features can comprise one or more datasets indicative of the presence or absence of fetal cells or maternal cells. For example, an input variable or feature can comprise a number of sequences corresponding to each of the plurality of features from fetal cells. The plurality of input variable or features can comprise one or more datasets from fetal cells and from maternal cells and the output values indicates the association of specific features with fetal cells.

The algorithm can comprise a machine learning algorithm, such as a supervised machine learning algorithm. The supervised machine learning algorithm can comprise, for example, a Random Forest, a support vector machine (SVM), or a neural network. The algorithm can comprise a classification and regression tree (CART) algorithm. The machine learning algorithm can comprise a linear regression or a logistic regression. The algorithm can comprise an unsupervised machine learning algorithm.

The plurality of input variables or features can also include clinical information of a subject, such as health data. For example, the health data of a subject can comprise one or more of: a diagnosis of one or more conditions, a prognosis of one or more conditions, a risk of having one or more conditions, screening or testing results of one of more conditions, a treatment history of one or more conditions, a history of previous treatment for one or more conditions, a history of prescribed or other medications, a history of prescribed medical devices, personal characteristics (e.g., age, race, ethnicity, height, weight, sex, geographic location, diet, exercise, smoking status, family history of genetic disorders), and one or more symptoms of the subject.

The algorithm can comprise a classifier, such that each of the one or more output values comprises one of a fixed number of possible values (e.g., a linear classifier, a logistic regression classifier, etc.) indicating a classification of the sample by the classifier. The algorithm can comprise a binary classifier, such that each of the one or more output values comprises one of two values (e.g., {0, 1}, {positive, negative}, or {high-risk, low-risk}) indicating a classification of the sample by the classifier. The algorithm can be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier can be configured to classify cells by assigning output values, which can comprise descriptive labels, numerical values, or a combination thereof. Some of the output values can comprise descriptive labels. Such descriptive labels can provide an identification or indication of the presence or absence fetal cells or maternal cells. Some descriptive labels can be mapped to numerical values, for example, by mapping positive to 1 and negative to 0.

The classifier can be configured to classify cells by assigning output values that comprise numerical values, such as binary, integer, or continuous values. Such binary output values can comprise, for example, {0, 1} or {positive, negative}. Such integer output values can comprise, for example, {0, 1, 2}. Such continuous output values can comprise, for example, a probability value of at least 0 and no more than 1. Such continuous output values can comprise, for example, an un-normalized probability value of at least 0. Such continuous output values can indicate a likelihood that a specific feature is associated with fetal cells or maternal cells. Some numerical values can be mapped to descriptive labels, for example, by mapping 1 to positive and 0 to negative.

The classifier can be configured to classify samples by assigning output values based on one or more cutoff values. For example, a binary classification of cells can assign an output value of positive or 1 if the sample indicates that the cell has at least a 50% probability of being a fetal cell, thereby assigning the cell to a class of cells receiving a positive test result. For example, a binary classification of cells can assign an output value or positive or 1 to a specific feature that indicates that the specific feature has at least a 50% probability of being associated with fetal cells. As another example, a binary classification of cells can assign an output value of negative or 0 if the sample indicates that the subject has less than a 50% probability of being a maternal cell, thereby assigning the cell to a class of cells receiving a negative test result. In this case, a single cutoff value of 50% can be used to classify cells into one of the two possible binary output values or classes of cells (e.g., maternal cells or fetal cells). For example, a binary classification of cells can assign an output value of negative or 0 to a specific feature that indicates that the specific feature has less than 50% probability of being associated with maternal cells. Examples of single cutoff values can include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier can be configured to classify cells by assigning an output value of positive or 1 if the sample indicates that the cell has a probability of being a fetal cell between about 50% to about 100%. The classifier can be configured to classify cells by assigning an output value of positive or 1 if the sample indicates that the cell has a probability of being a fetal cell of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of cells can assign an output value of positive or 1 if the sample indicates that the cell has a probability of being a fetal cell of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%. The classification of cells can assign an output value of positive or 1 if the sample indicates that the cell has a probability of being a fetal cell of about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%.

As another example, the classifier can be configured to classify cells by assigning an output value of positive or 1 to a specific feature that indicates that the specific feature has a probability of being associated with fetal cells between about 50% to about 100%. The classifier can be configured to classify cells by assigning an output value of positive or 1 to a specific feature that indicates that the specific feature has a probability of being associated with fetal cells of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of cells can assign an output value of positive or 1 if the cell indicates that the specific feature has a probability of being associated with fetal cells of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%. The classification of cells can assign an output value of positive or 1 if the cell indicates that the specific feature has a probability of being associated with fetal cells of about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%.

The classifier can be configured to classify cells by assigning an output value of negative or 0 if the cell indicates that the cell has a probability of being a maternal cell between about 0% to about 50%. The classifier can be configured to classify cells by assigning an output value of negative or 0 if the cell indicates that the cell has a probability of being a maternal cell of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of cells can assign an output value of negative or 0 if the cell indicates that the cell has a probability of being a maternal cell of at least about 50%, at least about 45%, at least about 40%, at least about 35%, at least about 30%, at least about 25%, at least about 20%, at least about 15%, at least about 10%, at least about 9%, at least about 8%, at least about 7%, at least about 6%, at least about 5%, at least about 4%, at least about 3%, at least about 2%, or at least about 1%. The classification of cells can assign an output value of negative or 0 if the cell indicates that the cell has a probability of being a maternal cell of about 50%, about 45%, about 40%, about 35%, about 30%, about 25%, about 20%, about 15%, about 10%, about 9%, about 8%, about 7%, about 6%, about 5%, about 4%, about 3%, about 2%, or about 1%.

The classifier can be configured to classify cells by assigning an output value of negative or 0 to a specific feature that indicates that the specific feature has a probability of being associated with a maternal cell between about 0% to about 50%. The classifier can be configured to classify cells by assigning an output value of negative or 0 to a specific feature that indicates that the specific feature has a probability of being associated with a maternal cell of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of cells can assign an output value of negative or 0 if the cell indicates that the specific feature has a probability of being associated with a maternal cell of at least about 50%, at least about 45%, at least about 40%, at least about 35%, at least about 30%, at least about 25%, at least about 20%, at least about 15%, at least about 10%, at least about 9%, at least about 8%, at least about 7%, at least about 6%, at least about 5%, at least about 4%, at least about 3%, at least about 2%, or at least about 1%.

The classifier can be configured to classify cells by assigning an output value of indeterminate or 2 if the cell is not classified as positive, negative, 1, or 0. In this case, a set of two cutoff values can be used to classify cells into one of the three possible output values or classes of cells (e.g., fetal cells, maternal cells, or indeterminate cells). Examples of sets of cutoff values can include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values can be used to classify cells into one of n+1 possible output values or classes of cells, where n is any positive integer.

The algorithm can be trained with a plurality of independent training samples. Each of the independent training samples can comprise a sample of fetal cells, associated datasets obtained by assaying the sample (as described elsewhere herein), and one or more known output values or classes of cells corresponding to the sample of fetal cells. Independent training samples can comprise samples of fetal cells and associated datasets and outputs obtained or derived from a plurality of different subjects. Independent training samples can comprise samples of fetal cells and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples can be associated with presence of the fetal cells. Independent training samples can be associated with absence of the fetal cells.

The algorithm can be trained with a plurality of independent training samples. Each of the independent training samples can comprise a sample of maternal cells, associated datasets obtained by assaying the sample (as described elsewhere herein), and one or more known output values or classes of cells corresponding to the sample of maternal cells. Independent training samples can comprise samples of maternal cells and associated datasets and outputs obtained or derived from a plurality of different subjects. Independent training samples can comprise samples of maternal cells and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples can be associated with presence of the maternal cells. Independent training samples can be associated with absence of the maternal cells.

The algorithm can be trained with between about 5 to about 500 independent training samples. The algorithm can be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The algorithm can be trained with at most about 5, at most about 10, at most about 15, at most about 20, at most about 25, at most about 30, at most about 35, at most about 40, at most about 45, at most about 50, at most about 100, at most about 150, at most about 200, at most about 250, at most about 300, at most about 350, at most about 400, at most about 450, or at most about 500 independent training samples. The algorithm can be trained with about 5, about 10, about 15, about 20, about 25, about 30, about 35, about 40, about 45, about 50, about 100, about 150, about 200, about 250, about 300, about 350,about 400, about 450, or about 500 independent training samples. The independent training samples can comprise samples associated with presence of fetal cells and/or samples associated with absence of fetal cells. The independent training samples can comprise samples associated with presence of maternal cells and/or samples associated with absence of maternal cells. The algorithm can be trained with between about 50 to about 500 independent training samples associated with presence of fetal cells or maternal cells. The algorithm can be trained with between about 50 to about 500 independent training samples associated with absence of fetal cells or maternal cells. The algorithm can be trained with no more than about 500, no more than about 450, no more than about 400, no more than about 350, no more than about 300, no more than about 250, no more than about 200, no more than about 150, no more than about 100, or no more than about 50 independent training samples associated with presence of the fetal cells or the maternal cells. The algorithm can be trained with no more than about 500, no more than about 450, no more than about 400, no more than about 350, no more than about 300, no more than about 250, no more than about 200, no more than about 150, no more than about 100, or no more than about 50 independent training samples associated with absence of the fetal cells or maternal cells. The algorithm can be trained with at least about 500, at least about 450, at least about 400, at least about 350, at least about 300, at least about 250, at least about 200, at least about 150, at least about 100, or at least about 50 independent training samples associated with presence of the fetal cells or the maternal cells. The algorithm can be trained with at least about 500, at least about 450, at least about 400, at least about 350, at least about 300, at least about 250, at least about 200, at least about 150, at least about 100, or at least about 50 independent training samples associated with absence of the fetal cells or maternal cells. The algorithm can be trained with about 500, about 450, about 400, about 350, about 300, about 250, about 200, about 150, about 100, or about 50 independent training samples associated with presence of the fetal cells or the maternal cells. The algorithm can be trained with about 500, about 450, about 400, about 350, about 300, about 250, about 200, about 150, about 100, or about 50 independent training samples associated with absence of the fetal cells or maternal cells. In some embodiments, the sample is independent of samples used to train the algorithm.

The algorithm can be trained with a first number of independent training samples associated with a presence of fetal cells and a second number of independent training samples associated with fetal cells. The first number of independent training samples associated with presence of fetal cells can be no more than the second number of independent training samples associated with absence of fetal cells. The first number of independent training samples associated with a presence of fetal cells can be equal to the second number of independent training samples associated with an absence of fetal cells. The first number of independent training samples associated with a presence of fetal cells can be greater than the second number of independent training samples associated with fetal cells.

The algorithm can be trained with a first number of independent training samples associated with a presence of maternal cells and a second number of independent training samples associated with maternal cells. The first number of independent training samples associated with presence of maternal cells can be no more than the second number of independent training samples associated with absence of maternal cells. The first number of independent training samples associated with a presence of maternal cells can be equal to the second number of independent training samples associated with an absence of maternal cells. The first number of independent training samples associated with a presence of maternal cells can be greater than the second number of independent training samples associated with maternal cells.

The algorithm can comprise a classifier configured to identify the presence or absence of fetal cells at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence or absence of the fetal cells by the algorithm can be calculated as the percentage of independent test samples that are correctly identified or classified as being fetal cells or being maternal cells.

The algorithm can comprise a classifier configured to identify fetal cells with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the fetal cells using the algorithm can be calculated as the percentage of samples identified or classified as fetal cells that correspond to cells that are fetal cells.

The algorithm can comprise a classifier configured to identify fetal cells with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the algorithm can be calculated as the percentage of cells identified or classified as not being fetal cells that correspond to cells that truly are maternal cells.

The algorithm can comprise a classifier configured to identify fetal cells with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying fetal cells using the algorithm can be calculated as the percentage of independent test samples associated with presence of fetal cells that are correctly identified or classified as having fetal cells.

The algorithm can comprise a classifier configured to identify fetal cells with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying fetal cells using the algorithm can be calculated as the percentage of independent test samples associated with not fetal cells that are correctly identified or classified as maternal cells.

Classifiers of the algorithm can be adjusted or tuned to improve or optimize one or more performance metrics, such as accuracy, PPV, NPV, clinical sensitivity, or clinical specificity (e.g., a performance index incorporating a plurality of such performance metrics, such as by calculating a weight sum therefrom), of identifying the presence or absence of fetal cells. The classifiers can be adjusted or tuned by adjusting parameters of the classifiers (e.g., a set of cutoff values used to classify a sample as described elsewhere herein, or weights of a neural network) to improve or optimize the performance metrics. The classifiers can be adjusted or tuned to reduce an overall classification error (e.g., an out-of-bag or oob error rate for a Random Forest classifier). The classifiers can be adjusted or tuned continuously during the training process (e.g., as sample datasets are added to the training set) or after the training process has completed.

The algorithm can comprise a classifier configured to identify fetal cell biomarkers with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying fetal cell biomarkers using the algorithm can be calculated as the percentage of biomarkers identified or classified as being associated with fetal cells that correspond to fetal cells.

The algorithm can comprise a classifier configured to identify biomarkers associated with the fetal cells with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying fetal cell biomarkers using the algorithm can be calculated as the percentage of biomarkers identified or classified as not being associated with fetal cells that correspond to maternal cells.

The algorithm can comprise a classifier configured to identify fetal cell biomarkers with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying fetal cell biomarkers using the algorithm can be calculated as the percentage of independent test samples associated with presence of the fetal cell biomarkers that are correctly identified or classified as being fetal cells.

The algorithm can comprise a classifier configured to identify fetal cell biomarkers with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying fetal cell biomarkers using the algorithm can be calculated as the percentage of independent test samples associated with absence of fetal cell biomarkers that are correctly identified or classified as maternal cells.

The algorithm can comprise a classifier configured to identify maternal cell biomarkers with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying maternal cell biomarkers using the algorithm can be calculated as the percentage of biomarkers identified or classified as being associated with maternal cells that correspond to maternal cells.

The algorithm can comprise a classifier configured to identify biomarkers associated with the maternal cells with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying maternal cell biomarkers using the algorithm can be calculated as the percentage of biomarkers identified or classified as not being associated with maternal cells that correspond to fetal cells.

The algorithm can comprise a classifier configured to identify maternal cell biomarkers with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying maternal cell biomarkers using the algorithm can be calculated as the percentage of independent test samples associated with presence of the maternal cell biomarkers that are correctly identified or classified as being maternal cells.

The algorithm can comprise a classifier configured to identify maternal cell biomarkers with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying maternal cell biomarkers using the algorithm can be calculated as the percentage of independent test samples associated with absence of maternal cell biomarkers that are correctly identified or classified as fetal cells.

Classifiers of the algorithm can be adjusted or tuned to improve or optimize one or more performance metrics, such as accuracy, PPV, NPV, clinical sensitivity, clinical specificity, or a combination thereof (e.g., a performance index incorporating a plurality of such performance metrics, such as by calculating a weight sum therefrom), of identifying the fetal cell biomarkers or the maternal cell biomarkers. The classifiers can be adjusted or tuned by adjusting parameters of the classifiers (e.g., a set of cutoff values used to classify a sample as described elsewhere herein, or weights of a neural network) to improve or optimize the performance metrics. The classifiers can be adjusted or tuned to reduce an overall classification error (e.g., an “out-of-bag” or oob error rate for a Random Forest classifier). The one or more classifiers can be adjusted or tuned continuously during the training process (e.g., as sample datasets are added to the training set) or after the training process has completed.

The algorithm can comprise a plurality of classifiers (e.g., an ensemble) such that the plurality of classifications or outcome values of the plurality of classifiers can be combined to produce a single classification or outcome value for the sample (e.g., to generate an ensemble output). For example, a sum or a weighted sum of the plurality of classifications or outcome values of the plurality of classifiers can be calculated to produce a single classification or outcome value for the sample. As another example, a majority vote of the plurality of classifications or outcome values of the plurality of classifiers can be identified to produce a single classification or outcome value for the sample. In this manner, a single classification or outcome value can be produced for the sample having greater confidence or statistical significance than the individual classifications or outcome values produced by each of the plurality of classifiers.

After the algorithm is initially trained, a subset of the inputs can be identified as most influential or most important to be included for making high-quality classifications (e.g., having highest permutation feature importance). For example, a subset of the panel of fetal cells features can be identified as most influential or most important to be included for making high-quality classifications or identifications of fetal cells. For example, a subset of the panel of maternal cells features can be identified as most influential or most important to be included for making high-quality classifications or identifications of maternal cells. The panel of fetal cell or maternal features, or a subset thereof, can be ranked based on classification metrics indicative of each influence or importance of each individual fetal cell or maternal cell feature toward making high-quality classifications or identifications of fetal cells or maternal cells. Such metrics can be used to reduce, in some embodiments significantly, the number of input variables (e.g., predictor variables) that can be used to train the one or more classifiers of the algorithm to a desired performance level (e.g., based on a desired minimum accuracy, PPV, NPV, clinical sensitivity, clinical specificity, or a combination thereof).

For example, if training a classifier of the algorithm with a plurality comprising several dozen or hundreds of input variables to the classifier results in an accuracy of classification of more than 99%, then training the classifier of the algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the algorithm with a plurality comprising several dozen or hundreds of input variables to the classifier results in a sensitivity or specificity of classification of more than 99%, then training the classifier of the algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of fetal cell or maternal cells features) to the classifier of the algorithm can be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

### Applications

Compositions, methods, and kits provided herein can be used for prenatal testing. For example, circulating fetal cells can be isolated from early stages in pregnancy, including early in the first trimester. The isolated CFCs can be used to sequence the genome of the developing fetus and determine whether the fetus has any genetic disorders or fetal conditions. The isolation and subsequent sequencing of CFCs can detect genetic disorders at an earlier stage of pregnancy compared to conventional prenatal testing.

In some embodiments, the fetal cells can be analyzed. In some embodiments, the fetal cells can be analyzed for fetal cell characteristics. In some embodiments, the fetal cells can be analyzed for fetal genomic information. In some embodiments, the fetal genomic information can be the whole genome sequence of a fetal cell. In some embodiments, the fetal genomic information can be the sequence of one or more fetal genes. In some examples, the one or more fetal genes can comprise at least about one fetal gene, at least about two fetal genes, at least about three fetal genes, at least about four fetal genes, at least about five fetal genes, at least about six fetal genes, at least about seven fetal genes, at least about eight fetal genes, at least about nine fetal genes, at least about ten fetal genes, at least about 15 fetal genes, at least about 20 fetal genes, at least about 25 fetal genes, at least about 30 fetal genes, at least about 35 fetal genes, at least about 40 fetal genes, at least about 45 fetal genes, at least about 50 fetal genes, at least about 55 fetal genes, at least about 60 fetal genes, at least about 65 fetal genes, at least about 70 fetal genes, at least about 75 fetal genes, at least about 80 fetal genes, at least about 85 fetal genes, at least about 90 fetal genes, at least about 95 fetal genes, at least about 100 fetal genes, or more. In some embodiments, the one or more fetal genes can comprise at most about 100 fetal genes, at most about 95 fetal genes, at most about 90 fetal genes, at most about 85 fetal genes, at most about 80 fetal genes, at most about 75 fetal genes, at most about 70 fetal genes, at most about 65 fetal genes, at most about 60 fetal genes, at most about 55 fetal genes, at most about 50 fetal genes, at most about 45 fetal genes, at most about 40 fetal genes, at most about 35 fetal genes, at most about 30 fetal genes, at most about 25 fetal genes, at most about 20 fetal genes, at most about 15 fetal genes, at most about 10 fetal genes, at most about nine fetal genes, at most about eight fetal genes, at most about seven fetal genes, at most about six fetal genes, at most about five fetal genes, at most about four fetal genes, at most about three fetal genes, at most about two fetal genes, at most about one fetal genes, or less. In some examples, the one or more fetal genes can comprise about one fetal gene, about two fetal genes, about three fetal genes, about four fetal genes, about five fetal genes, about six fetal genes, about seven fetal genes, about eight fetal genes, about nine fetal genes, about ten fetal genes, about 15 fetal genes, about 20 fetal genes, about 25 fetal genes, about 30 fetal genes, about 35 fetal genes, about 40 fetal genes, about 45 fetal genes, about 50 fetal genes, about 55 fetal genes, about 60 fetal genes, about 65 fetal genes, about 70 fetal genes, about 75 fetal genes, about 80 fetal genes, about 85 fetal genes, about 90 fetal genes, about 95 fetal genes, or about 100 fetal genes. In some embodiments, the fetal genomic information can be the chromosomal make up of a fetal cell.

In some embodiments, the fetal genomic information can be the epigenetic modifications of the whole genome sequence of a fetal cell. In some embodiments, the fetal genomic information can be the epigenetic modifications of one or more fetal genes. In some examples, the one or more fetal genes can comprise at least about one fetal gene, at least about two fetal genes, at least about three fetal genes, at least about four fetal genes, at least about five fetal genes, at least about six fetal genes, at least about seven fetal genes, at least about eight fetal genes, at least about nine fetal genes, at least about ten fetal genes, at least about 15 fetal genes, at least about 20 fetal genes, at least about 25 fetal genes, at least about 30 fetal genes, at least about 35 fetal genes, at least about 40 fetal genes, at least about 45 fetal genes, at least about 50 fetal genes, at least about 55 fetal genes, at least about 60 fetal genes, at least about 65 fetal genes, at least about 70 fetal genes, at least about 75 fetal genes, at least about 80 fetal genes, at least about 85 fetal genes, at least about 90 fetal genes, at least about 95 fetal genes, at least about 100 fetal genes, or more. In some examples, the one or more fetal genes can comprise at most about 100 fetal genes, at most about 95 fetal genes, at most about 90 fetal genes, at most about 85 fetal genes, at most about 80 fetal genes, at most about 75 fetal genes, at most about 70 fetal genes, at most about 65 fetal genes, at most about 60 fetal genes, at most about 55 fetal genes, at most about 50 fetal genes, at most about 45 fetal genes, at most about 40 fetal genes, at most about 35 fetal genes, at most about 30 fetal genes, at most about 25 fetal genes, at most about 20 fetal genes, at most about 15 fetal genes, at most about 10 fetal genes, at most about nine fetal genes, at most about eight fetal genes, at most about seven fetal genes, at most about six fetal genes, at most about five fetal genes, at most about four fetal genes, at most about three fetal genes, at most about two fetal genes, at most about one fetal genes, or less. In some examples, the one or more fetal genes can comprise about one fetal gene, about two fetal genes, about three fetal genes, about four fetal genes, about five fetal genes, about six fetal genes, about seven fetal genes, about eight fetal genes, about nine fetal genes, about ten fetal genes, about 15 fetal genes, about 20 fetal genes, about 25 fetal genes, about 30 fetal genes, about 35 fetal genes, about 40 fetal genes, about 45 fetal genes, about 50 fetal genes, about 55 fetal genes, about 60 fetal genes, about 65 fetal genes, about 70 fetal genes, about 75 fetal genes, about 80 fetal genes, about 85 fetal genes, about 90 fetal genes, about 95 fetal genes, or about 100 fetal genes. In some embodiments, the fetal genomic information can be the chromosomal make up of a fetal cell. In some embodiments, epigenetic modification of the genome of a fetal cell can be determined by bisulfite conversion, ATAC-seq, CpG island microarray, chromatin conformation capture, high resolution melt analysis, restriction enzyme analysis, immunoprecipitation, autoradiography, chromatin immunoprecipitation (ChIP) assay, DNase I hypersensitivity assays, next-generation sequencing, or any combination thereof.

In some embodiments, the fetal genomic information can be single nucleotide variations in the genome of a fetal cell. In some embodiments, the fetal genomic information can be copy number variations in the genome of a fetal cell. In some embodiments, the fetal genomic information can be insertions or deletions (indel) in the genome of a fetal cell. In some embodiments, the fetal genomic information can be insertions and deletions (indel) in the genome of a fetal cell. In some embodiments, the fetal genomics information can be sequence substitutions in the genome of a fetal cell. In some embodiments, the fetal genomics information can be sequence duplications in the genome of a fetal cell. In some embodiments, the fetal genomics information can be the chromatin structure of the genome of a fetal cell. In some embodiments, the fetal genomics information can be chromosome abnormalities of the genome of a fetal cell. In some embodiments, the fetal genomics information can be chromosomal rearrangement.

In some embodiments, the fetal cells can be analyzed for fetal gene expression. In some embodiments, the fetal gene expression can be the level of expression of one or more genes in a fetal cell. In some examples, the one or more fetal genes can comprise at least about one fetal gene, at least about two fetal genes, at least about three fetal genes, at least about four fetal genes, at least about five fetal genes, at least about six fetal genes, at least about seven fetal genes, at least about eight fetal genes, at least about nine fetal genes, at least about ten fetal genes, at least about 15 fetal genes, at least about 20 fetal genes, at least about 25 fetal genes, at least about 30 fetal genes, at least about 35 fetal genes, at least about 40 fetal genes, at least about 45 fetal genes, at least about 50 fetal genes, at least about 55 fetal genes, at least about 60 fetal genes, at least about 65 fetal genes, at least about 70 fetal genes, at least about 75 fetal genes, at least about 80 fetal genes, at least about 85 fetal genes, at least about 90 fetal genes, at least about 95 fetal genes, at least about 100 fetal genes, or more. In some examples, the one or more fetal genes can comprise at most about 100 fetal genes, at most about 95 fetal genes, at most about 90 fetal genes, at most about 85 fetal genes, at most about 80 fetal genes, at most about 75 fetal genes, at most about 70 fetal genes, at most about 65 fetal genes, at most about 60 fetal genes, at most about 55 fetal genes, at most about 50 fetal genes, at most about 45 fetal genes, at most about 40 fetal genes, at most about 35 fetal genes, at most about 30 fetal genes, at most about 25 fetal genes, at most about 20 fetal genes, at most about 15 fetal genes, at most about 10 fetal genes, at most about nine fetal genes, at most about eight fetal genes, at most about seven fetal genes, at most about six fetal genes, at most about five fetal genes, at most about four fetal genes, at most about three fetal genes, at most about two fetal genes, at most about one fetal genes, or fewer. In some examples, the one or more fetal genes can comprise about one fetal gene, about two fetal genes, about three fetal genes, about four fetal genes, about five fetal genes, about six fetal genes, about seven fetal genes, about eight fetal genes, about nine fetal genes, about ten fetal genes, about 15 fetal genes, about 20 fetal genes, about 25 fetal genes, about 30 fetal genes, about 35 fetal genes, about 40 fetal genes, about 45 fetal genes, about 50 fetal genes, about 55 fetal genes, about 60 fetal genes, about 65 fetal genes, about 70 fetal genes, about 75 fetal genes, about 80 fetal genes, about 85 fetal genes, about 90 fetal genes, about 95 fetal genes, or about 100 fetal genes. In some embodiments, the fetal genomic information can be the chromosomal make up of a fetal cell.

In some embodiments, the fetal cells can be analyzed for fetal protein expression. In some embodiments, the fetal protein expression can be the amount of one or more proteins produced from a fetal cell. In some examples, the one or more proteins can comprise at least about one protein, at least about two proteins, at least about three proteins, at least about four proteins, at least about five proteins, at least about six proteins, at least about seven proteins, at least about eight proteins, at least about nine proteins, at least about ten proteins, at least about 15 proteins, at least about 20 proteins, at least about 25 proteins, at least about 30 proteins, at least about 35 proteins, at least about 40 proteins, at least about 45 proteins, at least about 50 proteins, at least about 55 proteins, at least about 60 proteins, at least about 65 proteins, at least about 70 proteins, at least about 75 proteins, at least about 80 proteins, at least about 85 proteins, at least about 90 proteins, at least about 95 proteins, at least about 100 proteins, or more. In some embodiments, the one or more proteins can comprise at most about 100 proteins, at most about 95 proteins, at most about 90 proteins, at most about 85 proteins, at most about 80 proteins, at most about 75 proteins, at most about 70 proteins, at most about 65 proteins, at most about 60 proteins, at most about 55 proteins, at most about 50 proteins, at most about 45 proteins, at most about 40 proteins, at most about 35 proteins, at most about 30 proteins, at most about 25 proteins, at most about 20 proteins, at most about 15 proteins, at most about 10 proteins, at most about nine proteins, at most about eight proteins, at most about seven proteins, at most about six proteins, at most about five proteins, at most about four proteins, at most about three proteins, at most about two proteins, at most about one protein, or less. In some embodiments, the one or more proteins can comprise about one protein, about two proteins, about three proteins, about four proteins, about five proteins, about six proteins, about seven proteins, about eight proteins, about nine proteins, about ten proteins, about 15 proteins, about 20 proteins, about 25 proteins, about 30 proteins, about 35 proteins, about 40 proteins, about 45 proteins, about 50 proteins, about 55 proteins, about 60 proteins, about 65 proteins, about 70 proteins, about 75 proteins, about 80 proteins, about 85 proteins, about 90 proteins, about 95 proteins, or about 100 proteins. In some embodiments, the fetal genomic information can be the chromosomal make up of a fetal cell.

In some embodiments, a biological sample is collected from a pregnant subject. In some embodiments, the biological sample is a blood sample. In some embodiments, the biological sample is a tissue and/or biopsy sample. In some embodiments, the tissue and/or biopsy sample is from the uterus, placenta, umbilical cord, or any combination thereof. In some embodiments, the biological sample is a biological fluid sample. In some embodiments, the biological fluid sample is blood, saliva, interstitial fluid, umbilical cord blood, extracellular fluid, or any combination thereof. In some embodiments, CFCs are isolated from the biological sample collected from the pregnant subject. In some embodiments, the genome of the CFCs is sequenced. In some embodiments, the sequence of the CFCs determines the presence or absence of one or more genetic disorders.

In some embodiments, fetal cells are isolated from the maternal biological sample. In some embodiments, fetal cells are isolated from maternal cells in the maternal biological sample. In some embodiments, nucleic acid from the fetal cells can be extracted. In some embodiments, the nucleic acid from the fetal cells is genetically analyzed. In some embodiments, the nucleic acid from the fetal cells is sequenced. In some embodiments, sequencing data from the nucleic acid from the fetal cells is used to identify the presence or the absence of a genetic condition or disorder in the fetal cells. In some embodiments, the sequencing data is used to identify the presence or the absence of a genetic condition or disorder in a fetus associated with the fetal cells. In some embodiments, the sequencing data from the nucleic acid from the fetal cells is compared a reference sequence. In some embodiments, the reference sequence is a healthy (e.g., absence of a genetic condition or disorder) sequence. In some embodiments, the reference sequence is a diseased (e.g., presence of a genetic condition or disorder) sequence. In some embodiments, the reference sequence is from a mammal. In some embodiments, the mammal is a mouse. In some embodiments, the mammal is a rat. In some embodiments, the mammal is a non-human primate. In some embodiments, the mammal is a human.

Non-limiting examples of genetic disorders include 1p36 deletion syndrome, 1q21.1 deletion syndrome, 2q37 deletion syndrome, 5q deletion, 5, 10-methenyltetrahydrofolate synthestase deficiency, 17q12 microdeletion syndrome, 17q12 microduplication syndrome, 18p deletion syndrome, 21-hydroxylase deficiency, alpha 1-antitrypsin deficiency, achalasia-addisonianism-alacrima (AAA) syndrome, Aarskog-Scott syndrome, ABCD syndrome, Absnece deformity of leg-cataract, Aceruloplasminemia, Acheiropodia, Achondrogenesis type II, achondroplasia, acute intermittent porphyria, adenylosuccinate lyase deficiency, adrenoleukodystrophy, alagille syndrome, ADULT syndrome, Aicardi-Goutieres syndrome, Albinism, Alexander disease, Alfi's syndrome, alkaptonuria, Alport syndrome, alternating hemiplegia of childhood, aortic arch anomaly, peculiar facies, Amish lethal microcephaly, amyotrophic lateral sclerosis, angel-shaped phalango-epiphyseal dysplasia, Alstrom syndrome, Alzheimer's syndrome, amelogenesis imperfecta, aminolevulinic acid dehydratase deficiency porphyria, androgen insensitivity syndrome, angelman syndrome, aphalangy-syndactyly-microcephaly syndrome, apert syndrome, arthrogryposis, ataxia telangiectasia, axenfeld syndrome, Bainbridge-ropers syndrome, breae-stevenson cutis gyrate syndrome, beckwith-wiedermann syndrome, Benjamin syndrome, biotinidase deficiency, Bjornstad syndrome, Blepharophimosis syndromes, Bloom syndrome, Birt-Hogg-Dube syndrome, Brody myopathy, Brunner syndrome, CADASIL syndrome, Cat eye syndrome, CRASIL syndrome, chronic granulomatous disorder, campomelic dysplasia, captodactyly-taurinuria syndrome, Canavan disease, Carpenter syndrome, CDKL5 deficiency disorder, cerebral dysgenesis-neuropathy-ichthyosis-keratoderma syndrome (CEDNIK), cleft palate short stature vertebral anomalies syndrome, congenital muscular dystrophy-infantile cataract-hypogonadism syndrome, cystic fibrosis, Charcot-Marie-Tooth disease, CHARGE syndrome, Chediak-Higashi syndrome, Grebe-type chonodrodysplasia, cleidocranial dysostosis, Cockayne syndrome, Coffin-Lowry syndrome, Cohen syndrome, type II collagenopathy, type XI collagenopathy, congenital insensitivity to pain with anhidrosis (CIPA), congenital muscular dystrophy, cornelia de Lange syndrome, Cowden syndrome, CPO deficiency, Cranio-lenticulo-sutural dysplasia, Cri du chat, Crohn's disease, Crouzon syndrome, Crouzonodermoskeletal syndrome, Currarino syndrome, Darier's disease, Dent's disease, Denys-Drash syndrome, De Grouchy syndrome, dolichonychia, Down Syndrome, DiGeorge Syndrome, Distal hereditary motor neuropathies, distal muscular dystrophy, Duchenne muscular dystrophy, Dravet syndrome, Ectrodactyly-polydactyly syndrome, Edwards Syndrome, Ehlers-Danlos syndrome, Emanuel syndrome, Emery-Dreifuss syndrome, Epidermolysis bullosa, erythropoietic protoporphyria, Fanconi amenia, Fabry disease, Factor V Leiden thrombophilia, fatal familial insomnia, familial adenomatous polyposis, familial dysautonomia, Familial Creutzfeld-Jakob disease, familial episodic pain syndrome, familial thoracic aortic aneurysm and aortic dissection, Feingold syndrome, FG syndrome, FBXW7 neurodevelopmental syndrome, Fibular aplasia-ectrodactyly syndrome, Fine-Lubinsky syndrome, Fragile X syndrome, Friedreich's ataxia, G6PD deficiency, glactosemia, Gaucher disease, Gerstmann-Straussler-Scheinker syndrome, Gillespie syndrome, type I glutaric aciduria, type II glutaric aciduria, GRACILE syndrome, GRIN2B-related neurodevelopmental disorder, Griscelli syndrome, Gustavson syndrome, Hailey-Hailey disease, Harlequin type ichthyosis, Hemochromatosis type 1, Hemochromatosis type 2A, Hemochromatosis type 2B, Hemochromatosis type 3, Hemochromatosis 4, Hemochromatosis 5, hemophilia, hepatoerythropoietic porphyria, hereditary coproporphyria, hereditary hemorrhagic telangiectasia, hereditary inclusion body myopathy, hereditary multiple exostoses, hereditary spastic paraplegia, Hermansky-Pudiak syndrome, hereditary neuropathy with liability to pressure palsies (HNPP), heterotaxy, homocystinuria, Huntington's disease, Hunter syndrome, Hurler syndrome, Hutchinson-Gilford progeria syndrome, hyperlysinemia, primary hyperoxaluria, hyperphenylalainemia, Tangier disease, hypochondrogenesis, hypochondroplasia, immunodeficiency-centrometric instability-facial anomalies syndrome (ICF), incontinentia pigmenti, infantile cerebral and cerebellar atrophy with postnatal progressive microcephaly, ischiopatellar dysplasia, isodicentric 15, PRICKLE1-related progressive myoclonus epilepsy with ataxia, Jackson-Weiss syndrome, Jacobsen syndrome, Joubert syndrome, Juvenile-onset dystonia, Juvenile primary lateral sclerosis, Keloid disorder, KIF1A-associated neurological disorder, Kleefstra syndrome, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Gridle Muscular Dystrophy, Lynch syndrome, lipoprotein lipase deficiency, malignany hyperthermia, Maple syrup urine disease, Marfan syndrome, Maroteaux-Lamy syndrome, McCune-Albright syndrome, McLeod syndrome, MEDNIK syndrome, familial Mediterranean fever, Mekes disease, methemoglobinemia, methylmalonic acidemia, Micro syndrome, microcephaly, Miller-Dieker syndrome, Morquio syndrome, Mowat-Wilson syndrome, Muenke syndrome, type 1 multiple endocrine neoplasia, type 2 multiple endocrine neoplasia, muscular dystrophy, Becker muscular dystrophy, myostatin-related muscle hypertrophy, myotonic dystrophy, Natowicz syndrome, Nedamss neurodevelopment disorder, type I neurofibromatosis, type II neurofibromatosis, Niemann-Pick disease, Nonketotic hyperglycinemia, nonsyndromic deafness, noonan syndrome, Norman-Roberts syndrome, Ogden syndrome, Omenn syndrome, Osteogenesis imperfecta, Ostravik-Lindemann-Solberg syndrome, Pantothenate kinase-associated neurodegeneration, Patau syndrome, PCC deficiency, porphyria cutanea tarda, Pendred syndrome, Peutz-Jeghers syndrome, Pfeiffer syndrome, Phelan-McDermid syndrome, Phenylketouria, Pipecolic acidemia, Pitt-Hopkins syndrome, Polycystic kidney disease, polycystic ovary syndrome, porphyria, Prader-Willi syndrome, Primary ciliary dyskinesia, primary pulmonary hypertension, protein C deficiency, protein S deficiency, proximal 18q deletion syndrome, Pseudo-Gaucher disease, pseudoxanthoma elasticum, Retinitis pigmentosa, Rett syndrome, Roberts syndrome, Rubinstein-Taybi syndrome, Sandhoff disease, Sanfilippo syndrome, Schwartz-Jampel syndrome, Sjogren-Larsson syndrome, Skin fragility-wooly hair-palmoplantar keratoderma syndrome, Spondyloepiphyseal dysplasia congenita, Shprintzen-Goldbery syndrome, Sickle cell anemia, Siderius X-linked mental retardation syndrome, Sideroblastic anemia, Sly syndrome, Smith-Lemli-Optiz syndrome, Smith-Magenis syndrome, Synder-Robinson syndrome, Spinal muscular atrophy, Spinocerebellar ataxia, split hand split foot-nystagmus syndrome, SSB syndrome, Stargardt disease, Stickler syndrome, Strudwick syndrome, Tay-Sachs disease, tetrahydrobiopterin deficiency, thanatophoric dysplasia, thickened earlobes-conductive deafness syndrome, Treacher Collins syndrome, Tuberous sclerosis complex, Turner syndrome, Usher syndrome, Variegate porphyria, Vijoen-Kallis-Voges syndrome, von Hippel-Lindau disease, von Willebrand disease, Waardenburg syndrome, Warkany syndrome 2, Weissenbacher-Zweymüller syndrome, Weyer's ulnar ray/oligodactyly syndrome, Williams syndrome, Wilson disease, Woodhouse-Sakati syndrome, Wolf-Hirschhorn syndrome, Xeroderma pigmentosum, Fragile X syndrome, X-linked spinal-bulbar muscle atrophy, Xp11.2 duplication syndrome, X-linked severe combined immunodeficiency, X-linked sideroblastic anemia, triple X syndrome, XXXX syndrome, XXXXX syndrome, XXXXY syndrome, XYY syndrome, XXYY syndrome, XYYY syndrome, XXXY syndrome, XYYYY syndrome, or Zellweger syndrome.

In some embodiments, the genetic disorder is down syndrome. In some embodiments, the genetic disorder is Fragile X syndrome. In some embodiments, the genetic disorder is triple X syndrome. In some embodiments, the genetic disorder is XXXX syndrome. In some embodiments, the genetic disorder is XXXXX syndrome. In some embodiments, the genetic disorder is XXXXY. In some embodiments, the genetic disorder is XYY syndrome. In some embodiments, the genetic disorder is XYY syndrome. In some embodiments, the genetic disorder is XXYY syndrome. In some embodiments, the genetic disorder is XYYY. In some embodiments, the genetic disorder is XXXY syndrome. In some embodiments, the genetic disorder is XYYYY. In some embodiments, the genetic disorder is Tay-Sachs disease. In some embodiments, the genetic disorder is Angelman syndrome. In some embodiments, the genetic disorder is Cystic fibrosis. In some embodiments, the genetic disorder is DiGeorge syndrome. In some embodiments, the genetic disorder is Duchenne muscular dystrophy. In some embodiments, the genetic disorder is fetal familial insomnia. In some embodiments, the genetic disorder is Creutzfeld-Jakob disease. In some embodiments, the genetic disorder is Gerstmann-Sträussler-Scheinker syndrome. In some embodiments, the genetic disorder is Hailey-Hailey disease. In some embodiments, the genetic disorder is Hemophilia. In some embodiments, the genetic disorder is Huntington's disease. In some embodiments, the genetic disorder is Marfan syndrome. In some embodiments, the genetic disorder is Prader-Willi syndrome. In some embodiments, the genetic disorder is Sickle Cell anemia. In some embodiments, the genetic disorder is Turner syndrome. In some embodiments, the genetic disorder is von Hippel-Lindau disease.

In some embodiments, the one or more genetic disorders is chromosomal abnormalities. Non-limiting examples of chromosomal abnormalities include aneuploidy, copy number variant, single nucleotide variant, insertion, translocation, unbalanced translocation, rearrangement, subtelomeric rearrangement, unbalance chromosomal rearrangement, unbalance subtelomeric rearrangement, deletion, inversions, unbalanced inversions, duplication, telomere instability, telomere shortening, single nucleotide substitution, micro deletion, micro-insertion, short deletions, short insertion, multi-nucleotide changes, DNA methylation and, and loss of imprint (LOI). Non-limiting examples of chromosomal aneuploidy include trisomy 21, trisomy 18, trisomy 13, trisomy 16, and XXX. In some embodiments, the chromosomal aneuploidy is a complete monosomy. In some embodiments, the complete monosomy is monosomy X, monosomy 21, monosomy 22, monosomy 16 or monosomy 15. In some embodiments, the chromosomal aneuploidy is a partial monosomy. In some embodiments, the partial monosomy is monosomy X, monosomy 21, monosomy 22, monosomy 16 or monosomy 15. In some embodiments, the chromosomal abnormality is trisomy 18. In some embodiments, the chromosomal abnormality is trisomy 13.

In some embodiments, the genetic material from the maternal biological sample is used to detect fetal conditions. In some embodiments, the fetal condition is an abdominal defect. Non-limiting examples of abdominal defects include congenital diaphragmatic hernia, gastroschisis, omphalocele, and cloacal exstrophy. In some embodiments, the fetal condition is an amniotic sac problem. In some embodiments, the amniotic sac problem is amniotic band syndrome. In some embodiments, the fetal condition is a chest/lung condition. Non-limiting examples of chest/lung conditions include bronchopulmonary sequestration, congenital high airway obstruction syndrome, congenital lobar emphysema, congenital pulmonary airway malformation, and pleural effusion. In some embodiments, the fetal condition is a craniofacial defect. Non-limiting examples of craniofacial defect include cleft lip, cleft palate, congenital high airway obstruction syndrome (CHAOS), and neck masses (e.g., lymphangioma). In some embodiments, the fetal condition is an extremity/skeletal abnormality. Non-limiting examples of extremity/skeletal abnormalities include amelia, club and vertical talus, fibular hemimelia, and skeletal dysplasia. In some embodiments, the fetal condition is a gastrointestinal abnormality. Non-limiting examples of gastrointestinal abnormalities include duodenal atresia, esophageal atresia, fetal bowel obstruction, and tracheoesophageal fistula. In some embodiments, the fetal condition is a heart disease. Non-limiting examples include atrial septal defect, atrioventricular canal defect, coarctation of the aorta, congenital heart disease, double outlet right ventricle, heterotaxy syndrome, hypoplastic left heart syndrome, tetralogy of fallot, transposition of the great arteries, ventricular septal defect, and rhythm abnormalities. In some embodiments, the fetal condition is a neurological condition. Non-limiting examples of neurological conditions include agenesis of corpus callosum, encephalocele, hydrocephalus, spina bifida, and vein of galen malformation. In some embodiments, the fetal condition is a renal abnormality. Non-limiting examples of renal abnormalities include bladder exstrophy, bladder outlet obstruction, hydronephrosis, and prune belly syndrome. In some embodiments, the fetal condition is a tumor/abnormal growth. Non-limiting examples includes lyphangioma, neuroblastoma, and sacrococcygeal teratoma. In some embodiments, the fetal condition is a twin condition. Non-limiting examples of twin conditions include conjoined twins, selective intrauterine growth restriction, twin reversed arterial perfusion, and twin-to-twin transfusion syndrome.

In some embodiments, a healthy fetus is associated with an expected ratio of one or more biomarkers. In some embodiments, a healthy pregnancy-related state is associated with an expected ratio of one or more biomarkers. In some embodiments, the expected ratio of the one or more biomarkers is different in different gestational age. In some embodiments, the deviations from the expected ratios of the one or more biomarkers is associated with abnormalities in fetal health. In some embodiments, the deviations from the expected ratios of the one or more biomarkers is associated with abnormalities in pregnancy-related health state. Non-limiting examples of pregnancy-related diseases include miscarriage, fetus loss, preeclampsia, eclampsia, hypertension, preterm label, gestational diabetes, hyperemesis gravidarum, placenta previa, molar pregnancies, gestational trophoblastic disease, and hemolysis, elevated liver enzymes, low platelet count (HELLP) syndrome.

In some embodiments, the prenatal testing is utilized to monitor the health of the fetus during the duration of the pregnancy. In some embodiments, the prenatal testing is utilized to monitor the health of the mother during the duration of the pregnancy. In some embodiments, the prenatal testing is utilized to monitor the health of the mother and the fetus during the duration of the pregnancy.

In some embodiments, the prenatal testing is utilized to monitor the health of the fetus during the pregnancy or the health of pregnancy due to environmental factors. In some embodiments, environmental factors can negatively impact the health of the fetus during the pregnancy or the health of the pregnancy in the mother. Non-limiting examples of environmental factors that can negatively impact fetal health or pregnancy health includes: cigarette smoking, cigarette smoke, cigar smoking, cigar smoke, alcohol use, drug use, air pollution, water pollution, lead exposure, mercury exposure, nickel exposure, manganese exposure, inhalational anesthetics exposure, organic solvents exposure, pesticide exposure, radiation exposure, mental stress, and physical stress.

In some embodiments, the prenatal testing is utilized to monitor the health of the fetus during the pregnancy or the health of the pregnancy subsequent to a physical injury to the mother. In some embodiments, the prenatal testing is utilized to monitor the health of the fetus during the pregnancy or the health of the pregnancy subsequent to a mental change to the mother. In some embodiments, the prenatal testing is utilized to monitor the health of the fetus during the pregnancy or the health of the pregnancy subsequent to exposure of the mother to an environmental factor. In some embodiments, the prenatal testing is utilized to monitor the health of the fetus during the pregnancy or the health of the pregnancy due to the diet of the mother.

In some embodiments, the prenatal testing is utilized to monitor the health of the fetus during the pregnancy to monitor for the development of fetal conditions. In some embodiments the prenatal testing is utilized to monitor the health of the pregnancy to monitor for the development of pregnancy-related diseases.

In some embodiments, the prenatal testing is utilized to monitor the health of the fetus during the pregnancy or the health of the pregnancy for any reason or combination of reasons disclosed herein.

In some embodiments, a maternal biological sample is collected and tested one or more times during the duration of a pregnancy. In some embodiments, the maternal biological sample is collected and tested at least about one time, at least about two times, at least about three times, at least about four times, at least about five times, at least about six times, at least about seven times, at least about eight times, at least about nine times, at least about ten times, or more times during the duration of a pregnancy. In some embodiments, the maternal biological sample is collected and tested at most about ten times, at most about nine times, at most about eight times, at most about seven times, at most about six times, at most about five times, at most about four times, at most about three times, at most about two times, or at most about one time during the duration of a pregnancy. In some embodiments, the maternal biological sample is collected and tested about one time, about two times, about three times, about four times, about five times, about six times, about seven times, about eight times, about nine times, or about ten times during the duration of a pregnancy.

In some embodiments, a maternal biological sample is collected and tested about once a month during the duration of a pregnancy. In some embodiments, a maternal biological sample is collected and tested about once a trimester during the duration of a pregnancy. In some embodiments, a maternal biological sample is collected and tested about once a week during the duration of a pregnancy.

In some embodiments, a maternal biological sample is collected and tested at one week gestation. In some embodiments, a maternal biological sample is collected and tested at two weeks gestation. In some embodiments, a maternal biological sample is collected and tested at three weeks gestation. In some embodiments, a maternal biological sample is collected and tested at four weeks gestation. In some embodiments, a maternal biological sample is collected and tested at five weeks gestation. In some embodiments, a maternal biological sample is collected and tested at six weeks gestation. In some embodiments, a maternal biological sample is collected and tested at seven weeks gestation. In some embodiments, a maternal biological sample is collected and tested at eight weeks gestation. In some embodiments, a maternal biological sample is collected and tested at nine weeks gestation. In some embodiments, a maternal biological sample is collected and tested at 10 weeks gestation. In some embodiments, a maternal biological sample is collected and tested at 11 weeks gestation. In some embodiments, a maternal biological sample is collected and tested at 12 weeks gestation. In some embodiments, a maternal biological sample is collected and tested at 13 weeks gestation. In some embodiments, a maternal biological sample is collected and tested at 14 weeks gestation. In some embodiments, a maternal biological sample is collected and tested at 15 weeks gestation. In some embodiments, a maternal biological sample is collected and tested at 16 weeks gestation. In some embodiments, a maternal biological sample is collected and tested at 17 weeks gestation. In some embodiments, a maternal biological sample is collected and tested at 18 weeks gestation. In some embodiments, a maternal biological sample is collected and tested at 19 weeks gestation. In some embodiments, a maternal biological sample is collected and tested at 20 weeks gestation. In some embodiments, a maternal biological sample is collected and tested at 21 weeks gestation. In some embodiments, a maternal biological sample is collected and tested at 22 weeks gestation. In some embodiments, a maternal biological sample is collected and tested at 23 weeks gestation. In some embodiments, a maternal biological sample is collected and tested at 24 weeks gestation. In some embodiments, a maternal biological sample is collected and tested at 25 weeks gestation. In some embodiments, a maternal biological sample is collected and tested at 26 weeks gestation. In some embodiments, a maternal biological sample is collected and tested at 27 weeks gestation. In some embodiments, a maternal biological sample is collected and tested at 28 weeks gestation. In some embodiments, a maternal biological sample is collected and tested at 29 weeks gestation. In some embodiments, a maternal biological sample is collected and tested at 30 weeks gestation. In some embodiments, a maternal biological sample is collected and tested at 31 weeks gestation. In some embodiments, a maternal biological sample is collected and tested at 32 weeks gestation. In some embodiments, a maternal biological sample is collected and tested at 33 weeks gestation. In some embodiments, a maternal biological sample is collected and tested at 34 weeks gestation. In some embodiments, a maternal biological sample is collected and tested at 35 weeks gestation. In some embodiments, a maternal biological sample is collected and tested at 36 weeks gestation. In some embodiments, a maternal biological sample is collected and tested at 37 weeks gestation. In some embodiments, a maternal biological sample is collected and tested at 38 weeks gestation. In some embodiments, a maternal biological sample is collected and tested at 39 weeks gestation. In some embodiments, a maternal biological sample is collected and tested at 40 weeks gestation. In some embodiments, a maternal biological sample is collected and tested at 41 weeks gestation. In some embodiments, a maternal biological sample is collected and tested at 42 weeks gestation. In some embodiments, a maternal biological sample is collected and tested at 43 weeks gestation. In some embodiments, a maternal biological sample is collected and tested at 44 weeks gestation. In some embodiments, a maternal biological sample is collected and tested at 45 weeks gestation.

In some embodiments, the prenatal tested is utilized to select one or more therapeutic agents. In some embodiments, the one or more therapeutic agents are aimed at specific molecular targets. In some embodiments, the molecular targets are associated with one or more fetal biomarkers. In some embodiments, the molecular targets are associated with one or more maternal biomarkers. In some embodiments, the molecular targets are associated with one or more fetal biomarkers and one or more maternal biomarkers. In some embodiments, the one or more therapeutic agents result in an increase in the one or more fetal biomarkers. In some embodiments, the one or more therapeutic agents result in a decrease is one or more fetal biomarkers. In some embodiments, the one or more therapeutic agents result in an increase in the one or more maternal biomarkers. In some embodiments, the one or more therapeutic agents result in a decrease in one or more maternal biomarkers. In some embodiments, the one or more therapeutic agents result in an increase in one or more fetal biomarkers and an increase in one or more maternal biomarkers. In some embodiments, the one or more therapeutic agents result in a decrease in one or more fetal biomarkers and a decrease in one or more maternal biomarkers. In some embodiments, the one or more therapeutic agents result in an increase in one or more fetal biomarkers and a decrease in one or more maternal biomarkers. In some embodiments, the one or more therapeutic agents result in a decrease in one or more fetal biomarkers and an increase in one or more maternal biomarkers.

Compositions and methods provided herein can be used for isolating fetal cells from a maternal biological sample. In some embodiments, fetal cells are isolated from the maternal biological sample. In some embodiments, the characteristics of the fetal cells are studied. In some embodiments, the gene expression of the fetal cells is studied. In some embodiments, the metabolism of the fetal cells is studied. In some embodiments, the interaction between different fetal cell types is studied. In some embodiments, the interaction between fetal cells and pathogens is studied. In some embodiments, the characteristics and morphology of different fetal cell types are studied. Non-limiting examples of cellular characteristics to be studied include gene expression, metabolism, cytokine production, chemokine production, activation levels, cell surface marker expression, replication rate, response to pathogens, and homeostasis state. Non-limiting examples of cellular morphology to be studied include cell shape, size of cytoplasm, size of nucleus, number of organelles, size of organelles, structure of cell, color of cell, texture of cell, and size of cell.

In some embodiments, maternal cells are also isolated. In some embodiments, the interaction between maternal cells and fetal cells is studied. In some embodiments, maternal immune cells are also isolation. In some embodiments, the interaction between fetal cells and maternal immune cells is studied.

### EXAMPLES

### Example 1: Circulating Fetal Cell Marker Discovery

### Generation of Labeled Fetal Cells

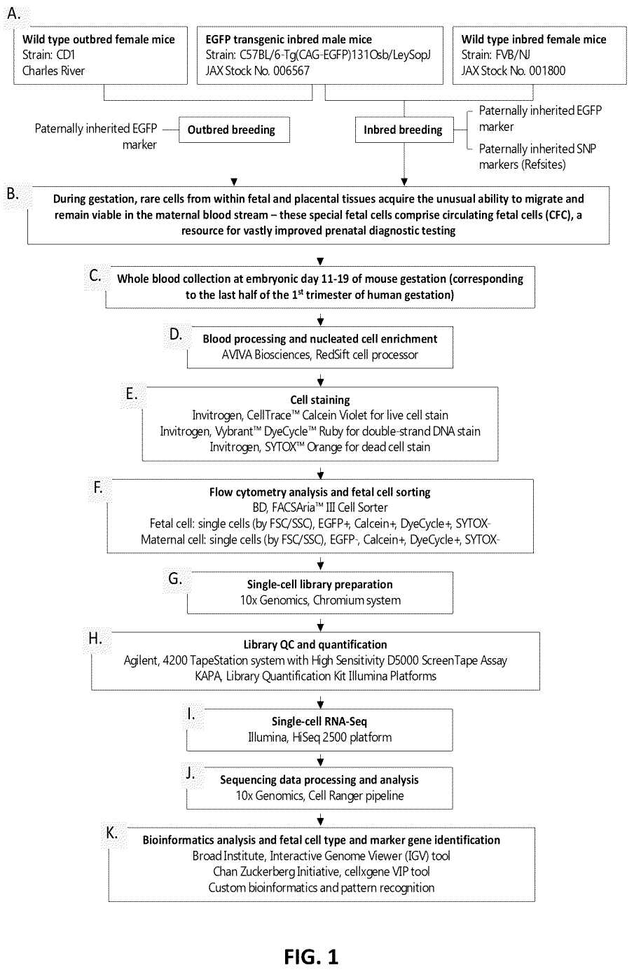

Fetal and placental cells were genetically labeled by using homozygous enhanced green fluorescent protein (EGFP) transgenic C57BL/6-Tg(CAG-EGFP)131Osb/LeySopJ male mice mated with EGFP-negative FVB/NJ females to yield fetal progeny cells that were identified and purified based upon EGFP fluorescence against a background of non-fluorescent maternal blood cells in addition to differences in 484 expressed single-nucleotide polymorphisms (SNPs) between the maternal and fetal genotypes. Transgenic C57BL/6-Tg(CAG-EGFP)131Osb/LeySopJ male mice were also mated with EGFP-negative CDI females to yield progeny cells that were identified and purified based only upon EGFP fluorescence against a background of non-fluorescent maternal blood cells. When crossed with EGFP-negative female mice, the resulting hemizygous fetal cells inherited one copy of the EGFP transgene, which allowed CFCs to be isolated from maternal blood by using EGFP as a fetal marker and a source of fluorescence for downstream fluorescence-activated cell sorting (FACS) purification (FIG. 1).

### Processing of Maternal Blood Samples

During gestation, rare cells from within various fetal and placental tissues acquire the ability to migrate from the fetal and placental compartment into the maternal circulation and then remain viable while suspended in the circulating maternal blood. These rare cells comprise circulating fetal cells (CFCs). Pregnant female mice were sacrificed between embryonic day 11 to 19, which spans a period that corresponds to the last half of the 1st trimester of human gestation. Whole blood was collected by heart puncture from pregnant female mice anesthetized with isoflurane inhalation. Collected blood was transferred immediately into EDTA-treated blood collection tubes and mixed thoroughly by gently inverting the tube. Blood samples were kept at 4° C. up to 24 hours to reproduce the conditions and treatment of clinical human blood samples.

Blood samples were depleted of red blood cells using the microfluidic platform RedSift Cell Processor coupled with the associated filter array kit, which isolated and enriched nucleated cells and removed erythrocytes, thrombocytes, and plasma components.

### Isolation of CFCs

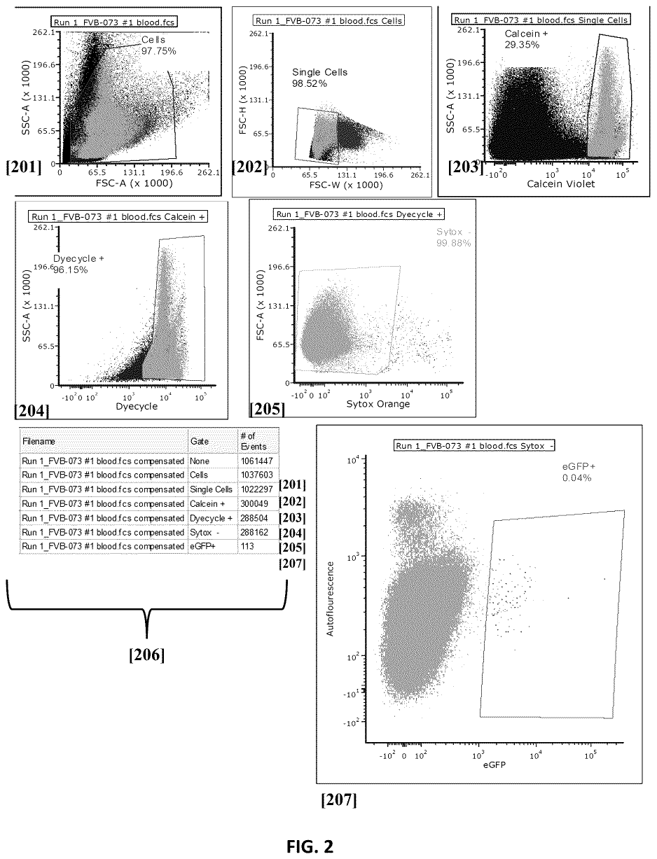

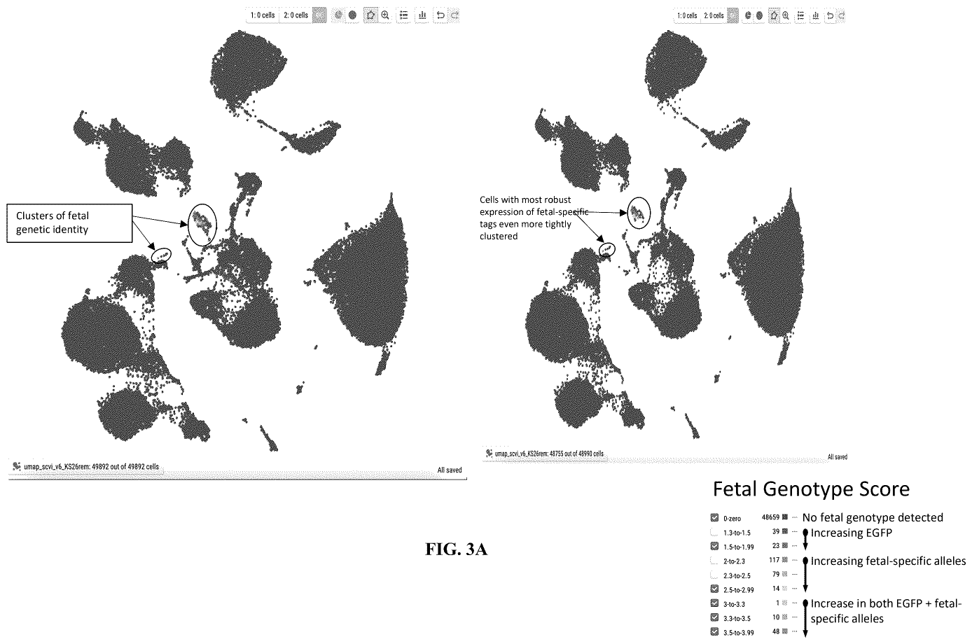

The recovered nucleated cells (including circulating fetal and extra-embryonic placental cells (CFC)) were then stained with Calcein Violet AM, DyeCycle™ Ruby, and SYTOX™ Orange for evaluation of active cell metabolism, presence of double-strand DNA in live cells, and cell death, respectively (FIG. 2). Each staining solution in DMSO was added to the cell suspension, mixed, and incubated for at least 15 min at room temperature. The sample tubes with the stained cell suspension were then kept on ice and protected from light until cell sorting.

Stained cells were analyzed and sorted on BD FACSAria™ III sorter equipped with BD FACSDiva™ software. Doublet and multiplet cells were eliminated by forward scatter (FSC)-side scatter (SSC) gating followed by FSC width-height gating. Four fluorescent channels, FITC, BV421, PE and APC were used for detecting EGFP (fetal gene expression marker), Calcein Violet (cell health marker), SYTOX Orange (cell death marker), and DyeCycle Ruby (nuclear DNA marker), respectively (gates shown in FIG. 2). The BV786 channel was also used to detect and discard autofluorescent cells. The stained cells from maternal blood of wild type pregnancies, where wild type C57BL/6J male mice instead of EGFP-Tg male mice were crossed with female mice, and unstained cells were used as an EGFP-negative control and an unstained control, respectively. In the case a wild type pregnancy was not obtained, non-pregnant female blood was used as an EGFP-negative control. EGFP-positive CFC-enriched live, nucleated, and healthy cells from maternal blood of EGFP-Tg pregnancies (EGFP+/Calcein Violet+/SYTOX Orange-/DyeCycle Ruby+/autofluorescence-population) were individually sorted from each sample and then pooled together in a well filled with buffer within each gestational age group in an effort to obtain approximately 1100 cell enriched-CFC pool for each gestational age group. If the number of EGFP-positive CFC-enriched live cells was far below 1100, EGFP-negative live maternal cells (EGFP-/Calcein Violet+/SYTOX Orange-/DyeCycle Ruby+/autofluorescence-population) were added as carrier cells to facilitate efficient single cell library reaction in the downstream Chromium processor.

### Single Cell Transcriptomics

Cells were processed using the 10× Genomics Chromium™ controller and Chromium™ Single Cell 3′ Library and Gel Bead Kit (v2, v3, or Next GEM v3.1), according to manufacturer's instructions. The libraries were next analyzed for quality and quantification using Agilent 4200 TapeStation Assay and KAPA Library Quantification Kit, respectively. These barcoded single-cell cDNA libraries were then used to prepare next-generation sequencing (NGS) cDNA libraries, and aliquots of the libraries were then sequenced on Illumina HiSeq instruments and the resulting data processed to analyze for transcript abundance and sequence on a cell-by-cell basis.

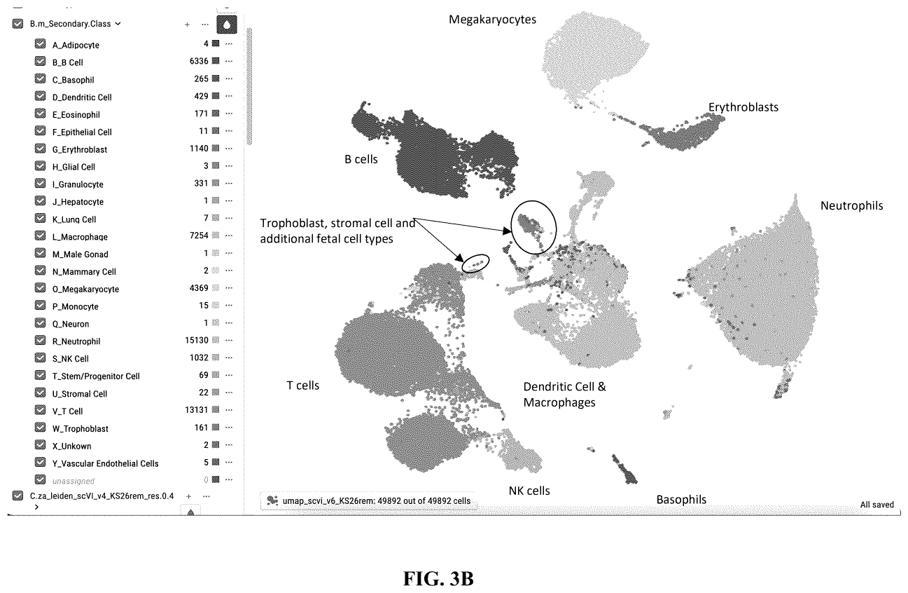

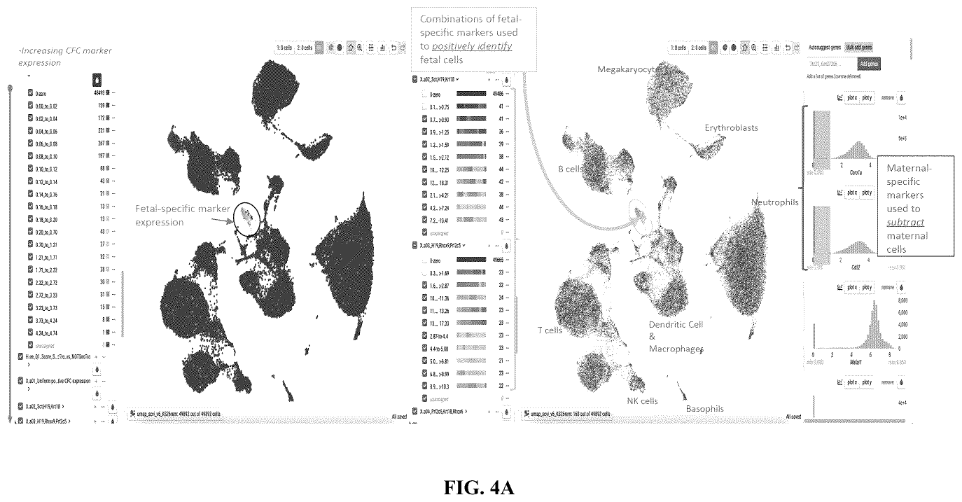

Single-cell transcriptomic analysis showed detection of EGFP+ cells and the allele status of a set of 484 paternal-specific and fetal-specific reference allele SNPs. This observation confirmed the successful isolation of CFCs from the maternal blood. The single-cell gene expression analysis of maternal blood enriched for EGFP-expressing fetal cells identified concentrated clusters of fetal cells marked by expression of fetal-specific alleles (FIG. 3A). In addition, single-cell gene expression profiles identified several fetal cell types in clusters that were distinct from the expected maternal cell types. Within the CFC clusters, trophoblasts and stromal cells were the most abundant CFCs as shown in FIG. 3B. In the cluster analysis also showed that fetal trophoblast cells, stromal cells, and additional fetal cell types clustered separately from maternal B cells, T cells, NK cells, Basophils, DCs, Macrophages, Neutrophils, Erythroblasts, and Megakaryocytes.

### Data Processing and Analysis

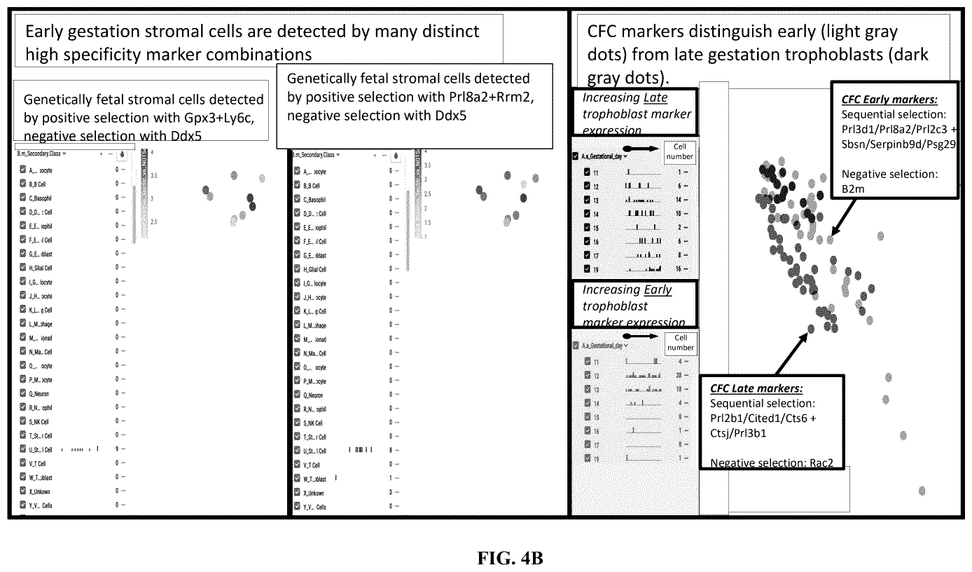

Raw sequencing data from each library was processed using Cell Ranger 3.1.0. software. Individually-barcoded single cell cDNA libraries were prepared and subjected to RNA-seq NGS as described above to characterize the gene and SNP allele expression profiles within each single cell. Determination of expressed transcripts of paternally inherited EGFP and reference alleles of SNPs (Ref) was then used to identify fetal cells and distinguish them from the EGFP-negative, Ref-negative maternal cell expression profiles. Analysis of the complete expression profile in each cell then enabled characterization of the cell type of each fetal and maternal cell, and comparison of these expression profiles allowed identification of differentially expressed genes that distinguished fetal from maternal cells (FIG. 4A); for example, combinations of fetal-specific markers can be used to positively identify fetal cells and maternal-specific markers can be used to substrate maternal cells from a sample.

The FASTQ files for each Chromium library were processed with Cell Ranger v3.1.0 to generate single cell gene counts. Short reads were mapped with the Cell Ranger count pipeline using the STAR RNA-seq aligner. Reads were mapped to a hybrid transcriptome reference constructed from all mouse gene transcripts of the mm10 genome assembly and the rabbit EGFP transgene sequence.

In addition, the Cell Ranger BAM file annotated with barcode information was used to extract reference and alternate allele read counts for fetal cell genetic identification. Fetal cells were identified using a manually curated set of 484 exonic SNP sites chosen from expressed SNPs where the allele inherited from the homozygous paternal (homozygous Ref allele) and maternal (homozygous Alt allele) inbred mouse strains differed. Analyzing progeny RNA at these expressed SNPs identified fetal transcripts and cells by detecting the inheritance of the expressed paternal Ref alleles that were absent in the homozygous Alt maternal cells, but present in all fetal cells. A barcode was then called to be positive for a SNP if at least two reference allele reads mapped to the barcode, or if the reference read count was at least 8% of the total read count in cases where the site had at least 15 reads.

The sequence of all 484 SNPs and EGFP-positive reads was then combined in a sensitive assay to distinguish fetal vs maternal genotype using the total number of positive SNP sites, and the total number of reference reads across all positive SNP sites to identify maternal cells (Ref-/EGFP-) and fetal cells (positive for at least some Ref and EGFP expression. This analysis also provided a list of the SNP sites and corresponding genes that were most frequently found to be positive in fetal cells. Additionally, this analysis provided gene expression profiles that enabled identification of fetal cells based on differential gene expression.

The bioinformatics analysis additionally used the Interactive Genome Viewer, Cellxgene VIP tool.

Single-cell data analysis was carried out for 1) each sample individually, 2) each gestational day integrative of samples per time-point and, 3) all samples (from all time-points) integrated. Data normalization, finding highly variable genes, scaling, dimensionality reduction, clustering, and cell-type annotation were the major procedures followed in analysis using the standard Seurat v4.0.2 pipeline. To retain maximum fetal cells that exhibited a low number of UMI/genes expressed, filtering based on the standard metrics was bypassed.

A reference-based cell-type annotation was used for annotation using studies that were comparable in cohort characteristics. Fetal and placenta samples from the studies GSE152248 (Marsh BP, 2020) and GSE108097 (Xiaoping Han, 2018) were downloaded and processed to prepare the reference data. These processed reference data were used individually and in combination to evaluate their performances, and then optimal reference data combination was selected based on benchmarks.

There are multiple assays or signals [hereafter referred as ‘meta’] to identify the fetal cells such as 1) EGFP expression, 2) presence of heterozygous SNPs (from paternal and maternal) [hereafter referred as ‘refsites’], 3) cells annotated as trophoblasts or showing strong signal of trophoblast marker genes (the trophoblast cell type is characteristic of fetal cells), 4) lower expression of selected negative trophoblast marker genes, and 5) low total UMI count, which signified fewer gene expression in fetal cells. While EGFP and refsites were strong and robust signals, dropout events lead to false negatives; the remaining meta covered most of the fetal cells. Hence, a combined scoring method was developed where each meta was scored individually and then scored in the combination. The desiR v1.2. R package was employed to score the meta individually with a defined range of values for each meta and the scores were in the range of 0-1.

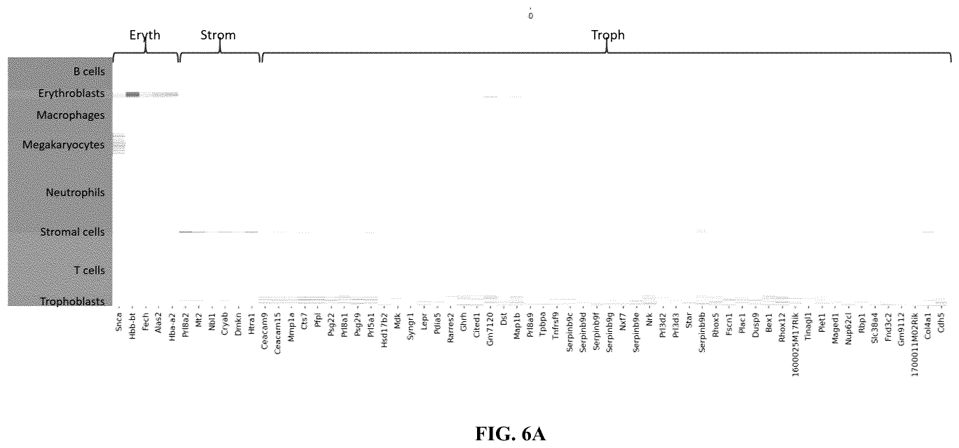

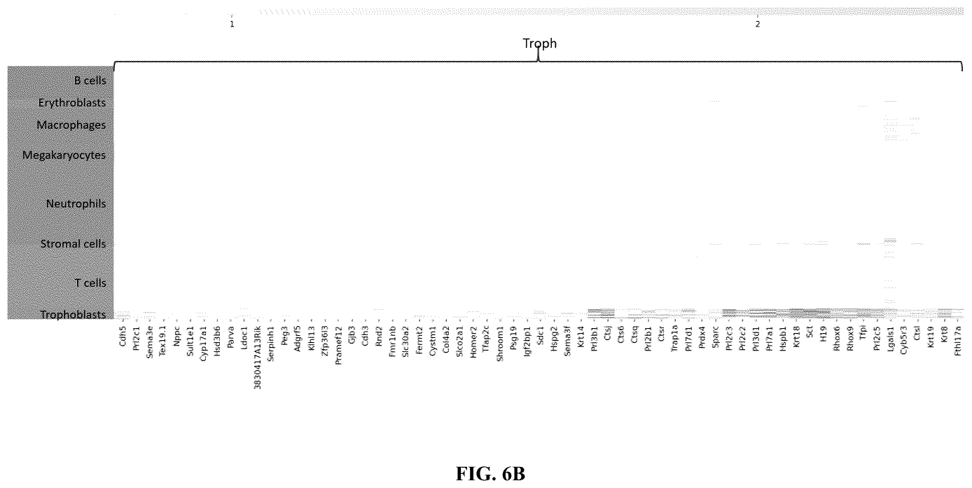

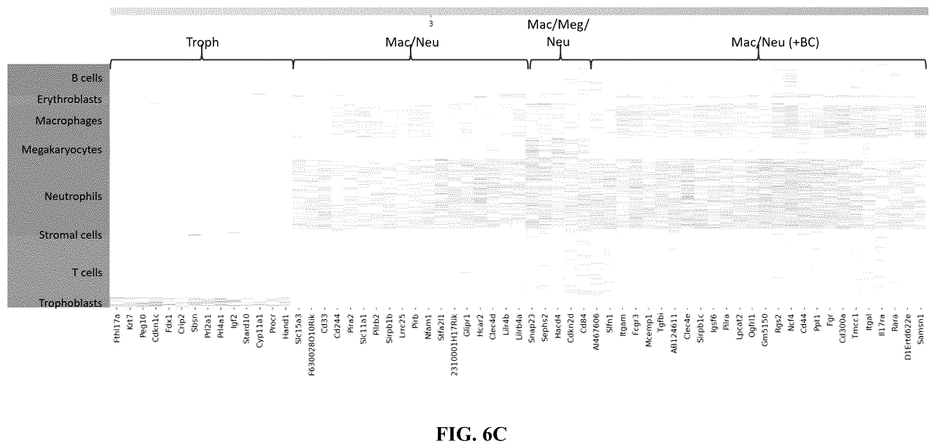

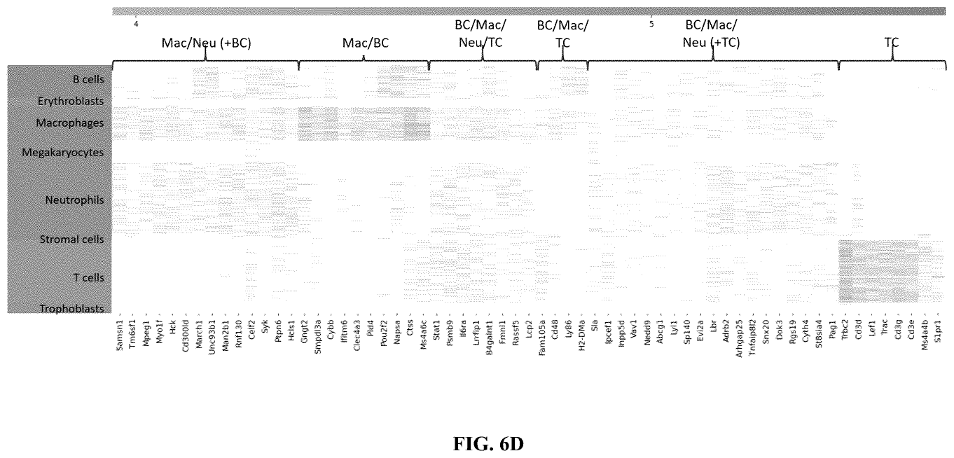

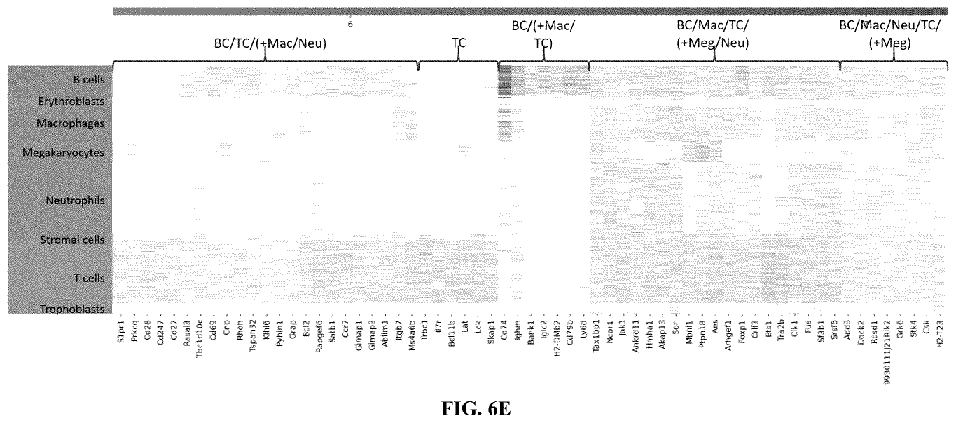

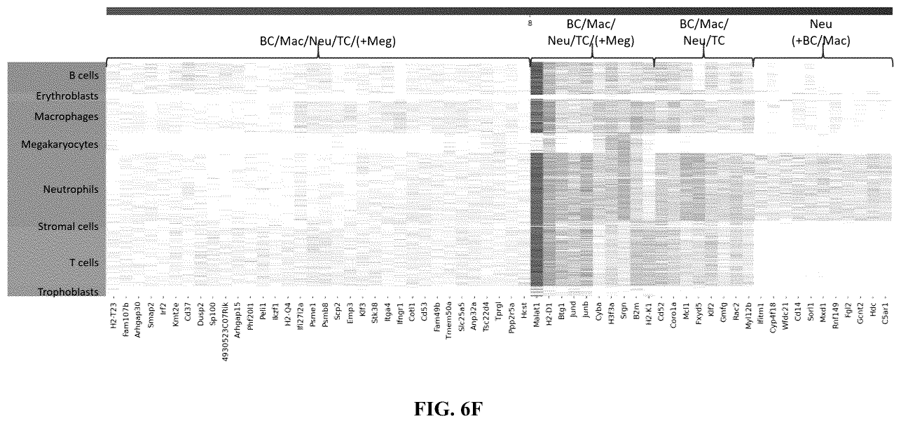

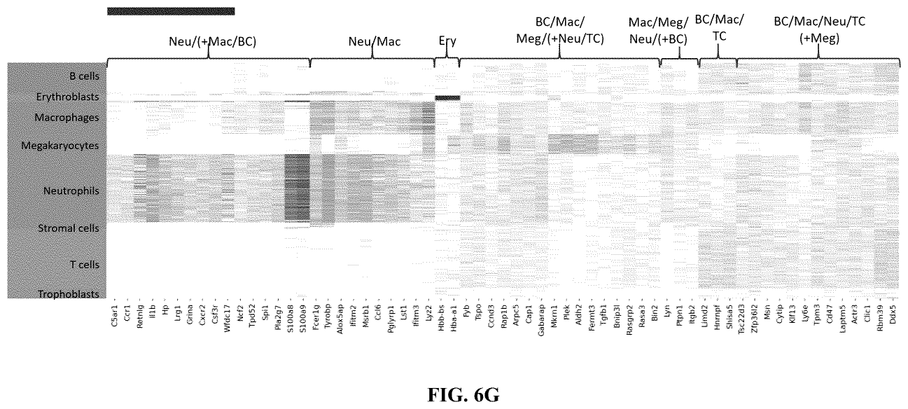

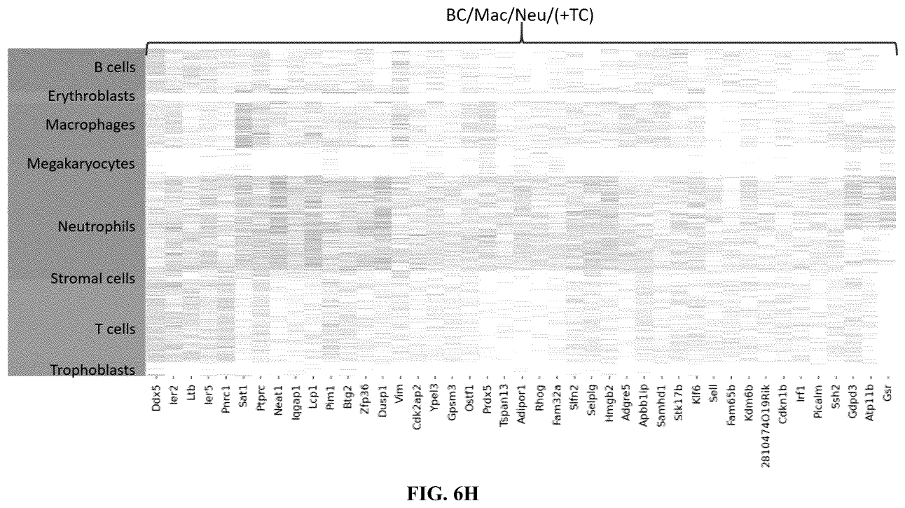

The average number of reads supporting the ref allele out of all refsites were weight normalized with total UMI count score. EGFP and the total number of refsites were scored individually and the individual scores were then combined to create a combined score. To identify noisy cell type annotations, scoring of each cell using pre-identified marker gene lists for each cell type was employed. Then the cells were scored for each marker list (FIGS. 6A-6H). To identify cells that showed multiple cell type characteristics, a ratio of these scores was created. The entire marker discovery workflow is shown in FIG. 1.

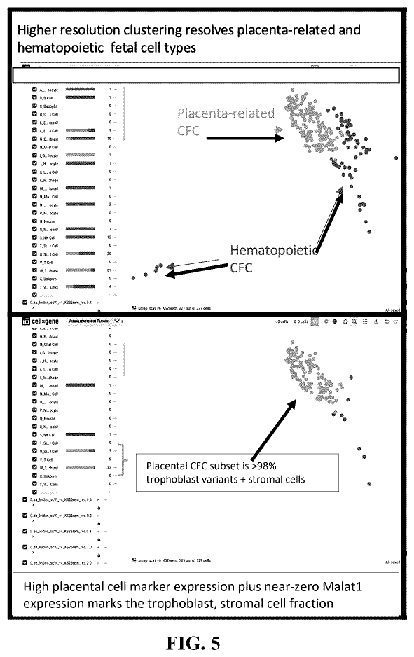

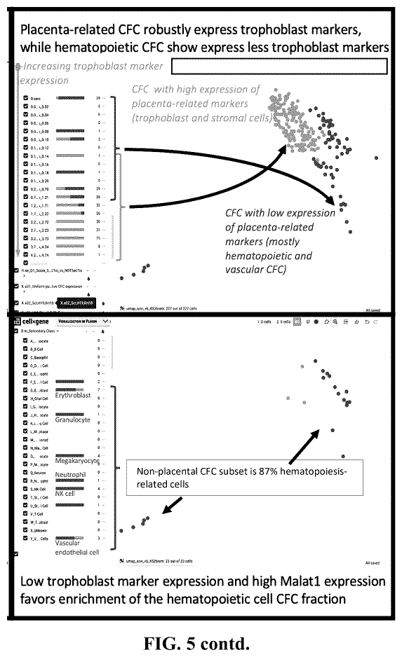

A list of maternal-specific markers and fetal-specific markers was generated using the data from the single-cell transcriptomic analysis. Analysis of the cluster of fetal cells identified in FIGS. 3A and 3B for expression of fetal-specific or maternal-specific markers found that the expression of fetal-specific markers was highly concentrated in the fetal cell cluster. Using a combination of fetal-specific markers and maternal-specific markers allowed for the identification of the CFCs at a high purity and yield (FIG. 4A). Additionally, analysis of fetal-specific markers allowed for the differentiation of stromal cells and trophoblasts from early gestation or late gestation (FIG. 4B). For example, early gestation stromal cells were detected by 2 isolation schemes: (1) positive selection with Gpx3 and Ly6c with negative selection with Ddx5 and (2) positive selection with Prl8a2 and Rrm2 and negative selection with Ddx5. The analysis found that a first positive selection with Prl3d1, Prl8a2, Prl2c3, a second positive selection with Sbsn, Serpinb9d, Psg29, and then followed by a negative selection with B2m identified early gestation trophoblasts, and that a first positive selection with Prl2b1, Cited1, Cts6, a second positive selection with Ctsj, Prl3b1, followed by a negative selection with Rac2 identified later gestation trophoblasts. Further analysis of fetal cells using higher resolution clustering showed the existence of two subclusters of CFCs, placenta-related CFCs and Hematopoietic CFCs, which were distinguished by differential expression ratios of a fetal-specific marker (FIG. 5). Specifically, placenta-related CFCs expressed trophoblast markers and had a low level of expression of the Malat1 biomarker, while hematopoietic CFCs had a lower level of expression of trophoblast markers and a high expression level of the Malat1 biomarker.

The fetal-specific and maternal-specific markers were plotted in heat maps to determine the expression levels of the different markers in both fetal cell types and maternal cell types. FIGS. 6A-6H show the expression level of each fetal-specific and maternal-specific marker identified in maternal cells (B cells, Erythroblasts Macrophages, Megakaryocytes, Neutrophils, T cells) and fetal cells (Stromal cells and Trophoblasts).

### Example 2: In-Office Prenatal Diagnosis Testing

A pregnant patient in her first trimester is deemed to be at risk for her fetus to have a birth defect or the pregnant patient is deemed to be at risk for a miscarriage. A blood sample is taken from the woman to be tested. The diagnostic testing is performed either onsite at the doctor's office/hospital or offsite at a diagnostic laboratory service. The blood sample is processed to enrich for nucleated cells and remove erythrocytes. The enriched nucleated cells are stained with antibodies and/or oligonucleotide-based marker detection reagents against H19, Tfpi, Hspb1, Sparc, Procr, Sct, Sbsn, Peg10, Cdkn1c, Cdh5, Ptprc, CD53, Cyba, Coro1a, and Itga2b. The stained cells are then sorted using FACS or MACS to isolate (H19, Tfpi, Hspb1, Sparc, Procr)-positive, (Sct, Sbsn, Peg10, Cdkn1c, Cdh5)-positive, and (Ptprc, CD53, Cyba, Coro1a, and Itga2b)-negative cells. The resulting isolated cells are viable circulating fetal cells (CFCs) isolated from the maternal blood. The isolated CFCs are subjected to either single-cell sequencing or bulk cell sequencing to analyze the fetal genome. The resulting fetal genome sequences are analyzed for any genetic defect. The results of the genetic analysis are then reported back to the patient and the designated attending physician.

### Example 3: At-Home Prenatal Diagnosis Testing

A pregnant woman in her first trimester is concerned that her fetus may have a birth defect or the pregnant woman is concerned that she may be at risk for a miscarriage. She orders an at-home prenatal diagnosis testing kit. She collects blood via a finger stick into a provided vial, and then sends the sample to a diagnostic laboratory service. The blood sample is processed to enrich for nucleated cells and remove erythrocytes. The enriched nucleated cells are stained with antibodies and/or oligonucleotide-based marker detection reagents against H19, Tfpi, Hspb1, Sparc, Procr, Sct, Sbsn, Peg10, Cdkn1c, Cdh5, Ptprc, CD53, Cyba, Coro1a, and Itga2b. The stained cells are then sorted using FACS or MACS to isolate (H19, Tfpi, Hspb1, Sparc, Procr)-positive, (Sct, Sbsn, Peg10, Cdkn1c, Cdh5)-positive, and (Ptprc, CD53, Cyba, Coro1a, and Itga2b)-negative cells. The resulting isolated cells are viable circulating fetal cells (CFCs) isolated from the maternal blood. The isolated CFCs are subjected to either single-cell sequencing or bulk cell sequencing to analyze the fetal genome. The resulting fetal genome sequences are analyzed for any genetic defect. The results of the genetic analysis are then reported back to the woman and shared with her designated attending physician if she requests.

### Example 4: Identification of Biomarkers and Biomarker Combinations for Enrichment of CFCs

To determine biomarker combinations that deliver high CFC yield and purity, CFC detection properties were characterized across millions of distinct multi-biomarker combinations that were created to comprise between 2 to 4 randomly selected individual biomarkers from the present disclosure. To increase the efficiency of this random search for biomarker combinations, the individual biomarker list was augmented by seeding with 4,000 2-4 biomarker combinations that had been preselected as performing well in CFC identification. CFC yield and purity was then measured for all random pairings of this starting set of biomarkers and biomarker combinations, and the resulting 11 million combinations were ranked by CFC capture performance across 5 different subgroupings of CFC cell types and in terms of percent of maximum CFC yield and percent of CFC purity for each of the CFC cell types detected. To rank the CFC detection performance of each biomarker combination, the expression of the biomarker combination was characterized across all of the 48,990 single cells in an experimental CFC-enriched single-cell RNA-seq library (e.g., SCVI v6 cluster 118 cell cluster), and the relative biomarker expression in the fetal and maternal cell types in this library was then used to calculate percent yield and percent purity delivered by each combination of biomarkers as detailed below.

### Method

Fifty 2D Matrix data files were prepared reporting on the correlation of fetal biomarker expression and maternal biomarker expression with gene expression, expression similarity clustering, and cell type classification data on all cells within the SCVI v6 cluster 118 cell cluster to characterize the fetal cell type and maternal cell type specificity and yield of each biomarker combination. These 50 files were then re-integrated into a single master table containing all 11,763,110 unique combinations of markers using Microsoft Power Pivot and Power Query for Excel. Each row in the master table indicated the fetal cell selection performance exhibited by a particular random pairwise combination of biomarkers, [Combination1] and [Combination2]. The master table included the following columns:
[Combination1]: preselected CFC marker gene combinations with 1-4 biomarker(s).
[Combination2]: preselected CFC marker gene combinations with 1-4 biomarker(s).
[Stromal cell marker-positive cell count, cluster 118]: number of cells in the SCVI v6 cluster 118 cell cluster that expressed stromal cell-specific biomarkers and biomarkers in the assigned pairwise combination, [Combination1] and [Combination2].
[Trophoblast marker-positive cell count, cluster 118]: number of cells in the SCVI v6 cluster 118 cell cluster that expressed trophoblast-specific biomarkers and biomarkers in the assigned pairwise combination, [Combination1] and [Combination2].
[NOT Stromal, NOT-Trophoblast, fetal genetic identity biomarker-positive cell count, cluster 118]: number of cells in the SCVI v6 cluster 118 cell cluster that did not express either stromal cell-nor trophoblast-specific biomarkers but expressed fetal cell genetic tag (EGFP transgene and/or Refsite (c57BL/6 reference SNP allele as explained in Example X)) and markers in the assigned pairwise combination, [Combination1] and [Combination2].
[All CFC biomarker-positive cell count, cluster 118]: number of cells in the SCVI v6 cluster 118 cell cluster that expressed All CFC biomarkers and biomarkers in the assigned pairwise combination, [Combination1] and [Combination2]. These values were the sum of [Stromal cell biomarker-positive cell count, cluster 118], [Trophoblast biomarker-positive cell count, cluster 118] and [NOT Stromal, NOT-Trophoblast, fetal genetic identity biomarker-positive cell count, cluster 118] of each assigned pairwise combination.
[Cluster 118 biomarker-positive cell count (biomarker-positive fetal cells)]: number of cells in the SCVI v6 cluster 118 cell cluster that expressed biomarkers in the assigned pairwise combination, [Combination1] and [Combination2].
[Not cluster 118 biomarker-positive cell count (biomarker-positive maternal cells)]: number of cells OUTSIDE the SCVI v6 cluster 118 cell cluster that expressed biomarkers in the assigned pairwise combination, [Combination1] and [Combination2]. These cells were primarily maternal cells.
[Early gestation trophoblast biomarker-positive cell count, cluster 118]: number of cells of 11-, 12-, and 13-gestation day libraries in the SCVI v6 cluster 118 cell cluster that expressed trophoblast-specific biomarkers and biomarkers in the assigned pairwise combination, [Combination1] and [Combination2]. These early trophoblast counts were corrected to exclude any stromal cells that might have been detected by each biomarker combination (e.g., corrected early gestation trophoblast biomarker-positive cell count=[#“Early gestation trophoblast biomarker-positive cell count, cluster 118”]−[#“Stromal cell biomarker-positive cell count, cluster 118”])).
[Late gestation trophoblast biomarker-positive cell count, cluster 118]: number of cells of 14-, 15-, 16-, 17-, and 19-gestation day libraries in the SCVI v6 cluster 118 cell cluster that expressed trophoblast-specific biomarkers and biomarkers in the assigned pairwise combination, [Combination1] and [Combination2].
[Number of markers in Combination1]: number of CFC biomarker genes in [Combination1].
[Number of markers in Combination2]: number of CFC biomarker genes in [Combination2]

Percent CFC yield and percent CFC purity were calculated using the formulas described below, with formulas configured to measure the purity of each fetal cell type detected by a given biomarker set relative to other fetal or maternal cell types detected by the same biomarker set.

| | |
|---|---|
| 1. | [% yield_All CFC] |
| | = ([#“All CFC biomarker-positive cell count, cluster 118”]/133)*100 |
| 2. | [% yield_Trophoblast] |
| | = ([#“Trophoblast biomarker-positive cell count, cluster 118”]/102)*100 |
| 3. | [% yield_Early Troph] |
| | = ([#“Early gestation trophoblast biomarker-positive cell count, cluster |
| | 118”]/57)*100 |
| 4. | [% yield_Late Troph] |
| | = ([#“Late gestation trophoblast biomarker-positive cell count, cluster |
| | 118”]/45)*100 |
| 5. | [% yield_Stromal Cell] |
| | = ([#“Stromal cell biomarker-positive cell count, cluster 118”]/10)*100 |
| 6. | [% yield_NOT-Str, NOT-Tro] |
| | = ([#“NOT Stromal, NOT-Trophoblast, fetal genetic identity biomarker-positive |
| | cell count, cluster 118”]/21)*100 |
| 7. | [% purity_All CFC] |
| | = if ([#“All CFC biomarker-positive cell count, cluster 118”]+[#“Not cluster 118 |
| | biomarker-positive cell count (biomarker-positive maternal cells)”])=0 then “0” |
| | else [#“All CFC biomarker-positive cell count, cluster 118”]/([#“All CFC |
| | biomarker-positive cell count, cluster 118”]+[#“Not cluster 118 biomarker- |
| | positive cell count (biomarker-positive maternal cells)”])*100 |
| 8. | [% purity_Trophoblast] |
| | = if ([#“Trophoblast biomarker-positive cell count, cluster 118”]+[#“Stromal cell |
| | biomarker-positive cell count, cluster 118”]+[#“NOT Stromal, NOT-Trophoblast, |
| | fetal genetic identity biomarker-positive cell count, cluster 118”]+[#“Not cluster |
| | 118 biomarker-positive cell count (biomarker-positive maternal cells)”])=0 then |
| | “0” else [#“Trophoblast biomarker-positive cell count, cluster |
| | 118”]/([#“Trophoblast biomarker-positive cell count, cluster 118”]+[#“Stromal |
| | cell biomarker-positive cell count, cluster 118”]+[#“NOT Stromal, NOT- |
| | Trophoblast, fetal genetic identity biomarker-positive cell count, cluster |
| | 118”]+[#“Not cluster 118 biomarker-positive cell count (biomarker-positive |
| | maternal cells)”])*100 |
| 9. | [% purity_Early Troph] |
| | = if ([#“Early gestation trophoblast biomarker-positive cell count, cluster |
| | 118”]+[#“Late gestation trophoblast biomarker-positive cell count, cluster |
| | 118”]+[#“Stromal cell biomarker-positive cell count, cluster 118”]+[#“NOT |
| | Stromal, NOT-Trophoblast, fetal genetic identity biomarker-positive cell count, |
| | cluster 118”]+[#“Not cluster 118 biomarker-positive cell count (biomarker- |
| | positive maternal cells)”])=0 then “0” else [#“Early gestation trophoblast |
| | biomarker-positive cell count, cluster 118”]/([#“Early gestation trophoblast |
| | biomarker-positive cell count, cluster 118”]+[#“Late gestation trophoblast |
| | biomarker-positive cell count, cluster 118”]+[#“Stromal cell biomarker-positive |
| | cell count, cluster 118”]+[#“NOT Stromal, NOT-Trophoblast, fetal genetic |
| | identity biomarker-positive cell count, cluster 118”]+[#“Not cluster 118 |
| | biomarker-positive cell count (biomarker-positive maternal cells)”])*100 |
| 10. | [% purity_Late Troph] |
| | = if ([#“Late gestation trophoblast biomarker-positive cell count, cluster |
| | 118”]+[#“Early gestation trophoblast biomarker-positive cell count, cluster |
| | 118”]+[#“Stromal cell biomarker-positive cell count, cluster 118”]+[#“NOT |
| | Stromal, NOT-Trophoblast, fetal genetic identity biomarker-positive cell count, |
| | cluster 118”]+[#“Not cluster 118 biomarker-positive cell count (biomarker- |
| | positive maternal cells)”])=0 then “0” else [#“Late gestation trophoblast |
| | biomarker-positive cell count, cluster 118”]/([#“Late gestation trophoblast |
| | biomarker-positive cell count, cluster 118”]+[#“Early gestation trophoblast |
| | biomarker-positive cell count, cluster 118”]+[#“Stromal cell biomarker-positive |
| | cell count, cluster 118”]+[#“NOT Stromal, NOT-Trophoblast, fetal genetic |
| | identity biomarker-positive cell count, cluster 118”]+[#“Not cluster 118 |
| | biomarker-positive cell count (biomarker-positive maternal cells)”])*100 |
| 11. | [% purity_Stromal Cell] |
| | = if ([#“Stromal cell biomarker-positive cell count, cluster 118”]+[#“Trophoblast |
| | biomarker-positive cell count, cluster 118”]+[#“NOT Stromal, NOT-Trophoblast, |
| | fetal genetic identity biomarker-positive cell count, cluster 118”]+[#“Not cluster |
| | 118 biomarker-positive cell count (biomarker-positive maternal cells)”])=0 then |
| | “0” else [#“Stromal cell biomarker-positive cell count, cluster 118”]/([#“Stromal |
| | cell biomarker-positive cell count, cluster 118”]+[#“Trophoblast biomarker- |
| | positive cell count, cluster 118”]+[#“NOT Stromal, NOT-Trophoblast, fetal |
| | genetic identity biomarker-positive cell count, cluster 118”]+[#“Not cluster 118 |
| | biomarker-positive cell count (biomarker-positive maternal cells)”])*100 |
| 12. | [% purity_NOT-Str, NOT-Tro] |
| | = if ([#“NOT Stromal, NOT-Trophoblast, fetal genetic identity biomarker- |
| | positive cell count, cluster 118”]+[#“Stromal cell biomarker-positive cell count, |
| | cluster 118”]+[#“Trophoblast biomarker-positive cell count, cluster 118”]+[#“Not |
| | cluster 118 biomarker-positive cell count (biomarker-positive maternal |
| | cells)”])=0 then “0” else [#“NOT Stromal, NOT-Trophoblast, fetal genetic |
| | identity biomarker-positive cell count, cluster 118”]/([#“NOT Stromal, NOT- |
| | Trophoblast, fetal genetic identity biomarker-positive cell count, cluster |
| | 118”]+[#“Stromal cell biomarker-positive cell count, cluster |
| | 118”]+[#“Trophoblast biomarker-positive cell count, cluster 118”]+[#“Not cluster |
| | 118 biomarker-positive cell count (biomarker-positive maternal cells)”])*100 |

After calculating the percent CFC yield and percent CFC purity, the master table was then filtered using cell-type specific sets of parameters shown below. These filters had the effect of excluding particularly noisy marker sets from consideration, and this filtration reduced the number of markers and marker combinations listed in the different categories.

### All CFC

[Not cluster 118 biomarker-positive cell count (biomarker-positive maternal cells)]: <=55

### Trophoblast

[Not cluster 118 biomarker-positive cell count (biomarker-positive maternal cells)]: <=51
[Stromal cell biomarker-positive cell count, cluster 118]: <=8

### Early Trophoblast

[Not cluster 118 biomarker-positive cell count (biomarker-positive maternal cells)]: <=29
[Stromal cell biomarker-positive cell count, cluster 118]: <=8
[Late gestation trophoblast biomarker-positive cell count, cluster 118]: <=8

### Late Trophoblast

[Not cluster 118 biomarker-positive cell count (biomarker-positive maternal cells)]: <=23
[Stromal cell biomarker-positive cell count, cluster 118]: <=2
[Early gestation trophoblast biomarker-positive cell count, cluster 118]: <=16

### Stromal Cell

[Not cluster 118 biomarker-positive cell count (biomarker-positive maternal cells)]: <=4
[Trophoblast biomarker-positive cell count, cluster 118]: <=2
[Stromal cell biomarker-positive cell count, cluster 118]: >=4

The effect of fetal cell-type specific filtering is summarized in Table 6 below.

TABLE 6
Cell-type specific filtering of cells in the SCVI v6 cluster 118 cell cluster following
purity and yield calculations.
Cell-type specific filtering

| | | | | |
|---|---|---|---|---|
| | Number of | | | |
| | cell-type | | | Number of |
| Number of | specific- | % of total | | % purity - |
| total pairwise | filtered | pairwise | | filtered |
| combinations | combinations | combinations | % purity filter | combinations |

All CFC

| | | | | | |
|---|---|---|---|---|---|
| Pos X Pos | 11763110 | 10926045 | 92.88 | 90 <= % purity <= 100 | 7351313 |
| | | | | 70 <= % purity < 90 | 3268282 |
| | | | | 50 <= % purity < 70 | 233945 |
| | | | | 30 <= % purity < 50 | 50013 |
| | | | | 0 <= % purity < 30 | 22492 |
| Neg X Pos X | 11763110 | 11761564 | 99.99 | 90 <= % purity <= 100 | 11667366 |
| Pos | | | | | |
| | | | | 70 <= % purity < 90 | 76220 |
| | | | | 50 <= % purity < 70 | 5692 |
| | | | | 30 <= % purity < 50 | 1902 |
| | | | | 0 <= % purity < 30 | 10384 |

Trophoblast

| | | | | | |
|---|---|---|---|---|---|
| Pos X Pos | 11763110 | 10823011 | 92.01 | 90 <= % purity <= 100 | 4888722 |
| | | | | 70 <= % purity < 90 | 5049329 |
| | | | | 50 <= % purity < 70 | 697120 |
| | | | | 30 <= % purity < 50 | 125772 |
| | | | | 0 <= % purity < 30 | 62068 |
| Neg X Pos X | 11763110 | 11644133 | 98.99 | 90 <= % purity <= 100 | 9409503 |
| Pos | | | | | |
| | | | | 70 <= % purity <90 | 1969513 |
| | | | | 50 <= % purity < 70 | 176991 |
| | | | | 30 <= % purity < 50 | 55212 |
| | | | | 0 <= % purity < 30 | 32914 |

Early Trophoblast

| | | | | | |
|---|---|---|---|---|---|
| Pos X Pos | 11763110 | 1063863 | 9.04 | 90 <= % purity <= 100 | 71099 |
| | | | | 70 <= % purity < 90 | 345185 |
| | | | | 50 <= % purity < 70 | 375032 |
| | | | | 30 <= % purity < 50 | 189566 |
| | | | | 0 <= % purity < 30 | 82981 |
| Neg X Pos X | 11763110 | 1112878 | 9.46 | 90 <= % purity <= 100 | 95718 |
| Pos | | | | | |
| | | | | 70 <= % purity < 90 | 399576 |
| | | | | 50 <= % purity < 70 | 443641 |
| | | | | 30 <= % purity < 50 | 128603 |
| | | | | 0 <= % purity < 30 | 45340 |

Late Trophoblast

| | | | | | |
|---|---|---|---|---|---|
| Pos X Pos | 11763110 | 520120 | 4.42 | 90 <= % purity <= 100 | 805 |
| | | | | 70 <= % purity < 90 | 19905 |
| | | | | 50 <= % purity < 70 | 102522 |
| | | | | 30 <= % purity <50 | 121787 |
| | | | | 0 <= % purity < 30 | 275101 |
| Neg X Pos X | 11763110 | 596159 | 5.07 | 90 <= % purity <= 100 | 1052 |
| Pos | | | | | |
| | | | | 70 <= % purity < 90 | 24221 |
| | | | | 50 <= % purity < 70 | 131971 |
| | | | | 30 <= % purity < 50 | 182347 |
| | | | | 0 <= % purity < 30 | 256568 |

Stromal cell

| | | | | | |
|---|---|---|---|---|---|
| Pos X Pos | 11763110 | 20984 | 0.18 | 90 <= % purity <= 100 | 14 |
| | | | | 70 <= % purity < 90 | 8135 |
| | | | | 50 <= % purity < 70 | 12722 |
| | | | | 30 <= % purity <50 | 113 |
| | | | | 0 <= % purity < 30 | 0 |
| Neg X Pos X | 11763110 | 339974 | 2.89 | 90 <= % purity <= 100 | 69110 |
| Pos | | | | | |
| | | | | 70 <= % purity < 90 | 260736 |
| | | | | 50 <= % purity < 70 | 10128 |
| | | | | 30 <= % purity < 50 | 0 |
| | | | | 0 <= % purity < 30 | 0 |

The filtered tables for each CFC cell type were sorted by percent CFC cell type purity and then subdivided according to the percent CFC purity criteria. Finally, each subdivided table for each CFC cell type was sorted according to its percent CFC yield.

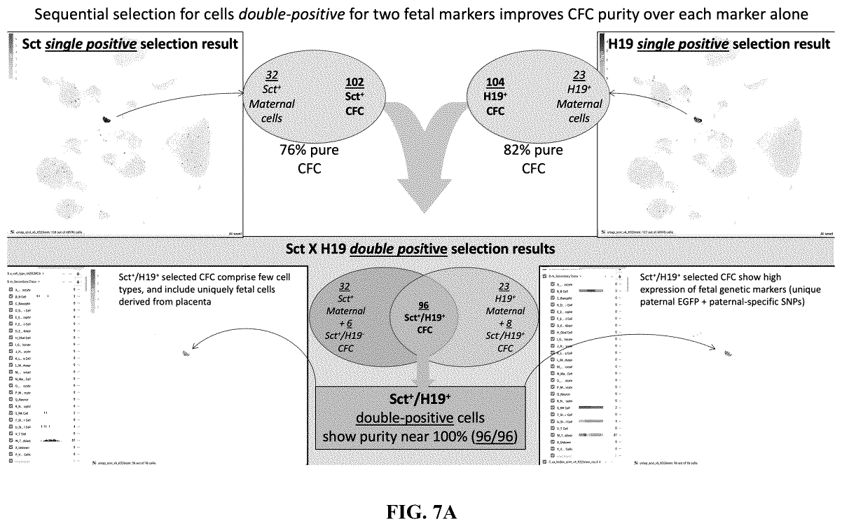

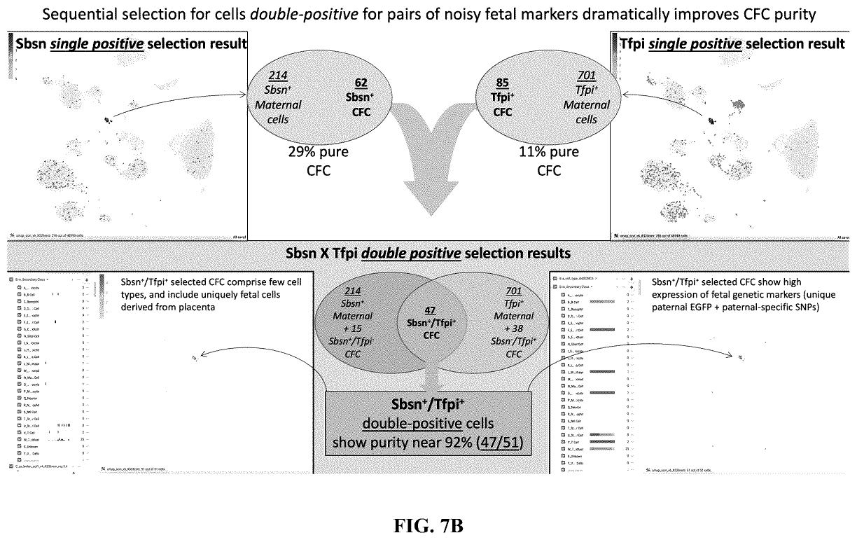

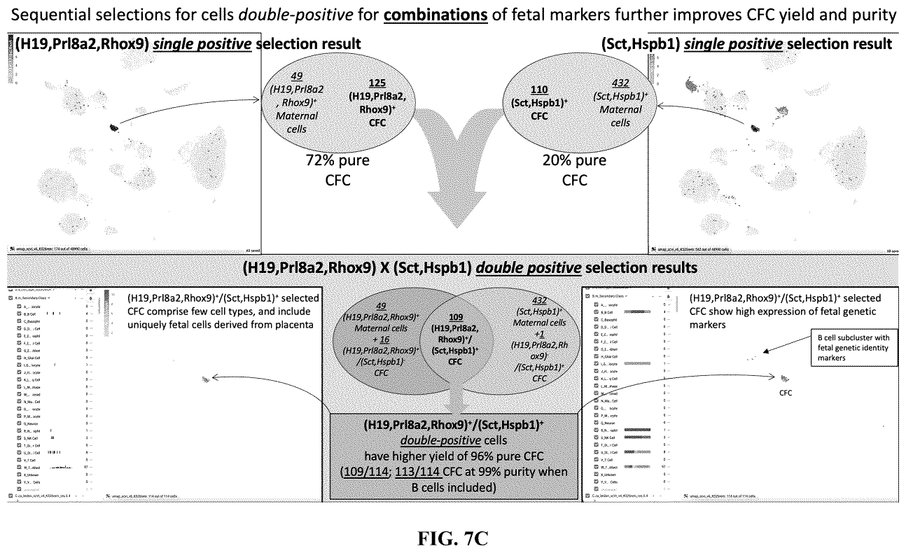

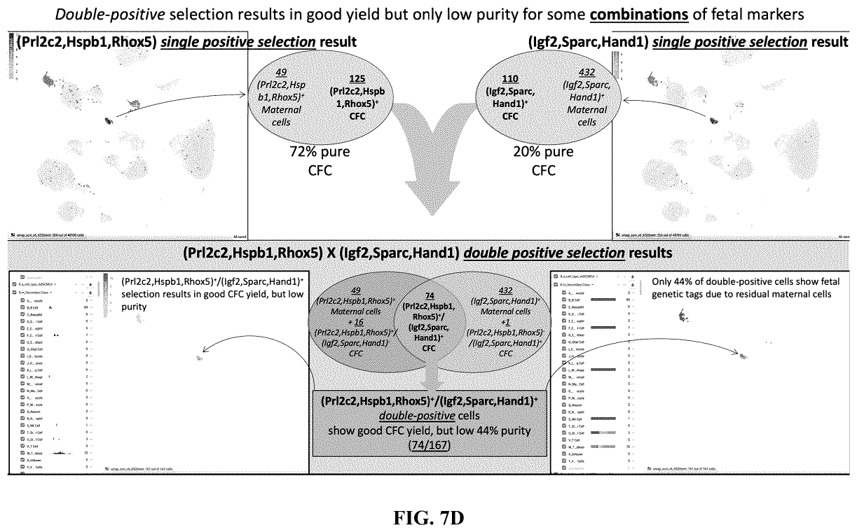

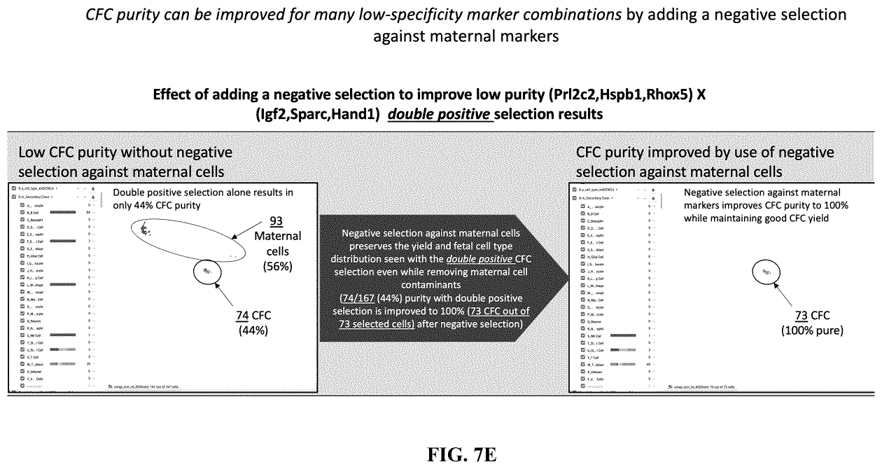

To determine ways to improve biomarker performance in identifying and purifying CFCs, the identified fetal biomarkers and maternal biomarkers were tested individually and in different combinations to check for fetal cell selection strategies that were effective in increasing CFC population yield and purity. FIGS. 7A and 7B illustrate how the specificity of CFC identification improved by selecting for cells double positive for two fetal biomarkers over the specificity achieved by use of either fetal biomarker alone. FIG. 7A illustrates that even highly specific individual fetal biomarkers improved CFC capture performance when used to find double-positive fetal cells as opposed to the use of the two fetal biomarkers (e.g., Sct and H19) separately to find cells expressing either one or the other of the individual fetal biomarkers Specifically, the use of two fetal biomarkers together (e.g., Sct and H19) resulted in the isolation of a population of cells that had near 100% purity of fetal cells. The Sct+H19+ cell population included a few cell types, including fetal cells derived from the placenta, and the cell population shoed high expression of fetal genetic markers. FIG. 7B illustrates that selective pairing of even noisy fetal biomarkers (e.g., biomarkers that detect larger numbers of maternal cells in addition to their intended fetal cell targets) identified high purity CFC that were double-positive when both fetal biomarkers only delivered low purity CFC populations when used individually. Specifically, the use of two fetal biomarkers together (e.g., Sbsn and Tfpi) resulted in the isolation of a population of cells that had a 92% purity of fetal cells. The Sbsn+Tfpi+ cell population included a few cell types, including fetal cells derived from the placenta, and the cell population shoed high expression of fetal genetic markers.

An illustration of the value of improved CFC yield to minimize false negative detection failures through the use of biomarker combinations and use of these biomarker combination to broaden CFC detection to include additional CFC cell types is shown in FIG. 7C. Specifically, the use of multiple fetal biomarkers together (e.g., H19, Prl8a2, Rhox9, Sct, Hspb1) resulted in the isolation of a population of cells that had a 99% purity of fetal cells. The H19+Prl8a2+Rhox9+Sct+Hspb+ cell population included a few cell types, including fetal cells derived from the placenta, and the cell population shoed high expression of fetal genetic markers. In FIG. 7C, CFC yield was increased by about 18% with the use of two different fetal biomarker combinations to purify CFCs compared to the purity of CFCs achieved by the use of the two single fetal biomarkers used in FIG. 7A. In both enrichment schemes shown in FIG. 7A and FIG. 7C, CFC purity was near 100%. This result demonstrated that biomarker combinations can outperform single biomarkers at comparable CFC purity levels. The two biomarker combinations used in FIG. 7C each had higher intrinsic maternal cell noise when used individually, but the sequential use of both combinations to define the fetal cells double-positive for both fetal biomarker combinations was effective in silencing the maternal noise.

The extra value in including a negative selection step to improve the double-positive CFC enrichment protocol utilizing noisy biomarker combinations is illustrated in FIGS. 7D and 7E. Individually noisy biomarker combinations had overlapping maternal expression that was not silenced by simply selecting for double-positive cells, as indicated by the low 44% purity of double-positive CFC shown in FIG. 7D. Specifically, the user of the combination of fetal biomarkers Prl2c2, Hspb1, Rhox5, Igf2, Sparc, and Hand1 in a double positive selection scheme resulted in a good yield of CFCs, however this cell population only had a 44% purity of fetal cells. However, this low purity was improved to 100% purity while maintaining CFC yield by including an extra negative selection step against maternal cell biomarker expression as shown in FIG. 7E, which removed the contaminating maternal cells from the double positive selection scheme shown in FIG. 7D.

Data on different biomarker combinations were calculated that ranked the performance of biomarker combinations in delivering fetal cell yield and purity. Separate tables were generated for All CFC (general fetal cell capture), trophoblast cells, early first trimester gestation trophoblasts, late first trimester gestation trophoblasts, and fetal stromal cells (also most abundant in early first trimester gestation).

These tables were organized according to the sequence of positive and negative selection steps used for fetal cell identification and purification. Selection strategies used are as follows:
1. Pos: using selection with a single positive selection with fetal biomarkers or fetal biomarker combinations (to yield only cells that express fetal biomarkers or fetal biomarker combinations)
2. Pos+Neg: selection with a preliminary negative selection with maternal biomarkers or biomarker combinations prior to positive selection with fetal biomarkers or biomarker combinations (to yield cells that expressed the fetal specific biomarkers or biomarker combination, while also lacked expression of the maternal biomarkers or biomarker combinations)
3. Pos+Pos: selection with two sequential positive selections with different fetal biomarkers or biomarker combinations (to yield only cells that expressed both fetal specific biomarkers or biomarker combinations)
4. Pos+Pos+Neg: selection with a preliminary negative selection with maternal biomarkers or biomarker combinations prior to two sequential positive selections with different fetal biomarkers or biomarker combinations (to yield only cells that expressed both fetal-specific biomarkers or biomarker combinations, while also lacked expression of the maternal biomarkers or biomarker combinations)

The fetal cell selection performance of pairwise combinations of biomarkers and biomarker combinations was ranked by highest-measured fetal cell yield and filtered for those combinations that simultaneously exhibited minimal contamination by maternal cells or other fetal cell types. Data on biomarkers targeting different CFC cell type groups were included in separate cell type-specific tables. The fetal cell identity of all these fetal cell type groupings were confirmed by the detection of fetal-specific genetic tags in most of the individual cells within each cell type group. Below includes a list of the different CFC cell-type cells examined.
All-CFC: This was a broad fetal cell target that used a wide biomarker net to capture as many fetal cells of all fetal cell types as is possible. The mix and relative proportions of fetal cell types captured varied with the biomarker combinations used.
Trophoblasts: These circulating fetal cells included multiple trophoblast variants, with the mix of trophoblast variants changing over gestation and characterized by distinct biomarker expression profiles. In particular, the biomarker profiles of early and late gestation trophoblasts were related but were sufficiently distinct to separately identify these trophoblast subtypes. Biomarker combinations in this trophoblast category were formulated to capture the largest number of trophoblast variants irrespective of trophoblast subtype.
Early Trophoblasts: A distinct subset of trophoblasts peaked in abundance from 11-13 days post-fertilization during mouse gestation. This early gestation time frame corresponded to 11-13 days after fertilization in mouse and mapped to 30-42 days after fertilization in human (6-8 weeks human gestational age). Early trophoblasts had many specific biomarkers that were unique to these early trophoblast variants. The biomarkers allowed specific detection of high purity Early Trophoblast populations with appropriate selection of biomarker combinations.
Late Trophoblast: Biomarker expression specific to late Trophoblasts was broadly detected beginning at 13 days after fertilization and predominated from 14 days onward. This late gestation time frame corresponded to 14-19 days after fertilization in mouse and mapped to 48-76 days after fertilization in human (9-13 weeks human gestational age).
Stromal Cells: Stromal cells were detected in CFCs at 11-13 days after fertilization.

The mapping of the developmental age of the mouse maternal blood samples to the corresponding age of human samples is shown in Table 7, below. Human development follows a similar path to that of mouse, although at a slower pace to reflect the much longer duration of human gestation. Note that all mouse samples were collected to cover a small range of ages corresponding to fertilization over a three-day window such that, for example, the sample called “11-day” could contain cells anywhere from 9-days to 11-days post-fertilization. Additionally, Table 8 below shows a subset of the different biomarkers and biomarker combinations that detected fetal cell populations in this Example.

TABLE 7
Mapping of mouse to human post-fertilization and gestational age.

| | Mouse fertilization | | Human fertilization | | | |
|---|---|---|---|---|---|---|
| | age range | | age range | | Human gestational | |
| | earliest | latest | earliest | latest | age range | |
| | mouse | mouse | human | human | earliest | latest |
| | fertilization | fertilization | fertilization | fertilization | human | human |
| | age (days | age (days | age (days | age (days | gestational | gestational |
| Mouse sample | after | after | after | after | age | age |
| name | fertilization) | fertilization) | fertilization) | fertilization) | (weeks) | (weeks) |
| 11-day sample | 9 | 11 | 20 | 30 | 4.9 | 6.3 |
| 12-day sample | 10 | 12 | 24 | 36 | 5.4 | 7.1 |
| 13-day sample | 11 | 13 | 30 | 42 | 6.3 | 8.0 |
| 14-day sample | 12 | 14 | 36 | 48 | 7.1 | 8.9 |
| 15-day sample | 13 | | 42 | 54 | 8.0 | 9.7 |
| 16-day sample | 14 | 16 | 48 | 58 | 8.9 | 10.3 |
| 17-day sample | 15 | 17 | 54 | 64 | 9.7 | 11.1 |
| 19-day sample | 17 | 19 | 64 | 76 | 11.1 | 12.9 |

TABLE 8
Biomarkers and Biomarker Combinations for the isolation of CFCs.
Biomarker Combinations

| Biomarker | Biomarker | Biomarker | Biomarker | Biomarker | Biomarker |
|---|---|---|---|---|---|
| Set 1 | Set 2 | Set 1 | Set 2 | Set 1 | Set 2 |
| Gpx3 + Dmkn + | H19 + Krt19 + | Gm26870 | Fnd3c2 + Hand1 | H19 + Prl7d1 + | Map1b + Prl2c5 + |
| Prl8a2 | Prl8a2 | | | Rhox9 | Rhox9 |
| Mt1 + Gpx3 + | H19 + Krt19 + | Gm26870 | Fnd3c2 + Rhox5 | H19 + Krt8 + | Map1b + Prl2c5 + |
| Dmkn | Prl8a2 | | | Rhox9 | Rhox9 |
| 4930486L24Rik + | H19 + Krt19 + | Gm26870 | Fermt2 + Rhox5 | H19 + Pawr + | Map1b + Prl2c5 + |
| Gpx3 + Dmkn | Prl8a2 | | | Rhox9 | Rhox9 |
| Ly6c1 + Mt2 + | H19 + Krt19 + | Gm26870 | Fnd3c2 + Prl2c2 | Dusp9 + H19 + | Map1b + Prl2c5 + |
| Dmkn | Prl8a2 | | | Rhox9 | Rhox9 |
| Rrm2 + Gpx3 + | H19 + Krt19 + | Cts6 | Prl7a1 | H19 + Prl3d1 + | Map1b + Prl2c5 + |
| Dmkn | Prl8a2 | | | Rhox9 | Rhox9 |
| Gpx3 + Dmkn + | Krt19 + Prl8a2 + | Zfp36l3 | Prl2b1 | Map1b + Prl2c5 + | H19 + Rhox9 + |
| Prl8a2 | Sct | | | Rhox9 | Sult1e1 |
| Mt1 + Mt2 + | Krt19 + Prl8a2 + | Cts6 | Hand1 | Elf3 + H19 + | Map1b + Prl2c5 + |
| Dmkn | Sct | | | Rhox9 | Rhox9 |
| 4930486L24Rik + | Krt19 + Prl8a2 + | Prnp | Ctsq | Krt18 + Peg10 + | Map1b + Prl2c5 + |
| Gpx3 + Dmkn | Sct | | | Sct | Rhox9 |
| Ly6c1 + Mt2 + | Krt19 + Prl8a2 + | Paxbp1 | Tinagl1 | H19 + Krt8 + | Map1b + Prl2c5 + |
| Dmkn | Sct | | | Peg10 | Rhox9 |
| Rrm2 + Gpx3 + | Krt19 + Prl8a2 + | Paxbp1 | Lepr | H19 + Peg10 + | Map1b + Prl2c5 + |
| Dmkn | Sct | | | Rhox9 | Rhox9 |
| Gpx3 + Dmkn + | Cryab + H19 + | Prpf4b | Syngr1 | H19 + Rhox9 + | Map1b + Prl2c5 + |
| Prl8a2 | Krt19 | | | Tnfrsf9 | Rhox9 |
| Mt1 + Gpx3 + | Cryab + H19 + | Ctsr + Zfp36l3 | Hspg2 | H19 + Krt18 + | Map1b + Prl2c5 + |
| Dmkn | Krt19 | | | Rhox9 | Rhox9 |
| 4930486L24Rik + | Cryab + H19 + | Dst + Map1b + | Lamb2 | Cited1 + Krt18 + | Map1b + Prl2c5 + |
| Gpx3 + Dmkn | Krt19 | Rere | | Sct | Rhox9 |
| Ly6c1 + Mt2 + | Cryab + H19 + | Dst + Rere | Lamb2 | Krt18 + Nrk + | Map1b + Prl2c5 + |
| Dmkn | Krt19 | | | Sct | Rhox9 |
| Rrm2 + Gpx3 + | Cryab + H19 + | Emilin1 + Rere | Lamb2 | Krt18 + Nup62cl + | Map1b + Prl2c5 + |
| Dmkn | Krt19 | | | Sct | Rhox9 |
| Gpx3 + Dmkn + | Cryab + Krt19 + | Map1b + Rere | Lamb2 | Krt18 + Sct + | Map1b + Prl2c5 + |
| Prl8a2 | Sct | | | Zfp36l3 | Rhox9 |
| Mt1 + Mt2 + | Cryab + Krt19 + | Trrap | Hand1 + Zfp36l3 | H19 + Oaf + | Map1b + Prl2c5 + |
| Dmkn | Sct | | | Rhox9 | Rhox9 |
| 4930486L24Rik + | Cryab + Krt19 + | Trrap | 1600025M17Rik + | H19 + Hsd17b2 + | Map1b + Prl2c5 + |
| Gpx3 + Dmkn | Sct | | Zfp36l3 | Rhox9 | Rhox9 |
| Ly6c1 + Mt2 + | Cryab + Krt19 + | Shroom1 | Ghrh | H19 + Phactr1 + | Map1b + Prl2c5 + |
| Dmkn | Sct | | | Rhox9 | Rhox9 |
| Rrm2 + Gpx3 + | Cryab + Krt19 + | Prnp | Ghrh | H19 + Rhox9 + | Map1b + Prl2c5 + |
| Dmkn | Sct | | | Serpine1 | Rhox9 |
| Gpx3 + Dmkn + | H19 + Krt18 + | Mllt4 | Ghrh | H19 + Krt8 + | Map1b + Prl2c5 + |
| Prl8a2 | Prl8a2 | | | Prl7d1 | Rhox9 |
| Mt1 + Gpx3 + | H19 + Krt18 + | Mt2 | Gm9112 | H19 + Krt8 + | Map1b + Prl2c5 + |
| Dmkn | Prl8a2 | | | Prl2c3 | Rhox9 |
| 4930486L24Rik + | H19 + Krt18 + | Mt2 + Des + | Gm9112 | H19 + Plet1 + | Map1b + Prl2c5 + |
| Gpx3 + Dmkn | Prl8a2 | Mfap5 | | Rhox9 | Rhox9 |
| Ly6c1 + Mt2 + | H19 + Krt18 + | Mt2 + Dmkn + | Gm9112 | H19 + Lama5 + | Map1b + Prl2c5 + |
| Dmkn | Prl8a2 | Prl8a2 | | Rhox9 | Rhox9 |
| Rrm2 + Gpx3 + | H19 + Krt18 + | Mt2 + Des | Gm9112 | H19 + Prl7a1 + | Map1b + Prl2c5 + |
| Dmkn | Prl8a2 | | | Rhox9 | Rhox9 |
| Gpx3 + Dmkn1 + | Krt18 + Prl8a2 + | Mt2 + Mfap5 | Gm9112 | H19 + Krt8 + | Map1b + Prl2c5 + |
| Prl8a2 | Sct | | | Trap1a | Rhox9 |
| Mt1 + Gpx3 + | Krt18 + Prl8a2 + | Mt2 + Dmkn | Gm9112 | Ceacam15 + H19 + | Map1b + Prl2c5 + |
| Dmkn | Sct | | | Rhox9 | Rhox9 |
| 4930486L24Rik + | Krt18 + Prl8a2 + | Mt2 + Prl8a2 | Gm9112 | Fthl17a + H19 + | Map1b + Prl2c5 + |
| Gpx3 + Dmkn | Sct | | | Sct | Rhox9 |
| Ly6c1 + Mt2 + | Krt18 + Prl8a2 + | 4930486L24Rik + | Gm9112 | H19 + Krt8 + | Map1b + Prl2c5 + |
| Dmkn | Sct | Mt2 + Des | | Prl2a1 | Rhox9 |
| Rrm2 + Gpx3 + | Krt18 + Prl8a2 + | 4930486L24Rik + | Gm9112 | H19 + Homer2 + | Map1b + Prl2c5 + |
| Dmkn | Sct | Mt2 + Mfap5 | | Rhox9 | Rhox9 |
| Ly6c1 + Mt2 + | Gpx3 + H19 + | 4930486L24Rik + | Gm9112 | Cts6 + H19 + | Map1b + Prl2c5 + |
| Dmkn | Krt19 | Mt2 + Dmkn | | Rhox9 | Rhox9 |
| Gpx3 + Dmkn + | H19 + Krt19 + | 4930486L24Rik + | Gm9112 | H19 + Krt8 + | Map1b + Prl2c5 + |
| Prl8a2 | Sbsn | Mt2 + Prl8a2 | | Serpinb9c | Rhox9 |
| Mt1 + Gpx3 + | H19 + Krt19 + | Ly6c1 + Mt2 + | Gm9112 | Map1b + Prl2c5 + | 3830417A13Rik + |
| Dmkn | Sbsn | Des | | Rhox9 | H19 + Rhox9 |
| 4930486L24Rik + | H19 + Krt19 + | Ly6c1 + Mt2 + | Gm9112 | Map1b + Prl2c5 + | H19 + Rhox9 + |
| Gpx3 + Dmkn | Sbsn | Mfap5 | | Rhox9 | Serpinb9f |
| Ly6c1 + Mt2 + | H19 + Krt19 + | Ly6c1 + Mt2 + | Gm9112 | Map1b + Prl2c5 + | H19 + Rhox9 + |
| Dmkn | Sbsn | Dmkn | | Rhox9 | Serpinb9c |
| Rrm2 + Gpx3 + | H19 + Krt19 + | Ly6c1 + Mt2 + | Gm9112 | Map1b + Prl2c5 + | H19 + Prl4a1 + |
| Dmkn | Sbsn | Prl8a2 | | Rhox9 | Rhox9 |
| Mt1 + Mt2 + | Gpx3 + Krt19 + | 1700011M02Rik | Serpinb9f | Map1b + Prl2c5 + | H19 + Ldoc1 + |
| Dmkn | Sct | | | Rhox9 | Rhox9 |
| Ly6c1 + Mt2 + | Gpx3 + Krt19 + | Prpf4b | 3830417A13Rik | Map1b + Prl2c5 + | Gm9112 + H19 + |
| Dmkn | Sct | | | Rhox9 | Rhox9 |
| Gpx3 + Dmkn + | Krt19 + Sbsn + | Prpf4b | Adgrf5 | Map1b + Prl2c5 + | H19 + Pr18a9 + |
| Prl8a2 | Sct | | | Rhox9 | Rhox9 |
| Mt1 + Mt2 + | Krt19 + Sbsn + | Zfp36l3 | Sult1e1 | Map1b + Prl2c5 + | H19 + Rhox9 + |
| Dmkn | Sct | | | Rhox9 | Serpinb9d |
| 4930486L24Rik + | Krt19 + Sbsn + | Ctsq | Pramef12 | Map1b + Prl2c5 + | H19 + Prl2c1 + |
| Gpx3 + Dmkn | Sct | | | Rhox9 | Rhox9 |
| Ly6c1 + Mt2 + | Krt19 + Sbsn + | Rarres2 | Pramef12 | Map1b + Prl2c5 + | H19 + Prl3d3 + |
| Dmkn | Sct | | | Rhox9 | Rhox9 |
| Rrm2 + Gpx3 + | Krt19 + Sbsn + | Ctsq + Rarres2 | Pramef12 | Map1b + Prl2c5 + | H19 + Krt19 + |
| Dmkn | Sct | | | Rhox9 | Rhox9 |
| Gpx3 + Dmkn + | Krt18 + Prl3b1 + | Zfp36l3 | Slc30a2 | Map1b + Prl2c5 + | H19 + Rhox12 + |
| Prl8a2 | Prl8a2 | | | Rhox9 | Rhox9 |
| Mt1 + Gpx3 + | Krt18 + Prl3b1 + | Lepr | Zfp36l3 | Map1b + Prl2c5 + | Ceacam9 + H19 + |
| Dmkn | Prl8a2 | | | Rhox9 | Rhox9 |
| 4930486L24Rik + | Krt18 + Prl3b1 + | Oaf | Zfp36l3 | Map1b + Prl2c5 + | H19 + Rhox5 + |
| Gpx3 + Dmkn | Prl8a2 | | | Rhox9 | Rhox9 |
| Ly6c1 + Mt2 + | Krt18 + Prl3b1 + | Cts6 + Prl2b1 | Zfp36l3 | Map1b + Prl2c5 + | H19 + Pfpl + |
| Dmkn | Prl8a2 | | | Rhox9 | Rhox9 |
| Rrm2 + Gpx3 + | Krt18 + Prl3b1 + | Ascc3 | Zfp36l3 | Map1b + Prl2c5 + | Fnd3c2 + H19 + |
| Dmkn | Prl8a2 | | | Rhox9 | Rhox9 |
| Gpx3 + Dmkn + | Ctsj + Krt18 + | Trrap | Zfp36l3 | Map1b + Prl2c5 + | H19 + Hand1 + |
| Prl8a2 | Prl8a2 | | | Rhox9 | Rhox9 |
| Mt1 + Gpx3 + | Ctsj + Krt18 + | Serpinb9g | Cts6 | Map1b + Prl2c5 + | 1600025M17Rik + |
| Dmkn | Prl8a2 | | | Rhox9 | H19 + Rhox9 |
| 4930486L24Rik + | Ctsj + Krt18 + | Zfp36l3 | Cts6 | Map1b + Prl2c5 + | H19 + Rhox9 + |
| Gpx3 + Dmkn | Prl8a2 | | | Rhox9 | Shroom1 |
| Ly6c1 + Mt2 + | Ctsj + Krt18 + | Cyp17a1 | Cts6 | Map1b + Prl2c5 + | H19 + Rhox9 + |
| Dmkn | Prl8a2 | | | Rhox9 | Serpinb9g |
| Rrm2 + Gpx3 + | Ctsj + Krt18 + | Car2 | Cts6 | Map1b + Prl2c5 + | Cyp17a1 + H19 + |
| Dmkn | Prl8a2 | | | Rhox9 | Rhox9 |
| Gpx3 + Dmkn + | Cryab + H19 + | Homer2 | Cts6 | Map1b + Prl2c5 + | H19 + Plac1 + |
| Prl8a2 | Krt18 | | | Rhox9 | Rhox9 |
| Mt1 + Gpx3 + | Cryab + H19 + | Oaf | Cts6 | Map1b + Prl2c5 + | H19 + Psg22 + |
| Dmkn | Krt18 | | | Rhox9 | Rhox9 |
| 4930486L24Rik + | Cryab + H19 + | Hand1 + Igf2bp1 | Cts6 | Map1b + Prl2c5 + | H19 + Prl8a1 + |
| Gpx3 + Dmkn | Krt18 | | | Rhox9 | Rhox9 |
| Ly6c1 + Mt2 + | Cryab + H19 + | Hand1 + Serpinb9d | Cts6 | Map1b + Prl2c5 + | H19 + Prl3d2 + |
| Dmkn | Krt18 | | | Rhox9 | Rhox9 |
| Rrm2 + Gpx3 + | Cryab + H19 + | Hand1 + Prl3d2 | Cts6 | Map1b + Prl2:5 + | H19 + Parva + |
| Dmkn | Krt18 | | | Rhox9 | Rhox9 |
| Gpx3 + Dmkn + | Cryab + Krt18 + | Hand1 + Pramef12 | Cts6 | Map1b + Prl2c5 + | H19 + Nrk + |
| Prl8a2 | Sct | | | Rhox9 | Rhox9 |
| Mt1 + Gpx3 + | Cryab + Krt18 + | Hand1 + Sult1e1 | Cts6 | Map1b + Prl2c5 + | H19 + Krt14 + |
| Dmkn | Sct | | | Rhox9 | Rhox9 |
| 4930486L24Rik + | Cryab + Krt18 + | Hand1 + Krt14 | Cts6 | Map1b + Prl2c5 + | H19 + Prl5a1 + |
| Gpx3 + Dmkn | Sct | | | Rhox9 | Rhox9 |
| Ly6c1 + Mt2 + | Cryab + Krt18 + | Hand1 | Cts6 | Map1b + Prl2c5 + | Cdh3 + H19 + |
| Dmkn | Sct | | | Rhox9 | Rhox9 |
| Rrm2 + Gpx3 + | Cryab + Krt18 + | Prl3d2 + Serpinb9g | Cts6 | Map1b + Prl2c5 + | H19 + Nppc + |
| Dmkn | Sct | | | Rhox9 | Rhox9 |
| Gpx3 + Dmkn + | Cryab + Krt18 + | Serpinb9d + Serpinb9g | Cts6 | Map1b + Prl2:5 + | H19 + Rhox9 + |
| Prl8a2 | Prl3b1 | | | Rhox9 | Trap1a |
| Mt1 + Gpx3 + | Cryab + Krt18 + | Krt14 + Serpinb9g | Cts6 | Map1b + Prl2c5 + | H19 + Prl2c2 + |
| Dmkn | Pr13b1 | | | Rhox9 | Rhox9 |
| 4930486L24Rik + | Cryab + Krt18 + | Hand1 + Prl2a1 | Cts6 | Map1b + Prl2c5 + | Fermt2 + H19 + |
| Gpx3 + Dmkn | Prl3b1 | | | Rhox9 | Rhox9 |
| Ly6c1 + Mt2 + | Cryab + Krt18 + | Prl3d2 + Prl7a1 | Cts6 | Map1b + Prl2c5 + | Fmr1nb + H19 + |
| Dmkn | Prl3b1 | | | Rhox9 | Rhox9 |
| Rrm2 + Gpx3 + | Cryab + Krt18 + | Prl7a1 + Prl8a1 | Cts6 | H19 + Hspg2 + | Map1b + Prl2c5 + |
| Dmkn | Pr13b1 | | | Rhox9 | Rhox9 |
| Gpx3 + Dmkn + | Cryab + Ctsj + | Prl7a1 + Psg29 | Cts6 | H19 + Rhox9 + | Map1b + Prl2c5 + |
| Prl8a2 | Krt18 | | | Tinagl1 | Rhox9 |
| Mt1 + Gpx3 + | Cryab + Ctsj + | Prl7a1 + Psg22 | Cts6 | H19 + Krt18 + | Map1b + Prl2c5 + |
| Dmkn | Krt18 | | | Sct | Rhox9 |
| 4930486L24Rik + | Cryab + Ctsj + | Prl2a1 + Prl7a1 | Cts6 | H19 + Rhox9 + | Map1b + Prl2c5 + |
| Gpx3 + Dmkn | Krt18 | | | Syngr1 | Rhox9 |
| Ly6c1 + Mt2 + | Cryab + Ctsj + | Prl4a1 + Prl7a1 | Cts6 | Map1b + Prl2c5 + | 1700011M02Rik + |
| Dmkn | Krt18 | | | Rhox9 | H19 + Rhox9 |
| Rrm2 + Gpx3 + | Cryab + Ctsj + | Prl3d1 + Prl4a1 | Cts6 | Map1b + Prl2c5 + | Adgrf5 + H19 + |
| Dmkn | Krt18 | | | Rhox9 | Rhox9 |
| Ly6c1 + Mt2 + | Gpx3 + H19 + | Cts7 + Prl3d1 | Cts6 | Map1b + Prl2c5 + | H19 + Krt8 + |
| Dmkn | Krt18 | | | Rhox9 | Serpinb9d |
| Gpx3 + Dmkn + | H19 + Krt18 + | Srrm2 | Cts6 | Cts7 + H19 + | Map1b + Prl2c5 + |
| Prl8a2 | Sbsn | | | Rhox9 | Rhox9 |
| Mt1 + Gpx3 + | H19 + Krt18 + | Dst + Plec | Cts6 | Dusp9 + H19 + | Map1b + Prl2c5 + |
| Dmkn | Sbsn | | | Krt8 | Rhox9 |
| 4930486L24Rik + | H19 + Krt18 + | Dst + Rere | Cts6 | Cyp11a1 + H19 + | Map1b + Prl2c5 + |
| Gpx3 + Dmkn | Sbsn | | | Rhox9 | Rhox9 |
| Ly6c1 + Mt2 + | H19 + Krt18 + | Pramef12 | 1700011M02Rik | H19 + Rhox6 + | Map1b + Prl2c5 + |
| Dmkn | Sbsn | | | Rhox9 | Rhox9 |
| Rrm2 + Gpx3 + | H19 + Krt18 + | Shroom1 | 1700011M02Rik | H19 + Igf2 + | Map1b + Prl2c5 + |
| Dmkn | Sbsn | | | Rhox9 | Rhox9 |
| Ly6c1 + Mt2 + | Gpx3 + Krt18 + | Cyp17a1 | 1700011M02Rik | H19 + Prl2b1 + | Map1b + Prl2c5 + |
| Dmkn | Sct | | | Rhox9 | Rhox9 |
| Gpx3 + Dmkn + | Krt18 + Sbsn + | Oaf | 1700011M02Rik | H19 + Prl2c3 + | Map1b + Prl2c5 + |
| Prl8a2 | Sct | | | Rhox9 | Rhox9 |
| Mt1 + Gpx3 + | Krt18 + Sbsn + | Ldoc1 + Serpinb9g | 1700011M02Rik | H19 + Rhox9 + | Map1b + Prl2c5 + |
| Dmkn | Sct | | | Sct | Rhox9 |
| 4930486L24Rik + | Krt18 + Sbsn + | Rere | Fmr1nb + Krt19 | H19 + Krt8 + | Map1b + Prl2c5 + |
| Gpx3 + Dmkn | Sct | | | Prl3d1 | Rhox9 |
| Ly6c1 + Mt2 + | Krt18 + Sbsn + | Rere | Ctsr + Rarres2 | H19 + Rbp1 + | Map1b + Prl2c5 + |
| Dmkn | Sct | | | Sct | Rhox9 |
| Rrm2 + Gpx3 + | Krt18 + Sbsn + | Rere | Krt19 + Rarres2 | Bcat1 + H19 + | Map1b + Prl2c5 + |
| Dmkn | Sct | | | Rhox9 | Rhox9 |
| Gpx3 + Dmkn + | Fnd3c2 + Hspb1 + | Rere | Cts6 + Ctsr | H19 + Prl3b1 + | Map1b + Prl2c5 + |
| Prl8a2 | Sct | | | Rhox9 | Rhox9 |
| Mt1 + Gpx3 + | Fnd3c2 + Hspb1 + | Rere | Ctsr + Prl2b1 | Cdh5 + Sct + | Igf2 + Trap1a |
| Dmkn | Sct | | | Tfpi | |
| 4930486L24Rik + | Fnd3c2 + Hspb1 + | Rere | Krt19 + Prl2b1 | Adgrf5 + Sct + | Igf2 + Trap1a |
| Gpx3 + Dmkn | Sct | | | Tfpi | |
| Ly6c1 + Mt2 + | Fnd3c2 + Hspb1 + | Rere | Cited1 + Prl2b1 | Cryab + Sct + | Igf2 + Trap1a |
| Dmkn | Sct | | | Tfpi | |
| Rrm2 + Gpx3 + | Fnd3c2 + Hspb1 + | Rere | Nup62cl + Prl7d1 | Hspg2 + Sct + | Igf2 + Trap1a |
| Dmkn | Sct | | | Tfpi | |
| Dmkn + Prl8a2 | H19 + Krt19 + | Rere | Pr17d1 + Slc38a4 | Plac1 + Sct + | Igf2 + Trap1a |
| | Prl8a2 | | | Tfpi | |
| Dmkn + Prl8a2 | Krt19 + Prl8a2 + | Rere | Ghrh + Prl7d1 | Sct + Slc38a4 + | Igf2 + Trap1a |
| | Sct | | | Tfpi | |
| Dmkn + Prl8a2 | Cryab + H19 + | Rere | Prl7d1 + Rarres2 | Lepr + Sct + | Igf2 + Trap1a |
| | Krt19 | | | Tfpi | |
| Dmkn + Prl8a2 | Cryab + Krt19 + | Rere | Cts6 + Prl7d1 | Plet1 + Sct + | Igf2 + Trap1a |
| | Sct | | | Tfpi | |
| Dmkn + Prl8a2 | H19 + Krt18 + | Rere | Prl2b1 + Prl7d1 | Sct + Sdc1 + | Igf2 + Trap1a |
| | Prl8a2 | | | Tfpi | |
| Dmkn + Prl8a2 | Krt18 + Prl8a2 + | Rere | Gm9112 + Prl3b1 | Procr + Sct + | Igf2 + Trap1a |
| | Sct | | | Tfpi | |
| Dmkn + Prl8a2 | Gpx3 + H19 + | Rere | Ctsr + Prl3b1 | Cdh5 + Sct + | Igf2 + Nrk |
| | Krt19 | | | Tfpi | |
| Dmkn + Prl8a2 | H19 + Krt19 + | Rere | Krt19 + Prl3b1 | Hspg2 + Sct + | Igf2 + Nrk |
| | Sbsn | | | Tfpi | |
| Dmkn + Prl8a2 | Gpx3 + Krt19 + | Rere | Prl2a1 + Prl3b1 | Sct + Sdc1 + | Igf2 + Nrk |
| | Sct | | | Tfpi | |
| Dmkn + Prl8a2 | Krt19 + Sbsn + | Rere | Ctsj + Hand1 | Cdh5 + Sct + | Fthl17a + Igf2 |
| | Sct | | | Tfpi | |
| Dmkn + Prl8a2 | Krt18 + Prl3b1 + | Rere | Ctsj + Nup62cl | Adgrf5 + Sct + | Igf2 + Nrk |
| | Prl8a2 | | | Tfpi | |
| Dmkn + Prl8a2 | Ctsj + Krt18 + | Rere | Prl3b1 + Prl7d1 | Plet1 + Sct + | Igf2 + Nrk |
| | Prl8a2 | | | Tfpi | |
| Dmkn + Prl8a2 | Cryab + H19 + | Rere | Ctsj + Prl7d1 | Plac1 + Sct + | Igf2 + Nrk |
| | Krt18 | | | Tfpi | |
| Dmkn + Prl8a2 | Cryab + Krt18 + | Rere | Gm9112 + Serpinb9d | Cryab + Sct + | Igf2 + Nrk |
| | Sct | | | Tfpi | |
| Dmkn + Prl8a2 | Cryab + Krt18 + | Rere | Gm9112 + Prl3d2 | Procr + Sct + | Igf2 + Nrk |
| | Prl3b1 | | | Tfpi | |
| Dmkn + Prl8a2 | Cryab + Ctsj + | Rere | Gm9112 + Serpinb9f | Lepr + Sct + | Igf2 + Nrk |
| | Krt18 | | | Tfpi | |
| Dmkn + Prl8a2 | Gpx3 + H19 + | Rere | 3830417A13Rik + | Sct + Slc38a4 + | Igf2 + Nrk |
| | Krt18 | | Gm9112 | Tfpi | |
| Dmkn + Prl8a2 | H19 + Krt18 + | Rere | Gm9112 + Ldoc1 | Lepr + Sct + | Nrk + Trap1a |
| | Sbsn | | | Tfpi | |
| Dmkn + Prl8a2 | Gpx3 + Krt18 + | Rere | 1700011M02Rik + | Plet1 + Sct + | Nrk + Trap1a |
| | Sct | | Serpinb9d | Tfpi | |
| Dmkn + Prl8a2 | Krt18 + Sbsn + | Rere | 3830417A13Rik + | Cryab + Sct + | Nrk + Trap1a |
| | Sct | | Serpinb9d | Tfpi | |
| Dmkn + Prl8a2 | Fnd3c2 + Hspb1 + | Rere | Gm9112 + Sult1e1 | Sct + Sdc1 + | Nrk + Trap1a |
| | Sct | | | Tfpi | |
| Dmkn + Prl8a2 | Ctsl | Rere | 1700011M02Rik + | Hspg2 + Sct + | Nrk + Trap1a |
| | | | Serpinb9f | Tfpi | |
| Dmkn + Prl8a2 | Krt18 + Prl8a2 | Rere | 3830417A13Rik + | Procr + Sct + | Nrk + Trap1a |
| | | | Serpinb9f | Tfpi | |
| Gpx3 + Dmkn + | Krt18 + Prl8a2 | Rere | 1700011M02Rik + | Cdh5 + Sct + | Nrk + Trap1a |
| Prl8a2 | | | Ldoc1 | Tfpi | |
| Mt1 + Gpx3 + | Krt18 + Prl8a2 | Rere | Cdh3 + Serpinb9d | Plac1 + Sct + | Nrk + Trap1a |
| Dmkn | | | | Tfpi | |
| 4930486L24Rik + | Krt18 + Prl8a2 | Rere | Cdh3 + Gm9112 | Sct + Slc38a4 + | Nrk + Trap1a |
| Gpx3 + Dmkn | | | | Tfpi | |
| Ly6c1 + Mt2 + | Krt18 + Prl8a2 | Rere | Gm9112 + Krt14 | Adgrf5 + Sct + | Nrk + Trap1a |
| Dmkn | | | | Tfpi | |
| Rrm2 + Gpx3 + | Krt18 + Prl8a2 | Rere | Gm9112 + Pramef12 | Cryab + Sct + | Fthl17a + Igf2 |
| Dmkn | | | | Tfpi | |
| Dmkn + Prl8a2 | Cryab + Krt18 | Rere | Cdh3 + Serpinb9f | Sct + Sdc1 + | Fthl17a + Igf2 |
| | | | | Tfpi | |
| Gpx3 + Dmkn + | Cryab + Krt18 | Rere | Gm9112 + Hand1 | Sct + Slc38a4 + | Fthl17a + Igf2 |
| Prl8a2 | | | | Tfpi | |
| Mt1 + Gpx3 + | Cryab + Krt18 | Rere | Hand1 + Serpinb9f | Hspg2 + Sct + | Fthl17a + Igf2 |
| Dmkn | | | | Tfpi | |
| 4930486L24Rik + | Cryab + Krt18 | Rere | Gm9112 + Serpinb9g | Plet1 + Sct + | Fthl17a + Igf2 |
| Gpx3 + Dmkn | | | | Tfpi | |
| Ly6c1 + Mt2 + | Cryab + Krt18 | Rere | Hand1 + Zfp36l3 | Procr + Sct + | Fthl17a + Igf2 |
| Dmkn | | | | Tfpi | |
| Rrm2 + Gpx3 + | Cryab + Krt18 | Rere | 1600025M17Rik + | Lepr + Sct + | Fthl17a + Igf2 |
| Dmkn | | | Gm9112 | Tfpi | |
| Dmkn + Prl8a2 | Gpx3 + Krt18 | Rere | Gm9112 + Nup62cl | Plac1 + Sct + | Fthl17a + Igf2 |
| | | | | Tfpi | |
| Ly6c1 + Mt2 + | Gpx3 + Krt18 | Rere | Hand1 + Serpinb9d | Adgrf5 + Sct + | Fthl17a + Igf2 |
| Dmkn | | | | Tfpi | |
| Dmkn + Prl8a2 | Krt18 + Sbsn | Rere | Hand1 + Ldoc1 | Procr + Sct + | Fthl17a + Trap1a |
| | | | | Tfpi | |
| Gpx3 + Dmkn + | Krt18 + Sbsn | Rere | Hand1 + Pramef12 | Hspb1 + Lepr | Fth117a + Procr + |
| Prl8a2 | | | | | Sct |
| Mt1 + Gpx3 + | Krt18 + Sbsn | Rere | Nup62cl + Serpinb9d | Cdh5 + Hspb1 | Fthl17a + P1et1 + |
| Dmkn | | | | | Sct |
| 4930486L24Rik + | Krt18 + Sbsn | Rere | Ldoc1 + Nup62cl | Hspb1 + Slc38a4 | Fthl17a + Procr + |
| Gpx3 + Dmkn | | | | | Sct |
| Ly6c1 + Mt2 + | Krt18 + Sbsn | Rere | 1700011M02Rik + | Plac1 + Sct + | Fthl17a + Trap1a |
| Dmkn | | | Serpinb9g | Tfpi | |
| Rrm2 + Gpx3 + | Krt18 + Sbsn | Rere | 1600025M17Rik + | Cdh5 + Hspb1 | Fthl17a + Procr + |
| Dmkn | | | Prl3d2 | | Sct |
| Dmkn + Prl8a2 | Fermt2 + Hspb1 + | Rere | 1600025M17Rik + | Sct + Slc38a4 + | Fthl17a + Trap1a |
| | Sct | | 1700011M02Rik | Tfpi | |
| Gpx3 + Dmkn + | Fermt2 + Hspb1 + | Rere | 1600025M17Rik + | Cryab + Sct + | Fthl17a + Trap1a |
| Prl8a2 | Sct | | 3830417A13Rik | Tfpi | |
| Mt1 + Gpx3 + | Fermt2 + Hspb1 + | Rere | Nup62cl + Serpinb9f | Plet1 + Sct + | Fthl17a + Trap1a |
| Dmkn | Sct | | | Tfpi | |
| 4930486L24Rik + | Fermt2 + Hspb1 + | Rere | Gm9112 + Prl2c1 | Sct + Sdc1 + | Fthl17a + Trap1a |
| Gpx3 + Dmkn | Sct | | | Tfpi | |
| Ly6c1 + Mt2 + | Fermt2 + Hspb1 + | Rere | 1600025M17Rik + | Hspg2 + Sct + | Fthl17a + Trap1a |
| Dmkn | Sct | | Serpinb9d | Tfpi | |
| Rrm2 + Gpx3 + | Fermt2 + Hspb1 + | Rere | 1600025M17Rik + | Lepr + Sct + | Fthl17a + Trap1a |
| Dmkn | Sct | | Serpinb9f | Tfpi | |
| Dmkn + Prl8a2 | Gpx3 + Krt18 + | Rere | 3830417A13Rik + | Hspb1 + Lepr | Fthl17a + P1et1 + |
| | Prl3b1 | | Serpinb9g | | Sct |
| Ly6c1 + Mt2 + | Gpx3 + Krt18 + | Rere | 1600025M17Rik + | Cdh5 + Sct + | Fthl17a + Trap1a |
| Dmkn | Pr13b1 | | Cdh3 | Tfpi | |
| Dmkn + Prl8a2 | H19 + Hspb1 + | Rere | Nup62cl + Pramef12 | Hspb1 + Slc38a4 | Fthl17a + Plet1 + |
| | Prl8a2 | | | | Sct |
| Gpx3 + Dmkn + | H19 + Hspb1 + | Rere | Nup62cl + Zfp36l3 | Adgrf5 + Sct + | Fthl17a + Trap1a |
| Prl8a2 | Prl8a2 | | | Tfpi | |
| Mt1 + Gpx3 + | H19 + Hspb1 + | Rere | Prl3d2 + Serpinb9g | Hspb1 + Lepr | Adgrf5 + Fth117a + |
| Dmkn | Prl8a2 | | | | Sct |
| 4930486L24Rik + | H19 + Hspb1 + | Rere | 1600025M17Rik + | Cdh5 + Hspb1 | Adgrf5 + Fth117a + |
| Gpx3 + Dmkn | Prl8a2 | | Ldoc1 | | Sct |
| Ly6c1 + Mt2 + | H19 + Hspb1 + | Rere | Cdh3 + Serpinb9g | Hspb1 + Lepr | Fth117a + P1ac1 + |
| Dmkn | Prl8a2 | | | | Sct |
| Rrm2 + Gpx3 + | H19 + Hspb1 + | Rere | Serpinb9d + Serpinb9g | Hspb1 + Slc38a4 | Adgrf5 + Fth117a + |
| Dmkn | Prl8a2 | | | | Sct |
| Dmkn + Prl8a2 | Krt18 + Prl3b1 + | Rere | Ldoc1 + Serpinb9g | Cdh5 + Hspb1 | Fthl17a + Sct + |
| | Sbsn | | | | Tnfrsf9 |
| Gpx3 + Dmkn + | Krt18 + Prl3b1 + | Rere | 1600025M17Rik + | Cdh5 + Hspb1 | Fthl17a + P1ac1 + |
| Prl8a2 | Sbsn | | Krt14 | | Sct |
| Mt1 + Gpx3 + | Krt18 + Prl3b1 + | Rere | Pr12c1 + Serpinb9d | Hspb1 + Slc38a4 | Fthl17a + Hspg2 + |
| Dmkn | Sbsn | | | | Sct |
| 4930486L24Rik + | Krt18 + Prl3b1 + | Rere | Krt14 + Serpinb9g | Hspb1 + Lepr | Fthl17a + Sct + |
| Gpx3 + Dmkn | Sbsn | | | | Tnfrsf9 |
| Ly6c1 + Mt2 + | Krt18 + Prl3b1 + | Rere | 3830417A13Rik + | Hspb1 + Lepr | Fthl17a + Hspg2 + |
| Dmkn | Sbsn | | Ctsr | | Sct |
| Rrm2 + Gpx3 + | Krt18 + Prl3b1 + | Rere | Ctsr + Prl3d2 | Hspb1 + Lepr | Fthl17a + Sct + |
| Dmkn | Sbsn | | | | Sdc1 |
| Dmkn + Prl8a2 | Hspb1 + Prl8a2 + | Rere | Ctsr + Sult1e1 | Cdh5 + Hspb1 | Fthl17a + Hspg2 + |
| | Sct | | | | Sct |
| Gpx3 + Dmkn + | Hspb1 + Prl8a2 + | Rere | Ctsr + Krt14 | Cdh5 + Hspb1 | Fthl17a + Sct + |
| Prl8a2 | Sct | | | | Sdc1 |
| Mt1 + Gpx3 + | Hspb1 + Prl8a2 + | Rere | Cdh3 + Ctsr | Hspb1 + Slc38a4 | Fthl17a + Sct + |
| Dmkn | Sct | | | | Tnfrsf9 |
| 4930486L24Rik + | Hspb1 + Prl8a2 + | Rere | Ctsr + Hand1 | Cdh5 + Hspb1 | Procr + Sct |
| Gpx3 + Dmkn | Sct | | | | |
| Ly6c1 + Mt2 + | Hspb1 + Pr18a2 + | Rere | Ctsr + Nup62cl | Hspb1 + Lepr | Procr + Sct |
| Dmkn | Sct | | | | |
| Rrm2 + Gpx3 + | Hspb1 + Pr18a2 + | Rere | Gm9112 + Krt19 | Hspb1 + Slc38a4 | Plet1 + Sct |
| Dmkn | Sct | | | | |
| Dmkn + Prl8a2 | Cdh5 + Hspb1 + | Rere | 3830417A13Rik + | Hspb1 + Lepr | Plet1 + Sct |
| | Sct | | Krt19 | | |
| Gpx3 + Dmkn + | Cdh5 + Hspb1 + | Rere | Krt19 + Serpinb9f | Hspb1 + Slc38a4 | Fthl17a + Sct + |
| Prl8a2 | Sct | | | | Sdc1 |
| Mt1 + Gpx3 + | Cdh5 + Hspb1 + | Rere | 1700011M02Rik + | Hspb1 + Slc38a4 | Procr + Sct |
| Dmkn | Sct | | Krt19 | | |
| 4930486L24Rik + | Cdh5 + Hspb1 + | Rere | Cdh3 + Krt19 | Hspb1 + Slc38a4 | Fthl17a + Plac1 + |
| Gpx3 + Dmkn | Sct | | | | Sct |
| Ly6c1 + Mt2 + | Cdh5 + Hspb1 + | Rere | Krt19 + Serpinb9d | Cdh5 + Hspb1 | Plet1 + Sct |
| Dmkn | Sct | | | | |
| Rrm2 + Gpx3 + | Cdh5 + Hspb1 + | Rere | Krt19 + Prl3d2 | Lepr + Sct + | Hand1 + Trap1a |
| Dmkn | Sct | | | Tfpi | |
| Dmkn + Prl8a2 | Ctsj + Gpx3 + | Rere | Krt14 + Krt19 | Hspb1 + Slc38a4 | Plac1 + Sct |
| | Krt18 | | | | |
| Ly6c1 + Mt2 + | Ctsj + Gpx3 + | Rere | Krt19 + Ldoc1 | Sct + Sdc1 + | Hand1 + Trap1a |
| Dmkn | Krt18 | | | Tfpi | |
| Dmkn + Prl8a2 | Ctsj + Krt18 + | Rere | Krt19 + Sult1e1 | Cryab + Sct + | Hand1 + Trap1a |
| | Sbsn | | | Tfpi | |
| Gpx3 + Dmkn + | Ctsj + Krt18 + | Rere | Krt19 + Pramef12 | Procr + Sct + | Hand1 + Trap1a |
| Prl8a2 | Sbsn | | | Tfpi | |
| Mt1 + Gpx3 + | Ctsj + Krt18 + | Krt18 + Prl3d1 + | H19 + Hspb1 + | Hspb1 + Slc38a4 | Sct + Tnfrsf9 |
| Dmkn | Sbsn | Sct + Tfpi | Prl3b1 + Sct | | |
| 4930486L24Rik + | Ctsj + Krt18 + | Krt18 + Prl3d1 + | H19 + Hspb1 + | Sct + Slc38a4 + | Hand1 + Trap1a |
| Gpx3 + Dmkn | Sbsn | Sct + Tfpi | Rhox9 + Sct | Tfpi | |
| Ly6c1 + Mt2 + | Ctsj + Krt18 + | Krt18 + Prl3d1 + | Ctsj + H19 + | Adgrf5 + Sct + | Hand1 + Trap1a |
| Dmkn | Sbsn | Sct + Tfpi | Hspb1 + Sct | Tfpi | |
| Rrm2 + Gpx3 + | Ctsj + Krt18 + | Krt18 + Prl3d1 + | H19 + Hspb1 + | Hspb1 + Tnfrsf9 | Fthl17a + Procr + |
| Dmkn | Sbsn | Sct + Tfpi | Krt8 + Sct | | Sct |
| Dmkn + Prl8a2 | Cryab + H19 + | Krt18 + Prl3d1 + | Fthl17a + H19 + | Hspb1 + Slc38a4 | Fthl17a + Lepr + |
| | Hspb1 | Sct + Tfpi | Hspb1 + Sct | | Sct |
| Gpx3 + Dmkn + | Cryab + H19 + | Krt18 + Prl3d1 + | H19 + Hspb1 + | Hspb1 + Tnfrsf9 | Fthl17a + P1et1 + |
| Prl8a2 | Hspb1 | Sct + Tfpi | Sct + Sparc | | Sct |
| Mt1 + Gpx3 + | Cryab + H19 + | Krt18 + Prl3d1 + | H19 + Hspb1 + | Hspb1 + Sdc1 | Fthl17a + Plet1 + |
| Dmkn | Hspb1 | Sct + Tfpi | Krt19 + Sct | | Sct |
| 4930486L24Rik + | Cryab + H19 + | Krt18 + Krt19 + | H19 + Hspb1 + | Cdh5 + Hspb1 | Fthl17a + Lepr + |
| Gpx3 + Dmkn | Hspb1 | Sct + Tfpi | Prl3b1 + Sct | | Sct |
| Ly6c1 + Mt2 + | Cryab + H19 + | Krt18 + Krt19 + | H19 + Hspb1 + | Cdh5 + Hspb1 | Hspg2 + Sct |
| Dmkn | Hspb1 | Sct + Tfpi | Sct + Sparc | | |
| Rrm2 + Gpx3 + | Cryab + H19 + | Krt18 + Krt19 + | H19 + Hspb1 + | Hspb1 + Lepr | Hspg2 + Sct |
| Dmkn | Hspb1 | Sct + Tfpi | Prl3d1 + Sct | | |
| Dmkn + Prl8a2 | Cryab + Hspb1 + | Krt18 + Krt19 + | Ctsj + H19 + | Hspb1 + Slc38a4 | Hspg2 + Sct |
| | Sct | Sct + Tfpi | Hspb1 + Sct | | |
| Gpx3 + Dmkn + | Cryab + Hspb1 + | Krt18 + Krt19 + | H19 + Hspb1 + | Hspb1 + Sdc1 | Fthl17a + Procr + |
| Prl8a2 | Sct | Sct + Tfpi | Krt8 + Sct | | Sct |
| Mt1 + Gpx3 + | Cryab + Hspb1 + | Krt18 + Krt19 + | H19 + Hspb1 + | Hspb1 + Lepr | Fthl17a + Sct + |
| Dmkn | Sct | Sct + Tfpi | Rhox9 + Sct | | Slc38a4 |
| 4930486L24Rik + | Cryab + Hspb1 + | Krt18 + Krt19 + | Fthl17a + H19 + | Plac1 + Sct + | Hand1 + Trap1a |
| Gpx3 + Dmkn | Sct | Sct + Tfpi | Hspb1 + Sct | Tfpi | |
| Ly6c1 + Mt2 + | Cryab + Hspb1 + | Krt18 + Krt19 + | H19 + Hspb1 + | Cdh5 + Hspb1 | Fthl17a + Sct + |
| Dmkn | Sct | Sct + Tfpi | Krt19 + Sct | | Slc38a4 |
| Rrm2 + Gpx3 + | Cryab + Hspb1 + | Krt18 + Krt19 + | H19 + Hspb1 + | Hspb1 + Slc38a4 | Fthl17a + Sct + |
| Dmkn | Sct | Sct + Tfpi | Sct | | Slc38a4 |
| Dmkn + Prl8a2 | Cystml + H19 + | Krt18 + Prl3d1 + | H19 + Hspb1 + | Hspg2 + Sct + | Hand1 + Trap1a |
| | Hspb1 | Sct + Tfpi | Sct | Tfpi | |
| Gpx3 + Dmkn + | Cystml + H19 + | Krt18 + Krt19 + | H19 + Hspb1 + | Hspb1 + Lepr | Adgrf5 + Sct |
| Prl8a2 | Hspb1 | Sct + Tfpi | Krt7 | | |
| Mt1 + Gpx3 + | Cystml + H19 + | Krt18 + Prl3d1 + | H19 + Hspb1 + | Hspb1 + Slc38a4 | Adgrf5 + Sct |
| Dmkn | Hspb1 | Sct + Tfpi | Krt7 | | |
| 4930486L24Rik + | Cystml + H19 + | Krt18 + Krt19 + | Cryab + H19 + | Hspb1 + Lepr | Plac1 + Sct |
| Gpx3 + Dmkn | Hspb1 | Sct + Tfpi | Rhox9 | | |
| Ly6c1 + Mt2 + | Cystml + H19 + | Krt18 + Krt19 + | H19 + Rhox9 + | Cdh5 + Hspb1 | Adgrf5 + Sct |
| Dmkn | Hspb1 | Sct + Tfpi | Sbsn | | |
| Rrm2 + Gpx3 + | Cystml + H19 + | Krt18 + Krt19 + | H19 + Prl8a2 + | Hspb1 + Slc38a4 | Sct + Sdc1 |
| Dmkn | Hspb1 | Sct + Tfpi | Rhox9 | | |
| Dmkn + Prl8a2 | Cystml + Hspb1 + | Krt18 + Krt19 + | Gpx3 + H19 + | Plet1 + Sct + | Hand1 + Trap1a |
| | Sct | Sct + Tfpi | Rhox9 | Tfpi | |
| Gpx3 + Dmkn + | Cystml + Hspb1 + | Krt18 + Krt19 + | Dmkn + H19 + | Cdh5 + Sct + | Hand1 + Trap1a |
| Prl8a2 | Sct | Sct + Tfpi | Rhox9 | Tfpi | |
| Mt1 + Gpx3 + | Cystml + Hspb1 + | Krt18 + Prl3d1 + | Gpx3 + H19 + | Cdh5 + Hspb1 | Plac1 + Sct |
| Dmkn | Sct | Sct + Tfpi | Rhox9 | | |
| 4930486L24Rik + | Cystml + Hspb1 + | Krt18 + Prl3d1 + | H19 + Prl8a2 + | Cdh5 + Hspb1 | Lepr + Sct |
| Gpx3 + Dmkn | Sct | Sct + Tfpi | Rhox9 | | |
| Ly6c1 + Mt2 + | Cystml + Hspb1 + | Krt18 + Prl3d1 + | Cryab + H19 + | Hspb1 + Tnfrsf9 | Plet1 + Sct |
| Dmkn | Sct | Sct + Tfpi | Rhox9 | | |
| Rrm2 + Gpx3 + | Cystml + Hspb1 + | Krt18 + Prl3d1 + | H19 + Rhox9 + | Hspb1 + Sdc1 | Plet1 + Sct |
| Dmkn | Sct | Sct + Tfpi | Sbsn | | |
| Dmkn + Prl8a2 | Hspb1 + Sbsn | Krt18 + Prl3d1 + | Dmkn + H19 + | Hspb1 + Tnfrsf9 | Fthl17a + Hspg2 + |
| | | Sct + Tfpi | Rhox9 | | Sct |
| Gpx3 + Dmkn + | Hspb1 + Sbsn | Krt18 + Sct + | H19 + Hspb1 + | Hspb1 + Sdc1 | Fthl17a + Hspg2 + |
| Prl8a2 | | Tfpi | Krt8 + Sct | | Sct |
| Mt1 + Gpx3 + | Hspb1 + Sbsn | Krt18 + Rhox9 + | H19 + Hspb1 + | Hspb1 + Slc38a4 | Lepr + Sct |
| Dmkn | | Sct + Tfpi | Prl3d1 + Sct | | |
| 4930486L24Rik + | Hspb1 + Sbsn | Krt18 + Rhox9 + | H19 + Hspb1 + | Hspb1 + Tnfrsf9 | Adgrf5 + Fth117a + |
| Gpx3 + Dmkn | | Sct + Tfpi | Prl3b1 + Sct | | Sct |
| Rrm2 + Gpx3 + | Tfpi + Cryab + | Krt18 + Sct + | H19 + Hspb1 + | Hspb1 + Sdc1 | Adgrf5 + Fth117a + |
| Dmkn | Dmkn | Tfpi | Krt19 + Sct | | Sct |
| Ly6c1 + Gpx3 + | Tfpi + Cryab + | Krt18 + Sct + | H19 + Hspb1 + | Hspb1 + Sdc1 | Procr + Sct |
| Dmkn | Dmkn | Sparc + Tfpi | Rhox9 + Sct | | |
| 4930486L24Rik + | Tfpi + Cryab + | Krt18 + Prl3b1 + | H19 + Hspb1 + | Hspb1 + Slc38a4 | Sct + Slc38a4 |
| Gpx3 + Dmkn | Dmkn | Sct + Tfpi | Krt19 + Sct | | |
| Rrm2 + Mt2 + | Tfpi + Cryab + | Fthl17a + Krt18 + | Ctsj + H19 + | Hspb1 + Tnfrsf9 | Fthl17a + P1ac1 + |
| Dmkn | Dmkn | Sct + Tfpi | Hspb1 + Sct | | Sct |
| Tfpi + Cryab + | Mt1 + Gpx3 + | Krt18 + Prl3b1 + | Fthl17a + H19 + | Hspb1 + Sdc1 | Fthl17a + P1ac1 + |
| Dmkn | Dmkn | Sct + Tfpi | Hspb1 + Sct | | Sct |
| Ly6c1 + Mt2 + | Tfpi + Cryab + | Fthl17a + Krt18 + | H19 + Hspb1 + | Hspb1 + Tnfrsf9 | Procr + Sct |
| Dmkn | Dmkn | Sct + Tfpi | Krt19 + Sct | | |
| Tfpi + Cryab + | Gpx3 + Dmkn | Krt18 + Rhox9 + | H19 + Hspb1 + | Hspb1 + Sdc1 | Fthl17a + Sct + |
| Dmkn | | Sct + Tfpi | Krt19 + Sct | | Tnfrsf9 |
| Des + Mfap5 + | Plac1 + Sct + | Fthl17a + Krt18 + | H19 + Hspb1 + | Hspb1 + Tnfrsf9 | Fthl17a + Sct + |
| Htra1 | Tfpi | Sct + Tfpi | Rhox9 + Sct | | Sdc1 |
| Rrm2 + Gpx3 + | Plac1 + Sct + | Krt18 + Sct + | H19 + Hspb1 + | Cdh5 + Peg10 + | Fthl17a + P1et1 + |
| Des | Tfpi | Sparc + Tfpi | Pr13d1 + Sct | Rere | Sct |
| Gpx3 + Htra1 + | Plac1 + Sct + | Fthl17a + Krt18 + | H19 + Hspb1 + | Cdh5 + Peg10 + | Fth117a + Hspg2 + |
| Des | Tfpi | Sct + Tfpi | Prl3d1 + Sct | Rere | Sct |
| Gpx3 + Htra1 + | Plac1 + Sct + | Krt18 + Rhox9 + | H19 + Hspb1 + | Cdh5 + Peg10 + | Plet1 + Sct |
| Mfap5 | Tfpi | Sct + Tfpi | Krt8 + Sct | Rere | |
| Mt1 + Gpx3 + | Plac1 + Sct + | Krt18 + Sct + | H19 + Hspb1 + | Cdh5 + Peg10 + | Hspg2 + Sct |
| Dmkn | Tfpi | Tfpi | Rhox9 + Sct | Rere | |
| Rrm2 + Mt2 + | Tfpi + Cryab + | Krt18 + Sct + | H19 + Hspb1 + | Cdh5 + Peg10 + | Adgrf5 + Fth117a + |
| Mfap5 | Dmkn | Tfpi | Pr13b1 + Sct | Rere | Sct |
| Mt1 + Gpx3 + | Plac1 + Sct + | Krt18 + Sct + | Ctsj + H19 + | Cdh5 + Peg10 + | Fth117a + P1ac1 + |
| Des | Tfpi | Sparc + Tfpi | Hspb1 + Sct | Rere | Sct |
| Mt1 + Gpx3 + | Plac1 + Sct + | Krt18 + Prl3b1 + | Ctsj + H19 + | Cdh5 + Peg10 + | Adgrf5 + Sct |
| Mfap5 | Tfpi | Sct + Tfpi | Hspb1 + Sct | Rere | |
| 4930486L24Rik + | Plac1 + Sct + | Krt18 + Rhox9 + | Fthl17a + H19 + | Cdh5 + Peg10 + | Plac1 + Sct |
| Gpx3 + Des | Tfpi | Sct + Tfpi | Hspb1 + Sct | Rere | |
| 4930486L24Rik + | Plac1 + Sct + | Krt18 + Sct + | H19 + Hspb1 + | Cdh5 + Peg10 + | Fthl17a + Sct + |
| Gpx3 + Mfap5 | Tfpi | Tfpi | Prl3d1 + Sct | Rere | Tnfrsf9 |
| 4930486L24Rik + | Plac1 + Sct + | Krt18 + Sct + | H19 + Hspb1 + | Cdh5 + Peg10 + | Fthl17a + Procr + |
| Gpx3 + Htra1 | Tfpi | Sparc + Tfpi | Krt8 + Sct | Rere | Sct |
| 4930486L24Rik + | Plac1 + Sct + | Krt18 + Prl3b1 + | H19 + Hspb1 + | Cdh5 + Peg10 + | Procr + Sct |
| Gpx3 + Dmkn | Tfpi | Sct + Tfpi | Prl3d1 + Sct | Rere | |
| Ly6c1 + Gpx3 + | Plac1 + Sct + | Krt18 + Prl3b1 + | H19 + Hspb1 + | Cdh5 + Peg10 + | Sct + Tnfrsf9 |
| Htra1 | Tfpi | Sct + Tfpi | Sct + Sparc | Rere | |
| Gpx3 + Htra1 + | Sct + Slc38a4 + | Krt18 + Sct + | H19 + Hspb1 + | Cdh5 + Peg10 + | Fthl17a + Sct + |
| Des | Tfpi | Sparc + Tfpi | Krt19 + Sct | Rere | Sdc1 |
| Ly6c1 + Mt2 + | Plac1 + Sct + | Krt18 + Rhox9 + | H19 + Hspb1 + | Cdh5 + Peg10 + | Sct + Sdc1 |
| Des | Tfpi | Sct + Tfpi | Rhox9 + Sct | Rere | |
| Ly6c1 + Mt2 + | Plac1 + Sct + | Krt18 + Sct + | Fthl17a + H19 + | Hspb1 + Sdc1 | Plac1 + Sct |
| Mfap5 | Tfpi | Sparc + Tfpi | Hspb1 + Sct | | |
| Ly6c1 + Mt2 + | Plac1 + Sct + | Fthl17a + Krt18 + | H19 + Hspb1 + | Hspb1 + Sdc1 | Fthl17a + Sct + |
| Dmkn | Tfpi | Sct + Tfpi | Krt8 + Sct | | Slc38a4 |
| Mt1 + Gpx3 + | Sct + Slc38a4 + | Krt18 + Rhox9 + | H19 + Hspb1 + | Hspb1 + Sdc1 | Sct + Tnfrsf9 |
| Des | Tfpi | Sct + Tfpi | Sct + Sparc | | |
| Rrm2 + Gpx3 + | Plac1 + Sct + | Krt18 + Sct + | Fthl17a + H19 + | Hspb1 + Tnfrsf9 | Sct + Sdc1 |
| Mfap5 | Tfpi | Tfpi | Hspb1 + Sct | | |
| Rrm2 + Gpx3 + | Plac1 + Sct + | Fthl17a + Krt18 + | H19 + Hspb1 + | Hspb1 + Tnfrsf9 | Fthl17a + Lepr + |
| Dmkn | Tfpi | Sct + Tfpi | Prl3b1 + Sct | | Sct |
| Mt1 + Gpx3 + | Adgrf5 + Sct + | Fthl17a + Krt18 + | H19 + Hspb1 + | Hspb1 + Sdc1 | Fthl17a + Lepr + |
| Mfap5 | Tfpi | Sct + Tfpi | Sct + Sparc | | Sct |
| Ly6c1 + Mt2 + | Hspg2 + Sct + | Krt18 + Sct + | H19 + Hspb1 + | Hspb1 + Tnfrsf9 | Hspg2 + Sct |
| Dmkn | Tfpi | Tfpi | Sct + Sparc | | |
| 4930486L24Rik + | Adgrf5 + Sct + | Krt18 + Sct + | H19 + Hspb1 + | Hspb1 + Sdc1 | Hspg2 + Sct |
| Gpx3 + Mfap5 | Tfpi | Sparc + Tfpi | Pr13b1 + Sct | | |
| Mt2 + Des + | Adgrf5 + Sct + | Krt18 + Prl3b1 + | H19 + Hspb1 + | Hspb1 + Tnfrsf9 | Fthl17a + Sct + |
| Mfap5 | Tfpi | Sct + Tfpi | Krt8 + Sct | | Slc38a4 |
| Mt1 + Gpx3 + | Adgrf5 + Sct + | Krt18 + Sct + | Ctsj + H19 + | Hspb1 + Tnfrsf9 | Adgrf5 + Sct |
| Des | Tfpi | Tfpi | Hspb1 + Sct | | |
| Mt1 + Gpx3 + | Adgrf5 + Sct + | Fthl17a + Krt18 + | Fthl17a + H19 + | Hspb1 + Sdc1 | Adgrf5 + Sct |
| Dmkn | Tfpi | Sct + Tfpi | Hspb1 + Sct | | |
| 4930486L24Rik + | Adgrf5 + Sct + | Krt18 + Prl3b1 + | H19 + Hspb1 + | Hspb1 + Tnfrsf9 | Plac1 + Sct |
| Gpx3 + Des | Tfpi | Sct + Tfpi | Rhox9 + Sct | | |
| Rrm2 + Mt2 + | Tfpi + Cryab + | Krt18 + Rhox9 + | Ctsj + H19 + | Cdh5 + Sct + | Ghrh + Igf2 |
| Des | Dmkn | Sct + Tfpi | Hspb1 + Sct | Tfpi | |
| 4930486L24Rik + | Adgrf5 + Sct + | Krt18 + Krt8 + | H19 + Hspb1 + | Cdh5 + Sct + | Hand1 + Igf2 |
| Gpx3 + Dmkn | Tfpi | Sct + Tfpi | Prl3d1 + Sct | Tfpi | |
| Des + Mfap5 + | Plet1 + Sct + | Ctsj + Krt18 + | H19 + Hspb1 + | Cdh5 + Peg10 + | Fthl17a + Lepr + |
| Htra1 | Tfpi | Sct + Tfpi | Pr13d1 + Sct | Rere | Sct |
| Ly6c1 + Mt2 + | Adgrf5 + Sct + | Krt18 + Krt8 + | H19 + Hspb1 + | Cdh5 + Peg10 + | Lepr + Sct |
| Des | Tfpi | Sct + Tfpi | Rhox9 + Sct | Rere | |
| Ly6c1 + Mt2 + | Adgrf5 + Sct + | Krt18 + Krt8 + | Ctsj + H19 + | Cdh5 + Peg10 + | Fthl17a + Sct + |
| Mfap5 | Tfpi | Sct + Tfpi | Hspb1 + Sct | Rere | Slc38a4 |
| Ly6c1 + Mt2 + | Adgrf5 + Sct + | Krt18 + Krt8 + | H19 + Hspb1 + | Cdh5 + Peg10 + | Sct + Slc38a4 |
| Dmkn | Tfpi | Sct + Tfpi | Prl3b1 + Sct | Rere | |
| Rrm2 + Gpx3 + | Adgrf5 + Sct + | Ctsj + Krt18 + | H19 + Hspb1 + | Cdh5 + Sct + | Hand1 + Nrk |
| Des | Tfpi | Sct + Tfpi | Rhox9 + Sct | Tfpi | |
| Rrm2 + Gpx3 + | Adgrf5 + Sct + | Krt18 + Krt8 + | Fthl17a + H19 + | Procr + Sct + | Fthl17a + Nrk |
| Mfap5 | Tfpi | Sct + Tfpi | Hspb1 + Sct | Tfpi | |
| Rrm2 + Gpx3 + | Adgrf5 + Sct + | Ctsj + Krt18 + | H19 + Hspb1 + | Cryab + Sct + | Fthl17a + Nrk |
| Dmkn | Tfpi | Sct + Tfpi | Sct + Sparc | Tfpi | |
| Des + Mfap5 + | Sct + Slc38a4 + | Ctsj + Krt18 + | Fthl17a + H19 + | Hspg2 + Sct + | Fthl17a + Nrk |
| Htra1 | Tfpi | Sct + Tfpi | Hspb1 + Sct | Tfpi | |
| Gpx3 + Htra1 + | Sct + Slc38a4 + | Krt18 + Krt8 + | H19 + Hspb1 + | Cdh5 + Sct + | Fthl17a + Nrk |
| Mfap5 | Tfpi | Sct + Tfpi | Krt19 + Sct | Tfpi | |
| Mt2 + Des + | Sct + Slc38a4 + | Ctsj + Krt18 + | H19 + Hspb1 + | Plet1 + Sct + | Fthl17a + Nrk |
| Mfap5 | Tfpi | Sct + Tfpi | Krt8 + Sct | Tfpi | |
| 4930486L24Rik + | Cdh5 + Sct + | Krt18 + Krt8 + | H19 + Hspb1 + | Procr + Sct + | Hand1 + Nrk |
| Gpx3 + Htra1 | Tfpi | Sct + Tfpi | Sct + Sparc | Tfpi | |
| Mt1 + Gpx3 + | Sct + Slc38a4 + | Ctsj + Krt18 + | H19 + Hspb1 + | Sct + Slc38a4 + | Fthl17a + Nrk |
| Mfap5 | Tfpi | Sct + Tfpi | Krt19 + Sct | Tfpi | |
| Mt1 + Gpx3 + | Sct + Slc38a4 + | Ctsj + Krt18 + | H19 + Hspb1 + | Adgrf5 + Sct + | Fthl17a + Nrk |
| Dmkn | Tfpi | Sct + Tfpi | Prl3b1 + Sct | Tfpi | |
| 4930486L24Rik + | Sct + Slc38a4 + | Krt18 + Krt19 + | H19 + Hspb1 + | Plac1 + Sct + | Fthl17a + Nrk |
| Gpx3 + Des | Tfpi | Sct + Tfpi | Prl2c5 + Rhox6 | Tfpi | |
| 4930486L24Rik + | Sct + Slc38a4 + | Krt18 + Prl3d1 + | H19 + Hspb1 + | Sct + Sdc1 + | Hand1 + Nrk |
| Gpx3 + Mfap5 | Tfpi | Sct + Tfpi | Prl2c5 + Rhox6 | Tfpi | |
| 4930486L24Rik + | Sct + Slc38a4 + | Krt18 + Prl3d1 + | H19 + Hspb1 + | Cryab + Sct + | Hand1 + Nrk |
| Gpx3 + Htra1 | Tfpi | Sct + Tfpi | Prl2c5 + Rhox9 | Tfpi | |
| 4930486L24Rik + | Sct + Slc38a4 + | Krt18 + Krt19 + | H19 + Hspb1 + | Lepr + Sct + | Hand1 + Nrk |
| Gpx3 + Dmkn | Tfpi | Sct + Tfpi | Prl2c5 + Rhox9 | Tfpi | |
| Ly6c1 + Gpx3 + | Sct + Slc38a4 + | Krt18 + Krt8 + | H19 + Hspb1 + | Hspg2 + Sct + | Hand1 + Nrk |
| Htra1 | Tfpi | Sct + Tfpi | Sct | Tfpi | |
| Ly6c1 + Mt2 + | Sct + Slc38a4 + | Ctsj + Krt18 + | H19 + Hspb1 + | Sct + Slc38a4 + | Hand1 + Nrk |
| Des | Tfpi | Sct + Tfpi | Sct | Tfpi | |
| Ly6c1 + Mt2 + | Sct + Slc38a4 + | H19 + Krt18 + | Hspb1 + Map1b + | Adgrf5 + Sct + | Hand1 + Nrk |
| Mfap5 | Tfpi | Sct + Tfpi | Sct | Tfpi | |
| Ly6c1 + Mt2 + | Sct + Slc38a4 + | Krt18 + Sct + | H19 + Hspb1 + | Hspb1 + Tnfrsf9 | Lepr + Sct |
| Dmkn | Tfpi | Sparc + Tfpi | Sct | | |
| Rrm2 + Gpx3 + | Sct + Slc38a4 + | Fthl17a + Krt18 + | H19 + Hspb1 + | Hspb1 + Sdc1 | Lepr + Sct |
| Des | Tfpi | Sct + Tfpi | Sct | | |
| Rrm2 + Gpx3 + | Sct + Slc38a4 + | Krt18 + Rhox9 + | | | |
| Mfap5 | Tfpi | | | | |

1-218. (canceled)

219. A method comprising: incubating a population of cells with a first isolation agent, a second isolation agent, a third isolation agent, a fourth isolation agent, a fifth isolation agent, and a sixth isolation agent, wherein the population of cells comprises a subpopulation of fetal cells and a subpopulation of maternal cells from a sample of a subject, and wherein the first isolation agent is specific for a first biomarker in the subpopulation of fetal cells, the second isolation agent is specific for a second biomarker in the subpopulation of fetal cells, the third isolation agent is specific for a third biomarker in the subpopulation of fetal cells, the fourth isolation agent is specific for a fourth biomarker in the subpopulation of maternal cells, the fifth isolation agent is specific for a fifth biomarker in the subpopulation of maternal cells, and the sixth isolation agent is specific for a sixth biomarker in the subpopulation of maternal cells.

220. The method of claim 219, wherein the subject is a human subject.

221. The method of claim 219, wherein the sample is a blood sample.

222. The method of claim 221, wherein the blood sample is blood plasma.

223. The method of claim 221, wherein the blood sample is blood serum.

224. The method of claim 221, wherein the blood sample is buffy coat.

225. The method of claim 221, wherein the maternal blood sample comprises peripheral blood mononuclear white blood cells.

226. The method of claim 221, wherein the maternal blood sample comprises nucleated white blood cells.

227. The method of claim 221, wherein the blood sample is whole blood.

228. The method of claim 219, wherein one of the first isolation agent, the second isolation agent, the third isolation agent, the fourth isolation agent, the fifth isolation agent, and the sixth isolation agent is a nucleic acid probe.

229. The method of claim 219, wherein one of the first isolation agent, the second isolation agent, the third isolation agent, the fourth isolation agent, the fifth isolation agent, and the sixth isolation agent is a ligand.

230. The method of claim 219, wherein one of the first isolation agent, the second isolation agent, the third isolation agent, the fourth isolation agent, the fifth isolation agent, and the sixth isolation agent is specific for a cell surface marker.

231. The method of claim 219, wherein one of the first isolation agent, the second isolation agent, the third isolation agent, the fourth isolation agent, the fifth isolation agent, and the sixth isolation agent is specific for a soluble cell surface marker.

232. The method of claim 219, wherein one of the first isolation agent, the second isolation agent, the third isolation agent, the fourth isolation agent, the fifth isolation agent and the sixth isolation agent is specific for an intracellular biomarker.

233. The method of claim 219, wherein one of the first isolation agent, the second isolation agent, the third isolation agent, the fourth isolation agent, the fifth isolation agent, and the sixth isolation agent is specific for a nucleic acid marker.

234. The method of claim 219, further comprising after the incubating, enriching the subpopulation of fetal cells from the subpopulation of maternal cells.

235. The method of claim 219, wherein after the incubating, the subpopulation of fetal cells is enriched from the subpopulation of maternal cells.

236-252. (canceled)
